SEQUENCE LISTING (42 sequences)

SEQ ID NO: 1 - DNA, 113687 bp, Homo sapiens
gaattcccggaatcctcctcggccccccttccagactcccgcctcactctgcctggcagtccagggacaggagccgcctaactacttccagatgtggacacagccacgaggcggtgggggggagggggtggagagatggagggagctgcagtggcgatggccctctgattgaacctggaggccaggctccgcgagctccacaccttagccctgcccctgttccaaactacttcagattctgactcccaagtctccactcactgccccctcctccccctacaatccctctcgaattccttcctggaatccagctaccaggcaagttcaggagcttggctctctcttgggttctatggggggtcaaggatctgggtgggtggggagacttaagagaccagaggacgaaggtgggggaatgggagcagtggtggcctgcaggagaccaaggtcttgcagcagatggggaggatttgggggccagagaagggtgaggcggtggggaatctggggagtggtgtgtcagcaagggaacccaaaatatctccaggcataaccccaaaggaatcgggggtctgaagttcactggagctgtctggaagatgccaagagacctacctggtgtagttgggggtctggccctgggcaaaaggcagcagggcgcaggcagtgaggaggggccccaggagggaggctgtggctgcaggcagcatggccccgggggaaaggcgctggggtcctcaagacagaggcagcagctgaattttgcagagcagcagcagcggtggcggcagcagcaggataatcagcgccagatggggcagccttgggtgtgtgggcgtggggactggccagtggggcgggcaatggggaaggcgggacagtggcgtgtcctgggacctctctaggtctaggacacataaatgttgaccttaggagggggaggagcagtgaggggcagaggccgtctcccaggcctcaagtaacgtcatcgggaaccccactctgtccagtgagggagtatctccgggaacttctgaggactccctagcccccatcctccccccgcccctgcttcctcagctcaggagccctcattgctcaccccctgcctgaccacccctgccagctcattttccacttcaatttctttttttttttttttttttttttgagacagagtctcattctgtgacccaggctggagtgcagtggcgcgatctctgctcactgcaacctccgcctcccgggttcaagcaattcttgtgccttagcctcccgagtagctgggactgcaggtgcatgcctccacacctggctaatttttgtatttttagtagaggcagggtttcagcatgctgtccaggctggtctcaaattcctagcctcaggtgatccgcccacctcggactcccaaagtgctgggattataggcgtagccaccacacctggcctccacttcacatctccattgccttctctccagacctgggccagactaggccaaggagaaggaattctcacccatgacctcttccaactacttgaaggtacaggaagcaaggcaggaggcatggggtgtggatgaagggagtgtccttgttactcaggaagaggtggaaggagtagaaaatagagcccaaacctgggaggtaaacagaagggagagaggaggggggctggcttcaggccgaagggagatgggtgggaagctttcgggggtcaggaggaacaggctgcctcaggatggggacaagtctagtgggcaggggatcccagtggggtagccaagagcatctgtgcatagagttgttccagaaggtagggccagaacttcccagtccaaacgaccaggtgtgctcttgggagcagggagtggacaacaatgtgaagggttcaggcagcatcagctctgctgcacctgagacctggagctgcttgtgctgcctgcccctgcctgggctcctgccctcctgcacccaccaagccgggcacccacccaccccatccctgcccctgtccctcccagcccccaaggcttccacaccaggcctcgctgcagccttttattctatccatcaaatctcttgtccatgatagtcaggttgggggcccctcctgggcagccccctgaacattcttaccctggttagccgtccccaccccctagcaaccccttcccacacgcatgtgcacaatggtcattgctccttagtccctggcctggggccggactccctcctccagggactaggctagcatgagggggattaggtctcaggggctgggggccctacaccacccccgcctccttcagccctctgcagcatgtccagggcctcccagaagaagtccttctgtgcggccatcaggggcatccaccctacgatctccttcatcttctccgtgcggtcctgcaacgtttggcaactgtggatctggctgaggtactcctcgcaggcctgggccatccccccggggtcctggggtgctctggcccccaggctgggctcaggagtccccaccaccctgctggctggcaggtggcgatagggggcgtgctgctcaatgtcgtggctggacaggatgtataggcactccctggtggcacagagggcacaggcacacagagggaccttggaggaggagagaaagggggcatggctgagtggtgctttccttgcagggtttcccagggtgcccgggctaatctgcctattgcattcttccctctcacaccagccctgagccggctaagtcccaggggtgtgtgaggttgggcaaggccagcacagtctgctaagggagaaagcagagagcgtggtgtgcggcagaggagggaggtggggtggtgctagggaaatgggaccccctgggtccctgcaggagccccagctacagcctgtgggcttcctggttcatggctctcatcctccctgggtgcccactctcctgaccccgctctgaccttgacttggagcttgacgaaggaggcactccccttgggccagcctgggaggcggcccccagccccacagccgcagcccagcagctccttgctgaactctgagctctggctcagcaccagggagtggttgaggccgcaccacagggtgatgggccgctgcaggtaacaaacctggggacacacggccagggtccctgagcagggagcaccaagggacgggggggctggggtgggaaaaggttgactggaaggctcggggcagaatgaactggggaggaggaaatgggagccagcactgtcccacgccccaggtctgccctgctgacccctgcagcccccatactctaactggtcacatagggtcttgcacttctgttggtatctcatctatgcccccagaaaatcagctccttgcctgagcccagggcctgctgtggtgtacccctcattctcagcccacccgtctatgccagtccagcctctcatcctgcctgccagcctcggggagacttgcttcttgtgcaattccaggttagaccccaacaccaccctgaggaaggactttcccttccactatccctcccttcctgctctagtattatcttttctttctttctttcttttttttttttttgtttgagacagagtcggaaggctggagtgcagcagtgcgatctcggctcactgcaacctctgccttcctgggttcaagtgattctcccacctcagcctcccaagtagctaggactacaggtccttgccaccacacccatctaatttttgtatttttagtagagaaggggttttgccatgttgaccaggctggtctcgaactcctgacctcaggtgatctgcctgccttggcctcccaaagtgctgggattacaggcatgaaccactgagcccagcctccactctagcatttttattttctcttttctttttttagagatggggtctcattctgttgcccaggctgaaatgcagtggtgcaatcatagctcactgcagccttgacctcctgggctcaagccattcttctgcctcagcctcctgagtagttgggaatacaggcatgtgccaccacacccggcatgtgcctataatttttaaattttaaatttttaaattttctatagagatagtgtctcactatgttgctcatggtgatctcgaactccgggcctcccacctcagcctctagcattttttttttttttgtagagacggagtctcgctctgttgcccaagctgaagtgcagtggcacgatttcagttcactgcaacctccgcctctccagttcaagcaattctccttcctcagcctcccgagtagctgggactatgggtgcacaccgccacacctggctaatttcttttgtattttagtagagacggggtttcaccatgttgcccaggctggtcccgaactcctgggctcaggcaatccacctgccttggcctcccaaagtgctaggattacaggtgtgagctagttgaaatgcttagcctagcattttcaacttatcacataattcttttccaaaatggccaccctgtttttgaaaaaaaagcttctacttgcttcctgccgcttcctgcctctctctcttaagcagcagagttggttacatgaccaatctgaggctgaccacattactgttccttcccacagccagtctatccagcacctccctgggatctcgcttttctctctattacattactgacatgctctctctagaggcacttggcctctcagtggctaaatcccagggcgtcttctgccccctcattctcctgagcctgttagcgctttaattctgaagacactgataagtaatttatggtcattctagaaaaattatgaaacattggcaagcaaaaaattaattttaaaatcattggaaatcccaccatctactattaacatcttgctagatattcttcactgttggctaaacaaacaaatacacataaattgtatattttttattttttagagatagggtctcagtgtcacccaggctggagtgcagtggtgatcacagctcactgcagccttcaactcctggcctcaagcaatcctcctgtctcagcctcctgagtagttgggattacagatgtcagccaccatgatgggcaagttgtgtatttttaaacaaaaaatgggatggggccaggtgtggtggctcacacctgtaatcccagcactttaggagtctgaggtgggaggattgcttaaagccatgagttcaatataagcttgggcaacatagcaagaccctgtctctaaaaaaaaaaaaaacaaaaaaaacaaaaaaaacaaaaacttatctgggcgtagtggcgcattcctgtagtcctagctgcttgggaggttgaggcgggaggatctcttgagccctggagttgaaagctgcagcaagctacgattgtgtcactgcactccagcttgggaagcagtggagaccatgtctcaaaaaagtaataataaaaataaaaagaggctgggtgcggtggctcacacctgtaatcccagcactttgggaggccaaggtgtgcagatcacctgaggtcaggagtttgagaccagcctggccaacatggtaaaactccgtctctactaaaaatacaaaaattagccgggcatggtggtgggcgctgtaatcccatcttggcaggctgaggcaggagaatcacttgaacctgggaggcagaggttgcagtgagccgagatcgcgccattgcactccagcctgggagacagagcaagactctatcttgaaaataaaataaaaataaaaataaaaataaatggtactatgtaaaccatttcataaaagtctttttcacctaaaaatatggcatgactatcttttccatgtcagtagtaaatgtatcttacattgtcacaccatcaattaaccctaccacccaccccatttggaacttccttctcatgtggcttctgggacagtgttagaggggtgcacggcctgtcttagaggcacctcttcatcccctgtctaccacagggcctacttctcagagttgcttttcagtctctacttctgtttgttcaaccatgttcccaggtagaatctaattgccagctagacgtttccaattgagtgtggtttttgttttgttttgttttgagacagagtctctctccgttgcccaggctggaatgttgtggcacgatctcggctcaccgcaacctctgcctcccaggctcaagcaattctcgtgcctcagactcccgagtagctgggactaaaagcacccaccaccacacctggctaattttttattttttctagagacagggtttcaccatgttgtccaggctgatctcatgctcctgggctcacgtgatccacccaccttggtctcccaaagtgctgggattacaggtgtgagatatctttcttccagagccacctctgtcttatacccctctcttctacatctaatcagttttgaggccctattcattttcttcccaaatagctcctgtatccattccttcccattctcgctgcagcaccaggcgcttgtctcaagtgctgtcatcactcaacagcctcctggcgggctcccctccttggcctcctctgcacaaggctcctgacacattctcagtcacctcttgggggttgccttctcctattccagaacctgcagtgactcttccccaaatctacacctctctaagcttgtgcccacctgtaccacctcatacctcagtgattcccaagatgccttctgtgctttagtgagacccgtcttgtcactgtcccaggtatgtgcaagtttcgtgcctatccccctcctcatcacacagaatgccttctctatccctacctaagtcttatgtaacccacgatgccctgctcacatcttttttctttttctttgagacggagtctcgctctgtggcccaggctggagtagggtggcacgatctcagctcactgcaacctcagcctcccaggttcaagagaatctcctgcctcaacctcctgagtagctgggattgcaggcatgtgccaccacagccagctaattttttatatttttggtagagacggggtttcaccatgttgcccaggctggtctagaactcctgacctcaagtgatctgtccacctcggcctcccaaagtgtcgggattacaggcgtgaggctctgcacctggcccctgctcacatcttacctcttctagaagtctctagcctgcccaggtcacccaccctaccccttcactaaattcctattgcatttacagtgtgcactgcacagaatggacctcgtagtgccaaccctgggtaagacatggggtagtaccactggcattacatactgagtggcttaacttcccaaaggaacagaaattggattctgtggaatctggagaagtggacagatctgggggttagggaattggaggaaaggggttgggatgagagttgctgggaaatagtctcacctgtgtgggttcccctcggtccattttgtcccctgttcctagctgcccgtatctgttatttccttgcatgaagattcggccaaattcatccaccaggccaaggtggttgtagccaagagcacagaataggatctagcgtgcaagatagaaaggggatagggattccattccctccagaggaactctgagttccagagtctccaagccactaacctgccatccccagctgatctctttctatatatatattttagagatgggatctcactatgttgcccaggctggtctagagctcctgggctcaagcgatactccctcctcagcctccctccgcaccgagcccccagctgatctcttttggcatacctctccctggggatcccctgcccctctggatcaataatctgattcctataatacttcagaccttccaatccctacagcgccacccctctggtttgccccgtccctccaggagcctaccttggcaggcagtgagagagcaagcggcatctgctggtccagggggtcaaaggcttgaagggtcccaaagagatcgcgatacacccctggggtatgcacctcaaaatacactcccccctggtctggggggtcaggagccctcagctcagcagctttgggcacccatttcctagggataaggccccacctgggatgaaagctacaagatccaaggagtctcatacccactaggaatgcatcaactatgatgcctgggaactgaaaacacatcttttgagaccgagtctccctcggtcacccaggctggagcgaagtggcacgatctcagttcactgcaacctctacctcctggttcaagtgattctcctgtctcggcctctggtgtagctgggattacaggcgcctgccatcacacccagctaatttttgtattttagtagagatggggtttcgccatgttggccaggctggtcttgaactcctgacctcaggtgattcacctgcctcggtttcccaaagtgatgggattacaggcatgagtcaccatgccggccctaaacacatcttttcactaggatgccctcaggggttcaccatgcagctggagtaagggtgaagggtgttgatgtttagagggtctaaccaaggctgcgggcttgagccggggggaggtggtccatacctgtgacgtagagggtgctgctctggttggaagtcatgcaggccacgcgcaggtgaggcaggtagtgggacaccttcctcagggccagctgaaccgtgtaggagcgtggctggtcaagctgggtctcattcactaccaaagagtagatctttccttcctctgggggtaggggaagcaattagttggggaggggattctccaatatccacataaacatcccaagagactccctaggccatatgggtgacacatttactgccctagagtggggctgagaaagaaataagggaagagtgaccccaagtcccccagactccagttcccaccataaatcagggaagtacttcagcatgagagatgagcagagagaaaaatctagcagatttgttagaatccgatttgagtgagaggtctggggattgagggccctgcctggcattcagaagtttagaggtggtcaagtgtggtggctcacacctgtaatcccagcactttgggaggccaaggcaggaggatcacttgaggcagggagttcgagaccagcctgggcaacatagcaagaccccatctctaccttgctccaaaaaagaaaaaaatattttaagaaaaagcctaaagggtctcaggcctggggatgtggttgagtataaaggacattgggaaagtagggagcagtttggagtatagtgaggtggttggcatggaagctggggacatctgatttttttttttttttttttttttggagatggagtctcactctgtcacccaggctggagtgcagtggcataatctcggctcactgaaacctccacctcctgggttcaagcgattctcctgcctcaggctcccaagtagctgggactacaggtccatgccaccacgtccggctaatttttttttttttttttttttgagacggagtctcgctctgtcaccaggctggagtgcagtggcgtgatctcggctcactgcaacctctgcctcctgggttcaagggattctcctgcctcagcctcccaggtagcttagactacaggcacgcgccaccacgcttggctaatttttgtatttttttttagtagagtcaggcagggtttcaccatgttggtctcgatctcttgacctcgtgatccacccgccttggcctcccaaaaactgggattacaggtgtgagccgccgcacccgacctaatttttgtattttttagtagagatggggtttcgccatgttggccaggctggtctcaaactccagatctcaggtgatctgctcacctcggcctcccaaagtgctggggttacaggtgtgagccaccacacctggacagggccttttattcatataaaatattaagcagaatcctagactaggagacagagaagggcagagctgtttagatggagactaggataaggtggcatatggaacccagtctgaggggcctccctccccctcctctgcagcagggtctgaggaatctgcaggaaacagtttgcaaaagccattgctttgggccacacctgtgaggagcagtagagcccgctgggtctcctgaccaaccagcacgatctgcttgaaggtcattgagtggtggaatgtcatcttgaagacccgctgcccactagactgcagatagacctcaacacagtcacaggcccggctgctggtggtacccaccacttcctgcggctcccgagtggccaagacgtagaggtatttacggtaaactgtgtcacaccttgggtccgaggcaaactgggggaaaagatgagggcgtctgggatctagagtttcaaaatgggatggggctggggcagctggatcctggggtgggaagtgaaatgcttgggtccagagttgggaaatgagcccttgatttcaggttcctatgtgggagggcagaagtcataggaaagacacctcaagactgatgggggcctgggtcctcaaaagagaggtggtgatatggttaggctctgtgcccccacccaaatctcatctcgaattgtaatacccagtgggaggtgattggctcacgggggcaatttcccccatgttgttttcgtgatagtgggttctcacgaggtctgacggatttataaggcagttttccctgctcttgcttgtgtgctctcgcctgctgccatgtaagacgtgcctctttcccttctgccatgattgtaagtttcctgaggcctccccagtcatgtggaactatgagtcaattaaacctctttgccttataaattacccggtctcaggtatgtctttttttttcgagacggagtctccctcttgttgcccaggctggagtgcaagggtgcgatctcagctcactgcagcctctgcctcccaagttcaagcaattctcctgcctcagcctcccaagtagctggaattataggcacccaccaccacacccagctaatttttgtatttttagtagagacgggtttcgccatttggccaggctggtctcgaactcctggtctcagctgatccgcctgcctcagcctcccaaagtgctaggattacaggtgtaagccactgcacccggcctctggtacgtctttatagcagtgtgaaaatggactaatacaggtgggctctcagctgccagggttctgctactcacatccttggctccacgacacaacacaacatagcgacaggcccgcttccactgcatctggccgagggtggagaccagggcatttttgaggaagaagagggtccccacgtagtcaagaatgaagacgtgatccttggtgggcaagaagcggcggtagccgtgggctagcaagggggccacgctcttgctgagacatcggcggcggcctccaaatgcctggaaatacaggcccttcgtgtctggaggaaggaaggagatggaaggttcaagggatgagatgacacgagggagatgagttggagcttgggtagatttcttagacacgctggtttgtagttcctccccatccccaggacaggggaacaaacagaccccttatgaaggagatgtggaaaataagcttagattatactcgacatgtgaactttagtctccaccccacctcagcccagggtcaaacatttgcttcttagtgagcagttctttggtgcaaccttgcatcagtgatgttgaggaaaactctaagaataaagacacagaggctgcgtgcggcatccacccccctcaccctgagcccctcctcccatcctcacagctcagtgaatttttgcacgcctgtaatcccagcactttgggagcccaggagtttgagaccagcctgggcaacgtggcaaaaccccatctctactaaaaatacaaaaattatccgggtgtggtggcgggagcctgtaatcccagctactcaggaggctgaggcacgagaatacttgaacccgggaggtggaggttgcattgagccaagattgcaccactgtgctccagcctgggtgacagagcaagaccctgtctcaaaaaaaaaaaaaaaaaaaaaaaatcaaagaaaaagaaagaaaagagaagagacagaaacaggagtgaaaaggaatgattgatgaaggccggtcatcctccctcacccctagctttggatctgttttctgggtcccccttctcttttaatctcccagtgagtgtaagaagtacaccctgggggatccctggaggggaagagggtttatggcatggttctcaggctaggggtgattttgtccttcaagggcattggttgtcacaattaagggggatgctgctggccatctggtgggtggaggccagggttgctgcttaaacatcccaccatgcagaggacagctccccacatcaaggaattattttgcccagaatatcaacagtgagttcagaagccttgggttagaggagggaaaactccaggtttggggagtgagggatgcctctagggaaaaagcgggaagtgtctgtgtacagagggagacttggcagggaggccgctaacgggggctgtgggtggattccgaagacttggaggaagtactcaagggagaatgtttgaactggacccgtttcttccggagaaaggggccctggaagcactgttgggagtgtgggggtcatcaggggactttagagagcaggaaggctaaggcgggacagaggcaaggagaacgggaggttctggggaaggtgtacagttcagaatggcagctctcttccagggccggactccagaaccctgatcttggaggcgcggactgagtctgcgacagatgcgtctccacacgccttccccatcgcacacttcgtggaagtagcggcaggtctggccgagggcaacaaggtctctgactgggaggaatgagatgatatgctccacctgggagggaggagggaagaagcggggagtctttggtggcccctcggtgatctggcccccaatcctattattctcaatccccctccccaaggactcaccagctctggggggaacaactggatggaaatcggatttcctttccccctcttctcttcaaccccaagctccgagccacaagaagggcagcttctcttcacctacccaagaaggggtgcatagggtcccatgagggtcccacatccttttttcccttctctccgcctaccttggggctcatctgactctagctgggcacagttatttcctcccctctctctccagacccatccctaacttctccaaataactcctcccgatctcctgcggccaatccccctcacccggagcccctcctcccgtccacacagcctggtgaatttttgcagcttccgtctctggcttcccctgaccatcctgattgccccctccctgctcctgccctcctgtctcctaggcccttggcccccccgttcctgctggctttcccacaccatgtccccagcctaggcttgggggacctctgaggcccctctgcctcctaggcccctcccccatggagtctggggctaacaaacctctctgtgacctcactgtttggccactgtgacctgttccctttaatcttgcccaggacccatctcttagacagctcctgggttctagctcagttctagccatcacatggctcaagggatatgcattttactggagaaataaagtgtgaattactggtaggtgacatctttatttcagggagcaggaggaagttttcctatttgaggcccttttagcactaactctgtagagaaacactagtgaattcctcacatttcagcaggccaaggcctctagggattggtagctcaagagtatagactcaaaaaactataaactcagtttgacaggtgagatggagaggtgactgtgctaataatcgtttagctcacaaatgattcagcaaactctttagacttgtgaaaaaacacatccaaaaggagactgcagcctggtgccggtggctaacgcccgaaatcccagcacttttgggtggccatggtgcaaggatggcttgaggccatgggtttcaagaccagcctggtcaacatagaccctgcgtctattaaaaaaaaaaaaaaacttaaaaattacccaggcgtggtgatgcacccctgtagtcccagctacacaggaggctgaggcaggaggattgcttgagcccagtttgaggcagcagtgagctatgatcgtgccactgcactccagcccgggcgagaatgatacccagtttcaaaaaaaacaaacaaaaaagaagactgcttttgctttcctgagggacagtaacatcattgcatgtataaaggacaacctattaaggtgggagaaagggatggatttcctctgaaagcttaggatttctcccagggtctctgatattagctttgtcttatgtggggtgacatttatcaggagaaacagcaggggtcagggttgtgtctgtcccactcaggccagatctacaccaataaatccaaggagtggctggctcttgcaggggctcaaaggagtcccatcctgccagaccaccagccctcaccacacagtgtcacagatccgttcattgactgcaccagactgaccacccaaattcccttatgtatggtgcctagccatggctaggctgggatgcctgagatggaattttaattcttcagcaactactcaggaatatcaacctctgagatgtgaactaaggagaaagcaacttcagtgacttatttctgggcaacctaccacatagggaggggactgcattagggactcatgccacttagaaaggaagatctccaagtagacagaaaggtattttccattcctcttgcatttgggtcccctttatccactccatttcaccccaggaggcaaacgagaggttgtgaatagaaaaagtccactgtgggccaggctttgtggctcacgcttataatcccagcacttcgggaggccgaggtgggagcattgcttgagcccaggagtttgagatcagcctgggcaacatagtaggaccctgtctctacaaaaaatacaattagctggcatggtggtgcgtgcttgtagtcccagttactcgggaggctgaggtgggaggttgaggctgcagtgagctgcgatcaagccactgcactccagcctgggcaacagagcgactctgtctcaaaagaaagaaaaagtccactgtgggccgggtgcggtggctcacgcctgtgatcccagtactttgggaggcagaggcaggatcgcttgaggtcaaaagtttgagaccagcctgggcaacatagcaaaaccccatctctacaaaaattgaaaataaattagccaggtgcagtggtgccgcccgtggtcccagctacttgggaggctgaggcaggaggatcacttgagcccaggcattcaaggctgcagtgagccatgactgcaccactgcactccagcctgggcgacagaatcagaccttcttaaaaaaaaaaaaaaaaaggccgggcgcggtggctcacgcctgtaatgccagcactttgggaggccgaggagggcatattgcctgagctcctgagtttgcgaccagcctgggcaacacggtgaaaccccgtctctactaaaatacaaaaaattagccgggtgtggtggcgggcgcctgtagtcccagctactcgggaggctgaggcaggagaattgcttgaacccggacagtggaggttgctgtgagccgagatcgcgccactgcactccagcctgggcgactgagcgagactccatctcaaaaaataaaaaaataaaaagaggaagggaacggaagggagagggagggagggaggaagagaggaagggagtgaaaacatccactgtgaaaactgagggagcgggaaagggctgtgcgtggaggatccagagacctggtttctagagcaagccagctgtcaggaacttgctgttcgctcttgctaacgcatcaacttccctctgcctcagtttctgcattcgtagcaccaagggcgtgttctaggcggtgagatcctttctaagcctctaaaaccccgtggtctggtcccctctccattcctgtccaggggagctcctcgggtgagacggcagaccgtgcaacgctgcacctacaccctccctcctcgagctgggcatctccaggctctgggaagccccccgcccgcccccagcccgggggtcccgagacccctgccagaggcttcacagtcggaggtccccccgctgccccttcagctgcgacaaccgccagtgcactggcctcaagcagtcgcccgaatcccctcaagccctggacgccggctagccccctaacacgtccagctcgcactcgccactgcagcggcgaactgcgttcccggccggccaggggctccgcgttcattcttaacctcagtcttaaagttctagctcacccgcctcctccttagcaaagggaccgccttctcgcccatgctattggtaggtagagccgtccctcagagaagaggcaggtccagattcccttcggaggctctaattggccttcttgacagtcactcgcctgtatctggcttacgattggtctttgggggacgaggactaggcaccccctgcttgcggtccctcggcccgttttcctgctagtggagtgccggtgtcccctcctcttggttggacctgattggctccacctcccgatctgagcattgtgattgggtgcagctgcgctgggcgggacggcctggagcgtcggccgttggcgagcgctctatccttgttcccctcccttctctcgtcagactccggcgccggagctccacccccactgacgggttctgattggccgcttctgacgcgtctcggggccacgattggtccgtccggtcccggacaccgctccctcccccttgaggcaactacggcggctccaaggagggggtgggggaaggagggacggccggtcccgtcagtcaggcagcgggagccgccgggagcggatggcggcggccgtagcggctccactcgccgccgggggtgaggaggcggcagccacgacctccgtgcccgggtctccaggtctgccggggagacgcagtgcagagcgggccctagaggaggccgtggccaccgggaccctgaacctgtctaaccggcgcttgaagcacttcccccggggcgcggcccgtagctacgacctgtcagacatcacccaggctggtgagtgcagccgggccccgggcccaaccgggtccctcatttgcatagccccaccccgggccccgcccggcccgggagtcgggcccgccccttcacctggcggccagggttgctgcccctcctgattgccctaaacctgcagccatcctctcccatctttgagaaccccctagacaatgaacgtgcttctcgtttgcacagaacgccctgcgcctccgagcgttgggagagattcctcctttctctttatttgcatatctgcgcacctgtaggatggtcccgcccctcacctggttggcttatttacatgatctcttaacctcagctccaatcggtctatgaagcagtccccacgagctcctctaataactgctttctctccctcttcatctacacctcttaacccccgtcccaccgagatccgagcagccccgggcacccccttcccagtgactggcccctaagcccgaggtttctgctttctgtgccgcaaccccgccccctaaggtcctggccctctccactgtccacgcagctctccagcctatgggaaagaaacaaatctctacctcccctcactcttttctccctgagctcttccgcctattatccccaaccggcttatctgagattcctcctcctccttcctaagcctgtgaggtcactgtggagcctgtgacctcatagggatcttagaattcccatttcagtttctgctttcccccgggggagggacttgaggccgttgctggagggaaggaaatcttatctcctagctgggagtgctggccagctagagactgtccttccacgcctccacccgcaggatctgtccaaccatgcaccctccaccctggtgtctgtggatggtagggggtgggggacttaactgtgacaggtgagtcaagcctcagctactcaggaggcagcctatgaccttgggccggcagctgggggtggggatcaggatcagggaggaggactgcattcccgaagcctcaccccagcccaggcctcttcctcctctcatctgcacggtgggggaccagagctggaggatagtgcttacttctccctaagcaggtgcctgctcacctgggaacactcaggccggcactggcacacatcctcttccctcttctggggagaaccagtcgaaacctccccactgatctcgcctgctctgcaacacttgagatggggaatgagtgagctccaggagccattcctgccccttacagccctgttccctcccacccccacctaaccccacactccagattgacctcagcagctgtgtgacactcactgtctgttcccttcccctcccccttggttgttttttgacaaagaccaaatgttattctttacttccttaggctgtctctccctgtgtggactcttggggaaaactacccggagctccccggcccctcccagctcccgcccccggccccctcctcccccagcattccggctcagggtgcagccgtgctcctgtgagcttgggggggggtcggaaatcccagcttcccaagaaccctcctctgccccactctctacacagggcccaggttctaggccaagtggggtccctcttgtccagaattctctcctctgtcccctctccgcaagcagggacagaaaggggaagtgacaaaaacatacagttgcccataccctgccctccatctctcctcgccctccaagcagacgaaaagcatcagtgccttgctgcccggactgccttgagccttcccctcccgtctcccatctctttctgagtgtcttcgctgcgtgtcgcctagtcttgggtccctgatctcccttggactaggctccctggggtgatagtaggaagaatcctgaggtggcggggaggggccatcttgcaaaggagaagtgcagctcattctctcccagaccacatcctgtgcccccagtgccctgcctcctgcctgagctcccttccgctcctagcttttgtccccgccaccgcctaccccatcagaactctgagaactcggtctccacccccttcgtccacatgcacacacacaaacacatttgcagagaaagcgggcaggtggaggcgaggctgggaggggcaaatgccatccccccaccctctccagaacggtcatctctgaggccagggcttctgtctgtgcaccagggctgggcagagaggaagagaaaggggtcccggggcatcaggctgtcatccaccctggtaaacacggggcctaccagagccagaacctgacagattgtttctacttcatttcctgtcacaagactcatttccttgggggatatacccggcttctctcctctgcttgcttttgctggcctgcccttccctgagaagggcccttggagaagctcaccaggtctgtggcctgggactccgcctcgcgggccctgggcccctcccaggacaggctgggccgaggaaacagaaaggcaggcttctccaggtctaagccgttagtgggtccgtgctacagggaatcccaaggctgccagcctccgcatagcaaggcagtgctccagataagagggcgattgtagaccccaggccctcccacactctccaccctcgccgtgcctgagatgctcacacagttgctgatctcttgaagtctgtgtcagtaagagaggaagcagagccgggctgctagggctagcagagggtaggaaggatgcctgccctgggccagagaagcccagatgggagggacctgggaactccagctcccatctccaaccaggtcttcttgcccttccctcccagcccttgggaatcagtaactttatgattctttggacctggcttgctggtccttggggctggcgaaaaactcgtccggtgtcggggactttatctctagaaccaagatagagtgacctgtggtttttcccgtgttgaaaacacactggagtagggaagaagggccccgaggagggatgggcactccctgtggtttgccccaggggtttcccatcttccctccgggagccccccagcccctcgccttactgttttcccatgagcactgacccctcctctcaccttcggtcctccgcctcacctggcatctctgaacaggaaccacaggagttagagattccagctgctctccaggatagccctccattgacattccccaacccccaagtctgaggcttctctagcccagtgacgctaaggcctttgccttccctgtgccattctggtcacgcctggaatgtgctcccgttggcagaaggctttgttatctctcctgggaggccacattgatggccttgaatctttaggtgtggaggtgagggagtgggcagccggctctggggggactgaaggtcctgtccgggctgggcctttccgcaactctccccgacacctccttttccccagacctgtcccggaaccggtttcccgaggtgcccgaggcggcgtgccagctggtgtccctggagggcctgagcctctaccacaattgcctgagatgcctgaacccagccttggggaatctcacagccctcacctacctcaacctcaggtagggaggccgagccagggccacgtgggagcgtgtgcttctcagtctcaagtactactgagtgacccacccagtgaccccgagaggatggcagtgctgggatgccatgcctggggagccagtccgctgcctttctcccttccacagccgaaaccagctgtcgctgctgccaccctacatctgccagctgcccctgagggtcctcatcgtcagcaacaacaagctgggagccctgccccctgacatcggcaccctgggaagcctgcgacagcttgtgaggatggggacgggaccaggaaagggacccctgagccaggcagcgggaccttctagggggaggttggggagggcctggagcaaagacagagatgggagggtgggttctgctgggcctggtctcatggccccttcccactgccttcctgataccaggacgtgagcagcaacgagctccaatccctgccctcggaactgtgtggcctctcttccctgcgggacctcaatgtccggaggaaccagctcagtacgctgcccgaaggtgagaagaatgaagaactggagaggaggtgtttgtatgtgcgtcccagttggctcaggctgcggtaacgaaaacactgcagaccgggtagcttatacatggcaggcattcatttttcacagttctggaagctgaagttcaggatctgggtgcttccatggtcagcttctggcagggtgcttttcctggctgcagactgccggctccttgtggtactctccgatggcggagagcagagacagccctctggggcctctttgataaaggagcacaaatctcactgatgagggctccaccctcatgatctcaattacttccctaaggcctcgcttcctaatcccatcactttgggggttaggatttcagcagaggactttttgggtgacacatttcagtccataacagtgtatgtgctttgaagaagttacaggccaggaatcttcagaggagaattacaccttcattgtccaggcttttgcagtatataaaacaaactgtaaacgatctgatttgatcttgacctagcaaccctctgatgctggcagggcgcagatgagcagctgtgttagaaacgtggacacaggcacagaaggggagctggtgggtgcatcagctctaggtagactggcaaggtcagaaggggccttcaaggtcacccagcagccctgctcaccccagcaggggctctcaaacacctgagtcgggatgctccgccactcctcccggagatgccagggccagtgccagagcccaggtggcccatctaatgtgcttgttgtgatgcactgatagcctctgccccaaagcaggcatgccttttgttcagcaggcagcagcatgtgtgtgtgtctgtgtgtgttatgtcttgtgtctgcgtgcatgtgtgtgtctgtgtgtatgtatgtgtttgtgtatgtgtgtgttgtgtctgtgcatgtgtctgtgtgtctgtgcatgtgtgtctatgtgcatgtgtatgtgtgtctgtgtctgttcgtgtgttgtgtttatgtctgtgttatttatgtcgtgtgtgtgcgtgtgtatgtttgtgtgtctgtgtttgtgtgtgtgttgtgtttatgtctgtgttatgtcgtgtgtgtctgtgcatgtgtatgtgtgtttgtgtctgtgtgtgtctttgtgtgtgtgtttgtgtttatgtctgttgtgtgtgtttaggtctgtgttgtgtgtgttgtatgtgttgtgtgtgtttgggtgtgtgttgtgtgggttgtgtgtatgtgttgtgtatgtttatgtgtgtgttgtgcatgttgtttgtgtctgtgtgtatgtatgttgtttgcgtgtgtgtgggttttgtgtgtgtgtgtgtgtgtgtgtgtgtgtgtgtgtgtgtgtgatgtgtgtatgattcctgtgttgggatcaggtagatgcgggtgatgtgactaagatctggggccatctcttagggatttaatggcttcttgaggagagacagtcatagggaagttccgtcagggggttccccagacgggggctagagggaggtggcactggactctgcgtgggggtcctggggaagacttcaccggaggcgcaggcgcagtgggagccgaacattgaaggagaattccccagccttctgcatccaggcagaggacccagcatgtgcaaagcatggggttaggaaggtgagccaggcatggcaggaaggcctgtgccatgctcaagtgtgaacttggttttgaagaacaaggagggagtgaggtggtcatccactttccctgtctccctactccctgaccctttctgcacctgccctcttccttcccagtcaaaaggggtaggaagggctctctctgggccataaaaagaaatccaagcaagatgtcctggggacagaggcaagagcaatgagttccatccaatctgggatgtctggaacgatccaccgaggaggtggtgatgcttgagctgaaccttgaaagatgcatggatggggaaaatggagttctcccagctgagcaaacagcacctgggaaggctcagaagctgagggcccggaggggtgggcagtgaggtgggaaacgcgggtggagccaggttgtggaagccttcctcatgctgggttagggagctgtgggttttattctgtgcttagggccgccatgaatccagactgtgccttttttttttttttttttgagacagagtctcgctctgtcacccaggctggagtgcagtggcgcaatctcggctcactgcaagctccgcctcccaggttcacgccattctcctgcctcagcctcccaagtagctgggaccacaggcgtccgccaccacacccggctaattttttgtatttttagtaaagacaggttttcaccatgttagccagggtggtctcaatctcttgaccttgtgatccacccgccttggcctcccaaattgctgggattacaggcgtgagccaccgtgccggtcccagactgtgcctttttaagatgagcaaaaggcacctttccctgtgggcaggcagcatttcatgcagggtgcagtgctgggcccgtgctggggaaaggggactcccgggcggccacaatccgctcaacatctgcagccctggttgcatgtcacaggaggtgtttgaacagggtttgtgccttgtgctgatcatttgaaaatggagggtgggttcgagtgtggagtgggcaggcgaggagatgaggaggacagacagggctgggcgggacctgacgtccggatgggccttcccggggaagctgagctgacgtccagatgggccctctcagggaggctcgcttagaccgtggaccagaatcatgtggagagaggagcagggaggtttctcccggggaatgacttgagccacccaggggagaggctgtgctgggcccagggtgactggccagctggagccaagggtgagggagtgagcacgaggccacagccctgagcatgttcctgacttttcccttgcccacctgttccagagctgggggacctccctctggtccgcctggatttctcctgtaaccgcgtctcccgaatcccagtctccttctgccgcctgaggcacctgcaggtcattctgctggacagcaaccctctgcagagtccacctgcccaggtgaggcgggtgcctggggggccacgaggacaggggtgtggctaggcaggagtggggttggtccccttggatagctgagcaggatccggggagctgccctccctgccaactccctggcgtgtgctgccccgcacacaggtctgcctgaaggggaaacttcacatcttcaagtatttgtccacagaggccgggcagcgtgggtcggccctgggggacctggccccttctcggcccccgagtttcagtccctggtaagtcctaggtggggaggaaggaagccctgggatactgaatgccccttcccggccccaggcaccccctgctttctggcaggaggatggggggctgttgtccagcatcaggctgaccagatccgcctggctggccccagccctgcagaggatctatttccgggacatcggtacgatggtgggctggactcaggcttccacagcgttgatagtggcagcaagaggtggtctggaaatgaggtaagggcgtcctgtccatgggtgattgagggtgtcagccctgggagagcacctctgaactggggacctccaaattgcagggtgccaggcagaggctggagctgtcctcttccttccctctgcccagcatcccagggaagggctggctcagcagagaccctgagcttagtcccacacaggaccccccaggtcctgagcccccatgtacagagaggccatctcccatctctgcctctgcctttacagtcaacagatgaattttcagagctgtcattccggatctcagagctggcccgggagccccggggacccagagaacgcaaggaggatggctcaggtgagtagcagggctggccgggagctgggctggaccagggcctgggtccccgctcacttgctgacatgctggcctccccagcggacggagaccctgtgcagattgacttcatcgacagccatgtccccggggaggatgaagagcgaggcactgtggaggtgagagcccagctatgcctctcccaagggaggggagctggaggaggggacagggcaggcgtgggcacaggcctgccctcagctgctctgctgttctctccttaggagcagcgaccacccgaattaagccctggggcaggggacagggagagggcaccaagcagcaggtaccccaagcccccaaactgacgccactgcccgatcctcccaccccccaaccgcctccttccctgaccccggggtctgcccctcttgccctgtcttggtccctcaggcgggaggagccggcaggggaggagcggcggcgcccggacaccttgcagctgtggcaggagcgggaacggcggcagcagcagcagagcggggcgtggggggccccgaggaaggataggtgggacccagggccgcccggggctgttgctggacactgagccctggaaatggggccaccgagccccgctgagccgttctgccttggggctagctgaccccacctctccttgttccagcctcttgaagccagggctcagggctgttgtgggaggggccgccgccgtgtccactcaagccatgcacaagtaggtttcctgccagcatcccctccccactcctcttcctatgaccatcatccctctaattcccttctgtctctcctcagcggctcgcctaagtccagtgcctcccaagcaggggctgcagcggggcagggagcccccgcccctgcccctgcctcccaagagccccttcccatagctggaccaggtgggacagggattttgggaggaggatggggcctgtgttggtgagggctggttgggagagagggacacagaggggatgcctgtctcacctgcccctgtcgctgtgtccctggttccggccccagcgacagcacctgctccacggccacttggctccattcagagaccaaacagcttcctcttccgttcctcctctcagagtggctcaggtgggtgtcctgccctccccagtgctgacccagtgcttgccagcaccaacggctacatcacccttctgccctgggagccctctgggtctgttgtaccttttgtacctttgcgttgaaaatcccacaagcaagccgggtgtggtggctcacacctgtaatcgcagcactttaggaggcccaggtgggaggatcgcttgagcccaggaattcaagaccagcctgggcaacatagcgagaccccatctccatgaaataaaaaaaaatcccacaagcagagaggcaggaggtgtaggtgccaggccactgggacccagctacagagtcagtggtgagcagtggaggcagccccatgcccccctttttctcctaggcccttcctcaccagactctgtcctgagacctcggcggtacccccaggttccagatgagaaggacttaatgactcagctgcgccaggtgagcagaggcgggagctgcgtgggcaggggcctgggccgggcacctccttgtgttgctgtccttgggaccagcctccctccacatcctctgtgcccactctggcacctcccctgaggacaggaagggacaggcacaagttccctgctagccctgagccccccactctccctgcctcccagacgcctctccttccctgacccctgctctatgtggtctacactgccaggtccttgagtcccggctgcagcggcccctgcctgaggacctggccgaggctctggccagtggggtcatcctgtgccagctggccaaccagctacggccgcgctccgtgcccttcatccatgtgccctcccctgctgtggtcagttggggctggggaagaggtgggggatgggccggggcgcctgtggcctctcccttaccacattttcttctttcacccaccccagccaaaactcagtgccctcaaggctcggaagaatgtggagagttttctagaagcctgtcgaaaaatgggggtgcctgaggtatgggggctgctttctaaagagtggtggcatgccggactcagccgagcatgtggcatggatggtgggcttccatttgcagcggatgtctgctctcagatgaaggcacaggctgcccctgccctgcccagcatgccccctgccctgcatgccccctgccctgcatgtcacctgtcctacacatcccctgccctgcaggccccatcttgtcctgcatgtcacctgtcctgcacatgccctgccctgcactcctcctgcactgcactccccctgcccagcacgtcagcatgtgggcgtggttctggccccagcacgtgagggcaggggtgggagggactgtcctgctgctgctgctgacgtctgtcccttgtccttgtccacctgctctcctctcccccaggctgacctgtgctcgccctcggatctcctccagggcactgcccgggggctgcggaccgcgctggaggccgtgaagcgggtggggggcaaggccctaccgcccctctggcccccctctggtctgggcggcttcgtcgtcttctacgtggtcctcatgctgctgctctatgtcacctacactcggctcctgggttcctaggccccaaaatcggccctccctcacccctttcccttcctctctatttataaggtccctgctccacccgaccccacctgcggtgccttcagccccaaccaaagacactagtgcacccccttcacagacactgacctcagaggccccactctggtgcccccagaccctgggcccccagcctctggcctccctccagtagccccacgagtccccacctctcagtgctgacggtgccttcatgtccccgccggccctgcccctgccctctgtaccccgtgaggggtggcaggagctggagtctcccccttcctcctgtgccctccccttccccccccaacagctgctatgggggggctaaattatctctattttgtagagaggatctatatttgtaggggttcggggcccaggccgggtccctatctctgtgtataaactgtacagaccgtggccgccctgcctgtgtgtgtgtgtgtgcgcgcgcgcgcgcgtctgctccgcgtgttggtggctgtggccatggctctgtgcccaccagcatctccctcctgagatgccggcctctcatgctcccggagcgtccgccaaccccccgtgtcacctcccttctgttatcgctgacagctttcttgcgtctcatttgtcgccgagccccgagcgcacggtgatgctcgggtctgcccccgaccccctgccacaggccggaagccgcagggggcaccgtggggaagctaacccggccccttcccccaggagtcactgtgccagccccaccacatcctggaagaggagggggccccgggaaggggcctcccctacatcgctgctgtcgtccacgccctgctggaccggccttaggggtgaaggtgggggcacagggcccaccccgcccgggaccccgggagcagaagacgcgcctgggctccgcgctctcagagaagcacgtggtgagggtggcctgggcctgggcacccttggcctgccggcctggctgcctctggggtcgagggtctgggtggaaggaccgcgggtgtcgcaggtgtcgtgtcagccgcaataaacagaagccagaagccctctgggtggccctcaggtgtaactgtgtctgagagcacgtgcgtcgcggggttgttgggggggcctctgtttccggaattcaccgcccagtcgccagggggcgcccgcgggagcccggcgggggcacgcgccacgtggccgggagacgcacagggcgccagcgtcgccgcgaggcctctggggtgggttcgccaggagggcacgttcgcgccgcgccgcggagggaggctgagagggccgggccgggcggcggggacacggcggtaccgtcctgcgctcgggaacgcggaatgttgccctggtcgccgccgcggtggggcgcgggcccggaggaggcgcccgagggaccaggccttccggcgggcaaggaccagggcgagccctggagcgccggagcggacgcccccagggagctggggacacccccatgggacaggtgaggggctcggacggagccgtggggggacagcgactgggggcctccatgccttcaccacgcgctctacggaggcttctccattgtccctgctggagattgggtccctcggtctcccttgcaccaggcacccttgggctccaggggagggtgcgtgggggcggtgtggctgcagcctcagtccctgcccctccagcccacagcccccatcccgtagcccttcctggatctggagagagcagctgctgctgccccaccacctgccaggcccggccactgagcagccccccggagccccaggtaggacaggccctgctgcctctactacccctaagagcctctcactgactgtgagcctgttataatccccaagcgtccccccagatccccgttccccccaggtggcctgggaggtggccccctcgaggatgactccactagcgccctgggaccccaagtatgaagccaaagcaggacctcggccggtgtgggtgagttgggggcaaacctgtgggactggctggggtgctcagggagctgtgcggtggcctgaggctccagtgctctgtcctcctcaccctagggggccaactgtagctcaggagcctcgttctcaggccggacgctgtgtcacccctcattctggccgctgtatgaagcagcctcgggcaggggtctcaggcccgtggcccctgccacagggcactggaatggacagcaggcgcccccagatgcaggtacctcccttggtcccccgggggcgcaaggctgccccaactcctctgggaaccagtgcccaggggtgggagcccgcagcccgccaccctagtggtgtttcctcctagggttcccggtggtgtgctgtgaagatgtcttcctctcggaccctctgctgccccgggggcagcgtgttcccctgtacctgtccaaggccccccagcaggtgagagctctttcccctccctgtccttaccccagcctccaggcttgcctggcaggaaacacattccattctctctcctcagatgatgggctccctgaaactgctgccgccgccccccatcatgtctgccagggtgctcccccgcccatcaccctcccggggcccctccactgcctggctcagcgggccggagctgatcgctctcactggcctgctgcagatgagccagggggagcctaggcccagctcctccgcggttggccccccagaccatacctctgacccacccagcccctgtggtagccccagcagttctcagggtgctgacctctctctcccacagaccccagacacccattgtccatagccttctcagggcagagtgggctggttgtgttgacaataaaacagtgttggtttgcaaaacaggctccgtgtggctgggagcagagtaagtcatagaggcaggaatggggtctctttctgtctcactgcagatcaccccctgctgtgttaggcggagggcgagggtatccatgacctaaggcatttctgggctgactgggcagagcccctttaatgagtgggtgggtggtctgcatggcagtgcagggctgaatgtataggaagcaggcgggggagtaagtaggggcacctcacttttaggggacactagccccacacctcttgtgctggggctccaggagttcagttctgggctgggtggggtagttgtctggctcagaggagacacaaagaatgcactgcaggtgtgtgggggagggtgccggtccatgtaaatgtcaccactggcagaaaggtggccctgccgcagcctacctgagactctagagattcttgcacgaactctgtagagaaacaggatcagggtttcagctgagaaagcccagaagtgtgtagtctcggtggtttgggcggggataggttctagaattttctcccataaaatagcccaacttcttggagaaccttctactcaagacttaggtgccacagctggaagggacacagggggcaggaccccagcttgttgccctgcaggtcacttgtccgaggtggcatctgggccttaaagcccctcacacggtggggagttaggaggcagggcgggggcgttacaatgccattccctccagtccctccctcaaccacccccccatcagtcagcaggagagagctgcctgggtctcaggtctcctcaccgcatagctatgtgggcggagcgtgtgggccctggggacgtcaccaagtgcccccagagaaaggaccaggtccagatcgacgtagctgaggcagctcgtcaggggctctggcctggcggtgccggttccgaggctctctggggccgataacttggtgcacacttcactccctagccagctctgaggggggcaggagcagcgctgagtccctacccatccccagccagcccccacatcgcgatgggcactcctgggcctgtgtccaggctcctgaacccagacttgtaggaatgggctcatagtcactccctgctcccttggttatgtagcccccacccagccccttagggtagaaggctgctgtccccagcctcgccaaggtcttaagggctgcttgctcgatcccctctcccagacaccctggacggctcaggccacttgactggtccgcagaagctggggtctcagggatggggaacagggagtctgaaggtgctcataggagggtccctgagcacctgggcctcgtcattgtgcctggagaactctttttttttttttttttttttttttttttttttttttttgagacagagtctagctctgttacctaggagtgcagtggttgatcttggctcactgcaacctccgcttccccggttcaagcgattctcctgcctcagtctcctgagtagctgggattacaggcatgtgccaccacgcccggctaatttttgtatttttagtagagatggggtttcaccatgttggccaggctggtctcgaactcctgacctcaggtgatacgcccgcgttggcctcccaaactaatgggattataggcgtgagccaccgggccgggcctgtgcccggagaactctaaacctgcccctttaatgtttgaactttcggcgacctgggggaggggcgagccacggagaaaccacagccagaagcccccgaggcccccggaagcccggggctcgggcctcaccggcatcaccgcgctcctcgagcagggtgttgtatcacgcctgctgctccgcctcgcctgccgccgccacgcgcaggctgctgtcgcgggtgtagaggacgatcaggtggtgctggcgcgcatctgcgcacttgctggtggtgcacgcacccgccaggctcaggctgcgctggagccgcgctcggcacgcgcgaaacttctgctcgctctcggaacactcgggacgcggcgggtctgcgcgcaggtggccgcagaaccgcacgtcggcagggcaggcccagggcagcggctggctgaggggcacggcggccattcccggctctccccggggccgccgcctgcgggcttcattcaggggcgaaggcaataaagtggctgcagaagaggcagagacggctgggtctcgtgtcttgagcacggggagaggccagcaggggtgggggtgaggtggcgaactcggcgggccgggagcagcggccgggcaggctgccacccgtcttgatgcccagcgtctctcccgggctcccgactggaagtttggggcaaggactctgcgcacagcgagtgtcccggtgactgttgcccctggcctgggctccccactacctccctccctccgccgaccgctccggaacccccaccccacgagactaatcctcacgtcgcccaggaactggagtttgagtagggaagtccaagagaggcggcctcgaccccaagactgagcctttataggggcagagagcgtgggggcgtctctaccgatcagctgactgcggtccggacacgtcgtcggggcaaccgagaaagcctcggtgtgaggccagtgatcggacttgtggcagcggtggtggcgctggcgattagagggctttggaacggggcctccatgcaccctgctggctcctgccacgccccgctcggcctgtaggtgtccttcctgtgtgtacccccttcccatccaggagtggttttgtgcccttccaagtaaccaaaggagccaccgccactgagcactgcgctggattatttctgagctggagattgagtttcttgaaagtttggggtgataggaggtgacttgggctaagtaagctcagggggtagtccctgtgcacagaccatacccgctggaacccagtcaagtggccactgaaggagacctcggaggaagaggccatctctcaggaagaccccttcgggctgccccattgctctgggcccctccttgccaccactgcccaaaaatactaccccgggaccacctgaaggctctgaccatggacgtgtggacttcctcatggaggtaccctttgcttcttgaaaaaattgcgattgggctggggactggacatttttgcactcagttctgatccaaggaccactgaccccctccaaggggatgggggtgaaggtgcaccagacaaaccagcaggtgacacctggtgggaccttagaggaccctgagatatcacccaaggcaagtaaggtgcctaaaggggaaagccgagggtcagggaggagaggccaacatgcagcccccataaagatggcattcagggaggagctccctcagggaaggaacaagatatgacaaagacagaacaagagaacagatgcaaggacccaaacctagcccaagggccagggcagcctgagcagggtgggcagggcttcccgccacacttcctgcttcctggggaaggggccccacgtgagtgggaatatgggaggcaagtgctgtgtgcatggagctggggtcctcacacacctattccaaggtcacattgagagggagatgcagggaaccaaccagcacccagccagcaggcaggtgggacccagggcctgctccaggtgacctctggtaagccaaagccccactggggtgcaggagaggctcgaagcacaggtggtgtggtgtggggcgggggggccgtggggcggggctcggcaactgcccagtgttgggtgttggccacagaggcaatggctccgtaagtggccaacaaaaccggactgtgttcaaggccatagccccacgtggccaagggccttgatacaggcaccatgtgcaaaggggcagggacacaaaggacatgtgcacacccctccccctcccagtcctgctgctgcctcaaagaggccaccaaagggcccctgtccctgctgcactccgggcccaggagtcttttttactccctgaggcttgaagatacctgaggaagatctctttattccttccccttcccactccccatcaaccaggtccccctgcagcacccttccttgtgaaatcacagctgctatgacagtgaccagctccaaaccaccctgtgtcactgaaccttgggtgctgggtgacacatgggcaggaaaggcaggaagagcagggatctgagggcctgggggctgggcaagcctcaggggtggggtgggtgctgatcaatgccaggtgatcaatgccaggtgattggacccactgagtaccaggacaaacctggagtcacgcaactggcctacatcagttgccttggcaacacactgcccttgaccctacccggaaaagatttatttcagccggtagccctggtaacttgagtggctccccagggacaggaagtggcagaacctcaactctgcctctcctgacctgaggggttcacttttcatccctgtcagggacggggcactgcgttctcagcccctatggaagaatgttatgggtttgggggaatccacggggaggagactaggctgttccagcgtgcccctccttcctcagcctgaggccttgctcctctgtggaaggcaacacagggacagagccacacattcagcccctaccctcagccctgctcccatcccctggcccacaggtctgacccgcaagctccgatgtggagacacttggtggctgactcctgcttcctcacacatggggatggcccacaggtgaccgactggctgaatgctggggccatgctagaggactgctgtctgcagagcatggtccctgcattggccagaagttggtgtgagcccaggtgagtcactctagccccgtggtgtgtgtggtgggtttgaatctgcagctaccaggcaatgttttctccatttcctctcaagccaggcctttctccattctttgctactctccaggagaggcggctcactggagggctggagcctgagctgctggagctgtccccggccaaggagagcagctcctagtgtcagcctgtgcccagggaaccctggggcccgaggctgacctggcgttaacttcttactgctcatccagctctcaccagccagcaggctggagtccttgtgggctggaggggagggagtctgcagggaggacccgggcctggacccagatagtgaggagatgccgggagacagcacagggtgacagaagcaggagaaagcccttcggtttagcctcagttccaaacctcggcgctcctggcattaagggaagggtgatggaagctggggggaaataacagaaaggctcctccccctaatacaccataacagattccagcttccctccctccggctctaagcagaaaggaaagaactggtaagagaattaggtagagggacacagagataatctagggtggcgaagaggggagaaagagaagagatggaacatgcaaaggccaggagcgaagcgggagaccacccaggagacagaaggagggcagaggctgggagaagaacaggaacagacaggatggggtcctgattgttggaaaagacagggaggccaatcccagccttgcaaatagcagcagctagaggttatttccagaaaaatctgcaaagtaaatacactgatcacccaactcccatatctctttgctgctctctgtggaagactcagacaaaccccaagaatgaatggccacggggccagggcaatggtggaaagggaggggcctcacctggccttgggccaaacagcaaggtggcctctgcccagccaggtgaggtgtttgccacccaccccctgaccaggtctgtcttgtcaccacacaaatagcccagacaccacagctggatgttgtcacaacatccccaggtggaaagagcctggggcagagaatgttccagacccaggaggggtctacttccagcaccacattggcaccaacctcctttgcccaccctggccccagtgggcattcccctcacatccaacaaactccagtccccggcagtggtggctcctctcccgccacgcttcttctcattgctaatgcgggactcttgttcagacagggacgggctctgccctgtctctggccaatggcagcattatcactggcccaggcagtccagcccagcagagccaggtcaaggatcagtcatgttgactgacctgcaacccctcaaagtggcctgaagcaaaaccacaggcgccgttttctctttcgttcaaatgcagttcttttccaacattgcctgcaatgagctttggtgcagagatttggggacagggaggcacatggagaaaaaaaaaaaaaaaaaaagaccaggcaattgcacaaagcctttccctggaaaacccaggctctggggatggtgggggtcaggacacatggctgccccccctccagccctggagctccggctttgatggtcgaggcacagatggggattcatcccattccccacatacgagagcagtctgggagcgggagatcggctgggtgtgtgtatatatgtatgtatgtgcatatagatagatctatttatatataataggctatattagaaaatcctctctatgtataaatataaaacactggtatccaaacatgacaccaacagctaactaaagtgcttgacaggagccgggcccgggtaagctcgatggtaaaccgctcatcctgggcgctggggaagcaaaacaatgggggaccccttcagcagggggtgctgcaggaggaaagtgtgtgctggccaagctgggccaccaagagcacttgggccacctgggccttctgggtctcaggactgtctggtcactgccccatcaacactgtgcctcatctttcccctaccttccccactgcgctcccgtccatgaggagcacagcgctggcgagctaggcccagggaagagctgttccacaggggactgatcatttgaaaaaaatccctccccctcaaccatcactcctccaggtcaggccctgcccgcactcctcctgggcttcctgcctgggtgggggtgggacgagggaggggagagaggctttgtggaatctgagggcagccacctttcacctgtaggccccaagcaaagcattagcacctccaccatccccaaggcccatagaaatgcccacaggcaagtggtcaccagagctctagggagcagaggggccccagaaggcagggaagaggccccccaaaaacacagtgctacaagaaggggttggtggttggaggtttggaggcgaatgggcttgatgagggtccagtctggaaagaaagaacaatacattttccagttaggggagaggaactggaagcttagaagaaaggcagccaggagtcctttctcctgaaaaatcgctatgctgagggcagtgggaaagtgagggagaaaaggccaggcacggtagctcatgcctgtaatcccagccctttggcaggccaaggtgggctgatcacctgaggtcaggagttcaagaccagcctggccaacacgggaaaaccctatctctactaaaaatacaaaaattagccatgtgcggtggtgcacacctgtagtccctgctacttgggaggctgaggcaggagaattgcttgaactcaggaggtagaggttgcggtgagctgagattgggcctgggagacagagtgaggctgtctcaaaaaaaaaaaaaaaaggaaagtgagggagaagggagccaagggcagggctggaacagcatgggagatgccagtgcccagaagagctgaggcccaagctcagccctggcttggacaacgcagaccttcacgaggaaggggcacaggggccacctcacatcagcctcttctggaaggattgtcccagagggaaagcccacgagccctaccacagcacccctgctggcacactcaccatgaaggggttggtggagatcccagctggctggctcagtggcctctgccccaacttggctgatcctaagtccccgaaggaagagcctagggggcgagagcagtcactcagctgacaccagcctgccttcctgcaggcggacagcaaggggggccctcctaaactgggggagtaagggaacagagcagtggaagtacccgggcgggtctggtgcttccctggtcccccctgctggtcagaaagggcactgcatgggctctgatgaggtcggaggaacaactaactgatctctatgcagagatgggatggggtgaaagggtttgtccatctacagctcttaccattctgctgctgaacaagcggggtctgcggggggaacagcggtgctgggaaggagctggcaaaggccccagtgggtggcactgcctgggggaagccaggcccagcactgctcatcccaaagccgggccctatggagtgaaagacgggaggctcaggtgaacttctggaaggtgggctggagaactgtttttcctaagtccctccaagatgggaaaatgggatatagttttttcctgacatgaggaacgagggacccagccatggaactcttctgcttctcacaccaccaaacaaccctgccctctccctgcccgggggtacgtggcagggagggcatggtagttccaggcacgggcgcggtgcaggactgggctagcctcccactaaactgcaaaaacagcacaaacttctaacgctgtctgtgcttcagttctcatctctaaaatggggacagtccgtctcatagggctactgtgaagatcaaatcagggccacgaagccctcagcactgggcccaccacacagctcttctgtgtttgctcttattcctctctccccagcctcaaggcttcagccagcatgagtctgaggggagagcagcagaggctggggagggccagggtgagagaagacaacaggcctggagttgatggaagcagggaacagcgtgagtgtttcaggtggcttaagcttcgggacacagaagggactggggctgggtgcccaaagggacactgctgagggctggagacaggagaatcgctgccactgtcagtcaactctaaaacagtcttcagtgcggtctccggtaagtagccacctgtgtgcttctaattctaaaatctggctctgtgacctgggtgctaggtggcctggcaccgtgtgggcaggggaagaaccaggctgtctgcttctcctgacagcgtgagacaaagggttaagggagaggctgcagggtaatgtgtgccttggaagtctcagatggtcaggacaagggaccatgctggaggcttacctggcgccaagccattgggctggaacgggttggtggaaggcagcggggactgggcggcgggagctgtgaaagggttagtgaaggctgcccaggtggaatggggagcggcaggaaagagagagatggttactttttttctcacgtgcttcatcctgtgatgcctgagcattcctctcaatctctccccttcccaccctgtccagagtccttagactcctcctggcctcaggtcccctcccctgggtctccacaggcaggactcacctccaaaggccagccctgtgctgctgccgccgccgccgcccatcgtgacagactggagcggggggacctggccagccatcccgaagaggctacaacacggggagagagaagtcatacacagctgccagggccaatcaaacctgagcaaggggccaggagatgccaaccctgctctccagggtgagtaagggagaggaggggaggggagaactgccaggagatgccgatggggctgggaggaacatggccaggcttcgggcggctctcctgccctacagcagactgcccactgtctgtacctacctgctaggaacacctgcagcgggcaccccgggtcccaggaagctgcccacgtctgcgaggctgtttggctgactggcgggtgccaggggagtggcaccaaatggagtcccctggctgctccctgggcagttgtggaaaatacaacagggagagctgaggtaagacagggcgggaggggcaggagagcagatggaggtggtggtggtgatggattggagtcatggcatgacaggtgcctgctgggttctctgaggcagcccattttctcagggaccacccagttcaggaggtcttgtctcccaccaagagttctgggcaaggtgacagcccagcccttctccgtgtcccctgcgctcaaagctgcgctgcggagaggaaagcagctcgtccccaggtcaggctgtcctcctcctgctccagtttggctttcatgtctcacttttccctcaagagatggaaaagcctgtcctttcctcaagcccgcatttccatttatcagaacatggcagggaggggcaggcagaggcaccatggaggacagcaagtcccatcacactcaggaggctcctggcctgtgcagcctccagctatcagcactgtcccttccccatttagccgctgcttctcgggaggccgccccagtgccctgagcctggaggcactgttcatggggccagccccgctgcggggttctcctgccctgcctcttcttcagagccaaaaagaaaaagcacagagggcaaggcccacatggaaatcttacaaaacagctgagggccaagagtcctggaaattccaagcaatcttgtctcaggggaagaggctgagtccctgtcattttaagaaagcctcctgtctgcacatcaaagtctcgtctctatcttgggaccctatcgaccacactgggctgctgtgaatgtaaaaggagggtccagatggaaagaggctttattgacaggacgcggcagttcatgcctgtaatcccaatactttcagaggccaaggcgagcagattacttgaggtcaggagttcgagaccacccttgccaacatggtgaaaccctgtctctactaaaaatacaaaaattagccaggcatggtggcaggtgcttgtaatcccagctacttcggaggctgaagcaggagaatcacttgaacccgggaagtggaggttgcagtgagctaggatcacaccattgcactccagcctgggcaataagagcgaaactccgtctcaaaaaaaaaaaaaaaagggagatctggccaggtgtggtggctcatgcctgtaatcctagcactttgggaggccgaggagggcggatcacgaggtcaggagatcaagaccaacctggctgacacggtgaaaccccgtctctactaaaaatacaaaaaaaaaaaaaattagccaggcatggtggcgggtgcctgtagttccaactactcaggaggctgaggcaggagaatggcgtgaacccgggaggtggagtttgcagtgagccgagatcatatcactgcactccagcctgggcgacagagtgagactccgtctcaaaaaaaaaaaaaaaaaaaaagggagatcttccacatgccaggcactatgcataaataacagcgacccgggccctaaatgggtgaagctgagaatctagtagggaagacgaagtgcccagtgcctacctgctgccaggtcctgaagccccgctccctctcactgagtgtcggcacttgtaagacagtgctctcctttgccacctccttcctaaatgcctgggctttctagatgaatgagtgctggctgccagcttcctcctgggcaaggctacaatgccactgtgcttacttggtcatcaatcctttctaatccacaggatgcaagacaggctagaaagccacctgggtggggccaggcccagtggctcacacctgtaatcccagcactttgggaggctgaggcaagcagatcgcttgaggtcaggagtttaagacaagcctggccaacatggcaaaaccctgtctctactaaaagcacaaaaataagctgggcgtggtggtgcatacctgtaatcccaactacacgggaggctgaggcaccagaatcacttgaacctggaagaggaagattgcagtgagccaagattgagccactgcactccagcctgggcgacagggcaagactgtttccaaaaaaaaaaaaaagccagcagcatggtgttggatcctctactgactctcccagccgaggcctttccctgcctgcctgcctccaagtacagtatgtggaatttccacgcaagcacggctccctttcctcggatggctgactctttgtgctgctgtggtcctgcatgttccaggagcttcacccagtgaggaccaggctgggagagaggctgcaggccatgctgtgcctccctgtggacactagggggcagaaccatccctagcccaggcagggttggccctttgcacagagggtgcattctcacaaaggaggcggtggcccaagaaacacctgcactcctgtttgcttttcatttcatgcactttttttttttttttttgagacagggtctcattccaattgcccaggctggagtgcagtggcatgatctcagctcactgcagcctcgaccttcagggctcagctgattctcccaccttagcctcctgagtagctgggactacaggcacgcaccactatggccagctaattatttgtatttttggtagaaatgaagttttgccatgttgcccaggctggtctcaaactcctgagctcaggtgatctgcccacctcagcctccccaagtgatgggattacaggcatgagccaccacacccggccttcatgcgcttcttataccacgcagctagatttctaccctgccttatcacagtttgagggagagaagcagttttgaaaacccctctctccatactccccctcagaaatataaaaataaaagcaggtttgtcagaagagacacccacctctcaaaatctcctctctcagttcacacccaacagctaagtcacactcacgggaacacctgcatgatcatggtgtagtgggaagcggttctgccaattatcagcattagatctgggccaagttgagccagtttcctgcaaagggaagctctttccttcctcatggacataatgcatgaaggcacccaacaatggcaggatcctcggaaatgttccttggctctgggtttggctgtagattgactgtttttgttttgtttgagacagagtcttgctctgtcacccagagtgcagtggcgcaatctcagctcactgctacctcagcctcccgggttcaagcaatcttcctgcctcagcctccccagtagctgggactacagtcacgcaccaccatgcctggctaatttttgtattttggtagagaggggtttcaccatgtaggccaggctggtttcaaacttctgacctcaaatgatgcaccccactcagcctcccaaagtgccgggattacaggtatgagccactgcgcccagtgactgactgctttttgagacagccagatagtggacaaaaggtttaatgcttgggacatccgtggagctgagcccttccttctctctgagtctgccccaaaaactgtctttacggtgaaaccctgtctctactaaaaatacaaaaaattagccaggtgtggtggcgggtgcctgtggccccagctactcaggaggctgaggcaggagaatagcgtgaacccgagaggcggagtttgcagtgagccgagattgtgccactacaccccagcctgggtgacagagtgagactccgtctaaaaaaaaaaaaaaacaaaaacaaaaacaaaaaaaactctgtctttagagcaatcaattgtactgggaaggcatttcagaagggacgcatctataacaactttatataggctgtttcctcgagtgctcccattcctaggagcacacctttcttactaaatcgaatattcctgcaccaatgtacaattcctgccaacagcgcacaagaaggtcattttcccccacataatactggccattatcaatctaaaaattttgccaacatgatgtgcaaaactatcttatcctagtttgcatttctctgattaattaggttgaggatattttcttttgtttttttgtttttcgagacaaagtctcgctcttgtcgcccaggctggagcgcaacagcacgatgtctgctcactgcaacctccgcctcccgggttcaagtgattctcctgcctcagcctcccaagtagctgctattacaggcgcctgccaccatgcccggctaatatttgtatttttagcagagacagggtttcaccatatcggcaaggctggtcttgaactcctgacctcaggtgatccacctgcctcagcttcccaaactgctgggattataggcatgagccaccgcgcctggcctattttcatatttttttttctgggtgttacctgcttatatgtcttgtccattattatattttcatattcacaggaattttaaaaatactttatggatactaatttccatctgttttccatgttgtatcttctctccatctgtttcatacctttaagctttgtttgtgtcatcttttatcatacagaatgtgacctatggccaggcatagtggcccatgcctataatcccagaactttgggaggccaaggcaggcggatcacttgaggtcaggagattgagaccggcctagccaacatggtgaaaccccgtctctactaaaaatacaaaaattagccgggcatggtggcatgcgcctgtaatcctgctactcaggaggctgaggcaggagaatcgcttgaatgagatcacgccattgcactccagcctgggcagcagagtaagactctgtctcaaaaaacacacaaacaacaaaacaacaacaaaaaccaaaatgtgacctatgtgatttgtctttgttagaatttatggagattttgtttcctatagtttatttcttacttgtctattagtcatgtgggaaagcatattatagtctcctagtcctttggcttattaaattctgattgtgtctaatagtttttgcttcgtgaatctcatgggtatgcggtcagttattttaagtacatgattttgagaacctctttgtgaattttaacttttttttttttttttgagacaagagtcttactctgtcactcaggctggagtgcagtggcaggatctcggctcgctgcagcctctgtctcccaggttcaagtgattctcctgcctcagtcccctgagtagctgggattacaggtgcctgccaccatgcccagctaatttttgtattttgagtagagacggtgtttcaccatgttggccaggcttatgtcaaactcctgacctcaggtgatctgcccacctgggcctcccaaagtgctggcatcacagatgtgagccaccacgcccagccaacttttatcaatataaaatgtttcttcttatagccctccaaaacgataaaataaaaacaaaagcagctaatcagctcacagtggcacagtgagaaggctgtgggcagagcatgagtcaagaagtctgggctctggtctcagcttggtcgctctgtggcctgcatgactagggaaattgcttaactgccttatctgtcaaatggccaaattttaagggttccttccagccctcaaaactctatgatcagaggcacttgggtaggctctggggtggggataataacttaccaaagctgtaactttcagttagcatccgactggctggcagtgagggtggagaagagaagggtgggtagaggaagggagaaagaaagagtgtatgtcactggcttttccccagacatctgagcctgacggcgagcactgcagctgccctcccatgtgtccaggatgtccaatggcagaaggcacccaaaggctatgaagactcaatgtgtagagcttaggaaggagcagaatgctttaagaaggcctgaagagtctcttgcaatggctaaataacatagtttatttggttttgtttgttcgttcgtttgttttgagatggggtctcactctgtcacccagactggagtgcaatggcacaataatcactcactacagcctcaaactcctgggctcaagcgatcctcccacctcggcctcccaaagtgttaggattgcaggcgtgagccattgtggctggcagcaacatagagtttacaggcctttggtcttagagggatggatgaggacttctggagactaccttgacccttgaccctttgggacagcgtactatgcagacaagagcaccttagagaaaaagtcacgtccattttcaatttttctaggactcaggtcttccatacttcctagtgaaaagtttcctagtgtataacccacacagctcctgctgcaatcttagtcattttcctttcatgaaaaagcagaacagtagagccccctctccctgacaagaatgtgtcagacacgctcatcctctcctggagctgcttctctttgtgctttgctgctttgagcagttctttaggatagtgaggatatgaatggtgatttatagacatgaacaaatggacacggttggaagagaaaggaagccagtggggcagagtttacctgcaggagttggctgggcctggaacgaggcttgaccagctggagggaggcttccaaacacagaagagctggggccactgctaaaggcatcaaagttggcaaagcctccttgggaaggtgtctggcctgtaggtggaagaaacataaaggttatatagaaaattgtcgggtgtggtagctcacgcctgtaattccagcactttgggaggccgaggtgggcagatcacttgaggtcagaagttcaagaccagcctggtcaacatggtgaaatctgtctctactaaaaatataaaaattagctgggcgtggtggtacatgcctgtaatcccagctaccttggaggctgaggcatgagaattgcttaaacctgggaggtggagctagcagtgagccgagattgtgccactctactctagcctgggtgacagaatgagactctgactcaaaaaaaaaaaaaaaaaaaaaaaaaaaaccacacacacacaacagaaaaagaaaagaaaatgtatatgcctatccatgtaatatagctactaattctaaaggccaaaggccactcttatgggacatctgcctttccacagggttctcttcagagggaaatgggtggggcctggggccaagagccagacccacacaggggaacaggggcacccagggcctccaggttcactccctgccctcttctgtccttactccatgaggcattaacatttttgtcagggcctagtttggagcatgaaaattttttttcatggaggaaggagcttgaatttttttttttttttttttttttttgagacggagtctcactctgtcacctaggctggagtccagtgatgcaattgtgattcactgcaacctccacctcctaggttcaagcaattctcctgccttagcctcccaagtagctgggactacaggcacatgccaccacacttggctaattttttttttctttttagtagagatgaggtttcaccatgttggccagactggtcttgaactcctgacctcaggtgatctgcccacctcagcctctcaaatgctaggattacaggtgtgagccactgtgtctggcttgaaaatttttaaaaagggaaaacagattctatgctgctgaaccctctactggctattgctgatttgggggaagaataatctttggtgaactggaacacatgataatccccctcctcccattttaacagagacagaaaataataaaaaaggctatttccagtgttctaaaaggaagtgctaggagccctcaactataatgttttaggatcaaacccccaaacataatcctggctgtacttatttgaccccagtcctcccagcctcactgcttcagagtgaccctcacatttgtttgcatgagaagatctagagccatggttacctccagggactggatctctcatgttgtaagcatatacgctgtagggagggtcccatcccaaaacccacttacccccaaaggcagggaatgcagcaaaagctggtgccatctggggtgcagcaaaggggtctccaccgatgtcagccagcaggtcagtactggcttttttgacagaggagtggggaggtggctgagtgctccgggcctgggatgtccgagcgtgagactgactgacgggcttggaggagaaggaatgcatacagcccttagaatcccagacatgccccaggccctcctctggctccccaggttgccctcagataggcaggtagagagagaagacagtgagtaactgccctcaacctagaagattcctgggcaggagctgccttagcccctctcagagctcatatcatcctcatcttctcagtacacctgctccaagaaccacacattttacagcacatgagccaaggtaaccctgagatatacaggaatactgcccaatactgcccccagggctgttactcctcaaccttcccaggtattggacaccatattccagccccttctgtgtttctttgatggaatgctcaggtttggagctgatattctgactttcctaaataccacttcctgttaggttctgacttccaaaagatatgaggtccagaagtgacaaatacaagatacaggttaacagcagagccgagacccaactcacagctcctgcactgttgtctgctaagtccggggaatctgattcaggaaactgacactcctgtctcccaaatctcaaaagaaacatgacgagcagatctgagagttacctggctcgaggtggaggcagcaactgagagagacggtgcaggatcacccagaagtgtccgaaggggcttcccttctgggatggagccctgcacaggggtggaggcactgcctttggtataagtgggccccttgacttggtctggggggacatacctttgagaaagaatagttaggctgtaaagagagggataaaactttggaccacgctcagctgagatgtttgggaagacacaggagtcaaaatctatgctcatttttctctactgattcccttttttgaagggaaccgagcaacttagcaaagttctgcctatcttgagcatccccttgaacatcttttggtttttttgagacagggtctcgctgttgcccagggtggagtgcagtggggcaatgttggctcaacacagccttaactacctaggctcaagtgattctcctgcctcagcctcccaagtagctgggaccacaaacaagtacatgctaccatgcctggctaattttttaattatttgtagagaagaggtctcactatgttgcccagggtggtctcaacctcttggcctcaggtgatcctcctgccttagcctccaaagttctgggattacaggcatgagccaccacacctggcaaaggtccccctcttttatttcctgggtgtttcttcagactccttcttctcagctccatagaaaagctgcctgccgcctcatcctctaggtgctcacaatccacacttccatcctgccagctcctgccgggacaattcccttccactacagtgtatgtggcccctactctgtaagtgtcagcttaatcctgaggacatccctaggagggactccacagtgtggtcttatccacatcaatctctttgttctctgcattctcttctctacctcacagttacctcccaacatgtacacaccccagagatcccacaagaagtgataccaggggagaggaaaggtaagggagaatggagatgctgtttccttcatcaggaagacaaaatatagttgtcccatggtatcctcagggaactggttccaggatccccctgacaattccataatcagaggacgggacactcaagtctcttatataatatggtgaagtatttgcatcaaatatgtatatcctccaatatactttaaatcatctctaggttacttataatacctaatacaatgtaaatgccatgcaatagttgctatattgtattgctttttatttgtattattattttttttttgagacggagtttcactctgtcacccaggctagagtacagtggcacgatctcagctgactgcaaccactgcctcccgggttcaagcagttctctgcctcagcctcctgagtagctcggattacaggtgcccatgaccatgcctggctaattttttgtatttttagtagagacggggtttcaccatcttggccaggctagtcttgaactagtcctgaccctgtgatccacccgcctcggtctcccaaagtgctgggattacaggcatgagccaccacacctggccttatttgtattattttttattgttgtattgttattttttgtgttccctacttttttttttttttttttttttttttgagacagtctctctctctttgcccaggctggagtacagtggcatgatctcagctcactgcaacctccgcctcctgggttcaagcaattttcctgcctcagcctccagagtagctgggattacaagtgtccacccccacacccagctaattttttttgtatttttagtagggacggggtttcaccatgttggccaggctagtctcgaactcctgacctcaagtggtctgcccacctcagcctcccaaagtgctgggattataggcgtgagccaccttgcctggcctattttcccaaatatttttgatgtgaggttgattggatccatggttgcagaatctacagatacagagggctgactgtacatataaatcatgaaacaaggccgggcacggtggctcacgcctgtaatcccagcactttgcgaggccaaggcaggcagatcatttgagcccaggagtttcagaccagcctgggcaacgtggtgaaacctcatttctacaaaaaatataaaaattaaggccgggcgcagtggctcacgcctgtaatcccagcactttgggagccaaggtgggcggatcacgaggtcaggagatcgagaccatcctggctaacacggtgaaactccgtctctactaaaaatacaaaaaattagctgggtgtggtggcgggcgcctgtagtcccacctactcgggaggctgagacaggagaatggcgtgaacctgggaggtggaacttggagtgagccgagatcacgccactgcactccagcctgggtgacagagcgagactctgtctcaaaaaaaaaaaaaaaaagaaatatatatatatatatatatatatatatatatatataaattagccaggcatggtggcacacacctgtagtcccagttactggggaggatgaggtaggaggatcacctgagctccaagaagctgagactgcagtaagctgtgactgtggcactgcactctagcctgggcaacagagtgagaccttgtctcaaaaaaaaaaaaaaatcataaaatgatgggttaaaggaataaaactataggatgtgtgcttccagccatctctccccatacataagatcagctgagctataaaaatgaacacccacaggcctacctcccgactccaatcagtctactatctctccaaaagagaggacataaaatctgcagggttagtgactgagaacaagttgtcctttcatgtagatttattttagccgtaactgaatgtgagtggaacagaatcgacccaagtctagtcttttgtgagaaggccaaattcaactctagaaactggactgtcagatgaagaatgtcttgaacacgtttccatagcaacctctttcctactccttaccatctcttcttctcatatttttcctggagaaactccttcactttctgaggatccctggaatctggtactaaagatgtccgagcatcaaacagacccaaccaaatcttccggcaaacctaaaagaatgaaggtgtcagaggagtgggagcagaaagcaggaaatacaaaaaataccacttcctttttttttttttttttttttttttgagacggagtctcgctctgtcgccgggctggagtgcagtggcacgatctcagctcactgcaacctccacctccggggttcaagcaattctcctgcctcagtctcccaagtagctgagactataggcatgcgccaccacgcccagctaatttttgtacttttaatagagatgaggtttcaccatgttggccaggatggcctcgatctcttgacctcgttatctgcccacctcagctacccaaactgctgggattacaggcatgagccaccgcgcccagccaccacttcctatttattcatcagtaaatacatattggctggatgtggtggctcacacctgtaatctcagcactttgaaaggccgaggtgggtggatcacttgagtacgggagttcaagagcagtctgggcaacacagcgaaacccactctctaaaaaaatacaaaaattagtggagcatggtggcatgtgcctatagttcgagctacttgggaggctgaggtgggaggatcgtttgagcccaggaggttgaggttgcagtgagccaagattgcaccattaagctccagcctgggtgacagagcaagcaagagcttgtctctgataaataaataaataaataaataaataaacaaatattgagcatggacacagtgctaggatctgggtctacctacaaaccagatatgattcctgtcctcgcagagtttataatccaatggggcagtcatatcttagataaataaaactaaagttataatcacattttgtgacatgtattgaaaaaggaaaatagagggccatgagaattatggagaattaaatttcactaagggtagggtccaggacaattttcagagctctggcttccaggacacctccagaggcccttgaggataccctctaagtaaaccaatcacccataagctggggtactgctaacgccaaggatagcaacttctgatcggcagatttagctagggaaggggtttaagtcactttccagtacaagatatgcacttaaatccaggttgtcgaggctgaaggcagcagagctggctccaggaaagcttcctcagaggggctggcccagtcaactctgaatgctcacgctctggagctctggtcattctacccctccattgctcaggtgccacatcctacaacgatattgctacttcccctgactcctgaaaaggaggcctagacaaaccatgatttgtataacagaataaagattctgtaaatatacctaaggaaagaaatcatacatatgaaaaaattaagagaaactcaaccaaaaataaaaccgtaaggtttccctacaatcttgaggcaaagccccacctgcagataggtgaccttctcaccactccatcggtggcagctcacctcatttccacgggattgcaggaatactacttcaggctcagtgaaagttgtcatggagattgacttgacacgatgaggggggttcagccctctcctatgggagagaaacaggaagaaagagaagtataatgcagttagcatggcaggctgttcatgggaggaggaggaggagggagaaagggtgaaagaatagcatataaactaagctcagatagaacccccccatctgaagctacttcctaattccaggaaagaagggaaatatttggaatattttggagatattagaagtctccaaagagaggccaggcacagtgactcacacctgtaatcccagcactttagaaggccgaggcaggcagctcacttgaggccaggagttcgagaccagcctggccaatatggtgaaaacctgtctctactaaaaatacaaaaattagccgggcatggtagtgcacgcctgtaatctcagctactcaggaggctaaggcacaagtatcgcttgaacctgggaggcagaggctacagtgagccaaaatcaccccactgcactccatcctgggcaacagcaagactctgtccccactcccaccacccccctccaacccccaaaaaaaagaatctccaaagagacagggaagagagagtcaggaacatcctgtgatcccagtagttctggagaggaaaggtagagcctgcccatccagaagcctcctctagcctggctcagatcccagtagtttcagtgttcagcctgtaggccgaggggggtacttctgggaactcagacacgtgaccacttcccacttcctactttatcattgttttagacctacagcccatccccaaggtcccagggcccacaaaaacacaaatccttcaagcatgagggaaggcaaaaatgaaggcccagtactattcttgataacactaacaaaaacaggatatggtgacaaaaaacagtctctagcccagctggtcctccttgtgtctacaaagaagggtggagtggtggtgggatggagagggtccctctctttcaggaagccatctctaggaaaagaaatatttgattttcttatttctgtatctctcaataaagcaggaagaagtcaagttcaaccatgagctataaggctggcatgggcgaggaatatgtaacctgtggcaatgcttttgtgcctcctctgtatgttctgttaactccctgggaatccagttctgcctccaggaaccctgtggagattataagagaagagaaggcttccttgtccccactcaactggtctcaggagtgttctaatacaggaaacttggggctaagccaggcagggcttcataggttcccctgtccttgcaccttaagtattattctggtctaggtctactaccctcactggattaaacaaaactgcccaaaccaaaacaagctagatcctgggacaggagggaagcagtctcagcagatgaggcctcttcagacttttactctcatggaaaaaaaaaaaatggccaaatcgagttgctcaaataaaaatccagaatagggtcctggctgacatcctcaggaagcgcaaagggggaaaaagagacaaaaatatttaacagatgaaaatcctgaaaattcacaaagaaggtggggaaaacatacattcacactaacaggtgcacacacagaatccaagtgctcctgccaagtctgggagaacagcagcgaggtgtaagcaaaagcagcggtggtgggggtggggggcagggagtgggcggactcagtacagagtgaacagtaagggcggccaccctgcggatgataaattactcattatgggattttaacaaaggacaggacactggaggctccagttgacgctgtttgcagcaattctgctgctagacctttaggtataaagggtctcaaaaatgctcttataaaagacaagcaatatcaaagaggactgatgcccccaaggaaagataagctcagggaaaaacgaaagcaaggctttgttactacatatcaaacaatagctcttaagcttggtctgccccgaagactgtcttccgatcacatttcagagatagtccattctctctcctaaggcctctcttgctaatggcttggtttggttttgcttttaattctagccagcctgcttttagactttctcaccttaaccactaggcccttccccttcagaatgtcagggctttctgaagtctagccagacctcattgtccctgtgatctcaaaggtcttctccttcacctcctacaaacaaaatttactttgccaaaacacaaggcattatgggaggcactgtgggggatacaaaaattataggacagagttatcttatcaccaggagctcacaagaagaacccaggaagcctggacaaaggtacttcttactgcagtagggtgctattcagtcaggcactgatggatcagggagaacgtgactccaggatgcagcagttgcggaaggaagagaaacagtcatggggaaggtaaaatgtattatgtttacactagaaaaacagattctaagcctatcacaaaaaggtcattgacatttcctgaatggataaccaggaactgttagcgaactgtaaacagggaggtatgcttccttgttagggggaggaggcaggactcaacccaggcatcaatcaaaatgggatgcttagaacagaacttatggttgagcctggactaaaagagtttatttttggtgggggtggcgggcggggggggcggcagacgtgacaggatgatgggaaggcagagaaaataagaaatgatttaggtgctttgggccaaatgaatctgatggaacaggatgtttccatagcacagtacaaaaagcattgctctgaccttgatgtctgtagactggattttgtacaaccagagccacagtctgggggcagggagaattctctacaaggcaacactgcagcctaggaagggggcctagttacatggcctatgagtcagagtttttctttctgaatcacagagaggccactgctggccaaaagaagcaatttagaaacgagagcccccttcctcctggtagctaaaaccatgtggcctaggtgtgctgcatttcactgggtacacaccagggtcatgcagcggaccagatgtgagtcactggcctggaattaaacaggcccagctctcccacttgcacccagctccccaacataactctcaggttcctcccaaaagaggtaatcacagcaaattcattctggtatttctaaccaggaagtgctggggaggaacagagatcaatttattttctcagaatagagtaagtttttccaatggaaagtacattattactaggactcaagcctccagccagtcaggttgtctttatccacttcagtttctttttcacagggcacctaaagggcatatgaagtattctgaattatttggcagctgaagccacttgtatgtgtagctgctaacaactgcccagtcctctctctttccctccctcccgtctcctctctgattccattaggcatagtactatttccacaaataccacaccttttgtttttctgtgatcatcaccctcatgctggcccatatttttgcttctatcttgcttcataaaagcatcaaacctgctcagctgctcagataagaaagttctgccaggtcaacaatggaaaaaaaccagttccacccagcacccaagttctttttcatttgtgccaaatttggacttgggaaatccttgcctttgaacaattgtgttcagattcatacaccccagatcgtgtttctttgctctgggatttcactcctctgcttctgaacaggatacaactaaaccagtttagctcttatcttggatgcaaacatcaccatgaagagtccctgttaggaacttttaatagtccctaagaggaaggctctcctattaccctgctccagggtcaatttcactaattttaggaaccctcttcctgatctaaattctgcttcaacccatttcctttgttcctcctcagcagcaatgataaacagctgtcctgttgattagagccaggtagactcctgatagcatccagccgcccttccctttaggctcaacctcaatcttttttacatgtctccctctgccctcacctctctcatccccacccccccgcccaccaccccacgtcgagctttttctcatccctttagtaatttatcacttccttcggggcttttcaagtggttcatgcaccccttatgaaacagcacctacatctgtaatttagtgagagactctgtccacctctgcccagcagaggtcaacatgaaaggtgacagaggaggggatggagggatgggactaaaacaggggcaaggtacatgtgtacctctccaacccagacagatggaaaagtactgaattattgaagaacatactgtacctttcctcccatggagattaaaatgcttcaactaagaaccaccagcaagcccgacagggaaagtaaggcacaaggagggcaggaacttgtcacaaataaagagctgtcaagtgcattcttgacacccagcccagagctctgcccaccttaattctgactcctgtgatcacagtaaagttgggcagaaatagaagagataagtgtctattacaaacacctgaagaacgatggattgaagatggacccaaacagccacatcaaggaaatatgtcatactttgctgaaaaaactgggtgtcaaagttagcgtccatccttgtgtcaatgagatctcaggcctcaacatccattttctttctgaggaacagtggttcattcacagacgcattcaggcaaaccagaacaagaaagaacatttaaagcactgctttgcaaatagatgctttttagcagcaatttttaccacgtcttaagattcaaatgacagattcatcctccaagaattagaaatgtaactaaacaaacaagcatatgtgtatgttggggaaattaaacagatccacatctaggttaaggaaattaggtactggaagaaaaccaaatgaataaaactaggtcaggtgtgggacctacaattgtggcagtatgtatgtaaatttaggaacagcagaaactagcagcctttctctcaacccctcacaccaacgctgtaggtttttgctacagctgagaaaatatttctcattaaaaaagagagaacaagaatgagttcatacatgcagggatgcaggcagagtcatacccacccaccaatattaaaatgtgtgtttttcttcagatcttaggaaattcaactttggctggaaccatcaactttgctacatcggccaatccaaccagctgggaattctagtaattttgctcactgcccccagacgactctatccccttatgaaggagacatatttgtaacccactgaacagaaaactggcctttaaaataatgtcatcctgctgtctcctttctgtggctacggtgctaatgtggtatttcaatttcttccctcaatatcctcttcattttaacaaagaatatttcttctcgaggccaggcacagtggctcacgcctgtaatcccagcactttgggaggccaaggcaggtggatcacctgaggtcaggagtttgagaccagcctggccaacatggtgaaaccccgtctctactaaaaatacaaaaattagctgggcgtggtggtgtgcacatgtaatcccagctacctggggaggctgaggcaggagaatcgcttgaaaccaggaggtggaggttgcagtgagccaagatcggaccactgcactccagcctgggcgacagagcgagactccatctcaaaaaaaaaaattaataataatatttcttctctttgcatcatacctcagtttggtatcactctaacccacagagcttcattttacttcatagcttttagcaatacagaacgatttctttatagtccatggcttccactaggatgtcagctccgtgaaaatgggaacttatcactactgtatctccagtacctaaaacagtgtctggcgcttgataggcactgaataagttcgtgttgactgaccgaatgaataaacatgcaggctgtcagccgaccacaattttcacttcccaccaacaccaaaccctaatctacaggtaatcaatcatggggatttaagatgctaattggtctccgtgccaccatggagccatgcctccagaaataagcatcccctgcctcacattctgccttacagcccacctgccagctcccactccttcccacagcatatcaaagggacacaggtaatactgtgttaagacccctttacaaggattatatgccttgcccttgtcatgctaattatagggaaactccagctttcttgcagagtttctcctgaaatttctcactctcaacataccttcaattcagcttcatttactggcctaacctcttgatctttcatttcactctcatttcctactaacaaagggttggtctagcttctcatttcccaaacaccaaaatatgctctctaaatcctcctctgtgagaatcactccctggtttagtctcataatgctttaccaaaaagctaagcaggctgggtgccgtggctcacgcctgtaatcccagcactttgggaggcgaggcgggcggatcacttgagatcaggagttcgagaccagcctgaccaacatggcaaaaccccgtctctactaaaaatacaaacattagccagatgtggtagcgcgtgcctgtagtctcagctactcaggaggctgaggcaagagaattgcttaaacccaggaggcaggggttgcaatgagccgagatcgcgccattgcactccagcctaagcaacagagtgagactccatctcaaaaaaagaaaaaagaaaccaggtttttaacgtgtcccagataccttagctttctttgtgtcctgattgaggggtattataagggaaatgttatctgtgggacactttaacaaagatctacaaaataatgataacaatgtcagcacttcccagttactcaattttatccatcagtttcatccttctgcttctcatagcaggatcttcaggaatcagctctttctctttctactttaccctatacatactccttttttttttttttttgtgagacagagtctcgctctgcgacccagactggagagtgcagtggtgcgatctcggctcattgcaacctccacctcttgggttcaagcaattctcttgcctcagcctcctaagtagctgggattacaggcacctgccaccatgcctggctaatttttgtatttttagtaagagatgagatttcgccatgttggccaggctggtctcgaaactcctgacctcaagtgatctgctcaccttggcctcctatacatactgagccaccgagcccgacccctatacatactcctaaacgttgctcaatctctccccagagttcttcacccactctcgagtctctatcctcttgcttcatttcagtccatctcagtttatgacacattactgtttgggttctcaatcattatcccaagagccacgatgacttttacttagaccaaagtcaagttcatccttacaacccacaagtgaggttgatcagaacccagagagaagtttagcaaaggaggttaaaacgaagtaaataattccaaacggtatcttgagaaaactaaatcctcattgctggtctgccatgatcatcactaatatcaaacaacaaatgttggaaaccgcaatgcccatatggcactggataagttacgaaacctctctttgcttctgttctcccatctgtcttgcaagaacaatagtgctcgctccctggctcctaaaggtatcaagacagggaaagcttttttttttttttttttttttgagacagtgtcttgctctgtcgccaggctgaagtgcagtggcgcaatctcagctcactgcaacctccacctcccgggttcaagtgattctcctgcctcagcctcccaagtagctgggagtacaggtgcgcgccaccacacccaggtaatttttgtatttttagtagagatggggtttcaccatgttggccaggatggtctcgatctcttgacctcatgatctgcctgcctcggcctcccaaagtgctaggattacaggcgtgagccacagcgcctggctggaaagcattttttaaaaaacaaaacaaaaacaacagtgtcttaaaataagactaaaaaattgtcaaaataaatcccttgacaaacttattaaaaaacagagatccaggcctgatatcagatgttaatcatgcatctaaccaaataatatcttaatttgggagtaggaggagtgcagaggggagtggagggggctttactatataaccagaaatctaggctagaattgtggctgtaaattcactctggcatgaccgaacagtaccaatgccaagagtagcactcagatgatgcaccaaagcccccccaaaatctagagtttcctggatgctaccaccaggatccagtcatggcaacagggatcttaaaggtgtaaatgattaaccttcaggatggctcacctagacgtgcaaacgtttgaaaaagctaagctctctaccagggtaacccattggctctcatctcttagatttcgtaagagttttaccttctgggtaacatttcaaagccctgctaagattagtatctacacctgaactctggaccatggctcccttccagccactatctctaggctgtcaatcggggttaattcgcttttttttttttttttaatcctgtactctaatcataggaatttcaaccaagtttcagctggcttctgcatctcacactccagcagggacacaagcccgaaggaagaaaggtgcccgtccatctccaggccggctttccccacctttttcctttttgctttccacctgcatgaagttccctctaaaggtgcatgtcattttctggccctttaagtcccgacaccccatcccactccgagttcccttccatcctccgcactttctccctccccacttgcagtttgcctcgaccccctcggggagtttctcggtccctctccgggcgtctttctgcacccgtacccttctctccgcagagcgactccggccttgcttcctctcccctgccctttcccctccccaccacaccgctgcagcagacgaccccgtccccacagccggcgcgttccctgacctcccctccgcccctcacctcggcccccttccctcgcgccccggagccccggcctccccaccccgcccccgggtccccccacgccgacctcggccactccctcccggtcactcacaggaggccggagcaggtggtgcacacgaagctgcccacggtgatatccacgtaggtgaccccgcgctgggcgcactcgaagcagtggcggttcccggcctggctgcagccacccagctcccgcacccgacggcaccacacctccgaggccgcctccgcctccgccttgcccccgctgaccccgccgcccgggcccgggcccttcttcgccgccatcaccatcgctgcccactccctccccgctagtcagcaatcaccccttcaaccactgccgccttcccgcctcctcagccgcctccgcacgcccggctcccctgacagaagctcgggagcgggcggcatccgcaggagacttcgccgtccaccaccctccttgcccgtcctgcacactgattggctaggtttctcccacctccttactccgattggccctccctgggacggcctccggttgctggagccgccaatgccccgccccaccgccttcaggcctcaggcctctcctattggccggcagagaccccagcagcgagaaccgtgcgtgccctctccttgtccccgccccctctcgccgctgcctccaccgtggcccgcctcctagttccgattggtgagtcctcggcactcgggctccgacgctaattttgattggctggcggggctgcaaagttagcgcgcgagacagaggaggtgccccgcgcgtgcgtagctgtcgtcctcccgactgatgacgttaattcccggcaaatttcaaaggcttactctagactgtgtagagtgtcatgcaaagccgaagagaagtggccatgtgaggggctgagcaggagggggaaagaagagagcgcttaagggagagagggagagggagcaagagtgctttactagaccagtgctctaattcctgagccacagagcgaagggagtgctttataaatgtcatttcattcgctccgcctatcccagaggtggaatccccattttacacccttaggttcaacaacttgcggcagatcacacgagtgcgtggcccaggattccactctgggtcttccaactttagaaaccctgcctttaaccctcctagtaggtggactattattcgattattcatttaacaaacactgagtgtctgctaggtttcaggcacgttctagacactgggaatagaatacagcagcgaagcaatcaaacaaaatattcttatagagcttagtttctagtagggggaagcaaacaatacacaaattaaagaatcaccagttagtaataaatcctacgcagagaattaaaatcacatcttggaaaagactttttgggtacttttttaattttttaatttaattttaattttttgagatggagtctcactctgttgcccagactggagtgcagtggcacgatgtcagctcactgcaacctctgcctcctgggtttaagcaattctcatgcctcagtctcctgagtagctgggattacaggcggccaccaccacccccagataatttttgtatttttattattatgattattattatttgagatggagtctcgctctgtcgcccaggctggagtgcagtggcacgatctcggctcactgcaagcttcgccttctgggttcacgccattctcctgcctcagcctcccgagtagctgggactacaggcacccgccaccacgcctggctaatttttttgtattttcagtggagacggggtttcaccgtgttagcaaggatggtctcgatcttctgacctcgtgatccgccggtcttggcctcccaaagtgctgggattacaggcgtgagccaccgcgcccggccaatttttgtatttttagtagagacaggatttcaccatgttggccaggctggtctcaaactcatggcctcaagtgagcctcccgccttggcgtcccaaagtgctgggattacaagcatgagccacaatgcctggccacctgttgggtacttcagattgggttggtcaccatttttaaaacaattactttttttttgagacagggtctcgctttatcacccaggctggagtgcagtgacacaatcatggctcactgcagcctcaaccttctggactcaagtgatccttccacctcagccttccaagtagctgggagtacagctgtgcaccaccatgcctggcgaatttttaaaaattttttgtagagattgggtctcactatattgcccaggctggtctggacctcctgggctcaagtgatccgcccacctcagcctccatctcccaggttcaagcgattctcctgcctcagcctcccaagtagttgggattacaggtgcgtgccaccatgtccagctaatttttgtatttttagtagagatggggtttcaccatattggccaggctggtcttgaagtcctgacttcaaatgatccacccaccttggcctcccaaagtgctaggattacagacgtgagtcaccgcacccggcctctttttgttttgttttgttttttttgtttttttttttttttttgagacaaggtcttgctctgctgcccaggttggagggcagtggcacaatcatacctcactgcagcctcaacctccccagttcaagtgatcctcccacctcagtctccagggtagctgagactacaggcgcacaccaccatgcctggctaatttttgattttttgtagagaaggggtctcactatgttgtccaggctggtggctggcagcctgctttctctctctctctcttgtttttttctttttgagacagggtcttactctgtcacccaggctggagtgcagtggcacaatcttggctcactgcaacctccacttcctggggcttgagtgctcctcccacctcagcctcccgagtagctgggactacaggtgcatgccaccatgcccagctaattttttgtatttttagcatgttggccaggctggtctcgaacacctgagttcaggtcatctgcccgctttggcctctcaaagtgctgggattataggtgtgagccaccgcacccggccccatttttcaattaatggcaccatgagccacctagttgcccaaagcaaaaatctagagctactctcttcaatacggcagccactaatcatatgtggctatttaagtttaaattaaattaaaatgggctgggcgtggtggcttatgcctgtaatcccagcattttgggaggccgaggcaggcggatcacgaggtcaggagttcgagaaaagcctggccaacatagtgaaaccctttaatctactaaaaatacaaaaattagccaggcatggtggcaggcacctgtaatcccagctacttggaaggctgagtcaggagaattgcttgaacgtgggaggtggaggttgcagtgagctgagatcgcgccattgcactccaccctgggtgacagagcgagactgtctcaaaaaaaaaaaaagaaagaaagaaagaagaaggaaagaaaaagaaaaggaaaggaaaaggggagactaaaggaggagtaggttagggcagggcctaacctactgggccatggaccagtacctgtctgtggcctgttaggaaccaggccacgcagcaggaagtaagtggtgggcaaatgagcaaagctgcatctgtatttacagccactacccattgctcccattaccgcctgagctctgcctcctctcagatccgcaacagcattagattctcataggagcacgaaccctattgtaaactgcgcatgcaagggatctaggtcatgcactccttataagaatctaatgcttgatgacctgtcactgtctcccatcacccccagatgggaccgtctagttgcgagaaaacaagctcagggctcccactaattccacatgatggtgagttgtataattattttgttatacattacaatgtaataataatagaaataaagtgcacaggccaggcgcggtggctcacgcctctaatcccagcactttgggagctgaggtaggcagatcacgaggtcaggaaatcgagaccatcctggccaacatggtgaaacctcgtctctactaaaaatacaaaacttagctggccatggtggcacgtgcctgtaatcccagctacttgggaggctgaggcaggagaatcgcttgaacccaggaggtggaggttgcagtgagccaagatcgtgccactgcactccagcctggtgacagagcgagactccgtttaaaaaaaaaaaagtgcacaataaacttaatgtgctcagatcatcccaaaatcatcccccaactcccacccgccggttagtggaaaaattatcttcctggtccgtggtgccaaaacggttggggactgctgggttagggggtgctgataggcagaaatcagtagcttggttttggacatgttgatttgagatccttgtgaggcatccagtgggctgataagcaggcaggtggatatgtatagactggatttccaggccaggccagagctggaaatgtaggtttaggagtcatcgcttagagccatgggactggttgcttaggtacagcatgtagagagagactagaagcaggcccagaagtaaaccctaggcacccaatgtgtactttgttccttttctccagttatcacagccacatgcatttcaacatccctaaaaaaaggcaacaccagaccgggtgcagtggctcatgcctgtaatcccagcactttgggaggccgaggggggtggatcacctgaggtcaggaatttgagaccagcctggctaacatggtgaaaccccgtttctactaaaaaatacacaaaattagctgggcatggtggcgcatgcctgtaatcccagctactcaggaggctgaggcagaatcgcctgaacccagaagttggaggttgcagtgagctgagatcgcaccattgcactccagcttgggcaacaagagcaaaactccatctcaaaaaaaaaaaaatacaaaaatcagctcggtgtgatggcgcatgcccgtaatcccagctactcaggaggctgaggcacaagaattgcttgaacctggaaggcagaggttgcagtgagccaagattgcatagctccagcccaggtgacagagcgggactctatccccctcacccccctcttcaaaaaaaaagaaggcaataccgtcacatccagatatgcagatggggcatctcagcctactaaacattccttctgaattgcagagttcacaatttccaagtgctgcccatgcctcctctagcttctcattagccaagggttatttcttttttcttttcttttcttttctttttgagatggagtctcactctgttgcccaggttggagtgcggtggcacaatctcggctcactgcaacctccgcctcctgggttcaagcgattctcctgcctcagcctcatgagtagctgggaccacaggcgcatgccaccatgcccggctaattttttgtacttttagttgagacaaggtttcaccatgttagctaggatggtctcgatctgctgatctcatgatcagcccacctcggcctcccaaagttctgctatacaggcgtgagccaccgtgctcagcattttttttttttttgagatagagtttcactcttgttgcctaggttgagtacaatggtgcgatctcgactcactgcagcctccgcctcccaggttcaagaaattctcttgcctcagcctccctagtagctgggattacaggcgcccaccaccacacccggctaattttttgtatttttagtagagacggggtttcaccatgttggccaggctggtcacgaactcctggcctcaggtgatccacccgcctcagcctcccaaagtgctggaattacaggcgtgagccaccgtgcccagctgagccaagggttatttcttatcatcttgcaactttgagcaaattggtaaatttaactaccaccaatagtgagtgaaaaaaattagaacgttcatgtagatagtgatggttttctgctgatttttgccactgggtgtggtggtgtgagggccgttgaggagctcaccctcctcccatctctactccacccaggtcatactggggagcaacactcccattttcccttcataatcagggggagcacagccagcatggggagaccttttgccctggtggcaggaggagtcctaaataaccaggtagaactcttattgggttccctggcaagaaccttgcccttgggcagttaaaccacagagcatgagtgacttggggcgtggaaagcaaatatctttttttttttctttgagacggagttttgctcttgttgcccaggctggagtgcagtggcgcgatctcagttcactgaaacctcccgggttcatgcaattctcctgcctcagcctcccgagtagctgggattataggcgcccaccaccatgcctggctaatttttgtatttttggtagagacggggtttcaccatgttggcgagactggtctcaaactcctgacctcaagtgatccagctgcttccacctcccaaagcgctgggattacaggcatgagccaccgtgcccggccagcaaaaaaattttagagagaaaaataatagtgacagctactccccactgtcccccttaactgcaacccttggtttctggacctctctattgactgaggaagactgggcgctgcaattcagtcactgtgaggatcacatatcactatcagcgagggagcgttgcagcctcacaagtgtggtctcgtggatactggctagatttgtttttggtacaccagttcattgttctacaaggccagctgtttctggtacatgggaaataggataatggggcccatggctggccaactgtcttgtttttcttttctttctttctttctttttttttttttttttttttgacacagggtcttgctctgttgcccagcctgcaatgcagtggcaccgtcatagctcattgcagcctctaattcttcggttcaagcgatcctcttacctcagcctccccaggaggtgggactacaggcgcacaccaccatgcctggctagcttttttattttttgtaaagagagagtttcaccatgtctcccagactggtctcgaactcctgtgctcaagcaatctgctcaccttggcctctcaaagtgctgggattaattataggcgtgagccactgtggccagcccatggtaacttggttttgtttgtttgttttgtgtgtgtgtgtatgtgtgtgtgtgtgtgtgtgtgtattgagatggagtcttactctgtcactcaggctggaatgcagtggcatgatttcagctcactgcaacctccgcctcatgggttcaagcgattctccctcctcagccccccaagtagctgggattacaggcacctgtcaccacgcccagctaatttttctatttttagtagagacgggtttcaccatgttggccaggctggtctcaaactcccgacctcaagtaatccacccgcctcggcctcccaaattgctgagactacaggcataagccatgtgcccatcctatggtaactttgtatcccagatatttacactaaatttcaggaacaagttaggagatgttccccggaagaagaataattatctgctgaaatgtggtcttgtttcaacaccccagaggcctccagggagattgtctactgggcatggcaaaggttgagtgggctagacccctctggcaccactggattagccaagccataagggcatcttgctttgtaacctggactaatttagaaacttcctctttcacacaggtgcggtggctcatgcctgtaatcccagcattttgggaagccgaggcgggcggatcacgaggtcaggagttcaagaccagcctggccaaccaacatggtgaaaccccgtctctactaaaaatataaaaattagccaggcgtggtggcacacacctgtaattccagctactcaggaggctgaggcacgagaatcacttgaatcagtgaggtggaggttgcagtgagccaagatcatttcactacactccagcctgggtgacaaagagagactctgtctcaaaaaataaataaataaataaataaaaagtaaaaatgcccaagtctatcctccagaaatgaaatctggaatgtctgagatgaaaagtacagtgaatgggactaacagcagactagatactacagaagagaatattatgaacttgaagacataacagtagaaagtataaaaatgatttatagagctaggcacagtggtgtgcacctataatcccagctacttgggaggctgaggctggaagatcagttgagcccaggagtttgagaccagcctaggcaacatagcgagaccctgtctcttattttttaaaatattggaaaaatgaaaaaagactgataaaaacccaaaacccacagatgaacagaacaccagtgagctgtgagacaacttcaggtggccccctcaggaattggtggcaacaggtataatacatctggggtaaaggtagaggttaaaaataagtatattgactgaaaatcagttttattttattttattttattttattttattttattttattttatttttaaaaattttttttgagatggagttttggtcttgttgccctggctggcgttcaatggcacgatctctgctctctgcaacatccacctttggggttcaagtgattctcctgcctcagcctcccaagtagctgggattacaggcacccgccaccacacccagctaattttgtatttttagtagagacgaggtttcgccatgttggtcagcctggtctcaaactcctgacctcgtgatccacctgcctcagcctcccaaagtgctgggattacaggcgtgagccactgtgccctgcctaaatgtcagttttaaaagacattagagccagatatggtggtgtgcacctgtagtcacagctattcaggaggctgaggcagaaggattgcttgagtccagaagtcctgggttgtagtgtgctctgctgattgggtgtctgccctaagttcaacaacaatgtgctgaccttccagaagcgagggaccaccaggttgcctaaggagggttgaacctgcccaggttggaaatggagcaagtcaaaactcctgtgctgatcagcagtggagtcacgcctgtgaatagtcactgtactacggcctgggcaacatagtgagacccgtctctaaattaaaaaaaaaaaaagggcattagactcacacaaacgttggttgcacttctatatattaaatatgagcaatgtcaaaaggaaattacaaaaacaattacattggccaggtgtggtggctcacctctgtaatcccagcactttgggaggccaaggcgggcagatcacttgaggtcaggagttcgagaccagactggccaacatggggaaacctcgactccactaaaaaaaaatacaaaaattagttgggtgtagtggcccacgcctgtagtcccagctacttgggaggctgatgcaggagaatggcttgaacctgggaggtggaggctgcagtgggcagagatcgtgccactgcactccagcctgggtgacagagtgagactccatctcaaaaaacaaacaaacaaacaaacaaaaaaaccccacaaaacataaaaacaattacatctacaatagcatcacacagaataaaatacttagggattaatttaaccctaattaaattaaggaggtgaaagacttgtaaaatgaaaactatacaacattgctgaaagaaattaaagaagacaaaaatttatggaaacacatcccagttcatgaattggaagagttaatattgttaagatgtgaatactaaccaaagtgatctacagatttagagccaggctcagtggctcactcctgtaatcccagcactttgggaggagtaggtaagcggatcacctgaggtcaggagttcgagaccaccctggccaacatggagaaaccccgtctctactaaaaatacaaaaattagccaggcatggtggcatgcacctgtaatcccacctactcagaaggctgaggcaggagaatcacttgaacccaggaggaggaggttgcagtgagccgagatcgcaccgctgcactccagcctgggtgacagagcgagactccgtctcaaacaaacaaacaaaaaccaacaaagtgatctacagattcagtgcaatctctatcaaaattccaatgatgtgttctgcagaaatagaaaagcccatgctaaaatttctatgaaatctcagcggactccaaatagccaaaacactattcaaaaagactaacaggctgggcatggtggctcacgcctgtaatcccagcactctaggaggccgaggtgggcagatcacctgaggtcaggagttcaagaccagcctggccatggtgaaaccccatctctactaaaaatacacaaaattaggcgggcatggtggtgcatgcctgtaatcccagctactcgggaggctgaggcaggagaatcacttgaacccaggaggcggaggttgcagtgagccaagatcgtgccattgcactccagcctgggcaacaggagcgaaactccatctcaaaaaaaaaaaagggactaacaaaattggaggactcacacttcttgatttcaaaacttattacaaagccatgtgatcaaaacagcatggtactggcataaagacagacatataggctcgggtgtggtggctcatgcctgtaatcccagcactttgcggggccgaggtgggtggatcatttgaggtcaggagttcaagagcagcctggccaacgtgctgaaacgccgtctctactaaaaaaaaaaaaaaaaaaaaaaaaaaaaaaatacaaacattagccaggtgtggtggcacgtgcttgtcttcccagctactcgggaggctgaggcaggagaatgacttgaaccagggaggcggatgttgcagtgagcctaaattgtgccattgcactccagcctgggcgacagagcaatactccatctcaaaaaaacgaaacaaaacaaaacaaaaaaaacctgtcttttcaacaaatggtattgggataactgcatatctacatgtgaaagaatgaagtggaactcttacctaacaccatatacaaaaattaactcaaaatggataaaagacctaaatgtcagacctaaaactaagaaagatttagaataaaccatagggcaagagcttcatgacattggattttgcaattacagttgacccttgaacaaagcgggagttagggacaaaggacttgaacagacatttctccaaagaagatacacaaatggccaataagcacacaaaaagatgctcaacatcattagtcattaggaaatgcaatcaaagccatggatgagataccacttcacatccactaggatggctgtaatcaaaaaagggaaaactagcaattgttggtgaggatgtagagaaattgtaacccttgtacattgctggtggaaatgaaaaatggtgcagctgccatggaaagcagtttaccagttcttcaaatagttaaatatagaggccgggcacagtggctcacgcctgtaatcccagcactttaggaggccaaggcgggcggattgcctgaggtcaggagattgagaccagtctggccaacatggtgaaaccccatctctactaaaaatacaaaaaataaaaaataaataaaaataaaaaattagccgggtgtggtggtgtacacctgtaatcccagctactcgggaagctgaggcaggggaattgcttgaaccagggagacagaggatgcagtgagccgagatcatgccactgcactccagcctgggtgacagagtgagactctgtctcaaaaaaaaataaagttaaatatagaattactatatgactcagctgtttcactcctagttatataccctaaggagtttaaaacagctattcaaatcaaaacttgtacacgaaagttcatagcagcaccatccatgatagcctaaaggtagaaataatctaaatgtccattgacagatgagtggatagagaaattgtggcatatacagaacggggcatggtggctcacacctgtaatctcagctttggaggctgaggcaggaggattgcttgatcccagtagtttgagacgagcctcgacaacaagtgagaccccatctctacaaaaattttaacaaattaaaaaattagccaagtgtggtggcacatgcctgtagtaccagctacttgggaggctgaggtgggaggatcacacagcccaggagcaagagcctgtcttaaaaagaaaaacaaaaacaaagagaagcaattggaaagaaacagatactatgtaaagagaagaaaattttacccaaaacccactataattaatatccttggagagataagataccattttcatgaaatatacacagaacgttattttaaaaggaatattcaggccgggtgcagtggctcccgcctgtaatcccagcactttgggaggccgaggcaggtggatctcctgaggtcaggagttcgaaaccagcctggtcaacatggcgaaaccccgtctctactaaaaatacaaaaattagccgagtgtgatagtgggagcctgtaatcccagctacttgggaggctgaggcaggaggattgcttgaactcagcagacggaggttgccgtgagcccagatcatggcattgcactccagcctgggtgacagagcaagaatccgtcaaaaaaaaaaaaaaaaaaaaaaaaaaacagaaaaaaaggaatattcagagaacaagaaagaaatcctggaaattaaatatataaacatgaaaacctcagttgaatggttggaagataaaattggagaaatcctgcaaaaagcagtttaaaatatgagatgaaaaaaatgattaaaggtaaataaaagaacagttcagtagatctaatatataaataatagaagttacagaaggagagaagagaaaaatggaggggaaaaatattactgaaaaatttagcaaaattttccagaattaaagggcatgagtttctagattaacaagaccttagtcaggtgtggtggctcacatatgtaatcccaacactttgggaggctgaggcaggtggatcacctgaggtcaggagttccagaccagcctggccaacatggtgaaacccggtctttactaaaagtataaaaattaggccgggcgcggtggctcacgcctgtaatcccagcactttgggaggccgaggcgggaggatcacgaggtcaggagatcgagaccatcctggctaacatggtgaaactccgtctctactaaaaatacaaaaaattagccgggtgcagtggcgggcgcctgctactcgagaggctgaggcaggagaatggcttgaacccgggaggcggagctttcagtgagccgagatagcgccactgcagtccagcctgggcaaaagagcgagactgtgtctttttactttttttaaaaagaagtataaaaattagcttgccgtggtggcgtgcacctgtggtcccagctactcaggaggctgaggcaggagaattgcttgaacccaggaaggagaggttgcagtgagctgagattgcgccactgcactccagcctgtgtgacaagagtgaaaccacctcaaacaaacaaacaaacaaacaaaaaaaaaaacaaactaaaaaacaagacctatgaagtgccatggtataactgataaaaaccaagaaccggctgggcgcggtggctcacgcctataatcccagcactttgggaggccgaggcggatggatcacgaggtcaggaattcgagaccagcctggccaacatggtgaaaccccgtctctactaaaaatacaaaaattagccggacatggtggcgggcgcctgtagtgccagctactccagaggctgaggcaggagaatggcttgaacctgggaagcagaggttgcagtgagccgagatcgcgccactgcactccagcctggcgacagagcgagattctgtctcaaaaaagcaaacaaaaaacctaagaacctcacgaaagcatatccttgtgaaatttagaatggtggagataaaaaaatatcttacaagttcctaagagtacgaacaaatgacctatattaggatttttaatagcaaccctggaaggtagaaggacaaggtgcattgcattcacaattttataggaaaggccaggcatggtggcccatgcctgtaatcccagcactttgggagattgagataggaggaccacttgaggctaggagttcgagaccagcctgggcgacattttgagattctgactctacaaaacagaaaaaaaatttgaaggaaatcattttcaacaagtaattccatacatggccaatctacaactatgaattattctagaataaagatattttcagtaattcaaggttcagatgcaagtgacagaaaacctaaaacaatagttacaattttctttctctctgacataaaagtctaggtcagtggccaagcgtggtggctgacgcctgtaatcccagcactttgggaggctgaggagggcagatcacgaggtcaggcgatcgagaccatcctggctaacatggtgaaaccccatctctactaaaaatacaaaaaattagcagggcgtggtggcacatgcgtatagtcccagctactcgggaggctgaggcagaagaatcgcttgaacccaggaggcggaggttgcagtgagccaagatcaggccactgcactccagcctgggtgacagagtgagattccatttcaaaaaaaaaaaatctaggtcagtatagtggctctgttccatgaaactctcagcaacccaagttcattccagccccaactctgcaatgcccagggtgtggccctcatcttcatggataggatggcagcatgtgagcaccagacactgggtgatagaagagatgatgccctctccctctccctctctttccacggtctccctctccctctccctctccctctctttccatggtctccctctcatgccgagccgaagctggactgtgctgctgccatctcggttcactgcaacctccctgcctgattctcctgcctcagcctgccgagtgcctgcgattgcaggcgcgcgccgccatgcctgacgggtttttgtatttttttggtggagacggggtttcgttgtgttggccgggctggtctccagctcctaaccgcgagtgatccgccagcctcggcctcccgaggtgccgggattgcagacggagtctcgttaactcagtgctcaatggtgcccaggctggagtgcagtggcgtgatctcggctacaacctccacctcccagccgcctgccttggccccccaaagtgccgagattgcagcctctgcctggccgctaccccgtctgggaagtgaggtgcgtctctgcctggccgcccatcgtctgggatgtgaggagcccctctgcctggctgcccagtctggaaagtgaggagcgtctctgcccggccgccatcccacctaggaagtgaggagcgcctcttcccggccaccatcccatctaggaagtgaggagcgtctctgcccggccgcccatcgtctgagatgtggggagcgcctctgccccgccgccctgtctgggaggtgaggagcgtctctgcccggccgccccgtctgagaagtgaggagaccctccgcctggcaaccgccccatctgagaagtgaggagcccctccgcccggctgccaccccgtctgggaagtgaggagcgtctccgcccggcagccaccccgtccggaagggaggtgggggtcagcccctgccaggccagccgccccatccgggagggaggtgggggggtcagccccccacccggccagccaccccgtccgggaggtgaggggcgcctctgcccggccgcccctactgggaagtgaggagcacctctgcccggccagccgccccgtccaggagggaggtgggggagtcagccccccgcccggccagccgccccgtccgggagggaggtggggggggtcagccccgtgcccggccagccgccccatccgggagggaggtgggggggtcagccccccgccaggcgagatgccccgtccgggagggaggtgggggggtcagccccccgccaggtgagacgccccgtccgggaaggaggcgggggggtgggggtcagatccccgcccggccagccaccccgtccgggagggaggtgggggggtcagccccccgcccggcgagacgccccgtccgggagggaggtggggggtcagccccctgcccggccagctgccccgtccgggaggtgaggggcgcctctgcccggccgcccctactgggaagtgaggagcccctctgcccggccagccgccccgtccgggagggaggtgggggagtcagccccccgcctggccagccgccccgtccgggagggaggtgggggggtcagccccccgcccggccagccgccccgtccgggagggaggtgggggggtcagcccccagccggccagctgccccgtccgggaggtgaggggcgcctctgcccggccgcccctactgggaagtgaggagcccctctgcccggccagccgccccgtccaggagggaggtgggggagtcagccccccgcccggccagccgccccatccgggagggaggtgggggggtcagccccccgcccagcgagacgccccgtccgggagggaggtggggggtcagccccccgcccggccagccgccccgtccgggaggtgaggggcgcctctgcccggccgcccctactgggaagtgaggagcccctctgcccggccaccaccccgtctgggaggtgtacccaacagctcattgagaacgggccatgatgacaatggcggttttgtggaatagaaaagggggaaacgtgggaaaaagattgagaagtcggatggttgctgtgtctgtgtagaaagaagtagacatgggagacttttcattttgttctgtactaagaaaaattcttctgccttgggatcctgttgatctgtgaccttacccccaaccctgtgctctctgaaacatgtgctgtgtccactcagggttaaatggattaagggtggtgcaagatgtgctttgttaaacagatgcttgaaggcagcatgctcgttaagagtcatcaccactccctaatctcaagtacccagggacacacacactctgcctaggaaaaccagagacctttgttcacttgtttatctgctgaccttccctccactattgtcctatgaccctgccaaatccccctctgcgagaaacacccaagaatgatcaataaaaataaataaataaataaataaaagaagagatgatgaagaaggcaccaggggcatgtgccagctatttcttcagaaagttcttccaaagaactttcacttctccttgcatcccattggtcagaatttggtcacatggccacaccaagctacaaggtagggtggaaaatgcagaaatatagactttattttgaattatcttttttttttttcttttttgagacagcgtctcactctgttgctcaggctggagtgcagtggcacaatcatggttcactacagcctggacctcctgggctcaagtgatcctcccagctcagctgctggagtagctgagactataggcgcatgccaccatgcccagctaattttctcattttttgtagagacagagtctccctatgttacccaggctagtcttgaactcctgggctccagtgatcctactgccttggcctcccaaagtgctgggattacaggtgtaagccatagtgcctggcctttggatggttttatgcctagataaaaactctattactatggaagaatggaaggatactggtggaattagtcacattctctgctacacagtcttgaacatttcctgaacattctcaggaagctatcagtagagtacaccacctacgtaaagcgataaagcaagaaagaagacttgggatccatgagtgagggtatccaattcaacaaagacgaagggagtccctagggtaatagtgagaggagaccccaaaatgacagctgagttgctgccctagaaagcagctagtccaaagtagagaagatcagaaggtaggtttcacagagagcaggcaataagatttttaaaaaggtaaaaaaaaaagaaacaaatcagaaggttcttggagggattccttcaagaagatgaaactgactgaagacctaataatttaccttcacctagcaggtgcagcaatatgccttcactgccctgtgacaatctactctgtatgtttgaacataatgagaagacttattttacagcagggattcaattaggattaatacctagaaacatcggcaaaccaaaagataaggcaattattaactccaagggaaacaaatggctcaattatgaatagcatatatacacttataaaaatataaccctaggacaggcgcggtgggtcacacctgtaatcccagcactttggggggccgaggcaggtggctcgcctgaggccaggagtttgagaccaggctgaccaacatagcgaaaccccgtctctactaaaaataccaaaaaaaaaaaaaattagctgggcgtggcagtgggcgcctgtaatcccagctacttgggaggctgaggcaggagaatcgcttgaaaccaggaggcggaggttgcagtgagccgagatcgtaccattgtactccagcctgggtgacaagagtgaaactccgtctctctctctctctctctctctctctctctctctctatatatatatatatatatacacacacacacacatacttacacacatatacgtatatatatgtgtatacatatacacacatatacatatatacttatatatatatgtatacatatatgtgtgtgtatatatatacacacacatacataaaatcctaaatctctttctaactaaaatgacaaatacagagcctccctgacttatgatggaactgtgtcctgattaattcattataaaatcaagaaaatccttaagtcaaaccatcctagctccctgacttatgatgggactgagtcctgattaactcattgtaaagttaagaaaatcattaagtcaaaccatcctaaattgggggactgtctgtgttcagaggatggtgggatgggaagtgtgtgtgtgtgtgtgtgtgtgtgtgtgactggtgtgtgtgtgtgtgactggtgtgtgtgtgtgtgactggtgtgtgtgactgggaagtgaaagagagctaaccctcactgtccacacggtaagtcaatagataataggtaatactgaaaaaaaaaaccaagagctaaagacataactgtattatttagacatataaatataaaccaaataacaaaagatcagctataagaactgaaaaaatgcttacttctgaggagctgaaaatggaggagagttgaaaaggatcttctcttttttctaacatgtctttggaagtatttaactctttaaattacgaccacgtccaaatttattaaaaataagacacaaatttggctgggcgtggtgactcatgcctatacttccagcactttggggggccaaggcaggaagatcgcttgaggccaggggttagacgccaggctgggcaacacaacaagactccatcactacaaaaaataaaaagtaaaataattagccagtgtggtagccagctactagggaggctgaggtgggaggattgtttgagcccaggagtttaaggctgcagtgagccatgatcatgccactgcactactgcagcctgggcaacagagtgagaccctgtttccaaacaaaacaaaacaaaacaaaacaaaacccaacctaaaacataagttttaaaaaatgaaaagataggctggccgcggtggctcatgcctgtattcccagcactttgagaggccgaggcgggcagatcacctgaggttgggagttcaagaccagcctgaccaacatggagaaaccccatctctactaaaaatacaaaattacccgggcggtggtggtgcatgcctgtaatctcagctactcaggaggatgaggcaggagaattgcttgaacccaggaggcagaggttgcagtgagctgagatcgcgccattgcactctagcctgggcaacaagagcgaaactccatcttaaaaaataaataaatatgaaaagataaattaatcagattttctttcttgggccttttaggtgagaaatgtggaaccgggtagaaggagacaaacactaacaagtggtaggtggtggctaaatcacattgacacttggagggagagggaacattcaccaattcctttttttttttttttgagatggagtctcgctctgtcgcccaggctggagagcagtggcacaatctcgtctcactgcaacctctgcctcccaggttcaagcgattctcctgccttagcctccctagtagttgggactataggcgtgtgccaccacacccagctaatttttttgtatttttagtagagacggggtttcaccgtgttagccaggatggtatccatctcctgaccttgtgatctgcccgcctcagcctcccaaagtgttgggattacaggtgtgagccaccgtgtctggcccattcgccaattcctattgtgaagcacctagagtttttcctggactcagaacttccttgggtttcaacctacatgaattctgattccactatggcttagttctaagagatcaggtctcccttatctatctctttctcttacttgagctttgttgagtgaacttttttgtttcttgcagtgaaaaaagccctgctaaaatagaggtagatgctgcttccagaattccttattagttgaaagtataaaatgtagcaagttggcctgatgcagtggctcattcctgtaattccagccctttggtaggccaagacaggatcacttgagctccggtgtccaagaccagcatgggcaacatagggagacccacatctctacaaataatttaaaaataatatgactgtgacaaatgattggaaagaaaaaaaatttaaaaattaataaataatttttaaggccaggcgtggtggctcacacctgtaatcccagcactttgggaggctgaggcaggtggatcatgaggtcaggagatcgagaccatcctgactaacacggtgaaaccctgtctctactaaaaatacaaaaaaattagctgggtgtggtggcgggtgcctatagtcccagcaactcgggaggctgaggcaggagaatggcgtgaacccaggaagtggagcttgcagtgagccgagatcgcgccactgcactccagccgggatgacagagcgagactttgtctcaaacaaacaaacaaaaataataataatttttaaaaagaaataaaaattagctgggtgtggtggtgtgtacctgtggtcccaggtactccacaggctgaggtgggaggctgaggtgggaggatcacttgagccctggaggttgaggcagcagtgggccatgatcatgccactgcactccagcttgggtgacagagcaagaccctgtctcaaaaaataaaataaataaaatgcagcaatttgtccttcattctctgccctcccatcccccacattttgaaggagatcttatattactaaggaatattcattgctaattattattcattactaatgaatattcattacaacttactattcattactaatgattttctaagctctactgatgtgacaggtccttgcatttctgccagttatatctcacaaccccattacatccacgtctcagcaagacctgggggatctttgatacttcaagtccaaggcaaatcagcatgatattgtcaatgacgatcaggatgacatcctacagaggagtcagttagtgtgatcaaccatcccagtttgccagggacctgggggcttcctaggatgcagaactttcagtgcccgaataacagtttcaggtaaacctagatggctggtccctaggtaaagtggactaggtccctgtgaaaggatactgctaccccttccaggtaaagataaatagctgggatgggtaaagagaaacagaggccaggtgcaatggttcatgcctataatcccagcactttgggaggctaaggagggagaattgcttggtgctaggagttgggagaccagtctgggccacatagtgaaatgccatctctttaaaaaaaaaaattaaaaaaattaaaaaaaaatttagccaggtgcagtggcatgtgcctgcagttttttatttgtttgtttgggtttttttgtttgttttgatttgttttgagacggagtctctctgttacctaggctggagtgcaatggcgtgatctcggctcactgcaacctctgcctcctgggttcaagcaattctcccgcctcagcctcccgagtagctgggaatacaggtgcccgccaccacgccctaatttttgtatttttttttttttagtagagacggggtttaaccatgttggtcaggctggtctcaaactgttgacctcaggtggtccacccactttggcctcccaaagtgctgggattacaggcatgagccactgtgtccatccgtgcctgtgggtttgaggctgtgatgagctgtggttgtgcctctgtactccactgtgggcaacagagtgagaccctttctctaaattgaagacagcatatcaagtacccacgtaattaagcccaaggtaatcctccattatttttcaagtcctgtccatctttggtactaaccagcctggagaaacagcctccaaggttcccatcctcctcttcaggaactctgggtcaagaaacttattcagggccaggcacagtggctcacacttgtaattccaacactttgggaggccaaggcaggaggagaccagcctgggcagcagagtgagatcccatctctacgaaaaaaaaaaaaagagagaagggatggttaaggctagaaggactgtgactatgtagtgtgttgacttgacttagatctttgttcattaagctaaggaattatcttgttttacaaaccaagctgaacccaggcccgcccatctgctctgttcctgccactacttcaaacagaagccaaaacctcaactcagtatgttaggccatttcttgcagcaaatttaccacacctacttggcttttgcctttttgtggccacagccaggggtgatattcaaaatatttcacagctggttaccacatggccggcagccatttagaacagacactgattttagcactaagtcctgttcctggagatcaacagctatgctggggattaactctttagttgctgaaagagagggaggtctggccagcacggtggctcatgcttgtaatcacagcactttgggaggctgaggtgggctgatcacttgaggtcaggagttcaagaccagcctggccaacatggtgaaactccatctctactaaaaaagctagccaggcgtggtggtgtgcacctgtaatcccagcaactcagaaggctgaggcaggagaattgcttgtacccaggaagcagaggttgcagtgagccaagatcgtgccactgcactccagcttggatgacagagcaaaactccgtctcaaaaaataaaaaataaaaataagaagagggaggtctgggggcctggaggcaggtggatggggtggggaactcaggaggctgttggcagtaatttaccaactgctaggaaagcttttcagtatttcaacaaccagtatcggcaggacatggtggcttatgtctataattgcagcactttgggaggccaaggcgggcagatcacttgaggtcaggagttcgagaccagcctggtcaacatggcaaaaccctgtctctactaaaaatataaaaattagccgggcgtggtggcacgcgtcggtaatctcagctacttggtaggctgaggcaggagaatcgcttgaatccaggaggtagaggttgcaatgagctgagattattccactgcattctaggcagggtgacagggtgagattctgtctcaaaaaaaaaaaaaaaaaagtatgatcaaccaggccacgtgcaggagaagggaaatgaggggatggggtggagagagggagagagacaaagagaaatgaagggagggaagaggcaggaaggaaatgaatgttctatccttgatttggtctgtcttccttgattacaaccccacaggattcgagtccacatagttgctgctgtaacaactccctagaaaattagcctctaattccttttttccctgcttggattttgcacaccatccatggttgcatgatatttccaaattcctgtgatcagatgttcctcattaggaagtgactcatcacatttgcttggcaatcatgaatacaggagaaattacttggaggggcccaggggactgggaagccccatgccctcaagatattgctcttccaacctccaggatttcagtctttgataatcaaaatcttaatgttaactacagaaagccagcacagttgtgaacttagcaaatactgtgatttgtcaatttacagtatctttttgtattataaaatatttggcaggtaaattttataatgtattttaaaatatacaaataactctgtattatacctgttgattatgatgcgtaaaaataaatgaacactgggctggatgcagtgtctcacgccagtaatcccagcactttgggaggctgaggtgggtagatcacttgaggtcaggagttcgagaccagcctggccaacatggtgaaaccatgtctctactgaaaatacaaaaattagccgggtgtggtggcacgagtctgtaatcccagctactcaggagtctgaggcaggagaatcgcttgaacctgggaagcggaggttgcagtagctgagatcgcgccactgcactccagcctgggtgacaaggtaagactctgtctcaataaataaataaataaataaataaataaataaataaataaacaaacaaaaaaacactggtaaacccaccactcaacataagtagaatgtagtcaatattcactttagagatctaatttttagtcttttccaatcactggctgatagtagtaaatgtgttttttggtaaggtacagtggctcatgtcccaaagtacccagcacttttagatgccaaggtgggaggatcgccagaggccagcagttcaagaccagcctaggcaacataacaagatctcgactctacaaagaaataaaaaattagccaggcatggtgacgtgtgcctgtagtccctcctactttgggggctgaggtggagggatcccttgagcccaggaggtagaggctgcagtgagctgtgatggtgccactgagctccagcctgggtgaacgagttagactctgtcaaaaaaaaaaaaaaaaaaaagtcaaaacaacaacaaagatgtgttttttttgctggtatacagaatggccttgaatgttgttttctttctctctttttttttttttttagatggagtcttgctctgttgcccaggctggagtgcagtggtgccatcttggctcactgcaacctccgcctcccaggttcaagcaattctcctgcctcagcctcccaagtagctgggattacaggcacatgccaccatgcccagctaatttttgtatttttagtagagacggggtttcaccatgttggccattctggtctcaaacccctgacctcaggtgatatgcctgcctaggcctcccaaagtgttgggaattacaggtgtgagccactgtgcccggctgaatgttgttttctgattctacccagtcatcctaattcctccttttttttttcatttccttccttctacttatctctctttctccgttttttttttttttttgacaaggtctcactctgtcaccaaggctggagtgcagtggcatgatcacggctcacggcagccttgatctcccaggctcaagtgaccctcccacctcagcctcttgagtagctgggaccacagagtgccactacgcccagctaattgaaaaaaaaaaaactgtagttatagagtctcactatgttgtccagcctgatctcaagctcctggcctcaagcaatcctcaccactcagcctccctaagtgctgggattacaggcctgagccatcacgcctgtccttatttcccaaagtgctgggattacaggcgtgagccactgcgcccagcccaaaactttactttcaaaggacatagtatctgaatactaagactttacgaactgttttatcaatccatggacttctaacctcccattcagggacatggaaatcatctctgactctctctcctgtgttactatgaagattccatgcctctttggagattctgttgcttgcaaccccgctcctggaatagaattctgtgcttgttagagtagtttggtatttcatttgcaagaaacaagaactaactgcaaattttagggggaaaaaagggcttggacataccataaggacataggtgggctggaactgaaaatctaagaatttgaggttggctgggaactggccactgtctctttcatctttcatggcccgcatagcctcctgcatggcatccttttctctctcactgttaccctcctaagcagtggggatttcattaggctggctaatcatttttagactctgaatgtcagagcccaagtcagtaataataataggagacttccataccccatttaaataatggagaagtcattaggcagaagatcaacaaggaaatagaagacttgaacaacagtataaactgatcagacctaacaggtatctataaaatattcaacccgccaactgcataatctatattcttctcaagtccacaaggacactctccagaatgaactatatgttaggccataaaataagtctcaatatatttaaaaagattgaaatcatacacagtacattctctaaccataataaaacaaaattagaaatcaataatagagcccaggcacagtggctcacacctgtaatcacaaaattttgggagaccgaggcaggaggatcacttgaggccaggagttcaaggctagcctggccaaagtgatgaaaccttgtctctactaaaaatacaaaaaattagccaggtaaggtggcatacgcctgtaatcccaactacttgggaggctgaggcacaagaatcgcttgaacctgggaggcagaggttgcagtgaacctcagtgaactgagattgtgccacagcactccagcctgggtgacagagcgagactctgtctcagaataaaataaaatgaaatataaaataaatcaataatagaaatttgagaaatttaaaactatgtggaaattaaacgcagtcctaaataactcataggtcaaagaagaaatcacggctggacatggtggctcacgtttgtaatcccagcactttgggagaccaaggtgggtggatcacctgaggtcaggagttcaaaatcagcctggctaacatggtgaaaccccgtctctactgaatatacaaaattagctgggcgtggtggtgcatgcctgtaaccccagctacttgggagggtgagacaggagaatcgctctaacccaggaggcagacgttgcagcgagcagggatcgtgccactgcactccagcctgggtgggtgacagagtgagactccatctccaaaaaaaaaaaaagaaagaaaaagaaatcacaagagaaattagaaactacttggagataaatgaaactgaatgatataccaaaaacttaaaggatttagctaaagcaatattttgaaggatatttatagttgtaaatgcctacagtgaaaaagaagaaagatctcaaattcaacaacccatttatttatttatttatttacttatttatttatctatttacttattttttgagacaaagtctcgctctatctgaggctggagtgcagtggcgcaatctcagctcaatgcaacctgcgactcccctgttcaagggattctcctgcctcagcctccttagtagctgggattacaggcgcctgccactgcacccagctaatttttgtatttttagtagagacggggtttcaccattttggccaggagggtctcgatctcctgaccttgtgatccacacgcctcagcctcccaaagtgctgggactacaggtgtgagccaccacacccggctaatttttgtatttttagtagagacagggtttctccatgttgcccaggctggtctcaaactgctaacctcaagtgatccgcccgcctgccttggcctcccaaagtgctgggattacagacaggagccactgcgcctggcctgcatgtcttctttgatctggccccaccctcctttacttactctgttccttcaaattggaatgtccttcagcctccacatctacaacaatggctggaattgtctccgttcttcaaggcctgattataattccatcatcttcatgtaaccttttttttttttcttcatcctactcttcaactgatggagcacaaagtctggtagaggagataaaggagacaattgggttctgtcttgcaatgtgataacactctggtgggtgtgtgcacagagtcctaggaaggcaggagggtgaggtagaaactgttgggttagttgaaagatagaaaattgcacactccagttgacttccacttctgagggcttagaactccatgaggaggcatcgatttcatcttaaaggatccctttctacactcatttcttttctttcttctctttcttttttttttttttttgttttgttttgtttttagagactgggtcttattctgtcacccagcctggagtgcagtggtgtgatcgtagttcactgcagcctccaactcctgggttcaagtgatcctcccatctcagcctcccaaatagctgagactacaagtttgcatcaccatgcctggctaattttttttacttttggtagacacaaagtctcactctgttacccaggctgatctcgaactccttgcctcaagcaatcttcccacctcggcctccgaaagcactagaatttcaggcatgagccaccacacgtggcctaacctgctgttttctcaagagcaggacagtttttaatttctggacctatcgcatctagcaaggttcctggccagaaaaagtgaaaataatttttttttttttttggagacagagtctcgctctgttgcccaggctggagtgcagtggcgtgatcttggctcactgcaacctccacctcctgggttcaagtgattctcctgccttagcctctagagtagctgggattacaggcatgcgccactacgcctggctaatttttttttttgagatggagtctcactctgttgcccaggccagagggcagtagagtgatctcggctcactgcaacctctgcctcccgggttctagcgattctcctgcctcagcctccagagtagctgggattacaggcacccaccaccatacccggctaatttttgtatttttaatacagacaggttttcaccatcttggccaggctggtcttgaactcctgaccttgtgatccacctgccttggcctcccaaagtgttgggattacaggtgtgagccaccacaccctgttgcctggctaattttgtatttttagtagagatggggtttttctatgttggtcaggctggtctcaaactgctaacctcaagtgatccgcctgcctcggcctcccaaagtgctgggattacaggcatgagccaccacacctggctgggacttgcttttaataaactaaatgtgatggaaatgacagtgtgtgatttctggggctaggtcataaaagacattgtgcttctgccttgctctctctctctcagagcccccactccgggggcaagccagctgccgcatcacgaggacatccacgcaacctatagagctgctcaggaggaactgaggccggctgccaataaccagcaccaacttgcctgccgtgtgagtgagccatctgggaaaccaagcctccagcctccatgaagcctgcaggtaacgtcagccctccagtcttccggtcttccagctgaggccccacacgtcgtggagcagagccaagccaatcccgtcatgccctgaattcctgatccacagaactgcgagagaatacatgaaaattgttttaagccactacgctctgagatagtttgttatgcagcagacaacaactgatgtaacagtggcaggtgtacacggagtgccaagggaatgcaaagaacggacacttaggggcttggagtggtggctcatgcctgtaatcccagcactttaggaggctgaggcatgagaatcacttgaaaccaggagttcaagaccagtttgggcaacatacgagacccctgtctctacaaaaaaattttaaaaattagctgcccacactggcacatgcctctagtcccagctactctggaggccgaggcaggaggatggcttgagtccaggaattggaggctgaagtgagctatgactgcaccactgcactccagcctgggagacagagtgagactgagaccctgtctcaaaaaatatatatattataaatacttaattctaaattttatgatataattgtattgagaggataggggaagggaaagagttttgtgtgggggtgtaaaagttaaatcctcatttcctgttttaagaaatcatagatcgtatctaaaatagaaaaatcaaggaatagctatacaagcaagttatttagaaactggagctgggctcatgcctgtaatctcagcactttgggaggccgaggcaggaggatcacctgcggtcaggagttcaagggtagcctggccaacatggtgaaaccccatctctacagaaaatacaaaaattagccgtgtgtggtggcacgcgcctgtagtcccagctactcaggaggctgaggcacgagaatcgcttgaacccaggaggcggaagttgcagtgagccaagatcatgccactgcactccagcctaggtgacagagcaagactctgtctaaagaaaaaaaaaaaaaaaacagaaaagaaaccaagaggcatatactggaagaaacaactgaagaagttggttgaatccaagaagcaggtcaagagtagaaagagcagggattgcttttcaccattgttaaccttttagtacaatttgactcttaaaattatgtatgtgtataacttgacaaaataaaaacaaactaaaaaaaggtcagagagaaggagcaataaaataggagatttaggtggagcgatggaacatgttgttctttgtattttattttatttattattattattattattattattattttgaaacagagtttccctcttgttgcccaggctggagtgcaggggcgcgagctcagctcaccacaacctctgcctcctggcttcaagcaattctcctgcctcagcttcccgagtagctgggattacagccatgtgccaccacgcccagataattttgtatttttagtagagatggggtttctccatgttggtcaggctagtctcgaactcccaacctcaggtgattcgcctaccttcacctcaggtgattcaccctcccaaagtgctgggattacaggcgtgagccaccacacccagcctgttctttgcattttaaattaaggggcagggcacatcaaactatttaatatgtgcaatgaacaataaaatatatccttaggatttatatggggcaattatagagcaaagatacaggaagagatcgatgtcaggagacagtgtgatggagaaacaaaggacagaaggaaagagaggggaagagatagaaacaaatgccaagacccaggaaaacacagaaagacataacccagcctcatataaactcaaagtgagaaggtggagagacataaagaaatccacaggtggctgggcgtggtggctcacgcctgtaatcccagcactttaggaggccgaggcggtggatcacctgaggtcaggagttcgagaccagcctcgctaacatggagaaactccgtctctactaaaaatacaaaaagtaggcgtagtggcacatgcctgtaatcccagctactccagaggctgaggcaggataattgcttgaacccgggaggtggaggttgcagtgagctgagattgggtcactgcaccgcagcttgggcgacacagcgagactccatcgcaaaaaaagaaaaaagaaagaaatccatgggctatggaagtatgtacgctcactcaggcacacaccaccaagcgtgatgaagacccaagtctacatagatagcacctcacacttttgttaaatagcacagaattaaagaattatcccagcctggacaacatggcaaaaccctgtctctatcaaaagtacaaaaattagccaggtgtggtggtgcgcgatgtagtcccagctacttgggaggctgaggtgggaggatctcctgaacccagcaggctgaggttgcagtgagccatgatcacaccaccgcactccagcttgggcaacagagtgagaccctgtcttacaaaaaaaagaaaaaaaaaaaaagaaaagaaagaaaaggaaaggaaaggaaaagaaaagaaaaaatgattgttgaggtaggaggcaagactcaactccagaggcagggcacggacaccagaccaaattgaggaccaactaaaaatgggttgggacagaagcagctttccataaagacatcccttccagagtgccatgtcagtttaccgttgccatgacaacacccagaagttatctcccctttccatggcaaccacctaccgacctggaagttaccaccctcatcctagacatttctgcataagccaccccttaatttgcatataattaaaaatgggcataaatgagtttaggtctgcctctcagctgctattctggacacagtgcctatgggtcatccctgctctgcaaggagccgtacctttgctgcggctgtgcactgaagcttcaataaaagttgtaacactggctggacacgagtgctcaaacctataatcccagcactttgggaggtggaggtgggcggatcactttagctcaggagttcaagaccaacctgggcaacatggtgaaacctcgactctacaaagtacaaaacatattagccaggcatggtggtgtgtgcctgtagtcccagcttctcagaagacaggggtgaaggattgcttgagcccaggaggcaaaagtttcagtgagacttgatcatgccactgcactccagcctgggtgacagagtgagaccctacctccaaaaaaaaaaaaaagtcttgttctaccagctaattggattggcagggacctcctttttaatatatatatataggtatgtgccaccgcacctggctaatttttgtttttctttttttttttttttgagatggagtctcgctctgttgcccaggctggagtgcagtggcgtgatctcggctcactgcaagtgggttcatgccattttcctgcctcagcctcccgagtagctaggactacaggcgcctgccaccatgcccggctaattttttttttttttttttttttttgtatttttggtagagacagggtttcatgtgctagccaggatggtctcgatctcctgatctcgtgatccactcaccttgacctcccaaagtgctgggattacaggtgtgagccaccgggcctggcctaatttttgtatttttagtagagatggggttttgccatgttgcccaggctggtcttgaactcctgggctcaagtgattcacctgcctcggcctcccaaagtgttgggattacaggcgtgagccgccacgccaggcctcatgagtttcttatgtagctgtatcaaacttgtttctcttttgaactgtgattacagttccagtgttcatatgttgccctgcaagtaggttataagtgcatgggggacaggaaccaagccttatctactgaagaatcttcctcgggcccagcttccagggaggctctgaatttgaagcccctctctgccattcacttgctgtgtgactttgggcaggttacttgacctctctgtgtgtcagagcttttatcttcagagataccactgggttgaatgacataaaattgccaatattctattatatttgaccccaaaatggcaatttcatatagttcaacctaataaaaccactacctcttaggtattttgagggttaattgtcatgtctattgtaccccaggcaagcaccatcctgctgagtacaaagtggacctgatagtaaagcccctcagactttgtggaacaaaagcagaaccctaggaacggcagagcaaccagttagaaagagcctggctcctgatgaactgcagcttccgtatctgttatggattgctaatgtctggacccttatatgagcaagatgtaatcccccatttaggttaaaccactgttctttgggtctgggttttttgtttgtttgttttttctttgagacggagtctcgctctgtctcctcagctggagcgcagtggcgtgatcttggctcactgcaatctccgcctcctaggttcaagcgattctcctgcctcagcctccccaagtagctgggactaaaggcgcccaccaccacgcccagctaatttttgtgtttttagtagagatgaggtttcgccatgttgaccaggctggtctcaaactcctgaactcaagtgatctgcccctcttggcctccctaagtgctgggattacaggtgtgagcaaccatgctcagcctgggtctgttttgaaagaggtaaactgacatcttaattaataatgggtgtgtgtcagagagaaataaaccatgtacttccacatagagatttctgccttaaggccgggcacagtggctcacacctgtaatcccagcactttgggaggccaaggcggaaggattatctgaggtcagaagttccagaccagcctgtacaacatgacgaaaccccgtctccactaaaaatacaaaaattagccaggcatggtggcgcgcgcatgtaatcccagctactcggaaggctgaggcaggagaatcacttcaacctgggaggcggaggttgcagtgagccaagattgcaccactgtattccagcctgggtgacagagcaagactccatctcaaaaaaaaaaaaaaaaagtttctgccttgggagactgaatgtgatgacactggctgagatggtcagtccaagacatgagggctggttggggaagtaatgagttcaattgcagatagatttttgtttttgtttttagagacagggtgtctgcagatagatttttgtttttgtttttagagacagggtgtctcgctctgttgcccaaggtggagtgcagtggtgtgatcatagcttactgcagccttgaaatcctgggctcaagcgatcctcccacctcagcctcctgagtagctgggactacaggcatgtgccaccacggctggctaattattattttttattttttgcagagacgggggtctctctatgttgcccgagccagtctcgaactcctggcctcaagtgatccatctgcctcaacaccctaaagtgctggcactataggcatgagccacggcacccagccactgtaagcctttttgcttcacctagccaccaatctaaatggggggaagtgttctccatttttttagattttcatatctgtaagtagaccaaccatgacagctttacgacccaaatacaaggctacatttgatgttactagcgagcaaaataacttcagatctacatgggcagtgaaataaatagtgcgcatctttaaaatttcccctctagttctaatcttttcaggtttccccctcattactctgaaatttctggaatgtgtacatgttgctctcacacccattcctgggctttaaggcaggaacatcaaaaaacatcagcatgaggtcaccacttggtcagtggaaaattttttttttttttctgagatggagtttcgccctgttgcctaggctggagtgcagtggcgtaatctcagttcactgcaacctccacctcccgggttcaagcaattctcctgcctcagccttccaagtagctgggattacaggagcgtgccagcatgcccagctaattttttgtatttagtagagttggggttttgccatgttggccaagctggtctgaaactcctgacctcatgatctgccccccttggcctcctaaagtgctgagattacaggcatgagccaccgcgcccggcgggtcagtggaaatttttttaaatggtggtgacaccatctctggtgcttccaccctgtggctcctggctcccagccatttttcatggtggcaaagactgcccccagggtggtggcagaggtcagtgtgtggcctggacgagtcccagccaacatgctcctcaccccatcctggacgcaacgtggaggtggtagagagagaggagcaagggaagctggacccccgcagagtggaccactactctacagggacatgcatgagggacacctggatctgaacagctagcactgagacactccaacacacggtacctggaaaccgcaggtggcactttgaggaaacaaccaggtacgtgcagggatttttttttttttttgagacagagtctgtcagcccaggctgcagtgcactggtgtgatctcagctcaccacaacctccgcctcccaggttcaagtgattctcctgtctcagcctcccgtgtagctgggattacaggcgcctgccaccacacccggctaatttttgtatttttagtagagacagggtttcaccatgttggccaggctggtcttgaactcctggcttccagcaatccacccgccttggcctcccaaagtgctgggattacaggcgtgaaccacctcattctgcctttttgtttttttttttttttttaaagagacagggtctccctctgtcccccaggctggagtgctgtggtgcaatgatagttcactgcggcctccatctcctaggttcaagtgatcctctcatctcagcctcctgaatagctgggactagaagggtgtaccaccatacctggctaattttttaattttctgaaaataaggtcttgctatactgtccaggctggttatttttagttagagagatgttgtgtgttttggaaggggcttcctagatcatctgctcccaatccttatttttttcaaaggagaaaaatgtttgactcatcatactgactttaaaaaaacccactttttattgtggaaaatttcaagcagactcacgcacaaacaaaacaatataatgaacacccatgtacccatcatgaaaaaattaccatgagttggtactcatggacattctttttcttttttttaagacagagtttcactctgtcgcccaggctggagtgcagtgacgcgatctcggctcactgcagcctctgcctcctgggtttaagcaattctcctgcctcagcctcccaagtagctgagattacaggcaccaccaccacgcctggctaatttttgtatttttagtagagatggggtttccccacgttggccaggctggtttcgaactcctgacctcaggtgatctgcctgcctcagcctcccaaagggctgggatcacaggcatgagccactgtgcctggactcgtggccattcttgtttcatcttttcctcatgtaccttctccttcccattttattttgaatcaaatcccagaccttgtataatttcttcattaatatttcagtattatttctttaaggacttttaaaaatatatacccacaatgcattatcatgcctaaaaattaataataattccttaataccatcaaatatatccagtcagtggtaatattcccaattgtctcataaatgtaataaatgaggggtttcattggtttgttttagagacagtctctctaggaaaaaaattcctggaagtggaatttctgagtcaaaactatacacattttatttttaaataaatatgaaactgaccttcaaaagggctatattaatttatatttacataatgaatgacttttctgcatatatttgccatactgatgttataaattttcttcagttttgtctgaaaggtgaaaagtattttattattgttttattttgcattatttcattaatgctaaggctgaacatgtttactcttaacttctgtatctggtaagttatttgtttatgttcttgttcatatctgtctgggttcctttctcctcttcttattgactcatgaacctatatactgtattaatctttttcatatgtgttagaaatatatttgtagtttgacttgctatatagaagtatttttgttttgttttaaagagatggggtctcgctatgttgtccaggctggtctggaactcctgggctcaagcaatcctattgtattaatctttttcatatgtgttagaaatatgtttgtagtttgacttgccatatagaagtatttttttgttttaaagagatggggtcttgctatgttgaccaggctggtctagaactctggtgctcaagcaatcctcctgccttggccttttttgagacagagtctcactctgttgcccaggctggagtgcagtggcgccatcttggttcactgcaatctctgcctcctgggttcaagcaattctcctgcttcagtctcccaattagcttggattacaggtgcctgccaccatacctggctaattttttttttttttttttgtattttagtagagacagggtttcaccaagttggccaggctggtctcaaactcctaatctcaggtgatccgcctacctctgcctcccaaggtgctgggattacaggcgtgagccaccttgcccggcccacattttccttttaatgtttgtttttacactgaagtcctttggcataataaatgaaatagagatgcagcttcccaccctcccccttctgagtagttaattctttaagaaacaactattaggccaagtcaggaggatcgctagaagccaggacggtgaggctgcaatgagctatgatcgcaccactgcactccagcctggatgacagagcaaaaccttgtctctaaaaaaatatataaaatatggctagacgcagtggctcacacctgtaatcctaacattttggaaggtctaggcaggaggatcgcttgaggccaggagttcaagatcagcctgggcaagacagtgagactctttctatacaaaatatttttttaggctgggcatggtggcacacgtctgtaatcccagcactttgggaggccaaggcgggtggatcacgagttcagtagatcaagaccatcctccctaacatggtgaaaccccatctctactaaaaatacaaaacattagctgggcatggtgacacgtgcctgtagtcccagctactcgggaggctgaggcaggagaattgctggaacccaggaagcggaggttgcagtgatctgagatcacgccactgtactccagcccgggcaacaaagtgagactccattttttttaattatcctggtgtggtggtgcatgcctatagtcccactcgggagactgaggtgggaggatcacttgaggccaggagttcgaggctgcagtgagctatggtcatgccattgcactccagcctgagcaacagagtgaaactcttgtctcaaataatggtaataaataaaaataaaatttagaaaggaacaattattgaataaggaatcttttcccaaccaatgtgcaatatcatctttataagtgctaaattcccatgtgcatttggggctatttctggacgcttcattccgatggattatatggattatgccagtcctgtgccaggacaagcatgctttgacttttatttcctgttttaatatttgatagggcaggtccccctattactcttctgtttcagaatgttctggtttttctcattatcattctgcataaactttcaaattattttatcaagtttcccaaataatccttttgggatttagagactgcattgaatttacggatttagggaaaactgacatcttctatatcatccaatcttcttatctaagaacaagttattaccatttgattattcaagtctttttaaattaattaattaattttttttttttttgagacagagtctcactctgtcgcccaggctggagtgcagtggcgtgatctcagctcactgcaagctcgcctcccgggttcaagagattctcctgcctcagcctcccgagtagctgggactacaggtgcccgccaccacgtccggctaattttttgtattttgtttagtagagatggggtttcactgtgttagccgggatggtcttgatctcctgaccttgtgatctgcccgcctcagcctcccaaagtgctgggattacaagtgtgagccaccacgcccggccctttttttttttttttttaagacaggttcttgctctgttgcccaagctggagggcagtggtgcaatcatagctctctgcagtgttgatttccttgggcttaagtgatccttacacctcagcctcctgagtagctgggactacaggcacatgccactatgcctagccttttttttttcttttaactttttgtagagacaaggtcttgctatgttgcccaggctgatctggaactcctgggctcaagtcatcctcctgcctgggcctcccaaagtgctgggattacaggtgtgagccactgtgcttgaccccacaagccttccttctccctcgctccacgatagctgtttgtcctgaaggttttccttttgtatttttgtatttatttttacttttatttttttgagatggagtctcgctctggcccccaggctggagtgcagtagcacaatctctgctcactgcaacctccacctcctgggttcaagtgatttgcctgcctcagactccagagtagcttggactacaggtactcaccactatgcctggctaatttttgtatttttagtggagacagagtttcaccatgttggccaggctgctctcgaactcctgacctcaggtgatccatgcgcctcagcctcccaaagtggtgggattactggtgtgagccaccacggccagccaaggtcttccctttttaagtgactcaaaatactttagtgttatcaactgatccaatataggttctataatttcttgttaatgttactcctatttattttaaggtttttattgctcttatgaatgggatatttccttccattaattttcctttcctttttttttttaatttttttggagacagagttttgctctgttgcccaggctggaatgcagctacatggtcttggctcactgtaacctccgtctcccagatgcaagtgattctcctgcttcagcctcctcaatagctgggacctacaggcacacactaccatgcccggctaatttttttcattttcactagagaggattttgccatgttgtccaggctggtctcgaactcccaagttcaggcaatccacccacctcggcctcccaaagtgctaggattacaagcgtgagccactgcacccagccccttccattaattaactttttttttttttttttgagacagagtcttgctctgtcacccaggctggagtgcagtggtgcgatctcggctcactgcagcctctgcctcccgggttcaagcaattctcctgccttagccttctgagtagctgggattacaggtgcccgccaccacacccagctaattttgtatttttagtagagacagggtttcaccatattggccaggctggtctcgaacttctgatatcaagtgatctgcccgcctcggcctcccaaagtgctgggattacaggcatgagccacctcacccggtctaattttctggctagttaaaaagagctactgcctgtttatatcttgggttttgttttttgttttgttttgtttttttcaagttggggtctcactctgttgcccaggctggaatgcagtgaagggatcatagctcactgcagcttccaattcctgggctgaagcgatccacccatctcagcctcctgagtagctccagctacaggtgctcaccactacatccagctaatttttattaattttttttgtagagatgaagtctagctttgttgcccaggcttgtctcaaactcctggcctcaagcgatcttctaaccttggcttcccaaagtgctgggactacaggcatgagccactgtgcctggcctgtatttcatccctagctcttttctctagccctgccctccttgcagaggtcagctgtgttgggcttgagtgccatttctatgtcaattactcctaatgtccatagtcttgtcctgtctcctgagctatcatcattttacttttaacttgtttcctaaatttcaaatactcacataccactgtcataatttttcatattctgtacattgcctgtactattatttattggcttaacattttttctttaaaccagctcattaaaaataactctagacttatcttgtgtaatcacattcatgaaataacaagtagccttatgtattggttataacttttctaatataagtgaaaataaatacaaaactattacagtttaaaaaaaaatgctcttcttcataaagtgtaaaaacatctcttgtggcatctaccacatttttttttttttgagacggggtctcactctgttgcccaggctagaatgcagtgaagcaaccatggctcactgcagccttgacctcctgagctcaagcgatcctcccacctcaacctcctgagtaactgggattacaaacgtgtgccaccaagcccaactatttttttttattattgtttgtagagatggggggttctatgttgcccaggctggtcttgaactcctggcctcaagtgatcctcctgcctcgggcctcccgaagtgctgggattacaggcgtgagccaccacgtctggctatccaccacattttccgaaactctgttctattcagtaacaaaccattcaacctaaatattcatcattgcttcaaattcaatgttcttcctccaaagtcactctcaagcccacctgcctcactctgtcagttccccgatcaccagacctccaaacttgggcttcttgtatgaccaattctgctccttcactcccatcctaaggtttcctttcagtggcctcttgcaactgtccctagccctccatcctcccaggcatcctccttatcctcctccctggtgacagatcacctcccagcaggattcccctggcttccctagtgaggacctgagtcttgccttgtggcccgcttcaccatggctggacccccttcttttttgagatggagtttcgctcttgttgcccaggctggagtgcaatggcgtgatctcggctcactgctgcaacctctgcctcccgggttctggcgattctcctgcctcagcttcctgagtagctgggattacaggcatgcgccaccatgcccagctaatttttttgtattttttagtagagacggggtttctccatgttggtcaggctggtctcgaactcccaacctcaggtgatctgtcccatggctggacctcttaatgccatcaccatcatttaaacatacttcctacctccccctgcccccatctcctactgttccatagacaatcaaaaccatgaccgtgaccttgggagtttgcctcttcccctccccaaacttcacctgtccttttttttttttttttttttgagacaggatcgcattctgttgcccaagctggagtgcagtggcatgatctctgctcactgcagcctctgccttctaggttccagccatcctcccacctcagcttcctgagtatctgggactaaaggcgcatgccttcacacctggctaatttttgtattttttgtagagatggggtttctctatgttgcccagactagcttacctgtccttttagggtaactttgcaggaagccttcttagatgattccagccctgtaccccctcttttttcttttctttcattgttgttgttgagacagagtctcgctctgtcgcccaggctgaagtgcagtggcatgatctcagctcactgcaacctccacttcctgagttcaagtgattctcctgcctcagcctcccaagtagctgggatatatcgccaccacacccggctaatttttgtatttttttttttagtagagataaggttttaccatgttggtcaggctggtcttgaactcctaacctcaggtgatctgcccacctcgacctcccaaagtgctggaattacaggtgtgagccaccacgcttggccctgtcccccctctcttactccttcaacagtttattgtcatctcagtggaggtcatctgagtagatcacaaggtacttgaaggcaagcttcatgcccaaatattccttctgcatcagtgcaatgccaagaacacattctgtgcacaaaaaaaatgctaatggagtcatggcccagtgcattcccaaagacttacctggaactccacctcgccctgtgacttggccacagctactcacccctccccgcacaaccttcaactcccaccaaccaggggcaagatgggaaaaggagcaagtgtaagaatagcagagcacgctgtgaaatagaagaagccagctttaggatcatgagtcttttaatatattattctgaaaagaaaggaattaaaaaaaagttcctgacatctcttctgttttgttttttaatttaaataaataccctgcccaccccctccacccgctaaaatttccctcctcctcccccatcatcctgcccatccctggcactagaccctcaccccagaactaaataagatgcctgattcagagcaggccacactgactaccaaattctggcaaattgtaaatactgtgaatggtcttgggccctctgcttccctgggccccttcttctctcggtccactccccatgcagacccctccttggctgtggggtggcatggacggcggggcctttaggcattggcattggggagagtgcaaacttggccctgattctccactcctagctcaggagtaggtcagaaaggcccagaatcgccctccagactgaaataaataacaaaataaataccccctccccaactcccggctctctagaaaagagctcctccatctggcactctagtccctctctttcagctcacccacccccctcttcctcccatcccttccttcctcccttccccatgctgacctctggcttggttcaggaggaggggtgtacaacgcccagcgggaagaacagccagagcgactctcgccccaacctcaagcaaaccctcctcctcctccgtttctcagcagcgctgttggcatcctaagcccaatgggcctggcagactctccccatcccggggggtaggttggagggcaggggttctccctggcactctcccacgtagtctttcaatgtctctcaaggcgaggctcccgggagagccaggcgtgcccgccccctccccagtccaggcgccccggtacccccccgcccgcctcagatggcggtgtcccagaggaccgtgtgctgccggcggcagggcggggtgccggacttgcggggctcgggtccccgccgccagctggggaggatgggcatgctctcctgcttgcagaggcctcggtccgggcggtggcgcgccttgatctccttgcacacgcagcgcttgcgctcgatcacctccagcagcttcccgtcacgctcgcgggccggctggggcggcaggggcagaggcagcggcagcgcggggtggggctgtgccgcgggcgggggagaaaggaaaacgcgggtaaggtcggggacctcagcatgaccggacgggtccgcactgccgagacccacgtctcccctcagaagcaagtccttgcttgccccccaacggcctctgctggctccctctggctgtggggcccatgggggccaaaatccaacttgcgcagctttggcccagccactccctagtcttcctagacaggtgcattttttaagttggtccccctagaagggatctccccttttttttctttcttttgtctttttgagacagggtctggctctgtcgcccaggccagagtgcagtggtgcaatcatagctcactgcagcctcgacctcctgggctcaagcaatcctccagtgtcagcctccctcccaagtagctgggactacaggcatttaccactacaccagctaattgttaaaaaattaaattaaagtttatttttgtatttatgggatcttgctttgttgccaagcctggtcttgaactcctggcctcaagggatcctctcatcttggcctcccaaagcactgggatcacaggtatcaactaccgtgtccagctggggcaagggtagacaaggtccctcccttcctcctttcctccctttctttctttctcttttctttcttgctttcttttctttcttgcttgctttctttctctctttctctccttctctctttcttccttctttctctcactctctctctttttctttcttttgacaggttctccctctgccacccaggctggagtgcagtggaacgaccatggctcactgcaacctccacctcccaggctcaagtgattctcctgcctcagccttcagagttgctggaaccacaagcatgcaccagcatgtctggataatttttaaattttttgtaaagacagggtctccctatgtcgcccaggctggtacagaattcctaggctcaaccacttctcttggctcggcctcccaaagtactaggattataggcgtaagctaccatgcttgacctctttatttttatttcttttgagacacggtcttgcccttacacccaggatagagtgctgtggtgggatcgtagttcattgcaacctcagcctcctgggttcaagcgatcctcccgtctctgcctcccacagttctgggattgcaggcacaagacatagtgcccagtggggaggtctttttcctaaatcgcacaaaggtgtgctgttggctagtggtggccctgactgatctccaagctgggccctaccttgctgcccatcttaagcaaaccctcaaacccttcccagatcctctcccaaccccctcactctcattcctgggttcccgctgtctgcgctggctcaggccccagtctgcctggcccctttttttttttttgagacagagtctaactctgtcgcccaggctggagtgcagtggcatgatcttggctcactgcagcctccgtctcccgggttcaagcaattctcctgcctcagcctcccgagtagctgggattacgggtgcctgccccaatgtccagctaatttctgtggggtttttgttttttgttttttggttttttttgagacagagtctctctctgttgcccaggccactggagtgcagtggtgcgatcttggcttactgcaacctccgtctcccgggttcaggcgattcttatgcctctgcctcccgagtagctgggactacaggcgtgcgctaccacgcccggctaatttttgtatttttagtacagatgggatttcactatgttggtcaggctagtcttgaactcctgaccttgtgatccactcgcctcagcctcccaaactgctgggattacaggcatgagccaccgcacctggccctaatttttgtatttctagtagagacagggtttcaccacattggccaggctggtctcaaactcctgacctcaagtgatctgcccgcctcggcctcccaaagtgctgtgattacaggcgtaagccaccgcaccctgcctggcccctttctttctttttttttttttcttctaagacagggtctcactctgtggctcagactggaacgcagtagcgtgatcatggctcaatgcaacctccacctcccagggtaaagccatcttcctacctcaacctccagagtagctgggaattcaggcttgcgacggcacacctgactacattttgcattttttatagagatgggatctcactatgtggtccaggctggtctcgacctcctgggctcaagcaatctacctgccttggcttctgaaagcattgggattacaggtgtgagccactgcaactagccttcttttaaaaaaaagaaaaaatagagacagggtctcactatgttgctcaagctaatgaacttctggcctcaagtgaccctcccaccttggcctcccaaagtgctgggattacaggcatgagccactgtgcccagccctggcccctttattgcttttctttgcatattcaagccttctccacctgctccttcacaaataaaagaaaaaccaagcctcacctggctcagggcatccctgggctggctgcagggccggggtgtgtggagcacgacctgccggactccggaggtggaggcggagcgccccaggcggtagcctcccgtgaagtctgaggtggaagaaggaggagtgaggagtgcaggggcctcaggtacccctcccctagttcccagtcccctgcccttgaatggtccaggctgcccctgcctcatcagaagggaaaaaggggtggagatgtggtatgaggatttagggccctgggcacagacgtgaacaggtgagaggaagtcaggacaggtgcagctagaccccgggagatcccccagccagccccgctgtgcacaggtgtgtgcaggccgcgtcacctccattgcggtggcaggcggggaaggtctggcagggaatgggcagcgcggtgcggctccctcctcggtctccatggtggatcctggccagcagtccctctgccccctggcttctcactgggatccccgatgtcccagggcccctgtcctcacgctccaccttgcctggaccctcggcactgccattccaggcccgggcaccgtcctcgacctctgctgggatgacaaggagagctgtgtgcctgcatgttggaggcgtcattctgtccctctggccacacatgctgcagtatgggtggaccaggagacacaattgccccttttgcaagtaacggctgggatggggctccttcacactcccccaacatacatacaaacacacacacaccccttcctttccacccattggtaccatctcttggggctgagaatcatgtctatgtgctgcatccaaacccttttccctggtcagaggggcctccccaaccgcagctttagcttccaagcccctccatctccagatagggcgagaatgaccatccccctgggatcaagacctgtagggggagaatcccaattcctgagcccagaagggggagcgctggggggaagggctgcctgtgaactaagctcatctctgagaggaggcctggcagagcattgcgttgccaggcaacggtaggggcctccctctcctctccctcctgtctggatgggttgccatggtaacagctgggaggcagctgtgtttagttcctggtgcgcacactcacaagcacacacccctgcagccccgggaagggaggggcagggagcagagcaggacagggccagatttgacccccttcgcctcccagaccttcctgcccctccagcgcacactgggggaccccggtctttctgtctatcgcccacccccacctctctggtctcctagaaaacatcccagactcaaggctgtgacttccaaataacccctccaacacctcccccaaaccatcgcaggcatccccagctgccccccacccctgatgccagcccccactccccacccctcactcactgtccaactcctttgcttttctccctccatagaggaccttcctctgcagggcccagcctgccccaaatgtcgcggcgcccacctcctcttcttcctcctctggtgtccctgcgctggcgatgacgtgggcacaggagatgctggcaaaagcacccccatcggtcccttgctcctgcagctggatctt

SEQ ID NO: 2 - DNA, 146407 bp, Homo sapiens
gatcctcccacctcagcctcctgagtagctaggaccctaggcatgtatcgccacatctaactaatttttttttttttttttgagtggagatggggtctcacaatgttgcccaggctggtctggagatgtggtctcactatgttgggcaggctggtcttgaactcctggcctcaagtgatcctccctcctcagcctcccaaagtgctgggagtacaggtgtgagccactgtgcccagcctggggaagggttttatgaggaaggtgatggcattgccctggggccctgggtctggttcaggattggccagggtgaatcaggtgaggggctcatcctggagggccatgggagatgggttgttaaacagactccacccctgctccaggcagtgtggtcgccatgatgtaatcagactccccaggcacagctcagaggggtgggcatggagggcttcctggaggagtgagccagttgggtgcagcaggatgggcctgtgggatggagcctggaagaagatgccagcctggggcattggagaagtgagcagggcagacccagagtctgaagagcctggaatgttctagaaggtatttatgttttatatgaagctgaccgacctggctttccctgctctttgtctgggcctctccgtgctcctctgagcagtgggggcaacagtatctgcagcgggccccagggtgatgggaggagcccccctgggcttcgtgtggcctggctcagggctcagtgccccggaccagcctcccagcactgccaggtctgccccaccagacccctggccgctccgctcttcccacagatggagactcaaggctggcctctgccatcgtggacacagtgctggcaggtgtcccaggaccccggggtccccctggtccaccaggtaagtgaatggtgggctggcctgcattgaggactccatccccccagggctggcctgagccatggggccttgaggggagctggaggtcggctggcggtccatggcttctgggcaatgctggggactgggtctgtcccacccgctgtgtggcaggaaatgaagcccctcgtttcctagtggcagtgcccctgggggactggatgcatgaaacccacgtgattctgctggaataagatgcctgcatgaggctgcccttgcatgggccagagtgggacacacaccagtgggggatggggtcaggacagaggtggcaggaagtccctacctttgaggcagaaaccaaggggttcaaaggggctgggacctggctgtcagtcctggcctgaccttccatcatccacattcctcattgggggaagaatgcagggctgactgccttgtccctttctgcccgggaagggagtcattcattcattcattcatttgtttattcactgattcattcattcttttattcattcatttattcattcattcgttcattcattcattcatttgtccattcattcattcgttcattcattcattcgtttattcattcattcattcatttctctgtgcgtctcacaaacattcattgaacatctgccactacatgtgccagacagtgatctaggttcccggaactcctgaataatcaaaacaaacatcatcgagaaactctcattctggtggaggacaggaatatcatcataaattgtgacttcgacaggatctgagggagctacaagggagaagctggggcgtggcggggaggcgccgtcaaaacgtgacatttcagaagatgctctagggaggagagggagtaagctcagagggtacctgggggccttcaggtgacatgccaggtctctggagaactggacagactcctgacatttgtcccctggagtgcctgtcctcacatgccctctccaaacctgggagccaggccatgctggacagcccaacatgcggctccctgcaccacagggagggatcatgagagaccctccctgggcccgtgtggggtcatgagagaccaagaccttactcttcctagcaggctggagattagtcatgaatctccctccctgagctgggcacacacatgagacccagcccggccctctgggtactccacatcccatggggcagacagacgtgtaaacctcagtggccatgggcggtggctggtttacataagggatttgcctggagaagcagtggaaagctgggaaggcttcctggaagaggtgatgctctgtgtcagactggtaggaaaaccagtaggaaagggtttgctgggagagtggaggaatagggaggctccaggaggaaggacagctttagcagagacgccaggagggtgtctggctggggcagggagggtgggaaggggccgggctgaggacttcaggagccctgaaagggatgtaactgggcttggatttttgagagtgtactcttgggggccataggaagggtcgatgatcaggaggctgttatagtaaccccagcaagagggatgggtcccaggtcccgaggcgggaaagaccggagagacgtaccaggggaccaactggctggggcgtgggagcagtctggacggacttcctcccttgcctgccgggtctcgggattggggctgcgtcgtggacgaggccctgggaggaggaggtgtgggagctgtgggcagccggtggagaggaccctgggctccgaggatggagacctcgttgagagggggtgtgcctccctcctccccacaccattcattcccaaaccctcctttacttcccatggagacagatcaagaacaggtgctggggtgggaggctgggggcgccccctctctggccccaggtgaccatctgtgactctgcccacaggtccccctgggcctcgaggtcccccaggacccccaggaacacctggatcccaggtaaggactttgccattttaggtagggtgtgggaggcggcgggtggcgggtggccttgtctccctaggggagaagaagcacctttttaccagcaagagtcactcaggaaggagccagtcggcctggccggtgagattttattttattttgttttatttatttttaaatttatttttgagatggagtctctctctgtcacccaggctggagtgcagtggcgccatctcagctcatgcaacctccacctcctggattcaagtgattctcctgcctcagcctccggattagccgggaccacaggcgcacgccaccacgcccagctaatttttgtatttttagtaagagatgggatttcaccatgttgcccaggctggtctcgaacatccgacctcaagtaatccatccgcctcagcctcccaaagtgctgggattacaggtgtgagccactgcgcccagccgagattttaattttattagcagcagtgactctttgctgagtatatcctcctcctgttatgccagggaggtgttagcatcactcgcatttcacagatgagctaactgagtcccagagagaccagatgctgcctgaggttgtacagagggtgtaggcagagttgggccaggtcacagaatctctgcctcagcccctcaccttgccctgagaattgcccatggggaggcggcaccccaagcctgactcagcccctaaccccaggttcgccctgcctctgcagctgtcagaagtcaggaagagcaggtacaaaggccctgaggcagggaaaaggctggcaggtagtgagcaatggagtgtgtggtgtcggatgaggcacagtgggcagggccttatctacgggtcccaggtgtgggcttttgttacagtggatggaattattgagtttcagggagggtgattccatgatcttatttacatctttaaaaagctcctctgaggggccaggtgagatggctgacgcctgtaatcccagcactttgggaggctaaggtgggaggatcgcttgagcccgggagtttgagaccagcctgggcaacatagcaagaccccatctctataaaaaatttaaaaattagcctggtgtggtggcgtgcgcctgtagtcccagctacttgggaggctgagatgggaggatggcttgagcccaggagttggaggctgcagtgagctctcatggcaccactgccctccagcctgggtgacagagcaagaccctgtctcattaaaaaaaattaattaattaattaaatatgtaaaaatagaaagtagactcggcatgtctggccaatggaataagtctggtgagaagggcagggaaggctgtagtttcactccctggtgtctgtcaggagcagctgggaggaccagggaggcatttcctgagaaggggaggcttcgggatggggggcaccaggagttccctttggagagagggggagtgggcacctcatggtcagctgggtggcgatgtcaggggacagctggacacgggcctggggctcccgggagccgtctgggctgacatcagcacacgtgggaccctggccatcgctgttagtatcactagttctctccacacaccggaccacctgctccccttctctgcctgacagccgtacgtggcattggcccattttgcaggtgagaagactgagggctcagctggggtccctagtcaatagcagctccaggtggctccaggactggaggcctttgagtctgcccttcccgtgccggccgggccatcctcctctcccttcctcttctgcagactcagagcacagtgggcggggaggggggtggccagagggcactggagacttggccaggaccggcaggcctcacacgcttcctttggttttctgcagggcctggctggagagcgaggcacagtggggccgtccgtaagtgtgggctgtgcagatgggcctgggccactcccaggcagaggctctgatgcaagcccagggcactgggtggcgggggttggtgggggggttcagccctgggtggccctgctggagctcaggcaacagggagggactgagaaaggactcaaatcgcctcctgctgcggggtgaggagggaagcgacctccagagagtgcgctgacagtcctgagtgcctgcagccctggattaatgcactgtgtgaccctggcctttcattcatgcgctgtgtggcctggggccataggttaacatgccgtgtgacctgggaagtcccttcttttctctgaacctcccagaaataggcttagacagccttcctttaatcgactttcttggaaacaagaacttatgagtctcccagcctgaccaacatggtgaaaccctgtctctactaaaaatacaaaaattagctgagtgtggtggtacgcatctgcagtcccagccactctgaaggctgagacagaagaattgcttgaacccggaggcagaggttgcagcgagccaggatcacgccactgcactgcagcctgggtgacagagtgagactccatctcaaaaaaaaaaaaaaaattatgagtatagactcctgcctctagggccaaggagtcaggcgcttccttgatgcccaagcctcactgtaggggtgtggggcggggaggaggagtggagctttgctgagcacctactatgtgctggccacagtggaggtgctctcttcccccacagcgcatgtaagcttcacagcgaccccaagagaccatgctttattatccccgtttgccaaaactggccactaaggctcagagaggttaagtaacttgcccaaagccacacagcgaggcagtggtggagcctggatttgaaccagggagtgtcaagctcccaagccatgcctcctcccgtgcttgtctagtgaccaaacacaacacaggagacacaatgatggcacttctaggtgccaggctctaatctaagctcattgtccctattgattaacttaatcctcctaacaacagatgagatagatactattactgatcccattttacagttgggaaaactgaggcaccaagaagtacagtgacccactcaaggccacacagtagtagctggacatttacatgttactgtgagctgggcatggtggtggctcatacctgtaaccccagtgctttgggaggccaaggcaggaggatcattttgaggccaggagttcaaggccaccctggacaacatagtgagacccacccccatctctataaaaatattaaaaaattagctgggttgtggtggtgcgtgcccatagtcctagttactcaggaggctgaggttggaggatcacttgaacccaggaggtagaggctgcagtgagccatgatcgcgtcactatactccagcctgggcaacagagcaagaccctgtctcaaaaaattggctgggtgcggtggctcacgcctgtaatcccagcactttgggaggccaaggcaggcagatcacctgaagtcaggagttcgagatgagcctggccaacatggtgaaaccccgtctctactacaaatacaaaaattagccagacatggtggcgcatgcctgtaatctcagctactcaggaggctgaggcaggagaatcgcttgaacacaggaggcagagattgcagtgagccgagatcacaacactgcaccccagcctgggtgacaagagtgaaactccatctcaaataaataaataaataaaatacaataaaatatgtgactttgaatctggtattactggccttgtgttggagacagacagcagcccttccaggcaggctactcaatcctggaagggcccaggcatgaacttgccggcttcctagcctgagtctcttctgccccagaagcagaatgaagcacttacagccaaccctggacacttccctgccatgccaggctgagcaggctgtgccccggagccgtggcctcccctgctgtgctcagcccagggcaggagtcccctgggcccagcacccgtttcccaggccctgcctgcccagcctgcccctgactccctgcaggcccccagggaactggagagggtggagaggtgcccccacctcccgttccagtgttgactgtgtgacttgcttcttagggtgaacctggcgtgaagggggaagaaggagagaaagccgccactgcagaggtaaccatggctgcccttcacgttccctcccgcctccttggctgtgagcactgtcacgggagggcaggtgccccacagatctgcgctgaccgtcagccagccccgagctgggtgctgggccaccgggtgtacagggcccctgccctgagaagttcacagccccatgggaactcagctgagtacagcagtgattgcaagagagtgagaaggtggctgggtggggatgccacagagggggtgattggcccttgggggggcttcccgggacagggagaggagtcgcaaacttgtaggaaagacagtctccctggaggaggcgtcaggctgcagagtccctctcaacggaccgggttctcccgtgaatggcgtctcggcatgggcgtgcttacaaggtcaagggtgctgggggactgaaggggacagggctgctggatgtggtagaggatggatggaggaagagggacttgcaaactgggaatctgaggagagaagattggagcaagcggaggctggggccagtgggagggggcagccagggcacgggcttggctgtggggacctgcggcttctgggaggaggaggggaggctgaagctggggtagtggatgaaggggatgaagggctcatctgaggtccaaagtcaaattcctcccctcagctggccctgagctaggtgctgggccaccaggtgtacagggtccctgccctgagaagttcacagcccagtgagggacacagctgaggacagcagtgattgccagagaatgagaaggttgttgcgtgggggtgccactgagatggggccggcagggggggccttgaaagctaaaagcgaaagtgcctgagcagggtttcccttgagtgtgtcaaagctgggtgggcaaaacagctgggagggccagggcgggtccaggggaccttcaggaagtccttgcaagaccagagaaagggaagaggctggaatcaaggccgctgtgaggggtgcaggaggtagtaagaggggctgaggcatgagcctgatacactggtcactcacagcagtactctgagaggtgactgtgaccccctccatttcataacagcaacctgcctggcctgtaatcccagcactttgggaggctgaggtaggagaatcgcttgagcccatgagatcaagaccagcctgggaaacctagcgagaccccatttcttaaaaaaaaaaaaaaaaaaaaaaagtaatgagacaggcgtggtagtgtgtgcttatagtcccagctacttgggaggctgaggcaggaggattgcttgagcccaggaggttgaggctgcagtgaactacaatcgtgccactgtactccagtctgggtgacaaagcaagaccttgtctctaaaacctaaaaaaaccccaaaaaacaaaaacaaaaaaaccaaccacctgtggcttaggaacatgaggctcagccaggtcccttggtctcccctgctccttctctgagttggggacacgacactgacatccctgtcccagaccctgtaattggctcaacttcccattgtctttggtcccctggtgggcagacctttctcctataacccagtaggatgctttttggtttccttttgtttcattttaagctaaattaatctgaacataagacagatctgtcatgttacaccagcaaaaggcacccattctgctgccaactcccagggctgggcaggatggggacagtggctttgtctcctcatcatcacttttcagagtggcctcgacacctgccttctcaataagtcagcaccctgtcttggctgactcaggtgtctctgtgcacaaggtcctctgcttgtgtgcagaccgggcccttgaaatacagactctgccatcacgccagcctgcctgcctgcactttgaaatggtggctttctctgctgtctttcttgcctggcatggccacacctaccctgccccttgcctgctgtttggtggcgagctcttgggcattcaaggaaggaaacaagcaaagaacttcctaccttcgggccctgggataggtgctctgtcctggttagggtattttcaggacatgtgggggaagctgacaggcagctatggtggaaggaaaagctcaggctggaggctgacaggtgtggatctgcagtctggatcggccgctttgccaggctgtgtggccgtggggaggttgcctgccctctctgtgtttcaagctctttgtctgtgaacagggtttgaagccctgtctggcacatagttgatgatgttcagtctatgcttatttgcagtggtgagggtgtcgggtgggaagaggcaggggtggggggctttgaggacacatacactgtcactgtatcaggatggccctgaccccttcctcatggccggccctgaccctgcctgtttcctccccgccagggcgagggggtgcagcagctgagagaggccctgaagatcctggcagagcgagtcctcatcctggagcacatgattgggatccacggtgagcagtgaccaggatcagcgggctgggaatctctctcccctccttcctctcccaatgctgccctcatggctgctgcctagggtctctggtctccctcccttgcccctagtctgaccctccccctcccctgctcaaggaccctcagtggctccctgctgcccttgacagacttccaagtgcatcagcccagtttcccagcccctccctagtatagccatggtgggcagagccctgcccagccatgttgaggcccctcccacctccatgcctttgctcgggccatgcctctgcctggtgaacactgactgccacttcgggacctgctcaaagatctcggcctctgtgaagccttctctgaaccctcctctttgtccctatggtgacataggggaggcttccattccatgctggggtttctgtgaccctctgggagtcccttgtgcagaacagtgaatgatacctctgcagggcctggagtagaggcccatcagcatttgctggaaaatgaatggatgaatgaataggtgaatggacaggtgggtgggtgagtgagtgagtgaacgaatggctggttgcctggatgaatgtatgcatgggtggatgcatagatagatgaatgaatgaatgaatgaattgttgaatggacaggtggataggtggatgagtgagtggatgggtagatgcatggatgaataaatgaatgggtggatggatagatggataaatggatggatggatggatgagtggacaggtgggtgggtgaatgagtgaatggatggatggatggatgatggatgagtgaacaggtgggtggataaatgagtggatggataagtgggcaggtgggtgggtaaatgagtgaatagatgcatggatgaatgaatgaatgggtggatgggcgggtgagtggacaggtaggtgggtgaataagtgagtgaatggatggatggatggatgatagatggatgggtaaatgagtgaatgaatggatagatgcatagataaatgaatgactgagtggatggatggatggatggatggatggatggatggatagatgcatagatgaatcaatgagtggataggtgggtgggtggataagtgagtggatggatgggtggatggatggatggatggatggatggatggatggatggatggatggatggacgggcaggtaaatgagtgaatgaatggatagatgcatagataaatgagtgactgagtgggtggatgaatgggtgaatggatgaatgggtggatacacagatggatgaatgggtcaatggacaggtggatgggtgaatgagtgaatgaatggatggatgcatggatccatgaatgaatgaatgcataaggggctgaaagcctgctgctgccttgcttctccacgctgggcaagagcatctctccgtggccacatatcatgatcaaatgtacccccaagtgttcctaaggctctacctcgagtccaccctcctgctctccttccagatcccctggcctccccagaggaggttctggccaggatgctgccctgagagccaacctcaagatgaagaggggtggcgcccaacccgatggggtccttgctgccctgcttgggcccgaccctggacagaagagcgtggaccaggccagcagcaggaagtgagagcccactgctccaggacaccctatcctggctagagacccagccccagaggcctgagccgccgctgtttcctaaagatgcccccaggggaactgggctccaggcatggatgattgtgaggacatggggggctttggggacagataatgtctccaggggcagggtctggaggggccaacaccctcatcagagccctcctctggcctgtcccctcccctacccccactcccggctggagacggggtctgggtgggctgggtgctgggaatgagaataatcctaatacccatcatttattgagtccctgctgtaactggccctgtccagggaactttcgttacgttgtctcattatttaacccttaggaggtaagcttattatccccacttcacaaggggaccgaggctcagacggtagaaataaccctctcttggccaccagatcacgggtggcaggggcaggatttgaacccaggaccctcagttcctgaagccatgttctctcgaccctgccagcttcccctcttcgagagaaaactcagacagggagggcgcagggccagaacggctccttaaggcaggcagggctgcggggaggctgggccagccagcctgagggggtaggcagggtcctgcagggcctggggcttcccttcctcatcccccttccccactggctgggggagcagcctggactgcagctctcagaggccagggagaccctgaataaatcactgccagccatacggacttgaggaccttgagagagaagcattgggggtgcaagtgtccaccaaaccagataggcatccctggccactgcctcccagtcttgctgagcccacccgctctctctgggtccatcccatccctgagccaggggaggcagcatttcatgggcatgtctatcagaggtgggacttgcagcctctgccccacgagtttgtctctcagtggactctataggtttgctacttttgcatgacacagcaagcccagtgtcccctttgcaagctgcagagagggaaacagagtcccaggcctgctgggaagagatgctcctgcctcccaccttccagagttgggggcctggcaggctccatggcaggcaccaggtccctagcagcccagaacagctctgactggagccctcaaggcctctggggccagggtctccgtgatcagctcagccctggtgttcctctcttgctgggctgggacctgggacacagccacggcagcaaactcagagaattgaaggtgctggcgccaccctggggcactctgtcttcacagcaggagtgactgtctccagtgctcttggtactcctgtttggctgtggcctggtccctctgggccctgggatcttcttctggcccttgaggcaggtctggagaggcagtctctgtcttcatggaggggtggtcagaggcgggcgggaccaccagcctgtagcgttattattatatgtgacaataaaggtgctctccccactgtgggtctgtttgtgtggtgacagctctgcaggaggctggtctgcatcttaccggccaggtgccagggaagctgtgagcagctccagccaggcccggggtgaccacagggcctctgtggcagggaggtccagctctgccctcaggacctccagggagggggctgggagtgacacacgataggcaagccccggattggggcttaacctgcaagggctcttggcttcacccagcaaagaattcaagggcaagctggaggcaagagaaacagctttattggaggggcaggactacagctccatgactgctcgtgcagagcagggcttctccccaggcagagaatagcagctcagggcagttctgcaccatatttatacctacttttaattgcatgcagattaagggctggtttatgcagaaatttctagggaaagggtggttatttccgggtcatcaggtctttgccatggaaaagggcagtggctcctgagtgttgccatggtgatggcaaactgacatggcacactggtgggcgtgtccaatggaaagatgcttccgctctgtccctgttttggctagtcctcaatttggtcctgtgtctgagccctacctccagagtcaagatctgtttcttacctccagagggtgagggaagccccttcagaggccacctcacccctccacccaaccacactgtctataaactgggcctggcccccagctctgcaagaaggtgagttaagttggggcggcctcagggctcttggaggagcaaccctgatcctgctccatgctcctgtccaggtctggggtcagtgacatatcagagaccccagggagacccatataccctgatgacaagtcccaggtacaacacagagacccaaaccactgctgaatcaccctgtgacccttgtaggtattaagctcttgccctgtgtattatgtctgttctcactctgccaataaagatatacccgagactgggtaatttatggtaatttataaagaaaaagatgtttaatggacttacagttccatgtggctggggaggcttcacaatcatggtggaaggggaaaggcacgtcttgcatggcagcaggcaagagagagaatgagaaccaagcaaaaggggtttccccttataaaaccatcagatctcgtgagacttacttattcactaccatgagaacagtatggaggaaccgcccccatgattaaattatctcccaacaggtccctcccacaacacatgggaattatgggagctacaattcaacatgagatctgggtgaggacacagccaaaccatatcaccctgtgtccagtgcccagtgctcagctacagacagaaccctgcctgcctctgcctggagcttgaaatcctatagagagggtgagcattgaacaccacaccaggccaggcatggtggatcacacctgtaatcccagcacttagagaggacaaagtgggaagattgcttgaggtcaggaattcaagaccagcctggacaacatagctagaccctgttgctacaagtgtttttttttttttttttaaagttagcctggcatggtggtggcttgtccctgtagtcccagatacttgggaggctgaagtgggacgatcatttgaacccaggagttggaggctgcagtgagccatgattccgccattgcactctagcctgggcaacagagcgagatccggtctctaaaaattaaacataaaaaaacaccacagcaatcatgtggaggaaggagattccaggctgctctgagagtccaggacccagagaccaaaagggcagggagagcagggaaacagaaagagagaaagagggagggaagggaaaagagagacagagcaagaggcgagagggagagagaaacaagagacagagacagagagatggcctgccagagcttcatctgtctttatttgctggtgaataggcaacacgttccttaagccggagcctcttcttccaagccccattggctctgggatacttcctccagccacaggtcctgggcccagcaggcacagctcattcagcaaacagggagacccggggtcgggcaggtgcctgaagtcaaaccttggcaggaaccactgggcaactgtcccaggcagggagtgtgagtaggtttaggctggagaaatgtcctcgccccttttattccttttcccagggcaggagttacaaaaccacgcatctttccttccttcctcctttccttcatgtaacaaatcttttattgatcaccttaattgagccaggccctgagaactttccatgaacaaaagacaggaatctctgccctcaatgaaccaacgcttcaaggggaagaagtcaaaataggcaataaacagcctggacaacatagtgagaccccatctctaaaaaaaaaaaaattagccagatgtggtggcatgcacctgttgtcccagccacttggaggctgaggcaggaggatcacttgagcttgggaggtcaaggctgcagtgagctgagatcacaccactgcattccagcctgggcaacagagcaagaccccgtctcaaaaacaaaacaaaacaaaaccaaaataggtggtaagtgtatgtatttttaaagccaataagatgataggctagaagcctagccacgtccagatgatcagcaatatggaagcgctaaggactgggagctgacaattagtcttattttttaatttttaaaacttattttaattttgtgtgtgtgtgtgaaaaaacacctcctctcctccttggggaagcagaccagcaggcattcattcccccagacccagtggcagagatgggatcctaagagagagatttgcctgcattacctaggacaccaaaacgtcctgccccagggctgcccccacttcccccaaggtcttccccttccttttgtcttaaaggcagactccacgcccaatgacacctttgtctttcaatcaaggggttgctgagtttggagggtgtgaactgagcttagacgctccaggtgggcaccctcaggtgggcacacgaagccccccgtggtgcaaggtggaagctagtgagggaagcagaagggctggaggatgacaggggctggctctcccggctgtcaccttccagcacaagactctgacagtcacaattccaacgtcaaacctggccttccaggtccttggaaaaagatatctgtcaaagtaggaagatagagcctgtgttacttaacagctggttagctcaatgtataactttttttctctctctctctctgtcgcccaggctggagtgcagtggcacgatctcggctcactgcaacctccgcctcccagttcaaacaattctcttgcttcagcctcctgagtagctgggattacaggtgcctgccgccacacccagctattttttttttcctgtatttttagtagggacggggtttcaccatgttgtctaggctggtctcgaactcctgacctcaggtgatccacccatctcagcctcccaaagggctgggattacaggtgtgagccaccgcacccagcctcgatgtgtaacttttaaatagactcatgggatgccttcatttttcctctggtccctggatcccgctatgggagagcctggatgggaccagcagagtctaggagggtagggaaagggctgtatgaggaatgtttggggaaaaacaacttccaagctcccgccagcagggtggacgggccagcccggagtgtgttcgatggagccagggtagcagatactgtgggtcccagagaaggtctgagacagagctgagggagaaggtctcccccaaggggctgggtgtgagttggtccttaatatggtgaggacagatccagtgctaaacagtgaaacatgtcacctcctttaacgtcacagccacctctctcattttggggtgagtcagctgagccccagagaggttacatagcttcctcaaggtcacacaggcggttggcggtgaagctgagttccagatcaggtctgtgggatttgggaagccatgctctgctctgcccccgagaaagcggttctggaaggaaaaaagagagttcacataaacccagggatggcgctgccgaagaggccaagtgtgagcaaaggctgggggcggataaagggcagaggagatttgggtgggggtggaacgaatctcccaggacactgaagcacctgccagggtggggatctccgaggaccgtgatgctggagggagaggaaactgaccactgagggtctcagtctggcctctccttgccatctggccgtagaatgacctttctgaagtgcaagcctggcgttgctcctccctcactcatgaatctgctgtggctcccctgttcctggcctcaagcctgagctctgccccctggtgctgtcacctgggtccctgtcctgaccaccttctgcacacatagtggctgcagccccaggagtcagccaggaggtctgcccattgtccggcctcatctctgtccagctgtggaggctggctccccccgcgcaaagcagtgctaagcgtctgcaggaaatgggacagcgctgttgggggaagggcctcaaagccccagggggagcccagctcttggccttgtgtgaccccctcaaatcttcacctcctctcacccctgcagccaaggagacagggcgggcccatctgatcctctgcccagcttcaggaaggttcttgcatgcatgtgtggacaccgccgcttgcacttccaagctgtccacagggcccaagactcggagtaggaagcaaagcaccccaggctcccagctaggacgctgcagggctggactgtggtcatcctcccctgcccaagattgccttccctcctccctctccacagcccctggggaaaggggactcaccagggtcagctcagtcctggcaggagcctccaggtcccctcctggctcacagctgagctttgagggccagatctggggacaggctggctagagccctcactgcctgggtgcccagaactcccagtctgaggccacctgcctgggcactaaggagggagaattagcataggcccctgctcacctcgccccgccctgctccaccctgtctccactttgcctggcccagacatggctgtctcgaatttcctgcctgagtctcctctgctctcctgcgcttcctgcctctgcctccggccgctgtttcctgcgcatacccccttctgtggcccggggtccagatcaaacctgacatttcttctgaaatgccaactggctctaggccctgctatacatgtgcgtgtgcatatgcaagtgtgtgcatgcataagaatgagtgtgttcatgtgtgtgcatgttctgtgtgtacacatatgtgcacgtgtgtgcatgtgtaagagtgtacatgcatgagaatgtgtgtgtgcgtgtccagtgcgtatacacatatgtgcacgtgtgtgtgcttgaatgtatgtgtgtgggtattcgtatctgctacggtgtgtacacacagtgtggaggtggctcgggctgggctgaacctcagaggttcccacgaaggcacgctcttcttagcctggtcctggcatgtcctgaagacccacgaggggcaactgtcctaccggaatggcagaaacagcccgactctggctttgggctcagccaagggtggtcaaggtgccattggggtcccccagcttcactcgtcacctccctaaatccaaagcgcaccctcccaactctgtctcttccagtcggctgtgagcgtcctgtgtgccaggtcacgtctgcgtgcccagctcaggccaggtgcagagcagatgaatgactgcaaaggaatcactggcacaaggcaggacattcacggtgggggcgtggcttcttccctgacactggcacatagcaggacattcacgatgggggcgtggcttcctccctgacactggcacatggaggacattcacgatgggggtgtggcttcttccctgacactggcacaaggcgggacattcacgacgggggcgtggcttcttccctgacactggtacacggtgggacattcacgatgggggcgtggcttcttccctgaccctaagaggccatgttggaggttatgggcaggggcaggtgggagccaagactggcctacatcagaacagcagccccctccccactccaaccctccacgcctgcagtttccaccacccccgagatttctggaacacctctctcccagaccggctttatggattatataaaaaaagaatttcatgaggttttcagttaattgaattccagggagatttattcccaatgttattaataaattctaatgccacctataactttcaattgccagttaattaacttgcaatgggtgcattgtaaaaggcaaattaaatactttttcaggcaggggctggcaaatttaatgagctgatgtgtcccaagggagacggccgggctcacacatcccatcaaatactcctcccatcagcagatgagagtgtgatctgggtgagtgacaactggacctccgcatcctcatctgtaaaaagggatcacaaaacctgcctccaggaccctcagatacctggagtggatgctgtgttttattcatacacaaatacaacgaaggggccctggggcggtgaactttatgtgtcaacttgactgagccatggggtgcccagacaactggtcaaacacgcctgccctgcccagcccaggtcaattcatctggtcagtctacaggggattgggggcaggccagcctctgcctccttctgagtagagaacaaggatggcagtaaggacggcagggaagacaaggagcaggggatcccagggaggaccaccgggctcactggagggaagagagtcctcccctaggaagggacacagctgaggagtctgagagatttagtaggagttgtctgggtgaaggagaaggcagggcaagttggggaggcacgagcacgtgcaaaggcccagtggtatgagaaggggaaaagcactggaaattgttggaaatgctctagggtggagccggcaggggaaatatgataatgtgggcttattacaaggctacagggatctcagtggaccccaaggacaggagctggagtcagaacaggtcccaagagagtagtactggatctgagttagtcccaaggcctcagtgtcagcaggagattactgattcccatgggctctgcctgttgcctgtttgtccctcttcttgtggcaatgtgacattttgtcttggcctcatagcttcagcatgctcatgacttctgcttcttctgatggcttgccatgacctctcactggctatccttcaacagcagtcagctttgcagtgacacaatattcaaactgggtgtcaagcaccttcattgtctccccagtgacccagcacgcaggctccctaaaggtagagttctcaggcagtacctggtgctgtaacaaatatcccattgacctgggggcgacatttctcacagctctaaaggataggaagtccaagatcaaggtgccagcagatccggttcctggtgagggctctctttctggtttgcagatggccgccttcttgctgtgtcttcacatggtggggagagagagaggaagcaagctctgttgtgtctcttcttataaggacattaataccattatgagggccccaccctcatgaactcatataaacctaatcacctttcgatggccccacctccaaatgtcatcacaatggggattcaggctttaacatacaaattttaggggcacataaacaagtggtctacagcagtagaatgccttgtccttgtaggcagacaaggacagacctcggtctggtctcccctaggcctagacacagatacttgataaggagggaggattctagggaaccaaggtcagaagcctcatagagcaggaagtatgggttttgtggcctgttgatggcagaagcttctatcttaggcttagagtctccagctaggctcttgctgggaaaacagtgcagtctttgctttcttttatatcagagctgcattttcaaacgtaaccatcccacacccactctccagctattcgaggactttgatgacagccctccagtttctcaggatgtaacagcagcaaatcactgtgcactttctcacttgctgcccatgatcccccatgctccaaccaccgggcaggcccatattgggcatgaaaggatcttggccttaatttttcttttgcctcgtggcgataaagggagcattttgtttttttcaaaagacaaaaccaaaaggtgcatcttaattagggaacaggtgcagtgagaccagactgaactctgccaggcagcagcctttcactttgtgcgcacaatgcttttaatgcagccccgttgccatggcgattaccatgtctcagcttactgaggtggcgttgcaaacctgtagggaagaaatggattattcaataaatgatgccgggacatttggttagctatttggaaggaaataaaattagctctttccttcacaccatacattaatataaattccagaggacctaaagaattaaatgtaagaaagggaaatcattaaaaagcaaacaaatgtttgatcttgggggtacatggaaacattttaaacctaaaagcagtagaaaaaataacaaagattaaggtagaatttaatacataaaatttaaatctccttgttttaaaaattatttagttaattttattgggagactacaaaaaagaataaaatggttgcaagaaatatgacaaaagattaatacctctaatatgtaaaaagtaattacaaatcaataaaaatgaacagtccaatagaaaaatagtgaatgagaaatgcccaaaagatgaaataaaaatatgtaataaaatatttttaaatattcagcttcattggtcatcaaagaaatgcccattaaaacatcaatgagattttctcctatagcttatcaaagaacgtttaaatataattatagcattcaatctcattgagtggagtaaaattgagtattcctattcatctcacatgtgaaaatacaagttggtataaaatttctaggaaaaaaattaagtttatgccacaagagattttaaaatgttcacactattcagctggtaatttcactccgaagatatcctaaggtgctgtccaggattgaagccaaaaatgtagatatgataatattcattataagtatttaatagaagtttataatttactaaaacacgtgggcatggttaaataaattatgatacataaatatgatgaactattgtgcagccattaaaataatattttgaataatacttaatggcatggaaatatgtctatgacataatattaagcggaaaatagtataccatttgggtttcttagctgacagcaatagaaacccactctggctgtctggataaagcagaaaaggaattcattggaagaaaattgtagggcttggaatcaacaggaacacaggagaactaaactgagaaagaaagaaaaatagagaaagaaatagaggaagagagagaaagcaaggaaatatagagaaaagaaagagagaaagaagaatgaaggaaagaaccaaggaaggaaggaaaggagcaaaaaggcaaaacagggcaaacatccatcaacagctatagttacggtaccaaagtagacatatggtatgatcccaagtcaaggcaaaattggctattcagaaataccaaattcatcccggaatctcagtgcttcaacccttgcaatgtttccttcttactcacacaaagtaagtacttaccaggttgggcagcctgtcaccatcttgtagcccagccattgaaagaacgtaggctctgagactgtcacaacagggaagacagagatggaggagtctccatgcgagtgatgctgtcatttctgttcacagtccattgggcagcactagtcatatggcccccaacctcactgcaatggaggctgggataaagaaggcagtgagccagttgaggctgcaatttgaaacacatggtacacttaatttaggataatttgaggaagatttaatttcaaggagactattttcaaaggtgtgggtgggctgcagaagaaccagaagggagaatgggagaatgtgatgagctagggctattaacagcccaaagagacgagggtcagattggattttcaaaatctcaactatatgctgtttataagcagcacattttgaataaaagatcactatagaagatttgaagcatacagttatccagcttcacccaatggacaatatatagaacacacgcctaccaattgcataatatgtgctcttttaaagtgtacatggaacattttccaaaatagaccatatgcctagccataaataaatctcaacaaatttcaaatgattaaaataataaaatatgttttctgactacattgaaattaaactagaaactaataacaaaaaaaaatagaaaatatcccaaacccttggaaataatccacattcttttagataattcaatatgtcaaagaagaaatcacaacataaattataaattcatggaaataaaaatgtaacgtataaaatttgtgggatgcagttagcacagctcttctaagaatatttacaactttaaatgaatatgttagaaaagaaaaaaggtagaaaatgaatgatctatgctttaatctcaagatgctaaagaaaaaacagcatatttagaaaaaggagaaggaaaactaaaggaaataataaagataatggcagaaatgaataaaatcaaaagcaaatgtatagagaaaatttaaaagccacacatcgattaagattaataaaactgataaatacttagcaggctggatcaagaaaaagaaatgagaaaacataaattagccatgccaggagacatcactacagatccccaagactttcatgatacagcaaaaggatattgtcaacagctttatgccaataatttaaaaatttagatgaaatggccgggtgcagcagctcacacctgtaatcccagcactttggaaagccaaggtgggcagatcacaaggtcaggagatcgagaccatcctggctaacatggtgaaaccccgtctctactaaaaatacaaaaaattagccaggcatgatggcgggcccctgtagtcccagctactcgggaggctgaggcaggagaatggcatgaacccaggaggcagagcttgcagtgagccgagatcatgccactgcactccagcctgggcgacagagtgagactctgtctcaaaaaaaaaaaaaaaattagatgaaatggataaattcattgaaaaatacaatgtactaaaatcattataagaaaaaataaaaaattgatatacttttatatctgctaaagaaattgaattcataattagaaacattcccacaaagaaaactccagcctcagattgcttaagtgatgaattctaccaaacattcaaggaaactcatagagacagacagacagatagtagattagtggttgccagcggctgggaagtatttgcttattctagacatgttatatcaatggaatcatacaatatgtggtcttttgtgtctggcttctttcatttagcatattttaaagattcacccatgttaaaaacaaataaataaaattttattcatttttatggctgcataatattctaccatatagataaaccagattttgtttatccattcctcagttgattgacgtttgggctctttctactttttgactattatgaataatgctactatgtacatttttgtacacgtttttgtgttgacacatgtttttgcttttcatggatagctatcttagagtggaatggcagggccatttggtaactctctgtttagctttttgaaacactgccagactattttccaaagtggctgcaccattttacaattccaccatcaatgtatgaggaccacaacctctctatattctcatcaacacttgttattgcctttttaaattttaggcaaattagtgaacatgaagtggtaggtcactgttttggtttgcattttcctaatgactaagaatactgaacatcttttcatgacttattggccattgtttatctttgaagaaatgtctcttaaaatcctttgcccacaacagacctctacctcccaccatataaaaaagtgaactcaaaatagattatacacctaaatgtgggagctaaaactataaaacgcttggatgaaaacattgatataaatcttggtgaccctggattaggcaacttgtggattgaagttctaattggtatgtttgcatttttactgttgagttgtaacagttctttgtatattcttgataaaaagtcccttatcaggtatatgatttgtaaacattttcttccattctctcattctgagggttgtctttttactctttgatagtacctttagagcacaaaggttttatttttgagaaactcctatttatctaatttttcttttctttcttacattttggatgtcacatcaaataaaccattgcttaatccaaggtcataaaaatttacacctatggtttcttctaaaggttttgtagttgaagctcttacatgtaggtctataatatattttgagatagtttttacatggtgggaagtaggagcctgctgtgggtggaattgcgcctccatcaaaagatatattagagttccagctcccaatacctgtaaatattaccttgtttggaaatagggtctttgtagatgtaaacaagataggattggacctatatccaatgactggtgtccttataagaacaccatgtacagggatatagagatacacaagaagaatgccatgtgccaatggaggcagatataagagtaaagtgtctgcaaaccaaggattgcaaagtatatacaagccagaagctaggaagaagcaaggaaggatcctccccaagaaccttcagagagagcaaggccctgttatgacaccctaattttggacttctagcatccagaactgtgtgacaatagatttctttgctcttaagccacacggtttagagtaatttgttatggcagtgaggaaactaataggggatccaacttcattctttttcaagtggatatacaattgtctcagtcttttgtttgaagagactattctttccccattgaatagtcttgacacccttgttgaatatcagctgaccgtaagtgtaagtgtttatagctggactccaattagattctattgagctacatgttttgttcttataccagcatgacattgtcttgagtatggtaccttagcagttttgaaattaggaagtatacatcttccaacttttgtttttctttgtcaagattgttgtggctagtcccagttccttgtatttccataataatcttggcattagtttgtcaatttctaccaaaatagccagctgaaattttgatagcgattgatcaatttggggagtattgctatcttaacaatattaactctttcaatccatgaacatgagatgtctttccatttatgtattttatttaattactttcagcaatgttttataggttcccacataaaagtcttgcactacttttgttaaacttaatcctaagtatttccttctttttgatgccactgtaaatggatttgctaaaactttttcaggtggtcacagtggctcacacctgtgatcccagcagtttgggaggccaggatgggaggaacacttgaggccaggagtttcagaccagcctgggcaatatagtgagatccgtctccataaaaaatttaaaaattagctaggtgaggtggcaaatgccggtagtcctagctacttgggaggatgaggttcaaggtttgcttcagctcatgagtttgaggctgcagtgggctatgattgaaccactgcactccagcctgggcagcagagtgagaccctgtctccaaaaaaaaaaaaaaataaacaaaataaaatgaaatattttcagattgtttattgctagtctatagaagtaaacttgaattttgtatgccaaccttatatcctgaaactgctgaacttgtttattagttctaatagtgtgtgtacatattctttaagattttgtatatacaagttcttgtcatctgagaatagaaatagttttatttctctttttccaatctggatggctttatttatttttcttgcctaattactctggctagaatctctagtacaatgctgaatagagatagtgagagaggacatcctagttgtgttcttgattttagggtagaaacattcatactttctccattaaatatgatgttagctatgggttttttgtagatgccctttttcaggttaaagaggttcccttatattcttagtttattgttttgttttgttttgtttttatcatgacagtgtgttagattttatcaaatgctttttctgcaaattttgagatggtcctgtgtgttttgtcctttcttctattaatatggtgtattactttgattgatttcagatgttaaaccaatcttgcattcttgggatgaatcccacctggtggtgatacataatcgtcttatatgttcctgaatttggttttccagtattttattgaggatttccatctatgttcataatggatactggttcataaatttcctttctggtgatgtctttgtctagttttattatcagggtaatactggcatcatagaatgagttggaaaatgttctattagctcatattttctggaggttttgtgaagaatttgagtttattatttgtgttagtccattttcacagtgctataaagaactacctgagactgggtaatttatgaagaggtttaattgactctcagttccacaggcttaccaggaagcatgactaggaggaggcctcaggaaacttatagtcatgatggaaggcaaaggagaaacaagcaccttcttcacatggtggcaggagagagagtgagggaagcaggaagtgccacacaattttttttttttttcaagacggagtctcactctgcaacctccgcctcctgggttcaagcgattctcctgcctcagcctactgagtagctgggattccaggcacatgcccagctaatttttatatttttagtagagacacggtttcaccatgttgatcaggctggtctcaatctcctgaccttgtgatctgccggtctcagcctcccaaagtgctgggattacaggtgtgagccactgcacctggttgagaactcactcaatatcatgagaacggcaatggggaaatctgccctcatgatccagttacctcctatcaggcccctcctccaattcaacgtgagatttgggtggggacacaaatccaaaccataacattctctgcatttttggtagaattcgccagtgaatccacatgagcctgtgtttctattctctattttatctacttcactccaacagatacaggagctagaaagaaattatttaggaagatagtgagggtaagagaatcctagtgtagcaggacgagccacagacaaaactcctcagacactgagttaaataaggaaggggtttattcagccgggggcattggcaagactcctgtctcaagagctgagctccctgagtgagcaattcctgtcccttttaagggcttacaactctaagggggtccacatgagagggttgtgatcgattgagcaagcagggggtacgtgactaggggctgcatgtaccagtaatcagaacgaaacagaacaggacagggatttttacaatgattttccatacaatgtctggaatctatagataacataactggttaggtcggggtcgatctttaactatcaggcccagagcatggtgccaggctgtcttcctgtggatttcacttctgcctttcagtttttacttctttctttggaggcagaaattaggcataagacaattatgaggggtggtctcttcccttactaggtaaggtttccttttaataaaaagcagcccccaaataatttcttttctaacaaaaagcagcctgaaaaatcaagcttcaagcatagataagcaagctcaaagcgtgcataggtaaatgccaacagctgtgccaatagaaaagggacacctggaagctaggtatattcaacatggaggttccttcttcccttttctttgttgccacatgtgtagtaaaaaagcaggcaacatggcattggccaagtagagaccccatctgcataacaaaagattagggtgggatggccagcttcttcgcacactatgcaaatgggacacctggtctgaccagtctctcatgtcctatgtaaatcagacactgcctcctcaagctcgtctataaaaccccatgcatttcaccatgaagcccaaagaccctacttgggagtccctctccctctgcagaagagagagcttttctctgcctttcacttattaaacctctgctcttaaactcattccttgtgtgtgtttgtgtattcagtttccctggtgtgaggcaatgaacctcgggtattttccccagacaaaccatgccccttcacaatctttattatttcctttcttcaacttgttttaggtttagtttagacttctttttcaagttacaggaagtggaagtttaagtgactgatttagatccttattctttttcatataggtgattacagctataaattttcctctaagcactgctttatggctgcattctacaagttttggtgagttgtttcatttttatttaaagcatgctctaatttccttgtggtatcttctttgatccattggttacttaagagtgtgatgtttaatttccacatgttcctgaatttcccaatttccttctgttatgcattctcatgagattagaaaacatactttgtatgattccaatctttttaaacttactgagtacttttttatggcctaacatatggtttacagtgcagaatgttccatgtacacttgagaagaacatgtattctgatgttgttgactggagtgttctgtaggtgtctgttaggttccactgaataatagcgttcctttaattcttaagacgtggtttcctctagtttttgcacatattcataacagtgtacttgaagactttgcctagtaattccagtgtctggacttcctcagagacagtttcctgtgaatggaccatactcttctgtttctctgcatgtctcataattttttgttgttgaatattagacatttaaaataatataatgtagcaaatttggaaatcagattccctgaccccccaccacaaagagtttgttgttgctattgctgtttgtttccttagtgatttttctggattaattctataaagtctgtgttctttttcatgttttgctgctaaaattcctgttagcttagtagtcaactaatgattgcacaggctttttttttttttttttttttgacagagtcttgctctgtcatccaggctggagtgcagtggcgtgatcttggctccctgcagcctctacttcctgggtttaagcaattctcgtgcctcagcctcccaagtggctgagactacaggcacacaccaccatgcctggctaatttttgtatttttggtagagatggggttttgcgtgttggccaggctggtctcaaactcctggcctcaagtgatccacccacctcggcctcccaaagggctgggattacaggtgtgaaccactgtgcccagcccagatatttctttaaatcccttaaccccataagtgtcccagcctttgctgaggaacactgtgtgtgtgtggagcctgacttcaatgctttatcaagcagtttgcagctctgccgtagccttcatttccttcttgtgcagagcctcaaggtcagccagaggtgagacatcagggtcttatcaggtctttctgtgcatgtgcactgctctgcacatgcatgaagcctctatcctatcatcccaggaatgtatcgaaacttttaaaacccccaaaggaaaaaaaaacccaaaggataactcatttcccagtttttcctttcatgattttacatagcctattgaccccaaatggtattgccacataggcagctacgatgttgaacagttactgctgattgtttttgacaagtgccctgaggccagggctggtcacacagagtaagctctgagtcaggtcagataaagacaatccctgggaacagagcttttcaggcagcttccagacaggtcaaatagtggccattttctggggatagagctttttgtgggctggtttcgcagacatcatagttgcaaggctgttggttttcatgcctgcaacagaactgtaaagagggctgtgggaataagacaagttaaaacttcacaaagcttgctgttcttactgcaatacggccttattttattttttttagagactgggtctcactctgtctcccaggctggaatgcagtagcatgatcataactcactgcaaccttgaactcccagcccaagtgatcctcctgcctcaccctccccagtagctaagactacagccatgtaccactgcaccctgatattttttaaaaaatatatagtatttgtaaggatggggtgtcactgtgttgcccgggctggcctcgaacgcctggcctcaagcgatcctcctgcctcagcctcccaaagtcagccttattaaaaaataataattgtaataataaacactcctcaggtagctgcaagcctttggttaatttgcagaagtatgaaaaaattgattttgactgtttttgccagcatgctcattgcttttatgaaggaacagatttcagaagtccttattctaattatcctctcattctggtcatgctcctccacctgtttttgtgcctaaagttttattggaatgcaggtttgcccattcatttacatagtgcctatagtggctttcacacacaactgaaaagttggacagttgcgacagaaaccatatggcctgcaaagcctaaaatatgtactatctggcctttaggaaaaggtttgccaagccccaccataaggcaagaaaatgatatacagattgcaaggaaaaagtaaagtgccatcatccacaggtgaaattttttggcatttagaatttttttattttttgatcatttattcatttatttagagacagggtctcactatgtcacccaggctggtcttgaactcttgggctcaagtgatcctcccatcttggtctcacagagtactaggattataggcatgagccattgcacccagcccagaaaattttaaaatctgtgaacaacttgttagaattagtaagtggctggacatggtgactcaatgcctataatcccagcgctttgggagactgaggcaggaggattgcttgaggccaggagttcaagaccagcctgggaaacacaacaagaccctgtctctacaaaaacaataaaataaaaatcagctgggtgtggtggcacctgcctgtagtcccagatactggaaggctgaggcaggaggatagcttgagcctgggagttggaggctgcagtgagctatgattgtaccactgtactccagcctgagtgacagagtgagactgtgtttccaaaaaaaaaaaaaatagtaagagaatttagcaagatcattgactacattagcatgcaaaactcagctgcaggacttatgaacacacccctccctgcctctctcagtgatcacgactaaaaattctgaacagaatgcctatagtaactgtctgagaactctgaaaagtagatcataggtggcagattaaggaaggaaatgaaaactgaaagaatgatgggcattgctgtgagttcctggactggttggttggttggtttttctccacattccataagatagattctagcacagctcaaatcctggagctgtgaagtggttggaggaaatgccttgtctttctggccagagaaccaggaatgggggacaaagagtcagggggtgggggcaactccaaatttttattttttatttcccttttgactgcatctccatttgctctcttctcttctgaaactcattttcaatggatgtctcctcgtaggttgaccctctagtgtttttgtcttttctctcctaatatccaatcactctcccatttctcctgggattttccgttgcacatgccatttcctcttcagggaacatcaaagaacactcttcctgcccctgcaaagagatgtctacttaactccaccacccaacattcagatggcttttgccatcttctctgactagccaagtctctctactggatgctgtatttagtccattcaaatgctgctaataaagacataccagagactgggtaatttgtaaaggaaagaggttcaattgactcacagttctgcatagctgcagaggcctcagaaaacttacaatcatggcaggaagggatgcaaacatgtccttcttcacatggtggcagcaaggagaagtgccttaacaaaagcagggaaagctcttataagaccatcagatcaggcctggcatggtggctcatgcctgtaatccctgcactcttggaggccgaagtgggcagatcacttgaggtcaggagttcaagacaagcctgaccaacatggtgaaaccctgtctctctactaaaaatacaaaaaattacctgggcatggtggcgcatgcctgtaatcccagctacttgggaggctgaggcaggagaactgcttgaacccaggaggcggaggttgcagtgagatgagatctagattgcaccattgcactccagcctgggcaacaagagcgaaactccctctcaaaaaacaaaaaaaaacaaaaccaatcagatctcgtgagaactcactaattatcatgagaacagcagcatgggggtaaccacccccatgattcaattagctcccaccggctttctcccacaccacatggggattatgggaactacaattcaatatgagatttgggtggggacacagccaaaccgtatcagaagcactatgtctttttcatttccagcactggttagagttgtcaccttgcatttacttgcacaattcttagattgctgccagtttcctgttcggtccataagccctatctgactttggtccatcattgtgcttccagaatctaaaatggtgtctgacatgtagagggcttccaaaaatgtttcctgaatgaatgatgaatgtctctatgtctttgcacatgttgttccccctacctgaaatatccaccctttccatcccctgtcagtacacctctgcattcctacaagaaccagcccaagtgtcaccacctctgggaggcctcccctggtgccctaggcagacacattgcttctgcagcattgtgaccatgggctggaagaaagcagccttgtcaccttgcactacagtagcctggggcataaatattgtctaagaaatcattgcagtttccatatttttatgcaataaggaagcctacttccagcatgaacccaacttagaaaatactctcagccaatatttcagatttcagcacaaccctccatcagggtgcatgacgtgaggactttacaaatgccgcggcagaacctatgggcctagatgagagggaaggggtggaagctgtgcaagggcccacagcacgggctgccctgtggaggcgggtctagatgctgctgctgatgcaggtctgatgtgtcagcacagagacccaccctgagccctggaggagtccagcccaacgcttagtagcaaggcatttacagaggatccattccactgtggagggagcatgatttatcctcgtaaataacacctactccacattgatatgtgtcttccttggcacaaccctgccagctctacacatgggatttcaaaagtgactgatttctcaatctagaatcctgcacaatgtcaccttggaccaagaaatactttttttttttgagacagtcttgctctgttgccctggctggagtgcagtggtgcaatctcggctcactgcaacctctgcctcccaggttcaagtgattctcctgcctcagtctcctgagtagttgggattacaggtgcccatcatcacgcctggctaatttttgtatttttggtagaaacagggttttgccatgttggccaggctggtctcgaactcctgacctcaagtgatccacccacctcgacctcccaaagtgctgagattacaggcatgagccactgcgccccgcccaagagatcaattttacagcaaaaggtccacaatggatacatggcaatgggatccattggtcctatcaacaactacatgagctggacacagtcagcctggatgaccattgaaatggcttattaaaggctaggctaaggggtcaatttgagctttctctgtggggtttggagcttgtattaaattaaaatgtatctggtggctcatgcctgtaatcccagcactttgggaccccgaggaaggaggaacgttttgaggccaggagttcaagaccagtctgggcaacacggtgagaccccaactctaaaaaatatatataaaatagccagccatgttggtggatgcctgtagtcccagctattcaggaggctgaggtgggaggatcgcttgagcccaggagttcaaagctacagtgagccatgattgtgccactgcattccagcctgggcaacagagcaagactctgtccccaactctctgcctccaagaaagtacctgggatttgttttaatcaggtatggtgaggccaacaggtcaggagatgaatgccgtgaagcaaaaatttttactcacccatccctagaaacaggaggccacgtggagccactcaggaaagcactcgggtaggtcaggagggagaggcggagggggaacggggcaagagccttcactgtggtttgtgcaagaaggaacgggtaaggctgggggaatgggttgattgggtttgggattggctcatttgagtaatttcagcagcctccagggtataggggccactcctagctgtctgatgtctggccctgggatgctgggggtagaggaatattgtctccaagagtgcaaaatccagatggagaaggtggttgagtttaggctctggattggtcggtttgcataccaaaggcgtgctagcgctcgcaggaaagttgtttaccatctctaggaattcactagcgctggaaggagcagtctctccaggattagcaaggcccccagaagtcaaagcatcataaagtacagaaaatttaaacaaatattactgatacgagctatcttctacaatgtgccgtacgcattgaaccaacagttgatgcaccgtatcccagtagctggaattgaaattttcccaaagcaaggactgacccctcccaaatcactcccagtgaccccagtggcaccagacagcccgattttccagttgccagagaaacacttccattagaaaattcactgaaatgggccaagtgcagtggttcacagctgtaatcccggcactctgggaggccgaggccagtggatcgcttgaacccaggagtttgagaccagcatgggcagcatggtgaaacctcatctctaccaaaaaaaaaaaaaaaaaaaaaaaaaaaaccaaaaagttagctgagttgtagtggtgtgcatctgtggtccatgctacttgggagactaaggtgggaggattaccgagcctgggaggcagaggttgcagtgagccatgatggcaccactgcactccagcctgggtgacagagtgagaccttgtctccaaaataaataaataaataataaaaccatgaaaagttcctctgaacctgcatctgtacatgtgtaaatgttatctgtcactttgggtttcttgtgccgatgaaatagaattcaaaggaaggagctgccatactaatgggggtcattactgagtattatgaggggtatggatggctgctgcacagtggaggtagggaggagtctgtctggaacatggggttttaccaggaccccaggagcccagcagaccatgaggatcttggtgcattcgtgcctggcgataacggtaaacagggggcagtcatgcctggcagagaacaggcaaccaaaggctggggtccctcagggatgagggcctgggtcaccccagcaggcaagagccttagaccatctgaagtgctggctcagggtgaaggaaatcttgcatgggttctctctagaggggactgatgacaaatattaattatggtcttgggaccagctgcagctgcccagaatgtagctcattccaacacatctttgtattcactctctctgtaggtcatgcctgtccactctcccgaagactcagtgccaccatggacttggggtgggcacacgtggatctgagtggtgcaaggggtggactgtggcagacactttctggtgctctgtgtcactttccattaatttccttctgatttctgcagcagcagtggtggtcagttacgggcaagcacaggcatgcagggagtgtcccacatcaagcatgtgacagcatcttttccctctctggctcagggtcctttcaacaacaagggtctgttgaggctatgagcaagctctgtccaggattgtgaggaagtcagtgtcctcaagggacaccctctacctctcggggggtggaggaacaggtggatacatgctccaggctccttcttgcagaaggacaattctgggagacattctgtactctttgcaagggttcctacagaatcaagccccctttgctgaagcagcgaccttgataactcaccctgacaccagcttttcctctttccctttaacactctttttttcttctgtctgttccttgaggtgcctcccaaataaactcccaactcccaagaccttgtcttaggttctgctatgaaggcccccaaacaaagacacccccgtgtttttgtttgtttgtttgtttgtgacggattttcactcttgtcgcccaggctggagtgcgatggcaggatcttggctcactgcaacctccgcctcccgggttcaagcgattctcctgcctcagcctcccaagtagctggaattacaggcatgcgccaccaagtccagctaattttgtattttttagtagagacggggtttctccatgttggtcaggctgttctcaaactcccgacctcaggtcatccgcctgcctcagcctcccaaagtgctgggattataggcatgagccaccacacctggcctgtttgtttgttttttgaggcagggtcttgctctgttgcccaaactggagtgcactagtgcaatcatagctcactgcatccttgacttcccaagttcaagtgatcctcccacctcagcctcctgagtagctgggactacacgcatgcatcatcatgcctagctaattttttgttgagatgggatctcactatgttgcccaggctggtctaaaactcctgggctcatggccaggtgtggtggctcatgcctgtaatcctagcactttgggaggctgaggcgggtggtcacttgaggtcaggagttcgagaccagcctggccaacatggcgaaaccccgtctctactaaaaatgcaaaaaaaattagctgggcatgtggtgcatgcccgtagtcccagctacttgggaggctgagacaggagaatcacttgaacccgggaggcagaggttgcagtgaactgagatcgcaccactgcactccagcctgggtgacagagcaagacaccatctcaaaaaaataaaaataaaaagtaaaactcctgggctcaagtgatcctcctacctcagctttccaaagtgctaggattgcaggcatgagccaccgtgcctagccaaagacaccctgtaatcaagcagtcctggtggaagggctggggtggcccagggcagcaggtgtctggccaacagtttctagtgttttgtgactgctacttactgacacacataaaagagacagtcatactggtgtgtactggtggaatgtcgactcttatcttcaacagtgccaaagttcagggtttagtaaaagtatttaaacagggcgtggtggctcacatctgtaatcccagaactttgggaggctgaggcaggtggatcatgaggtcaggagattgagatcagcatggttaatactgtgaaaccctatctctactaaaaatacaaaaaattagctgggtgtggtggcacatgcctgtagtcccagctactagggagctgaggcaggagaatcgcttgaacctgggaggcagaggttgcagcgggctgagatcatgccattgcattccatcctgggtgacatagcaagactctggccctccccgcaccccccgcaaaaaaaaaaaaaaaagtacttaaactgggcctggtggctcacatttgtaatcccagcactttgggaagctgcagcaggaggatagcttgggcacagaagttggagaccagcctgaggcaacatagtgagaccctccatctatacaaaaattacaaaaattagccaggcatggtggcatgtacctgtagtcccagctacttgggaggctgaggtgggaggattgcttgagccaaggaggtcgaggctgcagtgagccatgattgtggcactgcactccagcctggacaagagtgagaccttgtctcaaataaataaaaataaaaaacagtatttacaggtctgagaaccgcgcaggtcaagccattcataactagttctttcatgtattcataacattccaagacaaaacctccatttaggtgattatctgaatggctggggcctatgccactggtatacagagtatgccacttggtaagtggtctctcttttcctaaatggggcttcagccagaaatttgtcttgtttggttttgtagggtggtcagattgtgccaagcattacatatgatgcttgagtcataatcacgttgataacagaaaaagaaaccatttatgggcccttcctgtactaagcacttcatatgcaggatcttgtatgcctcagaattaccctccatggtagctattgctgttacttccaatttcttgatgaggacactggctcagagaggttaagtaacttgtctggggtcactcagcagttaggtgacagagctggtattcttgccctctccctgggctgttttcttagtagcacctgtgagctcccgcctggaagagatgcagtcatttccgctcacatcctattggctggaactggtcacatgaccccaggtgaattgggaaggaggctgggaggtagagaagcacgtggctaactggtgaacatttgcagctcagcatcagatacagccgggtgcaggcagggctctgtgagatgtggtcatctctgcacattgattgccatgcaaggcttgactcagagcatctgttggcctgtgtctgcttctccaacctgtgcacgcacaaatgggaccagccctcggcatctctgagagactagaagaggccttgaactccacttgggatcagctcttccgaacgcagattaattgctgttgatgttctttggacataattttccgcgagacaggatcatttggaaacccataaatacacattcagatgcacgtgatggataagaaactcggctccaacgagtcatttgagctgccataaatcttgtttgcattttcttccctggtgcctgtaaatcaggccctatctccgccttctgggtcctggtggcccctacctgccatccattacactgcaggtggggcatctgacagtcagccccacacgccgccctcatgccctacgcacatccatcacgtggccaaccctgcatcctgccaccctcctggggggccaaggttccctgaagttttacttgtctctcgtgcttttctgaatagcagttgtcccctacccccaaaatcacagattacacaagcctgattgggttttaacgacatgcaagttttaaaagcaaactggagagagctgctcctgggtttctaccttctccttggcctgtgcccctgtgagctgttttgttttcaacaagccaaccatgcaaagagcaggcaaaacctcacggggtttggaatcagacgcggtgggctgcaatggttctgtaattaagagatttcagccgagttctttctctgggcctcagcgtgctcatctgcaggatagagataacaaaccctacttcctcagattgtatctcagtgtaataatactcaggtaatcgacgtaagagaaacccccaaaactcagttgcttaacccaatgtacaggtttgatttttgccccatgcaaaatccagtatggcagcttccttctctcttacagctttgttttctggagtggatattcctctaagatcaccgcggcaggagaggagagagacagaaagaggaccttggctcctaactgctccagtccagaagagatgcagtcactttctgctcacatcctattggttggaactggccacatgactccaggtgaattgcaaaggatgctgggaggtagagaagcacatggctaattggtgaacatttgcattcctgcaagagtggttgtgagtgttcaatcacatgcagttctcggggcagggcaccgaaggggtctagcagagcttcgggatgtcagagatgagcctaaaataagattggccgggcgtggtggctcatgcctgtaatcagagcactttgggaggccgaggggggcggatcacttgaggccgcctgggcaacatggtgaaactccgtctctactaaaatacaaaaaattagctgggcatggtggcacctgcctgtagtcccagctacttgggaggctgaggcaggagattcacttgaacctggtaggtggaggttgcagtgagctgagatcacttcattgcactccagcctgggtgacagagtgagactttgtctcaaaaataaaataaaataaaattttgcctgtgacctgctctgggctgggcagcaatttttgttcctttggaatccaggcaggacccagaggtcagccctggatcagagggactgttcctcctttcttccttctcccaccctaaggatgaaatctgttgcttgtcctggtggaattctctttttccagctcccattctcaaaagcaccccataaaataccaaaaataaacactgttcatggcttccatttggggaggatttatctttttcctttaatacttcttaccacaacataccagtctttcatctttgcatgcctgggtctgtgaccctaccctgggaggcaggccaaaggcggtgtatgcaggcctcttgggtcaaatgaggacttacccctctgaatgctgaggaaagcacaggggactgtctggggaaggcagcacgcagtgaacagacagggagatgctgggtctgggcctggaaggatgcgtaggagtttgtcgagtggcaaggttgagggaacagacagggagacattgggtctgggccttgaaggatgcataggagtttgtcaagtgacaaaattgagtgtgttcctggcagagggagcaaagtgtgcagagagataggaatgggaatggactggcgtgttctgggagcaactggaagttccggaagtctggatgttagggttcctggggatggagaggccaggaggactgggttgtggaagaccagactttatctccaggacagtggggagccagggagcagtgcaagtgaggcggggacgtcccaggacacctgtccaccacggccaacttttgtgacagcactgactcttgtattggtggcagggtggcttgttttctccccacatttcacttcctgagcaaaggcagtcttgcaagaatctgaaaggaggcaccacataaaatgactctttttttttttttaagtaaaaagtcaacagttccgtgatctttgtaaaaattaatcaggaacacatgcgttctatttttcgtaaattgcaagggatcaaaaggaaaggaaaatgctcttagggtgaaagacaatgggagttccatcaccagctggaacactccacacactgtcttggagttggggggccttccctgcacccggcgtcctggtgtgcctccctggagagagagcatgggctgggcacgagatatggggtgggcttagatacgacccagacctgagatgctcccagacaggagcaggggaccaggcccagcagggggcaatgacaatctctggagggacagtccagggccgagggaacccagagaaggcctctaagtgtctcagacacaggaggaggaacaattggagaggagaccaaatgggtgggctggggatgtattaagactgtcttttttttttttttttttttttttttgagacagggcctctttctgtcacccaggctggagtgcagtagcacgatctcagctcactgcaacctctgactcctgggttcaagcaattctcctgcctcagcctgagactgtctagaatgtacagctaatgaatctctatttttctcttgtacttttctcccttactatctgaggatgggggcacaatggcaaaagccctgggcacagaaaatcaatccagcttttctcttacagtgaggccaatcccatactttgtagggtttttcatttttttttttctttttgagatggactctcgctctgtcacccaggttggagtgcagtggcacgatctcggctcactgcaacctctgcctcctgggttccagctgttctcctgcttcagcctcctgagtagctgagattacaggcatgcgccaccacgccctgctaatttttgcatttttagtagaaatgggatttcaccatgttggccaggctggtgtcaaacttctggcctcaagggatcagcccgcctcagcctcccaaagtgctgggattgcaggcgtgagccaccatgcccagccccaatcccatcctttgaacctcaatgccctcatctgtaaaatggagatcataatgagccctacttcccccaggggagctatggacaggttcacgctctccggggcctctctcacactggccttccccttgccggcctcccctgggtctatctgctgtgggggctgcagtcactcatgggcaggagaggtgaggaaatgcagttgcacaggcctagacccagctgatgccctgtgtgtccaaagcccagtggggcctgggcttctggtggccaggacgcccctatgtccaacccagggtggagaaagaggccaggaggacatggtactctgaaggccttttgtgtggcatcctcaaggaaccagatccagggctcctcctgcaccctgagccccaccacgggaccagagcagcacccagcctgccgccagccacccccgcggtggcctggtgccagccctgcaggcgggcagtgagataatccggctagacgggctgccatcttggcacgtcccaagacgcccgaggccacggggagcttcaaaaggaaaattaatgtctcttaccagcaattagaggaaattattcagatagaaatcttgttttctatctcctcatccttgtttttgaatccccagcctttgatttctttcttctctggggattctcagaaagacactctgagagcctgggctgtgcactgaggcctaatgagggcctggcacacctggcatcacagcctcgcccggggtagtcggcttctgaggcctgtgcctcctgaacccagccctgaggtcttccaaagaggctggcgggcccttggcagctgagggcagctctcccgaggtgggtgggcctctgctggggctggctccccctgatgcccaagggaggtggcaccgggctacaggcggacgtcatgcacctggggtgaaggggtgctgagggcactcttgggtggaggagaggcccaaggggtggactcagtgggaactgacacccttcctgtctcagcggggacccctcatgctctccgcccgtcagtctttccccactgcctcagatgcagcctcaacttcccaatctggcgctcaaagccctgcagatctatcctcgtcagcttccctggcccttccccccatcccctcctctaaatgtcccccgctccacttgcccagggtgctgggcttattccagacaggagctcagccgccatgacccacggtggcacacaccctgacctgacgtaaatgaccttcttctctttttctatctggcagactcctgcacctcctcaggtggcccagccctgcctcaccttctcagggatgctggcttggaccccacccaagagaacagttggcatctccatcacctagcccaggccactgtcctgagctaatcaggagctactcatgggctctcttccctagaaactcctcgaagccagggagaagggcggcttcatctccaggttcccagcaccaggtggcccagaatgggctttaataagctcctgcttgaatgagtgacacccaatgataatgatggtttgaataaataaaagaatgaaagaatgaatgggactgattagaagaggcaccaggaacaaggtgtcaagagactaatcttgaatcatgattcagtcacttcctccctgtgtgacatggagcaagttgctttacctctctgggcttcatttttcagttctgtagaatgggattaatagttttagccctgccttcaaaacagttgtgtggctaaaacaaaataaaggtgttgccaggcatggtggctcacatctgtaatcccagcactttgggatgacgaggtgggtggatcatttgaggtcagaagttcgagaccagcctggccaacatggtgaaaccctgtctctactaaaaatacaaaaattatctgggcatggtggcgggcgcctgtaatcccagctactcaggaggctgaggcaggagaattgttgagcccaggaggtggaggttgcagtgagctgagatggcactactgtattccagcctgggtgacagaaggagacttaatctctaaataaataaagaaataaataaaggggttgagcatgcaattatattaaaattacatgtaccactcatccctccctccatccatccatccatctactcattcatccaattatcaattcatccatccatccactcatccatccacccgtctattcatctattcacccatccaccatccatccactcattcatccatccacgcactcacacagtcattcaccccctcacccatctaggtatccatccaattatccacccatccatccattcatccatccattcactcatttatttatccatccaattatccacccatccatccattcactcatctatttatccatccagttatccacacatccatccattcatccacccatttatttatccatccaattatctatccatccatcatccatccatccatccatccatccatccatccatccatcctcttgtccattcctttaacaaccactcactgagacctcctgcatgccaggcctgtgttgaggaatgggggacagtgtggggtcagaagaagtacctgcccagggagaggtcatcagtggcagcagagaagcccagacaggagctaaaggaaggaggctgagcatgccatctcaagagtgcagcccaggctgggtgtggtggctcacacctgtaatcccaccactgtgggaggctaaggcaggcaaatcgcttgagcccaggagttcgagaccagcctaggcaacgtggtgaaactgtctttagaaaatattagccaaggccgggcacggtggctcatgcctgtaatcccagcactttgggaggccgaggtgggcggatcacctgaggtcaggagttcgagaccagcctggccaaaatggtgaaaccccgtctctactaaaaatacgaaaattagatgagtgtggtgtcgtgcacttgtaatcccagctacttgggaggctgaggcaggagaatcgcttgaacctgggaggtggaggttgcaatgagccaagattataccactgcactccagcctgggtgacagcgcaagactccgtctcaaaaaaaaaacaaaacaaaacaacaacaacaaaaaaaaaaacaaaaagaaaagaaaagaaagaaaggaaaagaaaaaattaggcaagcgtgatggcgcatgtctgtagtcccagctactcgggaggctgaggcaggaggatcacttgaccccgggaagcagaagctgaagtgagcccagatcacaccactgcactccagtctgggtaatagagtgagaccctgtctcaaaaacaaaacaaaaccaaaaaagagtactacccaaatctctgcttcccaccgcctcaaatgcacctgctcttgtcatgctcagccggcttcagaaaccctaggaggccaggaacacttctgccaggggtccagttgggaggacacctggagaacctggaggtggaggtaaagaggaagggaaaaccagacacagagaggtgaagaaatatgcccccaactactgttgaattgggatgtgagtcctgagccacattcttaaccaggtccacatcctggatcaaatgggccctcaagtccagtgcttggaaagcctccaataagtggctggccagcatctgttacatacttcccctgacgagacactcactacctttccaggctagtgaggctcatctgtgtacggctctgactgtgaaaagctctttcttggatagaggatccacattggccctggctcggcgcactggaccctgtattcctgctggcctggaatgtccgtgcagaaacttgaagacagtggtgtgtctctgagccttctctttcccaacatgggtactcttccctgctccccccaagtcctttcacctcccagctgtccgtctggcacagaagatcaagtgcattgttgagaccacaagggaggggaggcagcttaggagatcaggaaaaaaacttatgacactaatttagggtcttagcctcactcttggccttatcctgtgttttctagacattaactttcgtcagcccccaaatcttgatttaatcattccatcttaatgtattgtcttcattgctataaaatgccttgattccttttggggagtagagataagtaataaataaaggtaccagttaaccccccctaagttttgggggatgatcacatctgggtcctttctctcttgcctgcttcaagaaacaattgttaattcttttagtaagtttcaggccagttgatgtaatgacaatatcttgaaatcaaccatcgtggccaggcatgatggctcatgcctgtaatcccagcactttgggaggctgaggtgggtggatcacatgaggccaggagttcgagaccagcctggccaacatggcaaaaccctatctctactaaaaatacgaaaaattagctgggtgtcatggtgcgcacctgtaatcccagctactcgggaggctgaggcaggagaatcacttgaacccggaatctgggaggcggaggttgcagtgagctgagatcacaccactgcactccaacctgggtgacagaatgagactctgtctcaaaaaaaaaaaaaaatcaaccacagtgagggtctttacaccatgagaattggcaaacgctaccagtcagggctttccccactacattttccagcacacaactgaatatgcctctcatctttcttctccccatagagcagtcaagtgaggaggactgcctgatcctagagactctgatcctcccaagacgcagaccagggctctattctcacaggactttcccctcaagtgactctcctctgtgcacacatgctgtcttactctgtattcttttaggagccacagggcttcaagcatgcacccattcattcaacatatgttcaacaccactatctggcaggccccaggctggggacttgtgactggggacttacagattggacagagctgccttctctaccagtgccttgaggggactcactctatgtcatgtgcatgattcatgctatctcatttaaccctcacaacatccgggcccattttatagatgagaaaactgcagagcctggagtgccaagtgggtgatttctatctgttccactccgaagacccaggcttaccactccaaaatgacagtggtggtgattttttcttttcttgttcggtcttcactatggctctgagaggaagctgttcattgctctatttgccaagggggaaaacctgaagctcagggatgttgaccggtccaacttgcaagcttaatgaaaatgcaggactctgtctcctaagccaggtggtggtgcaaaatgagccccactcctggggtggaaccttgagttgggagccctgagttcttggctcagctccactggggatgtcaagggtgaccttgggcaagtcacttctcatgatctaagccattctgtgcccctcagccctgattgctctgagccccagaccctgccatcccctgtcacttgagcttcatcataagatgttccatgagcattgccacttattgcaacacagatggaactctctaagcctctgtttcctcatgtgtaaaaactggataacagtagtttctactttacagaatattgtgaggatacaataatatcatactctcaatggattcctcacactgcagtggtgtgatctcagctcactgcaacctccgcctcccggattctgggttcaagtgattctcctgcctcagcctcctgagtagctgggattacaggtgcactcaattaatgttagccattaactctctgggaacatgtccccactgtctagctggctgtggtactcatgtatgaagcccacaaacattcaagtatccaacgatcacctaaaatgcacacaagttcatccattcagtgttgacatggacagatggtatgatggattacagatggctataaattatttgtcatgcctttcaagaaatctatttcccctcctgtttagtctagactatcctcatggctgcttacccaatagaatgcaatggaaggaacagtggccagtcagggtcctagtctttgtgaagactggcagtttctgcttctccctcttggaacacttgctcttgggacactgcttctcagaaaccagccatcatgctccaaaagacgtacagaagccacatttaggcactctggttgacagccccgctaaagtcccaggcaacatccaacatgagctgccagtcatatggatgggccatcttggatattccagctcgctatagctcccagatggctgcatccccaaccaacatcacataaagcagaagaacctcccagccaagcacagtcaactcataggataatgagatttaagatggttattgctttaaaccactaagtgttgggacaatttgttatgcagaaaaccaaagtgttggggcaatttgttatgcagaaaaccaaaacatatagtatacccatttatgaaactttaaagtatttatttactactttttgaaaaagattagtcattctgtactacagaaaactataacaataatttgctgcctgtatttgtatagcctgcaaggtaagcaaggcttttacactataacatggctggaaaaaatcaaaagaagaatattattttgtgagctatgtgagaaatttatacaaaattcgaattttagtatccacaaataaagtttgattggaatacaggcatgctcattcatttatagcttgtccacagctgctttgtcatgagagcagtagagttccatggtggtatcagaggccatatggaccacagcacctaaaatatttactgtctggccctttccaggaaaagttcaccaactcccttctctagaccattaaatatgtatcatcattttgaacttaaaatcctgcatataagaacttgtatgtgtctgaaacttattgactgagtcctgtaaggaaaaagcaaaaaagcaacagtgggcatttttgcttatttatttttaatgtttatagagacacgatctcagtctatcacctggctggagtgcaattgtgcaatcataactcactgcagcctccatgtcccaagctcaagtgattctaccacctcagcctcctgagtagctgggactacaggtgcatgccaccatgcccagctacattttttatgttttattttgtagacacagggtcttgctatgttgtccaggctggtctcaaactcctgggctcaagcaatcctcccaccttggccccccagagttctgggattacaggtgtgagccacggtgccaggtctatttatttgtttgtaagtttgaaagctttcagaagtataagaatggcccaagctgggtgtggtggctcacacctgtaatcctagcactttgggaggcggagctgggtgaatcacttgaggttgggagttcgagaccagccttaccaacatggagaaaccccgtctctactaaaaatacaaaattagctgggtgcggtggcacttatgtgtaatcccagctactcgggaggctgaggcaggagaatcgcttgaacctggaaggcagaggttgcagtgaaccaagatcatgccattgcattccagcctgggcaacaagagcagaactccgtctcaaaaaaaaaaaaaaaaaaaagaatagcccaacagtgtgcaaacataggaggaatgcctagaaagcattcaaagagagatcaaaaggaaactctgattatgggggtgaacaggggactttgaagaaagaaaccagaaatggaaggggtcctagtgggaaaaggacattccctaaggccaggacagtatccatgcccccagccccttctatcctccccacccccactgtcctgccctggccagagctccaggccacactgttcccactgtagggcctacactagctgtctccatctttgtcccctgcccatcttcctccactagggctcagcttgacaacccttcctcatgcagcctaccacctctaagcctgcattccctcccttctctatgctctgaagttctgtatacaggacttgcctctaagatcccactggccaggtcgtaacgttgagacccaaccactcattctgctccctgggctgggtgcacctcgtgggtagtcattggccttatttatctctgggccccccgggaaggcatgagagccaagctgctgaatacaggggccctgcagcttgtactgagcatttgagtaccagacacttctaggatttcacgatggaagaccctatgatacggcatctccttcccatgccccactatcagggacaaggggtgcagagacaacggggcctcccagctggagggactgttcagggccagctctgagctgcctcccaccctcgcctagcctctggcagggcctctggccaggcaggaacggccaacagagtcacctcctgctgacaagcctggcgctgacagccaggatctgggagaacaagaagtccattcaaggcgcagggccgggcgggcccactttcaggtgtatgatctcacctctcggaaactggcaagccgccggagggcctcgtcaaggctttgatgaccctgtgctgaggacgcaaatcctaggcctggccgacaccctgctccaaggaccaatgactgaggccagagagggtcccagatggagcctgggtaggggtgaaactgcacaggcggacgggcagagcgtccaccctggagaagagacccaggcccaaccaggccccagagcctgcacgagcacctggagtggcagggacagcccagccccctcctgccccacttcctgggaagcagcagcttgtgtgttctggtcggtcggctttggcaaagctgactggtgacgtcaggcaggcaccccctctcatccagatcctgggccccccacacactgggcccagctgggggatggagctaaaggaactttgctcccagctcatcttgtttctgggctttgttgcccagtggccactggcaccctggtggaagggaccagccagggggagtagtaggccccatctcagctggaatctcccagggtaactgggcctgcagcttgctggagtccgtacttttctgtgagctcacagggcagatcccagaagccaaaactgacccagagggaatggaactcagatggtcaagaaaccactgccctctgccctcttggcatgtgaccttagttccacccatagagaccagatggcagtctctggcttttccaaggacccgcggggcaggaatgcaagacagagcacacactggcctctcactgtgaccagcagtggcctctgtcgtcacctctgtggacggcaacacgggccttccatccattccccaaaaggaaggcaaagccacagcacccatgcaggggccgaagcctcacgggaggttccccatgccttcgaaatgactactctgctctggccggaggacccgggtcctgccacctgacctgagagcctctctcagctcaggtcctgcctcctgatcactcgctgagcttggcctcccaacaactgtgatgacaacaggctgtctactgcctctcggagctcctcctgccctggcgggcctggctcattggtgccctggatggctccccatctcccagcaaccgggacttctccctcctccaggcccccgggactctgcctagcccatgccctgggatgtttatggatgtgtccactgcccagcttctcaagaacagaggctgggtgtgagcccacaccgattcctagtgcttagcataagcctggtccaaggaggcacccactggtggtttgcaaagtgaagtctctggtaattgaaagggcagtgtgaccacatccttggagtggaaagatgacaggcttcgagagcagacagccgtgggcagttgtggcccttggacaagtatcttagcttatctgagcctcagtttcctcatctgcagaatgggaatattagcacctattcccctacaaaatagcacagcaacacaaggctaaagagaggtgtttgttgtttttgtttttgttttgagatgaagcctcactctgttgcccaggctggagtgcagtggtgcaatctcagctcactgcaacctctgcctcccaggttcaagtgattctcctgcctcagcctcacgagtagctaggactacaggtgtctgccaccgtgccttgctaatttttgtatttttagtagagatggggtttcactgtgttggccaggctggtcttgaactcctgacctcaagtgatccgcccacctcagcttcccaaagtgctgggattacagcgtgagccaccatgcccggccctaaggctggagagagattttagagagaccaaccgcgtgaagccccgtctaccagatccagcacaggcgctcaaaaaaatcagtccctgctccagctctgggcttctctgtcactggccttctgtgcccagtcccccggaaactgatctgagctggggtgtgagccttcacagcagcgtgtagggaaagtgctgggccagggtcctccgccctgacttttgggctcatcctgccacttcctgcctatgtgtgcaaacgagagctgccttttctctctgaatcccagctttctcaactgtaaaatgggtactccagcctgcctggcccacctcacagtggaggtccaggtgctgtgtagactgttgagggccatgtgtcaggtggcccaggccacccagggactgggcaacctcactggcaggtgggcatagggacggctcttgtccaggagggcctgcctggatggtgagccagtgagagtcctcagtccccagccaggactgctctgggcccagcctggccggagatcccatcccaaaggcaatgaccatgcttcagcctctcgagtagctgggattacaggcacacaatcatacccagctaatttttttagtagagacggggtttcaccatgttggccaggctggtcttggactacagacctcaagtgatccgcgtgcctccgcctcccaaagtgctgggattacaggcgtgagccgctgcatctggccaggcctccagcttttatgggagactcacgtctgcccgcttgatggaagttctgggcagggcaggagggagaagaaccagtatttaccagaatggatgagggcattccaggaggggcctttcgctgaatggaggcagtgtggaaaaggggcaggtgggacccagacggtgcaggagcctgaggctgtccctccaggctgtcccagggatccagagcctcatcgaggccccatccttggctcttctaccgcccctatccatggaagccaggaatcttgttgccccactttgggagtcaaacttcacagctttgtctgtctccaagcctcccactggaggtgcttggaggcctcaaacctgcagccttgaatcacccaatgccctgatgggctgggctgcagagagggagggaaaggttatgggagtgctggcagtggaaatggctggaggtataatgtcattccatttgcagaccaggaaacaaaggatggcagaggggaccacacagccaggcagtgacaggtggggacagaaagggacacccccctgccacccattgtcctgggggtgctgcccaggttcctgagtgagtggttgggtcaggtcacctaaagaacacttggaagcccgcgaggagaggaaacccactttgaacaaatgtggagaaaggcgtggatcagagtctaacagctgaggtccccgatgactgtgtcccgctccatttagggcccgggacacacaaccctccttggtcccctcccgtggacatgggaccaggaccaaggtctcttccccctgaatatcccgccctccccagcctcctgccaactgcctgccttccagtgcccccgaggacacaggactgaggctggatagaggattgcttcgtggacctttgctggggacatgtggacaggcaggggccaccaactcagagcgaagacgtcacagatgtgtgaaaacactgaagatggggacacagaggcaccgagcgttaggtggctggagtcactggatcacagcgtctggagccaagggtttccactgttcccaggtgtaattcctccccgcagccgtgcaaggcgggtgccccagctgtactcacttggctgctctgtaggtcgcacagtcagtaagcggcagagctgggacaggccaggcaggtaggcgtcttgactcggtgccttaccctctctggcctcagtcttctcatctgtcaaatggtataataataggatgtactcctcagggttgctgggagggttacacacgttagtgcacactgcctggggctcggtgactgttctctaagtgtttgctgctatttttttttacttaaggaaacttaacatcgaataggttaagcagccggggcttgaatttagtcttctctgcaccaaagttgcgctgtggactcttgtgccgactgtgatctagagattcccaagccccagaggctttggggaaggcggagccaggagaggctgctcagctgtcagtagatggcgctgtggagccacaagggccgccctcgctgcaaaagcccgttgacgcaccaggctgtgcacagactggggtctccccgccctgggccaggccctgcaccagatatggccccaggagagggggtgccgctaaccctgtttccctgatgagggatatgaaggctcagagaggttgaaccactagccttgaggtcacacagctataaggggcagggctggggctccccaccccagatgcctcactgagctctccagccccacccccatcccacccctcaccctggccctcaagaggccacagtgactccctggcgcttgatgggagccaggcgcgtccctccttcccccatggctggacattccaatgaagctgaagccaccagctttgaagtgagagctaaacccggattagggcggctccaggctggcctctccttcccagccccattgccaggcccacgctcaccaaccacgtcaggccagagtcctggcggcacaaaggggtctggggcctagaggaggcaggacctgccccgcacaacacagtccctctcatagtctgttaagaagcctcgggggcccccacgacctgctactagcctgaaaccccacgtcacgcgggtgccagcacagaagctcccacggggctgggaggctgtgtgtccgtctgtctgtgagcagccacacttccctggcctcaccctgcctgtttgggttccaggatcaagcctctttattcaaggagatttggactcaagactggaagatgacactggtatagtgaagctacaccagttctggaacccgtgttccagaaggttctggaacatccaccttcaaattaatctctcacagcctcttcttcagaattcagctcttcccagacttcacttttttttttaattttattttattttacagagtctcgctctgtcgcctaggctggagtgcagtggtacaatcttggctcactgcaacctccaccaccagggttcaagtgattctcctgcctcagcctcccaggtagctgtgattacaggcatgagccaccatgcccagccttattttattattatctttttgagacggagtctcactctaccgcccaggctggagtacactggtgcaatctctgctcaccgcaacctctgccccccgctgggttcaagtgattctcccacctcagtcttcccagtagctgggattacaggcatgtgccaccatgcccggctaatttttatatttttagtagagacggggtttcaccatgttggccaggctggtcttgaactcctgacctcaggtaattcactcacctcggcctcccagagtgctgagattccaggcgtgagccaccgcgcctggccttttcccagacttcagtgattctcatttcaccttcatgacttttttttttgtagctaaatattgtttacatttatctttttccttaaatcgactcctatacatatgtttattttaaagaaagcttttataaaaaagaaaccttgctatcaaaaatgaaaagctagtcccctgtgccacatacagacaacatgcataaaaatgcatgccatggctgggagcagtgggtcatgcctataatcccagcactttcggaagctgaggtgggtggatcacgaggtcaggagttcgagaccagcctggccaacatagtgaaaccctgtctctactaaaaatacaaaaatcagccaggcgtggtggcaggtgcctgtaatcctagctactcaggaggctgaggcagaagaattgcttgaacccaggaggcagaggttgcagtgagccgagatcatgccactgcactccagcctgggtgacaatgcaagactccgtctcaaaaaaaaaaaaaaaaaatgcataccatgaaaacggaacaatgccgttcaattctggctggctgccggggcctagggaaggctctcagcctggtgtctgctcccttgctaaagagggcagtaggctggtcagagacctgtctgcaccaaatccagcatctctccttgatgcaaccaggagagaacattgtggaaggagactccctttcagcatctttcatttggagactggtcatcatctcctctttgtctgcatttctgccacccattcttcaagttatcacctcctccaggaagccctccaggctgaaaagggcatccccactgtgcttcttcagctccacaggcatttcctgacttggcatttgtcatcctggtttaataaatcacatttctggttatgtcactcccaatggagtgaaagtgtcccttttttattccttagagacagggtcttgccctgtcacccaggctagagtgcagtgggacaatcatagctcactgcagcctccaattcctgggctcagtcaatactcttgcctcagtctcccaagtagctgggaccccaggcacacagtactacacccagctttttttttttttttttcagagatggggtctcactatgttgcccaggctggtctcaaacccctggcctcaagcagtcctcctgcctcagcctcccaaagtgctgggattacacacgtgagccaccacacccagcaagggccaaacctttaccatggcccacacagttgtgggccactctcttccatccaccacagcctgtcctctggctgtctacctctctgtcaccttgagtccttcttgcatcatgatgtcctcactggtcatggcgttggccttctcctggatataagctcagtgtggtctggtgaggtgtttctgtccaccctcggatccccaggcctagcctgcttgaaggaggagagcagcctggaaacttggcatcgtcactgtctccattttccaaatggagaagctgagtcccagtgaggttggtgactttcccagggacacccatctagccgtggcagacctggatgcaaacccaggcctccggggccagcagggatccaccctcccctaaactccctccctctcagggctctacttcaccttgatcaagcgagaccttgtgtttgagagagacaggaagagaaaaacagacacggacagacccctgggatccccggaggcccaagccaacccccagtcagactgaaatccttagcccagcccgggcctcagctcacagcaggcatagagtacaattttccatccagactctagctatgggggcaccccctcggaacctcaccgcatgccttcaccagcccctgaggcctgaaattccatgaggacatttcattgccctgaagtgtggtgtcccaggctaaagacaccatcagcccctagtccccacttccaccccagccatgtcctggctgtgaaaaaggtgcagccgctcccagggttgccagaaaaacagagtgatggtggaagccggtgccaggcagggtccctccaccagccacgctgtcccgggaggggccccacccttctgcgatcagcagaggatccctgggagagtcgccgcccctccagctccaagcccctggggtccgtcctggaaagaccttcaaaggacttggccattcttgagtgagcgccaggacggaatctgctcccagagtcaatttaagcagccacacgggtggaaggaatttgctgtttctcttaacagcgaccccctcatgatttatgcgggggccactgtccgaaggcccccggctgatggctcctgagcggaaaaatgaaatgccaagacccaggcatcttgctgggctaattactctcaatgagacccagctggctgtgggagccggcggcgccggggcaggcccggatccctgtacccagctccctggggctacaaaggcctgtcccaaccctgcctgtgttccctgtctgtcacctaccaggccaagtgtggggctaccaaggattctaggagagccactttggccagataacaagatgcatcaccttggatgctgccaagcaagcccaggactggccatggccttgggtttgctgctgccctggggggtacatatccttgagacaccaggggccctggggttccattctgcttatgggaagactaaggcccaggcagacagggaaagtaccctgaagcctccaggaggctcaggaataattgcaattccagcctcagcttctgccctgcagtgtcttggaggcccaaagccacagagctgcttccagataactcccgtgattcggggctggctctctgggtgtcctggagacagtcaaatatttcccaagagcctcctgcatgccaggccccatgtagggtgctgggggcatagctgtgggcaaatccagcctactcccttccctcagggacttatggcactctgggggaagacagacaccacttaccaaaaagtgaaagttcttggtgccatgaaactacatggaaagtgatttggcctatgccgctgtcagggccttaggatacgctgctgacctgagaccagaaagctgagtagggagcatgatggttactactgggtgtcaacttgattgggttgaaggatgcaaagtattgatccttggtgtgtctgtgagggtgttgccaaaggagattaacatttgagtcagtggactgggaaaggcagacccaccctcaatctgggtgggcactatctaatcagctgccagtgaggccagaatataaagcaggcagaagaatgtgaaaagactagcgtggcctagcctcccagcctacacccttctcctgtgctggatgcttcctgccctcgaacattggactccaagttcttcagctttggaacttggactggtttccttgctcctcagcttgcagacggcctattgtgagaccttgtgatcctgtgagttaatacttaataaactcccctttatgtatatctatatctctctctatatatatatcctattttatataatgttatataatatatattatataattctttatatataaatctatatatatctctctctcttattagttctttcccttgtgatcctgtgagttactacttaataaactcccctttatatatatatcctattttacatatatcctatcctattatatatattatatataatacaataattctttatatataataattctctctctatatatatatatcccattaattctttcccttgtgatcctgtgagttaatacttaataaactcccatatatatatatatatacacacatcctattttatatatatatatcctatcctattatatatctatcctattagttctttacctctaagaaccctgactaatgcaggggggattgggcaaagggggcagggagtgttccaggtagagggagcagcatgagcaagactctgcaacaggaggaaacgtagggtccccaatgaaactgtgcagctggaacccagagcccagggcacggtggtgcagggaagttcaagaggccggcaaggggttcttttgtttgttttgttttgttttattatttcttttagagatagggtcttgctttgtttctgaggctggtctagaattcctgggctcaagcaatcctcccaccttggcctcccaaagtgctgggattacaggtgtgagccaccgcaccccgccacgttcttgtacctttagaaaggtgctttcagaaagaacactggaaagacgatgtgtccacccacatgctcattcttgctgatttctctctggcttttgggagcctcagccctctacccctggaagggcagctctgtggattaccccagctacagggacgggaggaagaactgtagagcctggggtcatccaggtattcctcaaggactggacagaaaggcgtggatattgaaggcagctagtcatgtgcccaggtcaggggcagtggagaccaccaatgaaccacagcttgggagagaggggcctccaggaggccatgctgtgtaactcccaagagtggcagagaccagggagttggagccgccagcagcctcacagccatcttgtgccttctgagaggccccctgcttttcttttccctggggccccacttgtgccgcatcactcccatgcctgtagcctacccccatcctggtatctcccagccagagaccattgagggcagaggtgaggtgttactggtgttattgatcaccctctctgagcactggtacagaggaggtatagacaagtggaggcactgaggacagaggaataaatgagatgaatgccccggcctcctctctgagctgacgtggagtccctctggccttccctaccggctggccctgtgtcttggaggagctgggctcctgcaggtgggcaggcctagctgcagggagcagctggcaagcagatgcctgtggcctgcagtaccttggcgctggcttggttctgcggggcacaaacaggtctgacaagttcctcagcagcagagacagggctcggtccgtgatttctaggaaagtcaaagtcttggagagcaagcggagccaggcttggggtggagggaggagctcctctgctatgtacctgtcccctgtgtcctgagcccagagctccagactgatggtggtgcttctctctcccgcctcccacagagggaacaggagctcagcagtggggaaagaggaggggagacctttcactgtggccaaggggtaggggagtgtggcccctgtggtgaagcagctaccgtcaaagccagggctggccctctgcagggtccaacatgccactctccttggcctccttcctcctggcctctcagggtctgaattctacccctcaacccacagcctccaatccttggctccagggacctgctcccccaacctcagccacccgattccagggtcacaccttagacctgctcttctcttccttaaacactcggttcttgtttcttacttttctttcctttccctccctccttccctccctcccttccctccctccctctctctctctttctttctttctttctctctctttttctctctctcttgctttttttctttttctttctttctctgtcttccttcctccctcccttcctccctcccttccttccctcctttcttttcttttcttctctttctctttctttctctcttcctcccttcctccctccctcccttcctccctccctcccttcctccctccctcccttcctccctccctgccttcctccctccctcccttcctccctccctgccttcctccctccctcccttcctccctccctgcctcccttcctccattcctccctccctccattcctcccttctcccttcttttcttctttcctcctttcctttcctttcttttctttatttttttttcttttctttttgttgatacagggcctcaggatctctctctatcacccaggctggagtgcagtggtgcaatcacagctcactgcagccttgaactcctgggctcaagtgattctcctgcctcagcctcccaagtagctgggactacaggtgtgtgccaccatgcctggctagtttttttgtatttttagtagagatgatgtttggccatgttggccgggctggtctcagatttctgacctcatgtgatctgtctgcctcggcctcccaggggccggggttacaggcatgcgccactgcatctgcccaacagttccttagatataacacccaaagcacgagcagaaatagctaaaatagacttcatcaaaatgtaaaactgttgtacatcaaagggcactatcaagagagtacaaagacatcctacagaatgggagaaaatatttgcaaatcacatatctaaaagggtccaggctgggcgtggtgggctcttgcctgtaatcccagcactttgggaagccaaggtgggaagactggttgagcccaggagctcaagaccagcctgggcagctctccaaataaataaataaataataaaataaaataatatagaaattaaccaggtggggtggctcatgtttatagtcccagctacttgggaggctgaggtgggaggattgcttaagcctgggagattgaggctgcagtgagctgtgatggtgccactgcactccagcctgggtgacagggtgagaccctgtctcaaaaataaataaatagacaaataaattaaaataaaaagatctagtatctagaatatataaagaacttctaaactcagcaacaaaaaagtgtaacacaattttaaaatgggcaatgaacctgaacagatttctcccaagagatacaaatggccaataaaaacatggtaagatgctcaatgtcattatccattagagaaatgcaaatcaaaacccaaatgaaataccacttcatacccattaagatggttataattttggaagaaaagaaaaacagaaaataacaagtgttggcaagaatgtggaaaaattgaaacccttgtacatcactggtgggaatacaaaacggtgtaacctttttgaaaagcaatttggcaattcctcagaaagttaaatatagagttaccatatgaaccagtaatttcactcctaggtatatgcctaagagaattgaaaacatgtatttacacaaaaacttgtacacaaatgttcatagtagcgttatccataatagctaacagaaatatctcaaatgtgcatcaactgatgaataaacaaaatgtgatctatccataaaatggaatcttatctggctacagaaaggaatgaagccaggtgtggtggctcatgcctgtaatcccaacaatttgggaggctgaggcagatggatcacctgaggtcaggagttcgagaccaacttgaccaacgtggtgaaaccctctctctactaaaaatacaaaattaaccaggcgtggtggcgtacgcctgtaatctcagctactcgggaggctggggttggagaatcacttgaacccaggaggcagaggttgcagtgagccaagatcatgccactgcactccagcctaggcaacaagagtgaaaccctgtctcaaaaataaataaataaataaacaaataaagtctacaacaaggatgaaccatgaaaatgttatgctaagtgaaagatgccagacacaaaaggccatgtattgcaggatcctatttatatgaatgtccggaacaggcaaatccatagagacgggcagtagtagcttcatggttgccaggggctgagtgcaggaagaataaggagtgaccactaatgaatttcattttgtggtgattaatattctgtttgtaaatagtggtaaaaggcagggcgcggtggctcacacctgtaatcccagcactttgggaggccgaggcggttggatcacctgaggtcaggagttcgaaaccagcctggccaacatggcagaaccccgtctctattaaaaatacaaatgttggccgggcatggtggcacgtgtctgtaatcccagctacttgggaggctgaggcaggagaatcacttgaacccggaaggcggaggttgcagtgaaccgagatcacgccactacactccagcctgggtgacagagtgagactgtctaaaaaaaaacaaaaaagaaggggggggttttatggtatgtgaattgtatctcaataaaaaagtttttttttaagtatgcactctttcgcctctttttccatacaacatagtatatatgagcaatcaaccaaactgttgtgtacatcagtagtttgttcttattgccatgtggtattcccatgtatgagtgccctatcatttatttttatttatttatttatttatttattttttcagacagggtctcgctgtcgtccaggctggagtgcagtggtgtgatcacagctcactgcagccgccaactcctgggctcaagtgatcctcccacctcagcctcctgaatagctgggactataggcatgcgccaccatgccaggctaatttttaaatttttttttatttgtagagatggggtcttgctatgttgctgggactggtctcaaactcctggcgtcaagcaatcctcctgcctcggcctcccaaagctctgggactacaggcctgagccactgtgcccggcctcttttcagatctttatggccatttggatattattattgtcgtctaccttttttggggttataatctttctattatagatatgtaggattttaaaaaaatacatcttctggatatgagatcttgtcatgatctacgttgccagtatcttttctcgtccggtgcctttttgcattcttaactgtatcttttggtgagcagaagtttttaattttgatgaagtccaacttatccacttttatgatcacagttatgtgtgtcccatttaagaaatccttgcctaccccctaaacatcccttgaagccacctcttcccttctgacgtcttcacctctagccagctctttgcagccagttttctcccgtgcttctccattctcctcccaccgatgtccccaccccactttttgccccctcctctgccttcctcactgcagtcagcaccatatcctgcccagggacagtcctggctcctctgagagtccctgggaccagcacagcctctggcatgcactaggtgcttaataaaagctccataaatgaaggcaatatcccgtggcttagcagggaatcgttaaaactgtcaattagctctaaacaccagctcctcgggaacatttgagcagagccagaggaacaaaaaaggtttggaaaaaccaacaagataggaatgtttcgttttctccctcgctggagcatttaccgcacaaactcagacctcattagcattatttattcttgctttgtccccaaccgtaggacaagggaagcctttttcctgcagcctctggctgcaagagctccctgtcacaccccacaacagtgtttgctgcccctgaatgtcggggagacttgtgatcccctaatccttctcttcaccgtgagtgatgacgtagcacaggtttctgtagtccaggttgccgcacacatctgggggaaatgctgcaaacatctgcttgacctgagaaggagcagagagtcagcctgcaccaggccaagtgggggccagagcagagaatccgccgtgacccaccagaatcccagtttacctcctcctcactgaagcggtctgcctgggtcataagtttttctttgatgctgttcaagggagagacacagtcatgttcagggaacgggatacccagcttgaacatgcagatcccaggggtgatgaggggcaagtgggggcagggggacatcctggaccaggaggacatcaacctggggctgaccctgccagcccccccgatggccaagtagcttctgaacctcggttttctcctccatgaagtgggctttcctcccagcatcaggtagggaccaaaagcaatgacctggggaccccaccatgagggctactctgatttcccatcatgcactataatcaagctctggatcattgaggggagacacctcaggtcccccaccgcagagcctcttttgcctgattctggacacactctttcctccagcttgtgacgttcactgacacctgcatagtgttttagagacaagatgcctgtgctctgccaagggtacaggaaggcctgcccaggtccgtgtcctcctgggcctgcaagggcctgccccctcaagcttggagggtcctgcagggttcttaagggagctcagtgggcaaagatgggatcagacggctgagtggggcctcgtccacccagagtgtcctctggcaccggctgcggcaggcctcgctaaggaccaatgcacattttccattatttatgtttctgtgggacagtcacagtttccagctctccaagccggggacttttcacagactctgtttcaaccagagcctccaccgtggcctgtctggcctgcctggcctgcagccacagatccaggtcccatgtcccaaggcagtgccttcagttcccaaattaggtccccaaagggtcttcccagccttgtccttgcactggtgagagagaaagtggttgacagtagagggagggcttggggttactccgggggtgactctgatgcctccccaggtccccaggatggaggtaggagccacagaccccgagggaggggtgaaggggaggactgtggactgcagcaaccctcagcatcccctccccatcccaggggagtatgtgactcatggtcttggagttgagaatgtgtgagacgggacagaccctctccacctccacccctgctgcgtctgccctctccctttgagatgcgagaatctcaaaaccagcaaaactagcctcaaaatgaaggctggcagcattacagtaattgatcaggaagcctgggcctggggaggaatctcacctgccccaggccaccctcaggtccagcaaggttctgaagggactgcgtcggcacagctcagaggtcagccctgaatcttgggtcctgggaaggctgccactcccccgcccactcctgcctgcaattcttggctctctgttcccttgctgtcactgggtggggcacccctaccctcatgatggcagccccggtgcctgccagacagctgcctgcagccacttccccttcatgcattccaagtgagctggtcactttccccaatccttactgccctcttgcccgaggcggtgctggctacgatctggctcagtcctctccccagaccctggatcctatgggcactggcccctggctctttgcttatctgtccgcccgactccatctcgacggccatgaccctcctgggagctgcaaccctctcctctccttctccaggctggcctccatggtgtccttgcctaactcctcctcaggttctcccatgctcccagggcagacgcagggtccaggggctagggtgtgagcgctcccccagaggccccgcctgccgctctgtgctcagtctcccctacaccacctccacccgcaccctccagctgcagccaactctgaacaaacatgcacatgccaagccctggccctccaagctctctgttccctcatctagaatgctgttccccccctagcctgggcaacctgcagtcattcttcaagatctgcctttgcccccacacccaccccactaggaagctctcctggagtgacccaccacttcttctttttttttttttttttgagacagtctcactctgtcgcccagtctggagtgcaatggcgtgatctcggctcactgcaacctccacttcctgggttcgagcgattctcctgcctcagccaccagagtagctgggactacaggcatgcaccaccatgcctggctaattttttttattattttcagtagagatggggttttgctgtgttggccagggtggtctcaaactcctggtctcaagtgatctgcccacctcggcttcccaaagtgctgagattacaggcgtgagccaccgcgcccagcctgacccaccatttcttatccatttctacttccacgcacacatagagaaggggacaccaaggcccagagaggggaagggacatcaagcgatatcactgggctgggcagcggccccccagctcctggcttctgggagaggagactgggcgaccatccaccctgaccccagcccaaagctggcttattcccctgagcattttgggggagtcaggggaaacttacacatcggccttgacgaaacctttcccttcagtgtcgaacactttgaaggcgtggagaatggtctcctctgggtccgtgcctataagcagcacatacagggcagggagagaggcaggtgaagagggccccacagggacaggcacaaggctgggcaggggctggggctgggggcaagtcccacggcccctgcacagaggcacagctctggggaacttcttccccaactttcactgcacagcgagggccgagctcacttgaagcaagaacaaggagggaaacaggtaattgtaatcatagacaagggtaccacatatatatattttgtgaacaacctgagcaactgtacacggagaccctgattgagcatttaatttgtgcttagcccttccctattttacagatggaaaagctgagacccagagaagttaagtgacttgcccagggtctcacagctgcatgaccagtggtctgggtgagggggcatgactctggggctcacgtgttacccaagctgtgcacaagaggccgtgggtggaggaaaaagagttctgtacgtggatgccaaaaggctggggcttgggcagtgtggcattggaggggtctgagagagattgtcccggccagaagtggggcaggtgggccaacaggccaagggaagggattcacgggggcagaagtgaagccagagaagttctgcttttatcccggtcacagcacagccttggggagagatgtcattagaagaaattaatctgatacccacaatgggattgaatgtgcagcccccacattgcaccaatgggaaactgaagcccagagagaggaagggtcttgcccaaggcttcacgggctgcgtgaatgacagcaggtgacctggccaccctcgcccggtgccctttccccttctgtcaccatgtcccccttgttcactgtggatgcgggggtgcccccttcctttcttcagtgctcaaatgtgtcattggtgaaaggggacagagatggtgcttgctacccacctcacaggagcagctaagataacataccattcatggtaaggttttaacgaacatgggttgatcagggctgcaaatacatctgagcgctctatgcctgcagctttaagattttatcccctggagctctgagagcacccatggagtagatgtggatatcttggtttatcatagaaaaaacaggctcagagatgtttcattacttgcccaagtccccactgataagagaagggaggatttaaacccacctctgtggactccagagtcccctgctccttttgtcaccctgtgctcttgccagtcagattaggtcagccagggccatgtgcctccctcatcagactaggtcagggccatttgttcctccaccatcatactaagtcaggaccatctgtgcctcccccattagagtgggtcatcgccatctgttcctcctgcatcagactgggtcagggccatctgtgcttcctccatcagattagatcagggcctcatctgtgcctcccccatcagactgggtcagggtcatctgtgcctctcccatcaaactggatcagggccatctgtgtctccccgatcagactgggtcagggccatctgtgcctcttccatcagactggatcagggccttctgtgcctcttccatcagactgggtcagagccaagtgtgcttcccccctcagattagatcagggcctcatctgttcctcccccattagactgggtcagggccatctgttcctcccccatcacaccagatcaaggtcatctgtgcctctcccatcagactgggtcagggccatctgtgcctcccccatcggactaggaactagccaggactgggcagtgtctgagtctctgggcatctctgaaactggcacagagtgaaaaaacctggcacacactggccttgctggcctgggaactcctagagggcagcgccatggactgcctctgggcagggtggcagggagctggtgtgcccagcagagaggctcagcccagggtgcagcgagggcagggctccaggccctcacagcctggccaagcaggcagggggtagcaggtgcctctaggcgggtgagggttgagtgaccagagcatcacgtggggcacccatgtgggatttgcctccgaggccacagcctcccaggtttcactgattagttgaaaattcccaacgtctttgaagaggcctcactgctgaggcaggtttgtggccggctccagcctatccccctgcggacaatgcacatggatttaggcacccagcgcagccctgccactccctgttaccgtggctggcagtcctcccttctctcctccccagaaagagcctctgttcttgagactgcaaacctcataaaatctgctatagagtggccacttcctcccatcatcgttgggaatctcatgacacagtctcctcttcccaattagcggggtgaggcggcgccggccccagcaccctgccctcccggctcctgtcccctcccaccagcctctcacgcagtctggccacgtctgcatgacaggctcagccggagccccagtcccggcgcccctgcaaaaatgagaaaaagctgcagccctgcagcccacaccaggccctggatcacagcttcagctcaaccatggaccgacacgagcttttagtctcaatcttcccatctgcgaaacaggtttggggcccactgtgagctgacagagtcccctccccttcccctgagcactcttggaggtctctctaaactcctgctctggcagcagggcccctcgcccatgcccaacaccaggggcgggaggtgctcacagcctcacctcctgggcccggccctgggccgtccaccctggcagaaccacaggccacttccccaggccctcccactggcctttaacacgcctccttcagttatgtgcctgtggataaaagaatcaggcctgtctgcttcctttgaagttgctggctggagtcacacatgtcttttccacagagcattcttctctcccggcctcctgggcttctgagctcttccaggaggcgcacatggtcctgggaataggccccgacaagctgcctggaggtgggcccagtgatgctcctgattcggctacacagccccaagttcaaagcctgctccagatgagcctgccctccccagccctgggtttcctcatctctcgcacccaggctgatggctgccttccttctggctctggatctgccaggacacggtgcgtggaggctgctctaccccacagcaggtcacgacccatgacagtactgatttttcaccccatcaaacctgtgatgtcaggtcggatcaccctgtggtggactgaatggtggcccctaaaaagagacgtccaccagaggccgggcacggtggcttacgcctgtaatcccagcactttgggaggccgaggaaggcggatcacgaggtcaggagatcgagaccatcctggctaacacagtgaaaccccatctccactaaaaatacaaaaaaattagacgagcgtggtggcaggcacctgtagccccagttacttgggaacctaaggcaggagaatggcgtgaacccgggaggcatagcttgcagtaagccaagatcacaccactgcactccagcctgggtgacagagcaagactccgtctcaaaaaaaaaaaaaaaaaagatatgtccaccagagcctgcaaatgtcaccttatttggaaaaaaggactttgcagatgtaagttaagcatcttgagatgcagagatcatcttagattatcccagtaggacctgaatccaacggcaggtagagtccaggcgcgagggctcatgcctataatcccagcagtttgggaggcggatatgggcggatcacttgaggtcaggagttcgagaccagcctggccaatgtagtgaaacggtatctctactaaaaatacaaaaaaatttagcggggcatggtggcgggtgcctgtaatcccagcttcttgggaggctgaagcgtgagaatcgcttgaaccttggaggcggaggttgtcctaagctgagatggcgccattatactccagcctgggcaacagagtgagactccatctcaaaaacaaaacaaaataaacaaagaggcaggtggagggaggttaaatagaagagggcggggccattgacggcgaggctgagatgggagaggtagagccaccagaggctgggagagacaacggattctccccaaatcctctgccagatttcgcacttctgggcttcaggactgtgagagaatacatgtctgttgttttgagccacctggtttcacagaatttgttacagcgggctcagaaaactattataagcccattttacagaccaaaaaactgaggccagaaagcagataaatctgtgcaaggtcaacctaggagtcaatgggaagccagggtctgaccctaggcctagccaaggcagggggttcaagggagggggcctggcaggggcagcctgagggtgcagagctgacagtgctcacttcccagggaccactgcctgggctgacccttccttttttttttttttttctttttttttttgtagtggagtctgtctctgtcacccaggctggagtgcaatggcaccatctcggctcacggcaacctctgcctcccaggttcaagcgattctcctgcctcagcctcccgagcagctgggattacgggcacccaccaccatgcccagctaatttttgtatttttaatagagacagtgtttcgccatgttagtcagggtggtcttgacctcctgacctcaggtgatccacccacctccgcctcccagagtgctgggattacaggcatgagccaccgtgcccggccttggcccttccttctgaccacggaaccctcaagctccaggatgtcactatgggagagctcccccgaggccaggacagaaaggagacacagatggctccaaccctccaccccatgcctaatgctgtgtctcgggcgggtacagtggttagagccagggtttggggttgaggttgcatcccggccactttttgctggagcatctggggcaggggtcgtgactcctctggatctgagtctcttcatttgtgaatgaggctccccgatccttcctctgggggctccagtgaccacagaaaaagacaaggggagtaacttgccctgcacaggggtcgaccacagcatcttttggaaacaccctccctccggcaccctctgcctatacccccagccccatgtgcagagcgggtaaaaggagctcacaggtaggaaggtagggaggcagcatggctgggggcagtgggatgggtgggcaaaggggttaaaggcagtgtttagtttttatggttgttgttgttgttgagacagggccttgctctgtcacccaggctggagtgcagtggtgcgatctcggctcactgcaaccttcacctcccaggctcaagtgatcctcccacctcagcctccaaagtagctgggactacaggcatgtgtcaccatgtctggctaatttttgtattagtagagatgaagtttcattatatcacccaggctggtctcaaactcctggactcaagcaaaccgcccgcctcagtctccctaagtgctgggattacaggcgtgagccaccatgcctggccaaggcaggggtctttatagttaccccagtctgcggtgaagagagggaaaccgaggcagggcccctgaaggtgcgtgcacctgggggctggtgtctggggccacatcatgctcccacctgtggtgtgtatgtgtgtcaggcccctgccttcctacatccgtccccctgccattccctgctgcccctccaggactccaggagcctggctcacccttcagcttctccccaaacatggtcaggaacaccgtgaagttgatgggtccgggggcctccttcaccatggcctccagttcctcgttcttgacattgatgcggcctgtgggaggtgagaggtgggggcagggaggataagagagatcagagcttgggcctcggaggatcccacaaagcctccccactcagctgctccacgtgccccccactcagccctaaggccctgacgcctttttgggggactctttagggggcatttgggccacagtcagcttccggctgcccccagcctcatgagaagggagcaagtcactctcatccccattttacggtgaagaaactgaggcccaagaagctgcagctgaggtgcacgggggctgcaggctcagggttcctgtgccccaggaggggatcagtggatgcctggatgctgggattccctgtggttccaggggacactcagattcccaggggatcagggcctggttacgtccgttctgccacctaaaccctgcataggcgacttcaggctcttccctggggccttagagggatcagaaacctcaacctgggagttgggtctttccctctgactcagcttctcccctgctttcctggcccctccccacacccagggtcccctacaccaggcccaggcccaggcccaggcccatgctgctactcctgtccctgaagtgttttccccttccctggccgaatccttctccaaccccccaggaaggtccccccctgactttccagacagtggctgcctttaccctgtgctccccagcacaggagcctctcacgctcaccgcgagccctgccctgctggccccaaccgcccactctgtctggctggcctggccctaatctcccccggctgctggctcacccacccagcgcggcaaaggtgtccctcaagtcctctttgtcgatgaagccatcacggttctggtccatgatggtgaaagcctggcagagacacaggtgctaagtagtcacctgcccttccaagcccggccacctcccccagggaccgtcctcctgccgactgctgcccaaggagcctcggggcaggtccctctggcagcccagctgggatgggctccatccccaacccctcatttcaggctcacagcaactccagtttgcagacagggaaactgaggctcagagagagaaaagtctggcccgaggtcacacagcaagcacagggtcaggtacagagagacaaagaggctgcctgagggcaacaggtcactcacccaggctctgggccatggccatggtggcctcaaaaaataatgtcattggccaggcttggtggctcatgcctgcagtcccagcaatttgggaggcagaggcaggaggattgcttgaggtcaggagtttgagacaagcctgggcgaaataatgagaccctgtctctacaaaacattaaaaaattagccaggcatggtgatgaatgcctgtagtttcagctactcaggaggctgaggtgggaggatggcttgagcccaggaatttgaggctgcagggagcattgactgcaacactgtactccagcctgggtaacagagcaagaccctgtctcaaaaaaaaaaaaaaaaaaaaaaacctaatcatttaaaaatgtagtaatgggcgctgggcactgtggctcacgtctgtaatcccagcactttgggaggccgaggcgggtggatcacctgaggtcaggagttccagaccagcctggccaacatggcaaaaccccatctctactaaaaattcaaaaaattagctgggcatggtggtgcgtgcctgcaatcccacctacttgggaggctgaggcaggagaattgcttgaacccgtgaggcagaggttgcagtgagccgagatcatgccattgcgctccagcctgggcgacagagccagactctgtcaaacaaacaaataataataataataatagtaataatgacatcttcagcccctcgagactgaggactgagcctctgcccagctccttgagcaactggcatgcattccaagaatcctcataacagccccatggggcagcagaccaacctctttaaactcctggatctgggactgatcaaacatggagaagacgttggagctggcggtgccttctgctcttttccgagctcttctcggtgcctgcgaggtagcagacaaggccttgcagcggcccctgcctcgagggtcagcccccaccccctgtctcacctcccagggcatcacggtccagaatgacaccgctttgcccttcccggtgcctacagcagacagacaaggctacccctgaagccttgcaggtaatcaggacgttttcccagcccatggcaagaagcctgcacggcgtgcacccccacagagctggacagctgcttcctggccctgtcccccgggctccggaaggtgggcagccggtgcccgtgctgcagaacagaggcgggcatctcttccttcagcctccccatctctaccccggcatcctccactacagctggaatctgaccttatcgccagctgctaaaaaccttcttcgtgttctcaggagaacaactgcgctcttagccccaccacaggatctgcctcttgccctgtcccagagctgctactccctctcacccaggccctctactctgtcccagagctgccttccctcccagcgtttgcccctgcctggaatgcgttgctctgttggaattccctgcttggaatgccttccctggagaatctatctagagactcctacccatcctgcaggctcagctccctgtcacctcctccgagaagccttccctgaccccaggccactctcccctccccacatggctccactgtggactgtcacccctacagcatgtatcacactgtcctgacatatctgtgcgtctgtgtcctcgactaagtggacatctcaggggtaaaacccactttgccccatctctgatctctcctatccccaccctgaacctggctcaaagtggggatcagccagtgcttggaaatagctgcccagaactcagggccaacccacgcagcaggcaaagcaggaggccacccacgttgagtagtcagatggtttctggccagccttcctgcctctgtgatgtcagggtgtctggaagtccaccattgggcatttgctgaaagccctaggtccccaagaccctcaagagtcctgttcctgggcagccatccttacagcccccaggggcgctgtgagccccagaatgggctctcgatgggggaaaagaattgctggattccagacggggaggtggacgggatggcacagaaatgggttcccaccaccagctcctgtctccgtcacaacttgtaccaccctctcctgccaagcccttgtctacctgcccccaccagagcagaggagtcccctcatcccctgctcccctcccccaacgcctgtgtggtctcccctgcccccagctcactgctgtccccacccccgcccaggagcccgcgtcgtcgagggcaggggcagaagcgccggctgacttataattagcttttactgggtgctctgtctctcctaatcctttgaaagggggctgccagggcggccgggacgttatgatgctgtaaaacctctgtaatatttataaatcctttcagacatcataaagaaaagaacttccccacagagttcatcccacataattacatatacatttcctccgattaataaaaaatgatcacttccgtctgtgggaggttctgccctttctctggcacacctggctgtggcctcggcgtcctcagtccctgccccccacgcccccagcctctcatgatccccctggttacacccccaccctacactgtcccagagctccacgagggagggagagatcattttctttacagcacaagagtcctaaaccctagggtctttgcagcccagctgtcttgaaaattccttttgagaatgggcccctgaaaaataaaattcccctgaaaggaacatctcagggaagaagggggggtcacccggatctgggttcaaagcctggcttggctgctcccagcctccgtgatgttaagaaattcacttgtcttctctgagcctgtttccccatttataaaacctagagcaggtcaacctcaaagcatcaggacatgtattagacacctacggacctccaggcaggtccgtcctggggacccagggctgaatcaggccccgtcttggtccccaagaagatcacagtcccgctggtgacaggcagtccatagaggctgactcggaggagggacaagcgctgagaagacaagacagagaagcccctaggccctcgggcagtcaggaggtgacaagccgaggctgacagccaagtcagagctctgtgccgagagggactaactaatgaggcaggtgagacacaggcacagagggacagccagattgataccccgcagggagtccccaggggcaggaacccagagcaagccctgcgtcacatgaggtgcacaggcaaaggtgaacagggggacctcggagctccagaggtgccacgctgccccaaatactgacccccggccctcccagggggcccctgccctcagagccactgcccctgtgaagcccccaggccctgcccacctcgtctctctccaccctcctcttcctcattccccagcagtccccacaaagtccaggaaagcagaggctcaaatcaggacccacgtggagaggagtcccgagtggggcaggtccaagcatgggatcaagagcaaagagcaggtcagaccagggacaacatcagattaaggtcaggaaggtcagtgagaggcaggatgaattcaggcaggaggtgaaggtcagaatcacacacagagtcaaggtcacagaacaaggtcaaagttgagaaatgaggccgggcgctgtggctcacgcctgtaatcccagcactctgggaggccgaagcgggtgaatcacttgaggtcaggagttcgagaccaacctggacaacatggtgaaaccccgtctctaccaaaaatacaaaaaaaaaaaaatagccatgtgtggtggcgggcgcctgtaatcccagttactcgggaggctgagacaggaacctcactcgaagccaggaggtggaggttgcagtgagccgagatcgagccaccgcactccaacctgggcaacagagtgagattctgtctaaaaaagaaaaggaattgcggagtgaggtcaggggcaggggcaggggcaggggcaggggtgggagggaggggaccgcagtgctgaagccaaagacacaacagccacaggcctgagctccccagggaggaggtggaggtgagaggaaggtagccgtggcagagcagaacaaccccagcctaccatggtctggagagctccctgaagctgcagttggcctggggatagctcagagatggcccttaaatagtggtcctcccagggcccctggcatgcaccgggcccagtgtggaaggtgatccatgacaggacggagacctccctgccaggcacctgccttgggccattcagagtggatagggttggggggaatccgtgggcaaataccacggacgcagggactgcctaagcctccaggatctccgagagccctgggccaactgttttctctagagccaagggaccaagacccaggaaggggcatttggccaaggacacagagtaagttgaggctgcctaccccaatgggaagacttggccttgagccgtcagggagtgttgcccagggtggggccacccatgctggctgggacccactcagactgcagtgtggccggacctctggagctgtgggactctgggtgggacatcaccctctaggaggctcagccatctcataggaataggggggagtgtatgaaacccccggtctgcttatggagagagtttctgcaccaacctggccctcgggggccccttctaccaatatgcctattatcagggtgtccattaagcacccccaggccaggggaccccctcagggcactgaaatgctcaatgaacagagaaagcaacaggacaaataaaagaaaaatggggtgggcgcagtggctcacgcctgtaatcccagagctttcggaggccaagagggaaggatcgcttcaggccaggagttcaagaccagcctgggcaacatagtgagacccctatctctacaaaatatcaaaaaaattagccgagcatggtggtgtgctcttgtagtcccacctactcaggaggctgaggcaggaggattgcttgagtccaggagtttgaggctgcagtgatctatgattgtgccactgcactccagcctgggcgacagagtgagatattgtctcttaaaaaaaagaaaacagaaaactggaggtgactctgatgaggaagaactgaaaaatgagacctccattccaccctggcctcacacacacacatccatccccaccaggcctggcccagcccaagaattctctagtagatctgccaaacagagcccacacctgccctggaaagcccctcctgtgtctagtgcagccccagcagccccagctcggcctcagaggctgccactgtccatctggggcccgccatatctgaacagctgccagggccggcagtgctgggttcaggcagtgagcccacgggtccccaggggctggagagcttaactgtccctggagctggcctcaggccttcagccccgggtacagctgatcccctgcctttggggaagggaatatcataataataataagaagagcagctaatatacaacacagaaggtcaggccatgtcttaaaacacgtgtcctcacattcaatccatatcccattttccagatgaggaaactgaggcacgcatgggttaagtcactcacccaaggcagataagaagtggggccagaggccaggcacagtggctcatacctgtaatcccagcactttgggaggccgaggagggaggatcacttgaggccaggagtttgagaccagcctaagcaacatggtgaaactctgtttctacaaaaaaattttttttaattaaaaaattagtcaggcatagtggtgaagtgaaaaagttcccttatcccgttcatagggcatgcgatgggggtgtggctcgcttcttccatgcccagctgctcaaacctctaggcggaccatgcagacaggcaggctatggggctccgaccccacggcagtgtctaggggtggatgcttacagctcctgaagccccagtgggcgtgtgttacaggatgctcttttacttttgccatctgtaggtggcttgtgttcatcagctcagttagaccctctgccttattgcaaggacaaagggctttttgtatcctggggttcttgccttggtgtactgtgaaaattgaatcacatgtgggcttggagaatgagtgcgaggttttattgagtggtggaagtagctctcagcagatgatggggagccagaagggggatggagtgggaaggtgatttttcccctgaagtcaaggtgctcagcagccagactctcctctgaccatccccagccaaattcccctgggcatttgtgttattcagccagtcgatggcctgccagcatctgccagtgctgtcggtgtgctcttccgatcctctgctcctcttgatgtccagccacctgtgtgtgtacccactggggtctcagggttttcataggcacaggatgggcgggggggcggggggcgtggtgggccagggtggtcttggaaaatgtgacatttgggcatataaacaggagtgcctgtcctcacctaggtccgtgggcacaggcccaagggtggagcccttgccagggaccctgcctttctcctctcagcacttccctgtcccccctcctgtatcagtggcacatgcctgcaggcccagctacttaggaggctgaggtgggaggatcacatgagcccaggaggttgaggcttcaatgagctgtgattgcaccactgcactccagcctggacaacagatcaaggtctcatttctaaaacaaaaaaaaaaaaaaaaaaaaagagagagagagagggagggccaggacaagagcccctctttctagtgtttccagaaggaagcttccaaaaggttctgctcatccaaactctcccctcctcctgctccctcccagtcatccctgactacagctatttctgatgtccctaaactcctgtgactgagtgctcctcccagagctggggccaggtccagccatgatgctgtgcagcccgctttctgcagcaggaaaccgtgtgcctcagtttccccgacagggctgctctcctggtgtcctggcgttcctctccggggagaaaaaccacacgttccacaggtgttcaacaagccaggcggaaaggagaacccagctctggggacgggagcctggattccagtcccagccgcctggacctgctacagaagggggcctgtgtgaccgcgacagtggctagggtgcaggctttccatgcccggtctgtgagcctggccctggggcctcctggctctgggcttacagagtgggtagcagacctataactcctgcttcctgcctctggcttcctgctgccctgaggcctctgcaagcaagggctcttgcctccggggagcacgcccaggaccgtggcaggggctcccaaaggccggggacaggcctgagcaagcggagagcccccccgcctgcccggaggaggggaagcggcgctggtgaatcacaccgggcttacaaattagtctgagtggatttcctcccctggcctcctccttcataggaccggaggcaggaagaaccggtgagaaatctggtagagacgtgaaaaccccggggtgagatggttgggagatgtttgttcctggggggcgggaagtgtgagggtgcagtggccttgagaagggggagccgtgagttctgcacccacagtgggaccctcagttttccccatctgagaaatggtgccagcagccaggaccccttgaggctgccatcgggaagcttagtgccattggaaggaggtgggctttggggtcatcggcctggggtttggagggcagagccttcccctgtccccctttccccctgtgtagatggagtccttgacagaacttgcagcctctccaagctgcagccccctcacccaggtgacactgcaatgagtcagttgtcgtggctgggtcacactgtttgactcaactgaccacagggccagggccagttcttggggtcagagacactctgagaggtctctgctcccagccaggggaccctggagagtaagtgtcctctggaactggaattccaggtctgcctggggttgagtcagagaatacttagcactctcggtttccagaggtaactgaggtcctgggagtggccaagccaggaagggagcttggaacgagcccagctgcctggcagggggacagctgttccccagacaacactggaacaagccggggagatgccttcccctttcaaccctcacccctaacctctcacctataactcctcagggtcagcaggctgtccctgtcccccagccccacagcccttgaccccttccctggccagccatcaccctgaccttggctgttctgcaaattcttcccgaaatctccttcctccaagcctttggccacattgtatgtttccctactcatcacaatcagtctccagcctgctttagaacacctctcggacggccgtggtggctcccgcctgtaatctcagaactttaggaggccaaagtgggtggatcacctgagttcaggaggtcaagaccagcctggacaacacggcaaaacgctgtctctactaaaagtacaaaaattagccaggcgtggtggtgtgcgtctgtaatcccagctactcgggaggccgaggcaggagaatcgcttgaacctgggaggtggaagttgcagtgagctgagatagcaccactgcactctagcctgggtaacagagtgagactgtgtctcaaaagaaaacacaaaaaacaaaaaacaacaaaaacaaaaggaagccaacagtgctgccttcagtctgtggccgaaggcccaagagctcctggcaaaccactggtttaagttcaagagttcaaaagctgaagaacttggagtctgatgttcgagggcaggaagcttccagcacagaagatgaaggctggaagactcagtgagtcttcttattccactttcttctttctgctttattctggcctcactggctgattagacgctgcccacacagattaagggtgggtctgcttctctcagtccaccgactcaaatgttaatttcctctaccaacaccctcacagacatacccagaaacaatactttgcatccttcaatccaatcaagttgatacttaatattagccatcacacctacctactgaatttgagagcctaggccactttctgaccttccctgagcaatcccaggctgccgtaaaaaatgcctgcaaattctttgacactttttccatggagaggcgggatctataacatcattccttgccaacaggcaggcttttgtgactgctttgaccaagaaagtagggtagaagtgtcactatgtgacttctgaggttaagtcacaaaaagccatggagcttctaccttatttgctgggactttgtgctggagccctgagtcactctatgacgagtctgactaccatgaggcggccatgttgtagggaagaccaagtcacacgaagaggccccgtgtctgtactccagtcagcagtccctgcctttgagtcatcatccgagcctcccagacaacaaacgtgtgaatgaccagtcttcaagtgattctggcaccagccatggagtcatccccatccttagagtcccccgagctgaggccctagacattatgacaagccaccctcaccatcccttttctgaatttctgatccataagaacccctgagcacactgctatgctttggggtttttttttgttttttgtttttctttgagatagagtctcactctgttgcccaggctggagtatagtggtgtgatctcagctcactgcaacctccacctcctgggtttaagtgattcttctgcctcagcctcccgagtagctgggattacaggcacccaacaccacgcccagctaatttttgtatttttagtagagactggtttcaccatgttggccaggctggccttgaagtcctgaccacaagtgatctgtccgcttcggcctcccaaactgctgggattacaggcatgagccaccatgcgctgtgctttgggctgattctttacaaaatatcataataaccatcacagtgagatttaaatgtgctctgtagggggacaggctacacagccatagtctactcctcagggctctggggaagatgagagcatgaatttgaaatcagatgaaagtgacttcaagtcctgctggaccacttgctctgtgacctcacccacatcactgaatttcccagaatcctcagcttctccacctgtcacatggaatcagttttcacttggcaagtttgttacatgcctggcgcttgtagactgcggctaccaatcctgtctggttattggcagaggacatgctgcctgtgacaagacactgagccaagagatgcctctgttgggcttccagaacgtgcttcctgtgccaaggctccagtgtccttagtggaagagagacgccctcacggcctgccctttctggcctggccagggcagcatgactcattggatgacccctcgccccatcccaaatgtcacagccctgcaccatggcttcccttagaccctgtggcccaaggacacacagtaggctgggtccacaggaaacacccaaagccacagaatgccatttgcccactcccccatctgtatctcctttacccatctctgtcacatattgagaaaatggctccagattgggggtgcacacagcccctgagctcctgcagccatcaaggctcctgacaggctggagccgaccgagaactagaggccggctcaggccagagcagccacacatagcatttcagacagaccctgactcagggccagttcgcctctttctcccttaagccagaggaaatagcctcgtatgtccatcaactctctcattgccaggtgggatgcctgagatgttgacagttagagacggtgggctgtcttttccgaaagcccctggtgtcccctcctccccagctcccgtcccatctggagccttcttccaactggagatcacttagaaaagacaaagaaacctttaaactaagaatgcctcctggaatgcctcttgcctgttttatagctgcagctcagcgggctggtgaatgtgctctctcgcccacagaagctcaagagaggcatcccattcaaggggcctgtggtcacagcatcctgctgtaaaaatagctgtggatggccaggcacggtggctcacgcttgtaatccaagcactttgggaggcaaggtgggaggatcactgaggtcaggagttcgagaccagcctggctaacattgtgaaaccccatctctgcttacaaaaaaaaaaaaaattagccgggtgtggtggcacacacctgtaatcccagctactcaggaggctgaggcaggagaatcatttgaacccaggaggcggaggttgcagtgagccgaaatcacaccactgcactccagcctgggagatggaggaagaccctgtctcaaaaaaaaaaaaaaaaaaaaaagaaaagaaaagaaaaaaaaagctgtagaatcaatggctgacaggaggagaaaggggtagggtttttctgcccagtgggactggaagtcaccctaaaagtgagtagatgaggtctctgagccaggtctgggaacatgtggctggatcttgtttgctcttttttttttttcttttttttgagacagggtctcactctgttgcctaggctggagtgaagtggcacaatcagggctcactacagcctcccagcctcaagcaatcctcccactttaacctcccgagtagctgggactacaggtgcacaccaccatgcctggctaatttgtgtatcttttgtagagacgaggtctcgctatgttgctcaggctaacctcaaactcctgggctcaaacgatcctcccgcctcagcctcccaaagtgctgggattacaggtgtgagccaccgcacccagccggcttcctgacttttgaggttttgggactccaactggcttccttgctcctcagcttgcagacggcctgttatgggattgtgtgagacaattctccttaataaactccctttcatatatacatccatcttattagttctgtccctctagggaaccctggctaatacagtaggggagccagaattcagatccaggcctgagggactctgtcccacagccaactatgtgagcccaggaagtccaggaaactcagtggggggtggcacagcatggcaggtggtgactctctggctacagccaggaaggcctgcatttggaaaggaggccaagaaaggaagagaggtcgggcccccttttgctgctgagagtgaccttgagaggaggctgggccttccttcaatgccagctccctctgactcagcctggactggggcatcacctctctgggtctgcgtgtccccacatgcccttccttaagcccctgacccaagccctgggggagggaggggcttacactgctccctcttgcctcactctccattgacagctgtagaaactgaggctcacaggctgggcgtggaggctcatgcctataatcccagcattttgggaggccgaggtgggtggatcacctgaggtcaggagtttgagatcagcctagacaacatggcaaaaccctgtttctagtaaaaatacaaaaactagccaggtgtggtgctgcacgcctataatcccagctactcgggagcctgaggcaggagaatcgtttgaacccgggagacagaggttgcagtgagcccagatcgtgccactgcactccagcctaggcaacagagtgagactctgtgaaagaaagagagaaagaaagaaagaaagaaagaaagaaagaaagaaagaaagaaaggaaggaaggaaggaaggaaggaaggaaggaaggaaggaaggaaggaaggaaggaaggaaggaaggggaagggaagggaagggaaggaaggaaggggaagggaagggagggaaggaagggaaggaaggaaggaaggaaggaaagaaggagaaagaaagaaactgaggctcaggagggctgagcctcctaagccagctgcttcaccaaccttctgccagctctagccctttcatccttctgccctgtcagtccctgccctctgcaagtgggacaggggtgaggggagtgtaggagctcaggtacgtgcattctccagcccccgtggcatttctgcaggggtttggagcagaagagaatgatgaggtgggcagaatccccggagcctgcgtggaggaggagagagaggatgagctgggccctgctggatgcctggtttccctatctttcccctaagagccaggccgacctcgtcctgctggacggctgaggaaactgaggcctggagcagcgaggggcctgctcaaggtgcaccactgcaatgccagccgcgactccacccaccccattccctaagggatgcctgggaggtgaataacctggaagttcaggtgggacctcccagaggatctaggcatccctgtagtggacagtggctcaggcaggcagtgcccaggctcggaaccctcccaggggcctggaggttgccaaatgggcccaatttgctgtgctaatgtgggctttctgctgcttggaacctgtgcacaccctcctccaagagctgagccagcagcaacaggacagactataattatgacaagttacatcctgttgtcctggctcagttattagccacatcgcccagcacccagcagcttgcactccgggggtctgccagaagcagggagggtgaggtgaaccatttgttaacactcgccccccatcctgccatacaacttctcccccaccccctgccaactagggatgccttttcctgctagttactcatcccctaaacctgacctcaatcaccctccaggggagatcgctgagctggacattccaccagcctctacagaccggggcacccacagcagggaggatggtggacagtagggagtcctcccgaaagatatcccacagcaaccctcatttttccgcctttaatcccacacttctggggctgggctctgagacaggaatgccatccatgtttgttccttgcacaactccagggaagccactcacatagactgcaacaacttagaaccttagactccaattaaactgtggcctctgcagttgggcagtgtacaacttgaacggctgtacatgttctgcaaggtacaatttctcaactccttccctcaagcaaataagacacaagtcctggaggcacagatctgtccacccagctgacaaattctggggctgcatttccagaatgttctatgtgaagtgggaaaaggattctttcttttctctcctcctctgggccagtgtcatcatgggagttctgagtacagttgcaagcctgctctgggagagaaacagatacatggaaggagaaattccaggagctcgggagacagcaaggacccagacccacattgtctgagcctggctgatgttcttcagtcagcagagcttctcggacctcaagtttagagagttagattcatcatgaattttggttcatggaacacccgatcattgacccatagctggccctttttatctccatctggttgattaattgattgattggctgatacaatgaacatccatgaacaccacccaactcagggactagaatgtcaccaataacttatgtcacttgtgccttctgatatagtttggctgtgtcccccgccaaatctcatcttgaattgtagctctcacaattcccacgtgtcatgggagggacctggtgggaggtcattgaatcatgggggtgggtctttttcatgctgctgttctcatgatagtgaataaatctcatgagatctgatggttttataaagaggagtttccctgcacaggctatttctcttggctgctgccatgtgagacgtgcctttcaccttccgccatgattgtgaggcctcctcagccacgtggagctgtgagtccattaaacctatttcttttgtaaattacccagtctcgggcatgcctgtatcagcagtgtgaaaacggactaatacatattcctacccacctgccctccttcacacagaggaacgaccatcctgaacttatgcctattattctcaccctgattttatgacatctacatgtacacttaaacaatacatacttgaattttgcttgtttttaaacttttttttttttttttttgagatggaatagtgctctgtcgcccaggctggagtgcagtggcatgatctcagctcactgcaacctctgcctccagggttcaagcgattctcttgtctcagcctcccaagtagctgggactacaggtgcacaccaccacgcccagctaatttctgtatttttagtagagatggggtttcaacatgttggccaggctggtctccaactcctgacctcaggtgttctgcccgcctcaacctcccaaagtgctgggatttcaggcgtgagccaccacgcctgggcgatttttttttaagacagagttttactcttgttgcccaggctggcatgcaatggtgtcatctctgctccctgcaatctccactttctgggttcaagcgattctcctgcctcagcctcccaagtagctaggattacagacatgcgccaccacgcccggctaattttgtatttttaatagagacagggtttcaccatggtggtcaggctggtcttgaactcctgacctcaggtgatctgcccgcctcagcctcccaaagtgctgggattacaggtgtgagccactgctccccgtcactacatgatttttattacaaaaaaatttcttggccaggcttggtggttcacagctgtaatcccagcactttgggaggccgaggtaggaggatcgcttgaggccaggggttcaagaccagcctgggcaacacagctaaactctgtctctactaaaaataaataaataaataagtaaataaataaataaataaataaaataattagctgggcatggtggcatgtgtctgtcatcccagctactcaggaggctgaggtgggggaagatcactggagcccaggggtttgaggctgcagtgagcaaagacggcaccattgcactttagcctgggccacagagtgagatcacttctcaaaaaatgtgtatttttctttctttagtaataactaacctcagcttactgtaacttttttactttataagccttccaattctttttaacttttttactcttttgtaataacatttactttaaaacacaaacatatcatacagctgtacaaaaatattttcttaggccgggcgcggtggctcatgcccgtaatcccagcactttgggaggccaaggtgggcggaacacttgaggtcgggagttcgagaccatcccagccaacatggtgaaaacccgtctctactaaaaatacaaaaaattagcccaggcatggtggcgggtaactgtagtcccagctacttgggaggctgagggaggagaatcgcttgaacccaggaggtggaggttgcagtgatctgagatctcgccactgcactccagcctgggtgacagagcaagtctccatctcaaaataaataaataaataaaaataaaataaatttttttctttatattcctattctataagctttttgctatttttaatttttgtttttacttcttaaacttttttgttaaaaacgaagacacaaacacacacattagcctgggcctacatgggatcagcatcatcaatatcactgtcttccacctccacatcttatcccactgaaaagtcttctaaaactcctggggctgtcatcgcctatgacaacaatgccttctggatacttcctgagggaactgcctgaggccgttttatagttaacttctttttttaataagtagaaggagtacactataaaataatgataaaaagtgtagtataataaatacataggccgggtgcagcggctcacgcctgtaatccccacactttgaaaggccaaggcgggcagatcacttgaggtcaggagttcgagaccagcctggccaacatggagaaacctagcaaaaaatacaaagattagccaggcttggtggcatataaatctcagctactcgggaggctgaggtaggagaatcacttgaacctgggagtcagaggttggagtgagccgagatcaagccactgcactccagcctgggcgagagagcgagactctgtctcaaaaaaaaataataataaaatacatacatacataaaccagtagcatagtcatttgttatcattatcaaatattatgtactatatatcattgtatgtgctagacttctttttttttttttttaagacagggtctcgttctgttgcccaggctggagtacagtggtgcagtcacagctcactgcagcctcaatctcctgggctcaagtgttcctcttgcctcagcctcccaaagtgctgggattacatgtgtgagtcactgcacttggccttgcttatttttaaactttaaataaagtttaaagttagcaaaagtctctagaagttgccttttcactggacatgacacaatcaggcatcacttaatgacagagatagattttctgagaaatatgttcttaggtgattttattgttgtgtgaacatcctacagtgtatttgcacaaacctagatggactagcctcctacacatctaggctagagggtctaacctgttgcttctgggctacagacctgtatagcgtgtgactgcattgaatgctgaagacgcttgtaacacaggggtaagtgggcaggcatttggccatctcacactatttagatgagggattcctccttcaccaatggggtgaataggagattatcagggcacatgtgacttgctttgatcaatggcatgagaggagaaatgatgggccagtcccaaatctaaagccttgactctctccttctgccttcaccccatgccatgaccaagagagaagctgcaccctcagcctgggacccaggaagaatatactcagagcagagcagccctggccaatccccagactctcagcttgagtcaggacttcccaccaagctcaacatagccccaacctagatcaaccaagccccagccaagctgcagagccattagcattaacgaatgtagctttgagccaccagagatgggggtgatctgttactcagcattatggtgtcaacggctaatccattgaatcaaaattcacttatgttgctatatgacgccacatgtggcttattcttaattgtgtacctataggcccattaatgtatccacttttcttattgatgagtatttgagttgtttcaatcttttgctaccataaatagcactgtcttaaccttcttttaaatttctcagccaggtgcgatggtatacgcctgtaatcccagcactttgggaggctgagaagggcagatcaactgaggccaggaattcaagaccagctcagccaacatggtgaaaccctgtctctactaaatacaaaaattagccgggcatggtggcgggtgcctgtagtcccagctgctcgggaggctgaggcagagaattgcttgaacctgggaggcggaggctgcagtgagccaagattgtgccactgcactccagcctgggtgacagagtgagattccatctcaaaataaataaataaataatcttgatgtgtgtgggcaagagtttctctggagaatatagctagaagtagaaacgcacatattcaactttacaagatagtatcaaattgtttttcaaagaggctgcaccaattgtcactcctaccagtcatccatgagacccggctcatttcatcctatccaatatctgctgtcagagttgggtggttttttatttttgctttgttttgttttgggttttgttcttgcttttcctttctttctttctttctttcttttttttttttttttgggacaaagagacagggtgtcactctgttgctcaggctggagtgtagtgttgcaatcatagctcactgcagcctcaaactcctgggctcaagcaatcctcctacctcagcctcctgagtaactgaaactacaggtacacactccatgcctggataatttttatttttttatttttgtagagatgggggggtctcgctatgtttcccaggctggtctcaaactcttggcttcaagcaatcttcttgcctctgcctcccaaagtattgggattataggtgtgaaccactgcacctggcagaaaaatatttttttatgaatgtagtgtagcccaagttctccatcttaatgtagccaaatttttctttgctgttttggtgtgtctcacaccacttcactaatagcaagcaacattaatggcaactagatgctaaatgttctataacagggataagttgaattatgtcaaatccacacaatgaaatactatactgcaggccaggcatggtggctcacgtctgtaatccctgcactttgggaggctgaggtgggcggatcacctgaggtcaggaattggagaccagcctggccaacatgatgaaactccatctctgcttaaaatgcaaaaattagctgggcatggtggtgtgcacctgtaatcccagctattcaggaggctaagacaggaggatcgcttgaacccgggaggtggaggttgcagtgaactgaggtcatgccactgcactccagcctgggtgacagagcaagaccccgtctcaaaaaaaaagaaaagaaaagaaaagaaaaatattatactgtagttaaaaatcattatttcaaacacatatgatattaaaagatattcacattgtaataagtaaaacggctaagtaacaaaatttgattctaaatgtgttaagataaactgaaatgtcttccacctgtcattagacccagacttagaataaaaatcaatctaatccaagctgagtccatccacgcaggttagaaaagagccctttattgctagagagggttttatggtgatgtaagtcaggcttcccaggaaaatctacggaggggacaagaccaacgccgtataaatgagggaaacagattcatctttttttacagagtcttttttttgagaccgagtctcactctgtcacccagagtggagtgcagtggtatgatctcggctcactgcaacctctgcctcccaggttcaagcagttctcttgtctcagcctcctgagtaactgggattacaggtgtgcgccaccatgcccgcctaatttttgtatttttagtagagacggggtttcaccatgttggccagacttgtcttgaactcctgacctcaagtgatccacccacctgggcctcccaaagtgctgggattacaggtgtgagccaccgcacccagcccaggttcatcttgatttgaaattggccatgttacttctgagaaagaaatgatgaaagcagtggtcattttggtgagatgtgcttagttcttgccctcctgagggtggtccctgccgccctctctgggaggatcctgaggtggtctggagtgccaatctttgttccccagcaaagcaccagaaggggccagtggagggtaggggagacagggagagtcaaaaatttctgtcatataagattagaaacaagtcaagtgtggtggctcatgcctgtaatcccagagttttggggggccgaggggggaagatcgcttgggcccaggagttcaagaccagcctgagcaacatagcaagaccccatctctacaaaaaaatgtaataattagctgggtgtgatggcacaggcctgtagtcccagctactcaggagtctggggtggaaggatcctcgagcccaagagttcaaggctacaatgagctatgatggcaccactgcactccagcctgggtgaccgaggaagaccctgtctctaaaaaataaagtcaagagggcaggcacggtggccagcactttgggaggccgaggtgggtgggtcacttgaggccaggagttcgagaccagcctggccaacagggggaaaccccatctttactaaaaatgcaaaaattccccgttgtggtagcgcatgtctgtaatcgcagctacttgggaggctgaggcaggagaatgcagtgagccaagattgcgccactgcactccagcctgggtgacagcaagactgtctcaaaaaataaaaataaaataataaaataaataaaatcagtaactatctattggaaggtggaacttctttgagatctgcatcaaagttacatttgtataacaaatgccctaatttatttatgttgtctatttcaggtggctgggcgcaggtggtggggggaagcccctacagagattggagggggagctaaagattccctaagccctcacttgtgctgtgtgtggtgtaggtgaaagtgacaaggggtttctttctccacaggggaggagaggcgtatctaagaccgggatgtatctaacatggggacttagagggagggggtgcagaggagaaaggaaaacaggaagagggtcttctccttctctctgctccacctcagagccgctagcagagctgggcacgtggcgagttctccgggaaacctcccaggctccgctcacgctcccacatggccccaggtgacctcggggtgcccgcgctgtggggagccttcaaggcgcccgagtgaacctggggactgtttgaagtcccggcaccttctaaggattccgtttaacaagctgggtgccgtgagcacttggatgaaaggcgctatgtaaagacatgatatcataacctattaaagctcgttttcttagaaacaggaaggtccatttgccaagagtaaacagtgagggcaagccctgcccgggcacaggatcccacagttgagaaagtcattctgggtgggctcaggagccagcgtgggtcagagcgtgggggtgcagaggggttggaggtctcccccacgccccaaccccaggagggagaagtcagcagaaacagcagctggggccccagagggctggagaattccagcctagcaggaatgagggaattactggcccggtctggatggagggggccactccgtcaaactcagaaacaccccgacatcctctgcaggatgtcacctccatgaagggacaaatggggggtgctggccctgtaattcccctttcattaattctacaaatgtcaggccgggcacggtggcttacacctgtaatcccagcactttggaacaccaaggcgggtggaccacttgaggtcaggcgttcgagaccagcctgggcaacatgttgagatccctgtctctattaaaaatacaaacaagtagccagatgtggtggtgtctacccatgatgccagctgcttgggaggctgaggcaggagaatctcttgaacccgggagctggaggctgcagtgagctgagatcgacccactgcactccagcctgggcgtctcaaacaaacaaacaaacaaacaaacaaaaacattttttggaagaggtggtgtctgagcttcacctggaaggataagtaggacttgagggacagaacaggaagggacacaagcaccttgaatatgtccagggatttccagaggcaaaatgcacttcactcacccagcaggagggccctgggctggaagcagggccgggcttcccatcccttcttgagcatgaacaagccaaggcttgcagtgggcctgtgacccacccctgaccagaccccccacctcccacccagctctcccctaccccccaccacgccaggccctggcactgaggctgtaccgcgtctccccgtcctgcagagtccaatcagctcccccaaactcaacctcaggccgcgaccctggcgaaggcccggaggagccaggcgctgcttcccagcaactgcgtttcctcagccttgtttacagttgttgaactattttcgtcaaattaattaaatacatgctaaatcctctccaatttgttgtgcgataaattaaagatgcgaagattgtgaaacgctcaagaaaacccactgatgctgataaaagccggcaccacagccccgagtcaagcatcaggcagtccagagtctgcttctaccacccggctgaatcagagtggggttctgaggtctcagcggccagggtcccagggagacttcatgtctacccacaaaggtgactcaaggcccaggggtggcccatgtcactggagagcacagcgcatggctgtcaggggcacaggacacaaagtccttccccagagtgtaccgcagggcttcctacaacccctcaggtcagcagcttgcaggaatagcaacagcttgaccaggcatggtggctcacacttgtaatcccagcactttgggagactgaagtgggaggatcacttgagcccaagagtttgagatcagcccgggaaatacagggagaccccatctctaccaataattttttaaaaaactagccgaggccaggcgcggtggctcattcctataatctcagcactttgggaggccaaggtgggtgaatcatttgaggccaagagttcaagaccagcctggccaacatggttggccatctctactaaaatacacatctctactaaaaatacacaaaattagctgggcttagtggcatgcacctgtaatcccagctactcaggaggctgaggcaggagaattgcttgaacctgggaggcagaggttgcagtgagctgagctcacctcattgcactccagcctgggtgacagagcgagactcgatctcaaaaaaaaaaaaagaaaagaaaagaaaaattagccaggcgtggtggtgtgcaccatgagtagtctcagcgactccgaagctgaggcaggaggaccgcttgagcccgggaggtcaaggctgcagtgagctatgatcacgccactgcactccagcttggacgacagagttaagaacctgtctccaaaaagaaaagaaaagaaaagaaaggaatagcaacagctaatgttgatctgagatcgcagtgcacagtaactcacgtaatcctcatgcaactctccaagggggcaggtgctgctattatcccattgcacagaggagaaaatggaggcacaggaagtaactggccaagttcacacagcctgggaggacaaaaggcctggatagaggctgggctgtccagctgtagagtctgtgctggtaaccactatgtacacgagatcctcctcgagatcctcctctagctctggcctgtgccaggcatggctttggcagaatcagccaccacaggccagaccacacccctaggaggcctctggggtctgggacacacaccatcccacccaccccacagctagcatctacctcgttctagtcttgttctgtgtaaagtgtaccccattggcctacatgagagggcaggtcacctatgaaaatcccttcactcagacaggggcctcctgcacctgcccgggaaaaaaagagaggcatgagtgttccagccagagaaactgaggcacagagggtcttgcatctgtgagtccgtggcctaagccccctttcctggagcaaggtgatgcgtagattcaaagtgaccccacagccattgcccatgtggatccagtggaaccccaggctgtgtgtttgtttgtttcgagacggagtttcactcttgtcacccaggctggagtactatggagtgatctcggttaactgcaacctctgcctacagggtttaagcaattctcctgcctcagcctcctggaattataggcacgcaccaccacacccagctaatttttgtatttttagtagagagggggtttcaccacgttggtccggctggtctcgaactcctgatctcaagtgatccacccacctcggcctcccaaagtgctaggattacaggcatgagccaccacgcccggcctgtgttttctttattccatgttgtggatgaagagactgaggtccagaaaagaacagggacgcgtgcccaacatgcaaagagagtgggtaggagagctaagttcagacccgggaagggctggctgagctgctggtacccaaagcccaggtcccatgctggggctccccggtcactgccatcactccactcccccaaggtggcaggcagtctgggtgtcttggggcccagggtacctttcacagagccttgattccactctttgaggtgggcacgggtgtaccccacccgctaagcagcactgagaggccagaaacaactgtggatttgaaaacatctcgtaaactccaaatcccaaaaaggatggtgattgttggaaatgttgatgctggagaagaggatggggacaaattaggtgagttccaggccagtgcctcttgccaagacacatgcaccccctcaacagacctcattcctttaatatatatatatatatattttttttttttttttttttttaagagacagggtctcagtctatcacccaggctggaatgtagtgatgcaatcatagctcattgcagcctcgaactcctgggctcaagtgatcctcctgccttaccctcccaagtagctgggactacagaagtgtgccaccacacctggctaatttttttgtatttttagtagagacggggtttcaccatgttgaccaggatggtctcgatctctcaaccttgtgatctgcccccgcttggcctcccaaaatgctgggattacaggcgtgagccatcatgcccggaagcatttttttttttttttttagctcatcagctatcattagtgtcagtgtatttcatatgtatggcccaagacagttcttcttccgatgtggcccagggaagccaaaagattggacacccctggattggagggacaccagtggggaagagccaggaggatatgggaccacctgcgatcaaggagaaagctcacccccacttcctgtgctgtggccaatgggtggtgccagtcctgctccttcaaggaagcagaaccagggggggtatttattatagaataagaatgagcattggccaggctcagtggctcatacctgtaatcccagtattttgggagcctgaggcgggcagatcacttgaggtcaggagttcaagaccagcctggccaacatggtgaaaccctgtctccactaaaaatacaaaaattagccaggcatggtggtgcatgcctgtaatcccagctactcaggaggctgaggcaggaaaatcacttgaactcgggaggcggaggctgcagtgtgctgagatggcaccactgcactccagcctaggcgacagagcaagactccatctcaaaaaaaaaaaaaaaagagggggagatgtactggggaccagggaaaagtcagcggccagccctcccgcagcagggaggcaggcagaagagctggagcttgtggggaatcccggagcccggcggatgtggctgctgcaatatgaaggcagggccagtgtagacgcttatggaggtctcttcacaccatctctgtaagatcacagccaagcgtccagggaaggcacgggtcactgtgacttcttaacagggccagcagcctgggagaagagccagccaccaatcagggaataggacatgcagaagagtgtggataaactggaacctgcgggcacctctctgtccgtctctctgccctgcctctagccatgatgatgctcagagcacaaagttcgctttagtccaactctaactcagacccacagcgggaaggggattctaggaaacaaggctccagcttggccaacttgactcagtacaaaaccaccacagcaggaagagatggagctgcttgctgagaccaggcgccggggatgggggcaggttcagggatgcctggtatggttgtgtatgttgttcactgctcaagagcacctggctagggagccaggaggggctgaagctgaccctggcatgaagccatatctacctggaggaaggggcgtctttttctaacttgcataagataccattcaaacctgggcaggatgagggaaaggcgtggtgtgctggttgaggtggaaggtgtgggggtgtccataaggttaaacttctcccagcatccatcatccccctctgtgaccaacactgtaagggccccatacacagggcccttgctgtcagttggactaaccagtaacaggcttgtggcatatggcgatggtcctaaaattcccagagggcaggcagtgcaaaagcaggcagagccagctaacttggagggaggattatgccttaggcagagctgtttcagatagaacgagatgtcttttgaggtagtgagctccccgtcactggaggtattcaagcagagccagatggatgcttgcagggtttggtgcaggggatgtcatgaagctccaggtggcagttcagccacttgctcctcctggacagtgagtcagacatgaacaagacttcagaggattggtggcaggtctcgggcagggttggggggagttcctctccagccctcccccaccctttgtctctgctcagttctttttggaaccagtttaggtatttagaagattaatttcatatttgtgcaaagcaggggattaggaggggaggagactgaggctctctgcaggtcgttaatctaaaaaaagactgcaaaatcgctctggccacgagtcacagctgagggtgccactcaccgccagcaagccagggcttttcatcagtagtttgctttcattttctgccgtctcaggaccaacactcttgagctctggggaaggtcaaaggcagaacatggtggggtggcctccatgccacagagcaaggaggctggagtgccacccccatcacgccaaggtccagactgcagacctcaaacagaattactgccatgaggaccaccctgattcctcagtccactgccacctcctgcctcactcttcggtgctcctggagaaaccagaggctcagagagggcatggattcaccccagcacacacagcaagttgctgattcctgactcagagctcctcctcgatgctgccacttagagttgaggtggtgggagctgctggagccgttctgggactatctggaagcacagcccatacatccaggagtctagggggattttttttttttttgtaacagcattattgggatagaattcccaagccatacaattcatccatttaaagcagggatgtccaatcttttggcttccccgggccacactggaagaagaattgtcttgggccacacctaaaatacactaacactaatgatagctgataagctaaacacaaacaaacaaaaaaactgtcgcaaaaaaagtctcataatgtcttcagaaagtttacgagtttgcgttggattgcattcagggccgttctgggccacgtgtggcccatgagcctcgagttggacaagcttgatttgaagtgtacaattcaataaactatcgaacttttgtatattcagagttgtaaagccaccaccaaaatcaattttagaacagtttcatctccccagaaagcaaccctgtactcattggcagtcactcctcacttccttccccatcccagctcctcgtaaataatctactttctggccaggcagggtggctcacgcctgtgatcccagcactttgggaggccgaggcaggtggatcccttgaggtcgggagtttgagaccagactggctaacatggtaaaaccctgtctttactaaaaatacaaaaattagctgggtgtggtggcgcacatctgtaatcccagctactcaggaggctgagacaggagaatcacttgaacctgggaggtggaggctgcagtgagctgagatcacgccactgcactccagcctgggcaacagagcaagactccgtctcaaaaaaaaaaaaaagacaagctcaggaaagattttggctagtaaactagaagggaaaaaggaagaggctggaaatgtcggggtttgcagggtttgaaaagccaactgtaggctgggtgcagtggctcctccctctaatcccagttctttgggaggccaaggtgggaggattgtttgagcccaggagttggcgaccaaccctggcagcatagccagaccccatctctacaataaattttaaaaattagctgggcatggtagcatgcacctgtagtccaagctactcgggagactgagtgggaggatcacttgaagccaggggttcgagaccagcctgggcaacatagcaagtctctgttggagtccaaggctgcactgagccatgatcgcaccactgcactccagcttgggtgacagagcaagaacatgtctcaaacaaaacaaaacaaaatagaagagagagagaaaagaaaagccaactctttctggcttttaacttcatatatgttaaatgtcaataaatttaccttcgtgtatgtaaagtgtcaattaaaaagtgcccttgacgaaacaaagttaattcaggagacagaagataacttgaacaaaaactagttaacatccccaggggattcactaagtaaacaaaaaaagagctctggggaattcaaaatatctttgctagaatttttaaaaggtaacaaaagagttggtgattaaagctgaggaatctcccagaaaggagaaaaagagagatgggagataaaagatcagacgtagaggggaaaatccaaccgctatccctgggcgttccagaaggacagaacagagaatatagaaggaaataagtgatcaaagacacacaagaagagaagttcccagaacagaaggctaggagccttcagattgacggccatgcaatatgataataaaactaaagatggcgaggtgcggtggctcatgcttgtaaccttagcactttgggagaccaaggtgggaagattgcttgaggccaggaaatcaagaccagcctgggcaacatagtgagaccccatttctatttaaaataaaataaaaatttattttgtttttatttttatttatttatttattttgtggcagagtctcgctctgttgcccaggctggagtgcaatggtgcgatcttggctcactgcagcctccacctcccgagttcaagtgattctcctgcctcagcctcccgagtagctgggattacaggtgcccaccaccatgcctggctaatctttgtatttttagtagagacaggatttcactatgttggccaggatggtctcaaactcctgacctcaggtgatccgcctgccttggcctcccaaagtgctgggattacaggcatgagccatcacacctggccaataaaataaaattttaaataaaactaaagaaaaggcatgaagaaacatcactgagaaacttcagtaggtgtaccagtgataaacagatggtcctaaatatttccagtgggcagaggtaaaagcaagtcactacaaaagaactgctgatcagctggacacgatggctcacgcctgcaatcccagcactttgggaggccaaagtgggtggatcatttcaggtcaggagttcaagagcagcctggccaacatggtgaaaccccatctctactaaaaatacaaaagttagccgggtgtggtgacgcatccctgtaatcccagctactcgggaggctgaggcacgagaatcgcttgaacctgggaggaggaggttgcagtgagcagagatcatgccactgcactccagcctgggcgacagagagagactctgtatcaaaaaagaaaagaaaagaaagcaacagagctgtgtcttctcggttctcaaggataagttttgttttttgttttttgttttttttgagatggagtctcgctctgtcgcccaggctggagtgcagtggtgcgatctcggctcactgcaagctccacctcccgggttcacaccattctcccgcctgtctcccgagtagctgggactacaggcacccgccaccacgcccggctaattttttttttttttttttttttgtatttttagtagagatggggtttcaccgtgttagccaggatggtctcgatctcctgacctcgtgatccactcgcctcagtctcccaaagtgcagggattacaggtgtgaaccacagagcccggccaagggaaaatatttttaacctaaaattctatacccaatccaactattgtgaatgcggaaaatctgagacgggtctgccttaatttagaaagtttattttgccaagattgaggacacgcacccgtgacacagcctcagaaggtcctgatgacatgtgcccaaggaggtcggggcacagtttggttttacacattctagggagacatgagacatcgatcaacatatgcaagatgaacattggtttggtccggaaaaatgggacaactcaaagcaaaggtgggaagactcaagcaaggagggggcttccgggtcataggtagataagagacaaatggttgcattcttttgagtttctgattggcctctccaaaggaggcaatcatgatgacacgcacctgtaatcccagcttctcggaaggctgaggcgggagaattgctccaacccgggaggcggaggttgcagagagccaggattgtaccactgcactccagcctgggcaacagcacaagactccatctcaaaaaaaaaaaaaaaaagatatgcctttatctcagtgagcagaggggtgactttgagtagaatgggaggcagggctgggaggcagaggctgcaatgaaccgagatcaggccactgcactccagcctgagcgtcagagtgagtgagactctgtctcgaaaaaaataaataaataaaaataaaaataataaaatgaatttaaaaatcgaaaaatgcttttgtagagatggggtcttgttatgttgcccaggctggtcgcaaactcctgggctcaaacgatcctcccaccttggcctcccaaagtgctgggattacaggcgagtgccactgtgcctggcctgtgaatgctttttgctttagggtccagtttttctttctctggcagtttctaaatgttgttcttttgcctgcattttctgcatcttgagttcttctgatgtttcagaaatgacatcacatctatggggaccttgttttccctgggtttctggtcactggtgttcactactcacccatcctcttttgctaatctggatcctggcatctggacaccccctggactgcagctgctattgcctgcatcccatttctccctctttttttctttcttggtttttttttttttttttttgagatggagtctcactgtgtcgcccaggctggagtgctatggctcgatctcggctcactgcagcctctgcctcctggtttcacgcatttcttctgcctcagcctcccgagtagctgggactacaggtgtgagccaccacgcccggctaattttttattttttagtagagacagggtttcaccatattggccaggcatgtctcaaactcctgacctcatgatctgcccgcctcagcctcccaaagtactgggattacaggcgtaagccacggcgcctggccctccctctttcttggtttatttatttacttttccttctgggagggatggatagacaatacattcgtgaaaattttggaagtctaaaaaataatatggtccccaggcttgttcaatagtgtgaaaggaaaataaatcctggggcccccacatcgctaagctaaagagaaaagtcaagctgggaactgcttagggccaacctgcctcccattctgttttgttttgtttttttgagatggagtcttgttctgccgtcacccaggctggagtgcactgaggtgatctcagctcactgtaacctccgcctcccgggttcaagcgattctcctgcctcagcctccctacaggtgcccgccaccatccttggctgattttttgtatttttagtagagacacagtttcatcatattagccaggctggtctcgaacttctgacctcaggtgatctacctgccttggcctcccaaagtgctgggattataggtgttggccaccatgccctgcccccccaaattttttttaattagccaggtgtggtggagtgcctgtagtcccagctacttagaggctgaggtgggaggatcacttgagcctgggtggttgaggttgcagtgagctttgatggtgccactgcaatccagcatgggcaacgaagtgagaccctatctcaaaacaaacaaacaaacaaaaattctggggtcacgttatggttgatgagcaagccaaggtgtgacagtcaagggaagcatctgagtggggtggagaggaagtcatttgcctcactgtgagataggtgaccctcgaggctcagaggctgtgagggtggaggagtgagaggaaccccacaaatggggctaatgtcttcactgaatgaggagttatggggatcaatgaatgagcctccaaggatcctcagagacgtttctgctgcctgaccaggccagcatcccacagccaaggaggctgggctctgtatttggttcattgatccaagttccctctagaccccgggatcatcttcctaccccaccccacactgatccctcccacctctcatctcagcccactccagttctgacaaacacattccccaagacctcatcaaatcccacatcagtacatgctcgggaaggagtggagttggttccagagaggttggcagtgaactggagtgagagatggacagagaaattaagggaattttcctggagggaagggcttgagggtggaggttgggaggctcagctgctagaagatccgaggaagaggacgcagaccaactgtggtctgggaccccagattgctgagccatcagcaaggggagggtgctgcagggagttccatttgggttcaaataaggaagccctcttgaaggagtgagttctccatccaagggggtgtgtaagcagaggctgcacatccaccccgctgggatggtgctggtaggattcaggcatgagatgaggatctccagccctgagactctacgaaaaggagatgaagagcaatgggagggaggggagagcaagacagtgcccgaggctaaagaacagacgccactatctcagctgtggaagcagcagctggagatggaacaagcccttatgtgtggggaatttgcctcagcagatgaggtacagcgaggatgctcatgatatctcctgaaatgaatacaatctggaaaaaaaacaaattcaacagaacccattttttttccactaataaaccaagccaacttcagtgccatcctaatttatctgttgagaaagaaggtctttattctcaggaggtgggaataccacttctttattatcatcatcatcatcatcatcatcatttttttttttctgtttgagatgaagtcttgctctgtcgcccaggctggagcacagtggcacgatctcagctcactgcaacctccatctcccaggttcaagtgattctcctgcctcagcctcccaagtagctgaaattacaggtgcatgccaccacgcccggctattttttttttttatttttaatagagacagggtttcaccatgttgaccaggctggtcttgaactcctggactgatgtgatctgcccgcctcggcctcccaaaatgctgggattacaggcatgagccaccgcatgcccagccagactcccaagttttctttgtcaattcttgctttaaatatcttgaggctatgttattaatgtataaaagataaaaatgttctctctttctgatgaatttacttttttatttttcattgaccctctttatctctagtagtgcattagagcttaacgtctattttttctaacattaatatagctacctcaccttttttctttatttgcctagagcatatttttcatccttagatttctaatccttctgtgttcttatttttgtgtgtcttatgtttttgtgtgtcttataaataccatatacttgaattttattttaattttgtttgacaatcttcgtcttttaactggagagtttagtttagtttgtttatttatttatttatttatttatttagagatggagtcttgctctatcgcccaggctggaatgcagtggctcaatcttggctcactgcaagctccgcctcccgggtttacacggttctcctgcctcagcctcctgagtagctgggactacaggcacccaccaccacacccggtgaaatttttgtattttttagtagagacagggtttcacggtgttagccaggatggcctcgatctcctgacctcgtgatccgccagcctcagcctcccaaagtgctgggattacaggcgtgagccaccacgcctggcctatttatttatttatttattttttagatacagaatctcactctgtcgcccaggctggtgtccagtggcacaatctaagctctctgcaacctccacctcccgggttcaagcgattctcgtgcctcagcctcccaagtagctaggactacaggtatgtgccaccatgcccccgctaattttttgtattttagtagagacagagtttcaccatgttgcccaggctggtcttgagctcctgagctcaggcaatctgtctgcctcattctcccaaagtgctgggataacaggcgtgagccaccacgcccagctagtccatttacattgaagtgattggtgagcaattagggtttattgctatcatcttattatattttcctatttgactcatgttttctccttcttcgtccctctcttttcttgtcttcctttggtttattagttagatattattgtaataatgttgcataacagaaaacttcaaaatctccgtggtttacaacaccaaaaacgatttctcactcgaagatctgaggctggctaccgctctgctaagtcttctaattgttctatattgtgggtctcctctaagcatattttcacagggagcaggctgaagcaggagaggctacctagggcatgtttttctcacagcagtgacaggcacagagggacaaagctaaatcatgcaaggacatttaaaacctcagtttgcagccagctgcagtggctcacacctgtaatcccagcactttgggaggctgagatgggcggatcacttgaggtcaggagtttgaggccagcctggccaatatggtgaaaccccgcctctactaaaaatacaaaaattagccaggcgtggtggtgtgtgcctgtcatcccaactactcaggaggctgaggcatgagaatcacttgagtctgggaggcagaggttgcagtgagtcaagatcacgccactgcactccagcctgggcgacaagagtgaaagtccgtctcaagaaaaaacaaacaaaccagaatgtagattttccccatgaaagacagttttgtagggccatttcaaaatacatcaaagaaatatatttgtgggtaaaatactttgatttctttcagggcctgctatctgtcatgctggtatcttattgctaaagagagtctgttttgtcagtcttaaggtctctgttttaatgtgaatgctggtcagctgtacctgaattccaaagggaggtgggtataatgacgcatgtctgaccaaagccattcccatcacagcctgaactagtgtttcagatttactttagaatgcccttggccaagagaagggaactattcacttggctggtgggggtgcttagaattttatttttggtttacagctcctaggagaaatatgtgtgtaggtatgtacacccaccacacacacacaccccgcactcatatagattataatataaaaacaacacattctggctgggcgcggtggctcacgcctgtaatcccagcactttgggaggctgaggcgggcggatcaccggaggtcaggaattcgagaccagcctgaccaacatggtgaaaccctgtctctactaaaaatacaaaaattagccgggcatggtggagggtgcctgtaattccagctactcaggaggctgaggcaggagaatcacttgaacctgggaggcggaggttgcagtgagccgagatcacgccattgtactccagcctgggcaacaagggtgaagctgtctcaaaaaaaagaaaaaaaagagagacaagaagaaggaaatgcatttaatacacctaatttaccaaacatcgtagcttagccaagcctacctcaaacacgctcatgtaaccaagtatcgccatttttctaagagatagtttatttttctctctcccttcttttctctttcctcctttacgctgcttcctatttagctatttagaaatgcaattatagccttttacctcccctgcaccagatactctctattttaggcatccccagcccccaggccatgttaggaacctgttaggaaccgagctgtgagtggcaggtgagtgagcattactgcctaagctccacctcctgtcagatcagcgatgccattagattctcataggagcgcgagccctattgtgaactgtgcatccgagggatctacgttgtatgttccttatgagaatctaactaatgcctgatggtctgtagtggagcagtttcatcccaaaaccatcccctgcatccgtgggctatggaaaaattgtcttccatgaaaccagtccctagtgccaaaaaggttgtagactgctgccctacaggcaagttcatctaaccatgtgcttagaagctccagagcggaactctctcccaccacgaagttgccttggcagacaacagtccatctgcagcccaaagtgtgcccgctacgaaactctcccctacctggagagtttttggccacttctccaatgtagttctatccacaaaggtgccagcagtcaccagctcaagggcctggtcatgctagcaccaaagctatcatgaagagccccccaacctgcttgcttccttccctgcatgtcactcatgccaagcccccttctaaaagcacctgccttctgctctaaaagtgaagggatacccttaaggcaggaagcctgtacttctccccctaagctagctttggaataaaaagtcactttcttggccaggcgtggtggctcacgcctgtaatcccagcactttgggaggccgaggcaggtggttcatctgagttcaggagttcaagaccagcctggacaacatggttaaactctgtctctactaaaaatacaaaaattagccaggcgtggtggtgcgcacctgtaatcccagctacttggaaggctgaggcaggagaatggcttgaacccaggaggtggaggttgcagtgagccaagattgcaccactgcactccagcctgggtggcagagcgagactctgtctccgaaaacaaacaaacaaaaagtcactttcttttttttctttttcttttctttttttttttgagacagagtttcactcttgttgcccaggcaatggcgccatctcggctcacagcaatctccgcctcctggattcaagcaattctcttgcctcagcctcgtgaatagctgggattacaggcatgtgccaccacgcccacccagttttgcatttttagtagagacagtgtttctccatgttggtcaggctggtcctgaactcctgacctcaggtgatctgcccacctcagcttcctgaagtgctgggattataggcgtgagccagcaagcctggcaaaaagtcactttctttatatcagactttgctcttgttcatcggactctacaagcagcaagtgactgaacccgcaattcggttacactcagaacactgacattagcctacggttgggcaaaatcatctggcaacacagtacactgtacagtatcattgttgactctcatggttgcctggctgactgggagttgcagctcactgcaagagagtatcatactgcatatcgcttgccccggaaaagatcaaaattcaaaattcgaagtatggtttctactgaatgcatatcatgaagtcaaaaaatcctaagttgcaccattgtaagtctgagactttgtatgtaatctttcatcctctgccagaaatatctcttcagtaattgtccagtgctttaagtatatttccagggagctggggagtagcagggcaaagggccttgggcagaatggtctatgcctttgtgcctccgaatttcatctgtcaaatagaaaaaagaatccaggtcagatgagcaaatggaatcgtgtcaaagtgctcagcaaacaataatgtacaagaaagtgcttagcaaacaataatgtacacgaaagtgcttagcaaacaatagtgtacatgaaagtgcttagcaaacaataatgtacacgaaagtgctagcaaacaataatgtacacgaaagtgcgtagcaaacactaaggtacacaaaagtgcttagcaaacaataatgtacacgaaagtgcttagcaaacaataatgtacacaaaagtgcttagcaaacgataaggtacacgaaagtgcttagcaaacaataatgtacatgaaagtgcttagcaaacaatagaagtgttaataccacctgcattcttgcaatagcactgaaactacaacttcagtgacactctggttctaagcatgattttcagttattcccaaggaactgaagttgatgtagcagtgaatgtcccaggagcaattagacccacttcacttctcttgattgtcatttaatattcttttattaagcatcagttgtgagtgcaacctggagaaccacttgatgtcagagacatcagaactgagctcaagagacactcagatacctgatccagacctcaaaggaaagatctgtccaaagttgagaaactaagagaatgatctgtccacctagacaacgccaggccctaggagaaagtctgtccactcagaccctacaggagaagctcagggaacagtttgaccacacaggagctgctgaagaacagccagtcataggtgttgggggagaagggtgccgggaaagaggcaggaggaagatgtgcttccaggggggcagaagttaatggcgcccaatgcagctgagattgcaagtatgacacagactgagaattacccattgggtttgggccatgatgtggtttggttgtgtccccacccaaatctcattttgaattgtagttcccataatccccatgtgtcacaggagggacccagtgggaggtaatcgaatcatgtgggcagttaccttcatgttctcatgatagtgagtgagttctcatgaaggctgatggttttataaaggcttttcccccttttgctcggcacttctccttgctgctgccatgtgaagaaggacatgtttgcttacccttccaccatgattgtaagtttcctggggcctccccagccatgctgaactgtgagttaattaaacctctttcctttacaagtcacccagtctcgggtataactttattagcagcatgagaacagactaatacaccccatgttccatctcatttcatcttcaagcacccctctggagatgggttcagagaggcaagtggcttgccaaaggtcacacaggtgagaggagccagaggcagaatttagaggctggccaggcacagtggctcatacctgtaatcccaacactttgggagactgaggtgggaagatcacttgaggccgcaagctcaagaccgagcctgggccacatagcaagaccccatctctacaaacgaaaaagaaaaaatgatccgggcatggtggtgcatgcctgttgtcccagttactcaagaggctgaggtgggaggatcacttgagcccaggaagttgaggctgctgtgagccatgatcacgagactgcactccagcctgggcgacagagtgacactcttgtctaaataaattaattaattaaataaagcctcagtttgtgttacctccactcacaatcattggctaaagcaagtcacaccgtcagactcaatatcaaaggtggggggagtttgaaaaccaatagcctcaaattctttggttttgaaacttgaatgtgatgagaatctccaggctactcattaatttgcatctgagatgggactcagaattccactgcatacccacgaggccccagtagcatgcatggcctccttcctactgacccttgtagccgggggtccctgagttctgttgggctcccctcaccaccctgcctgcaaacacatctgagtatatcaccgaggttgctccccaagggaggagggtggcccccagggcactcgcggcccctcccctctgcctggccctcccctgagctctctgcagattcctcattagcaagtgcaaagccagccccttcacgccttcccaggccttgcaaggactgagagaggctggaatgcaagccccgaaccaccctccagcaatccttcattatcccttccctcgcaggtcccaccaccaggtaattccattgttaattaaaatcctgcagcctttaatatctcggctcataattgctgttttccccagccctggattggctgtgcaggcttcgggggggacccgggtttttttttcctgggatctgctttgtacaaccaccggctgtctatttcctcaacctcagccaaaaaccttcttctccgtgggcttcaatagtggagcaatggggaccaccctgggggccatattagcaccccctggacacctcctaggaaaggaggccaaggaaggagtcagtctcttcccccaaccccaggcaggcctccaagtacaacccccagcaacacagcctgggagacagagacagccagctcctgtacacgcaggccacgaaggctccagagtcccgcagaggccccctgaggccatgttcatgggcaacaaggatcttcctcatccacgtatcatttgctgctcccccgagccccgagcccccattgttcagctaaagaagaaaaagatcagagagggaggtgaatttcccaaggccacacagcagagccaggaggcctagagtgcagaccctctgctcttctgaccataccctctgcccgttgaccttcaggccctttctcaccttcctgctctcagcgttgatacttttcttcctcctctgctagcctcactcacacttagcccggcccctgtctcttcctctgccattcccattctagctgaggatgtgaatgctgtagatgcccacaggggaggcagggggccaacaatgagaaaggacagaggactgtgactattggtcacagatttcaaaggctggaggaccacagtggagccaggagggggacgcgctgaggttagccctgtcttttttttttttttttttttttttttttgagatggagttttgctcttgttgcccaggctggagcgcagtggcacgatctcggctcaccacaacctccgcctcctgggttcatgcacttctcttgcctcagcctcctgagtagctgggattacaggcatgcaccaccacgcccagctaattttgcatttttagtagagacggggtttctccatgttggtcaggctggtctcgaactcccaacctcaggtgatccacccgccttggcttcccaaagtgctgggattacaggcgtgcgccaccgcgcccagcatgctccatcttaatcactctgtgctgttgacccaaaggccccgtaaggtctgaaggatcaacccccatcccctgcccactgggaaggtgaccaggtattgctctctgtggaagacagcacctcactcaaggccaggcccctcttgggcaactgtgcagatgggggcagagcttggctggcctggctccagctttggagggtctgagggtggaccgagctcttgtcacaactgccctgctcctccctctgtggcccctgctgccatcctcctcccggcaggtgttgaccccaaggccatatgaccataaaacctccatccagagtctgcatcccgggggacctggccttctagagccatttttctttttgggatttctagggcctgcttcctctcttctcccttcccacagggaccccagaccctgtgcaccatgacctattacccacgcctgtcccatgctcctggctggtctgtcctttcagaactggagaacggagacgtatcctctccactctcaagaacctcagctcgaccgggtgctgcagatcctgcctgtaaccccagcactttgggaggctgaagcaggaggattgcttgagcctgggagttggaggccagcctgggcaatgtagcgagaccctgtttctacaaaaagtttaaaaagtagctgggcgcatggtgcatgtctgtagtcccagctactcaggaggatgagcagggaggatcattgagcccaggagtacgaggctgtagtaagccatgattgcaccacagcactctagtctgggcaacagagtaagaccctgtccccccaccaaaaaaaaaaaacctcagctccccctaacagaaagtctccaggggccgaggaccccttcagagcccttgatgtttctgaggtcagctgtgtcatcccaaatttccaggaactggaaaatctgtcacctgtcccagttgcatagctgggaggtgttggagatggagttctgaacacatctggttctcatctaatcctcagctcacagctgcctcagtcagcctccaacaagattcctctgtcactggtggctctgcgagaaaaccaatccctgaactgaccagctcttctgactgctcagaagtaggatcatgtttttgtttttgttttgagatggagtttcactcttgtcatccagtctgaagtgcattggcacgctcttggcccactgcaacgtccaccttccacattcaagcaattctcctgcctcagcctcctgagtagctgggattacaggcacgtgccagcatgtccagctaatttttgtatttttagtagagacggggtttcaccatgttggccaggctggtctcgaactcctgacctcaggtgatccgcctgccttggtctcccatagtgctgggattacaggcatgagccaccgtgccaggccagaagtaggatcgtgtttacagagcactgcttgcattcactcattcattcattcattcaataaacatgatgtcttgagcatcccttgtgccaggcaatgtgtttgttgctggggacaccttagggaacaagatggacaaagccctgatcctcttggagcttctattctagggagaaagatacacaatcaacaagataagtaaaataacatacaataggttagatagcgctaagtgctaaggaggaaaataacagggaatatggacaggaaatgcctgcggtgggttagagggtgaaagttgagtcagggtggccagggaaggcctcaatgggaaggtggcttttgacaaaagggtgcaggaagaccaggtgcagtagctcacggctgtaatcccaaaactctgggaggctgaggcagaagtttgagaccagcctgggcaatgtagcaggaccctgtctctacaaaaaaacttttttaattagctgggcctagtggcacgtgcctgtagtaccaactactcaagaggctgaggtgggcggatcacttgagcctgggaggtcgaggctgcagtgagctatgatggtgccactgcactccagcctgggtgacagagtgagaccctatcacaaatgattgagagagagagagagagtgaaagggtgaaagagtgaaagggtgcagggcccagctgggaacaataggtcacacggagccctttctctggactcttggtggggaagttcctccaaggaagaagttcctccaaacagaggctaagcagagaggaacggctgctgccccagcttctcagtctggttgtcctgacaggggcagcaggaagggtgcacggacacccacctgagtgaggagcttgctaggcagggggcccccttgtcccagtgcttccaccagtgcacccaacctgagccccacccaggtcctgctcacctgctgaatggccttccaacactgaagacctcagtcagctgcttcctagcaaggaaacacctgcagcctccttgcaacaagtactatatattggaagggcaccctgagattccaggtgaaggaaatcggtcacccagaggtgttgtgtgttccctgcaggagataatgcagcttcaaaccaggtcccccagcctccgtgaggaaagtgccctgcactgtcatcctgaccacagttcctggaaacccacgaggaaattggcacattctgcagatctgatctgctggcttcatctctgtgaggcacctcatcagtgacaacacccagatccaggctagaaccctgtaacattccactagtgaatctgtgctggctgccccttcgtgtttgctaacaagctttctgaggtatcagggagggacaccaagctctccccgctgtgtgacctgagtgtcgtgtctctctttagctcccatgtgtggagggaagaaagggaaaggtgccagctgatatcaaaatggacagagtaaaaccatgacaaatacaagacaaactacccaaagtcctttggaaccattctggccttgggaggtgtctgaatccacaagaaagactcagtgagagttaaaaagaagcattctgctcaagactggagagcagctgggatgaggctaagtagtaatggacaacagctacaaattcatgagaatgggaactgccatggattggacagcagggccctcgtgtgtgtgtgtgtgtgtgtgtgtgtgtgtgtatgtgtgtgtgtgtttaagagatggagtctcattctgtcacccaggctggagtgcaatgatgtgatcacagctcactgcagcctcaatctcctgggtttaagtgatccttccacctcagcctcctgagtgcctgggactataggcatgcaccaccatgcccggcttatttttaaaacttttttgtagagatggggtctcaaattcctagcctcaagcaatcctcctgcctcagcttcccaaactgctgagatcacaggtgtaagccaccatgcccagaccacacacactttttattcatgagaaaagtgaggctcagataaattaagtatcttcttcaagtggtgtggtagggatttgaacctgcatctcttcaaatttcaagaggcatcttttctcctttgagacagccaaacccctcaggatgtgcgtgttttccataggtggagggagcaccttgagtgccagcaggaggcagtgtggagcagaggaaaggccaaggctttggactcagacccacctaggattgcatctcaatgctgtgctatcttgaacaagttactaagattctctgaatcctgttgttgttgttgttgttgttttccttattggtgctgcaataagttatcacaaacttagcagctgaaaacaacacaaatgtattatcttatcattctggcagtcagaagtatgagatgggtttcacagagctgaactcaaggtgtcggcaggactatgtggatttctggaagcttgaggagataatctgttttcttaattttcctttttcttttatttttttattttgtatttctttattttttttttgagacagcatcttgctctgttgcccaggctggagtgcagtggggcaatctcagctcactgtaactccacttcctgggttcaagcaattctcctgcctcagtcttccaagtagctgggattacaggtgtgcgccaccatgcccggctaatttttttgtatttttagtagaaacagagtttcactatgttggccaggctggtctcgaactcctgacctcaggtgatccacccgcctcagcctcccaaagtgctgagattacaggagtgagccaccatgccatgcccggccttaattttccatcttataagactacccacattccttggcctgtggtccccttccatcttccaagccagtgtattagtcccttctcgtgctgctatgaagaaatacccgggactgggtaatctataaagaaaagaggtttaattgactcacagttccgcatggcgggggaggcctcagaaagctcacaatcatggtggaaggcaactcttcacagggcggcaggagacagaatgagtgtcagcagaggaaatgccagatgcttataaaaccatcagatctcgtgagactcactcactatcacgagaacagcatgggggaaactgcccccatgattcaattacctcccaccaggtccctcccacaacaggaggggattatgggattacaattaaaaatgagatttggggccaggcgcagtggctcacagctgtaatcccagcattttgggaggccaaggaaggcagatcacctgaggtcaggagttcaatatcagcctggccaacatggtgaaaccccatctctactaaaaatacaaaaattagccaagcatggtggaatgtgccagtagccccagctactcgggaggctgaggcaggagaatcacttgaacctgggaggcggaggttgcagtgagccgagatcacgccactgcactccagcctgggcaacagagcaagactccgtgtcaaaaaaaaaaaagaaaaagagaaagagagatttggatggggacccaaagccagaccgtatcagccagcaaggtagcatcttcagatcttctaatgctgacctcctcttctgcctcctgctttaaagaacctgatcacggctgagcgcagtggctcatgcctgtaaccccagcactttgggaggccgaggcgggtggatcacctgaggtcaggagttccataccagcctgggcaacatggtgaaaccccatctctactaaaaatacaaaattagccaagcgtggtggctcatgcctgtaatcccagctactcggaggctgaggcatgagaattgcttgaacctgagaggcggaggttgcagtgagctgagattgcgtcattgcactccagcctgggcaacgagtgaagctctgcctaaaaaaaaaaattaaaaaaaaaaattaaaaccagttttaaaagcgattatctcaccaaggttatcaaaatctttaaaggagccggggccagatgctccctgccacccgcaaacccacccctggtctgctgtaggcaagctgggtcacagcaggcttggcaataagggggtccccaggggaagtgctccctccaactggaggctcctgcccgggctgtggggatgccctcctgggaggtgaactggctggagttgtgtgccaagacttaattaaccctggagcaaccagatatccaaggacaactctggagccgctgagagtgacccaacctagatctggcacttcccccaaacagccccaggtgcaacagactaggggggccacctcccagtcagccacagccagctggctccaggcctctggaagtgctagtgcttggggtccactgagctttctgactcctcctcagatattttaaaaatcctagcctaaatgaatttgctacttgcagtcttgtgcctttaagtgaaattcaagccactgatttccagagtctaagtcaccagctggttttacaaatagacatgcaaggtcctagaatcttccagctgtttgccttaaaagacaggacaccagatttcttttatgaggcaacccttgagcccctacaggcctagctttggaaggaaggaaagatttaggctgcttaagtcttattgcagccactcatggctaccatagacagttgtgcagcctgcgcactacacaaatgcctctgggatgtcagggggagcgaacatgggaattaagatccagccacactgcactcaccaatggcatatatgatctcagactggcccctcccagagggaggtgcgcacacagcgcaggctcaaactctgcttctcacactagctgtccatcatgcacaaaacgtggcctacccgcacaaaatcaggatgctgctcaaatcaaatgtgacacagctcagaagctcacaaaactcaaactggtctattcccccgtgtgtttgaggatcacaagcttagatgtcaaggcaatgtcgaatttccatctgggtgcctggacgttactctcaatttctgtgttcattgtgtctcaaccagtaactgtttgcccagtgctgggcctagaggcagccctgctctcacccattgtgggtttcctgagtatactctggggcctcctaacctcactctttggtagatagtcagaaatcaaagccaggggtaagttctgagtccagaagaagaggcagtgaggacctccttccacaggccccatttttcttcctttcttttcttccttccttccttccttccctctcttccttccttccttctttcccttcttccttccttcccttcttccttccttccttctttcctttctctctctctttcttttctttctttctctctctctctttctttctttcttacagagtctcactctgttgcccaggctggagtgcagtggcgtgatctcagcttactgcaacctcacctcctgggttcaagcaattctcgtgcctcagcctcccaaatagctgggactacaggtgtgcgccaccacacctgactaatttttgtatttttcgtaaagatggggtttcaccatgttggccaggctggtcttgaactcctggcctcaagtgatccacccaccttggcctcccaaagtgctgggattatagccatgagccactgtgcctggcccaggcctggctttctgttacgcctggacgtgctctcagcagcttactagctggtatggagaggacgtttcagaggaaacacagactgtctcggctctgaaaagctgcctagattctgtcctgtttgtgagaaggctggcactgaaacctccacatcaccctgaagtcctggtctagacccaggaggcatcagcagtgtgaggtgctaggctagttatctgttgtttcctaacaagccactcaaagcactgtggctttacaaattttatgattctcgtcctcatggtttcatagagcttactggtttcagctgggtggttggttctggcctggggtcacttgtggagttacagtcagatggcatcagtggctagtgtcatctgactagaagtccaggatggtctccttactcacatgtctggtggctcagctagaagacccagggcctggccagatcactctctctccctgacagacttctccatgaggctccttgggcttccttccaacatggccacctcagaggagtcagacttgttacacagtggctggcttcccctagaacaggtgttaccagagagccaggtggaagctgcaaggctacttctgacttagcagggcacacagtcctgcccactggaggtatacccagggccagcccctatgtattcaaggggaggggattatataagggcatgacaaccaagaggcagactgggcgcggtggctcacgcctgtaatcccagcactttgggaggcagaggcgggtggatcacaaggtcaggagatcgagaccatcctggctaacacggtgaaaccccgtctctactaaaaatacaaaaaattagccgggtgtggtggcgggggcctgtagtctcagctactcgggaggctgaggcaggagaatggcgtgaacccgagaggtggagcttgcagtgagccgagatcgcgccactgcactccagcctgggcgacagagccagactccgtctcaaaaaaaaagaaaactaagaggcatggtttattggggccctctctggttactatccatgacaagggtgggcaggtccagggtgggacaacagagaacaggtggcctgactcttccctccagctaggactgagctgggcttttccacaagcaccatcatccatttatttcacaattgttctgtcacctgccacagagatgactcagcccctgtgctcaccaactatctttcctataggaaatgatccttccaggtggagaaacagtgcaagatattcccgtcctcagtattcaatccaaatcactgattcaatatctgcatatttgataacaggcctgtaatccaaatgctttgggaggctgaggcaggaggatctcttgaggccaggggttccagaccagcctgggcaacatggcaagaacccatctctataaaaaagattggcgtgttagtgcacaccaatagccccagctactcaggaggctgaggtgggaggatcatttgactccaggaggtcgaggctgcagggagctatgatcacaccactgcactccagcctgggcaacaaagaccctattgcaaatatatttatatcatatttatatttatattatatatttatttgtattatataattatattataaatatgtcataaaatataatataaatatgaatttatctttataaacatatataatatatatatatacacatatacagtattgactatagcccactctgtttttcagctagttcagaaagatccacaatttttttcctcaagccacagcctccaaatgattgaactctttcgtggatttccagccagaccaaggacatgcacagctctctatccatccacccaggaactctaaacaagctcctcccctcccaagtcttggttttcttctggaagaaagggggtttatggagacagagaggttgaccagatggctggactccaagagccaccattatgatcaaggagctccagctaccttcatactcttgggcaggttcctcccttatcagagtcttaatctcctacctggatcacagaaagtcagacaagatgactctatgggccctcccagcaccaccatgcttcagactctcatccgacccccttctgcacatccaggaatataaccattgccttgggattgggatttcagcccagatgttcagtgtagcctgttctcccccagattccactcctctgaagttcaaacaattgcaaacaggcagacactgcaatggaagctgatctgaatccattctctggctgggtgcagtagttcacactggtaaacccagcactttgggaggccaaggcaggaggatcacttgagcctaggaatttaagaccagtctgggcagcatggcaaaaccgcatctctacaaaaaataaaaataaaaattagctgggcgtggtggtgcacgtctgtagtcccagctatttaggaggctgaggcaggaggatcacttgagcccagcaggtcaaggatgcagtgagctgcgatcacaccactgtgctccagcctgagcaacagagtgagacactgtctcaaataaaataaaatgaaatattaataaaataaaaataaatccagccggacgtggtggctcacgcctgtaatcccagcactttgggaggacaatgtgggtggatcgcaaggccaggagttcgagaccagcctggccaacaaggcaaaaccccgtctctactacaaatacaaaaatttgctgggcatggtggtgcatgcctataatcccagatacttgggaggctgaggcaggagaatcacttgaacccgggaggcggaagttacagcgagccaagatcatgtcattgcactccagcctgggcaacaagagcaaaactccatctcaaaaaaataaaaaaaataatttaaatccattttctggcccctagctaccactgagtgatggggccagggtgggcagcagcttagtgtagcagaaaagattgagatgaggggtctgcccggctgggtgacaatccaggctctcctacttggtgacggcatgaccttgaggcatttcttaacctctctgaacctcagtttccccatttgcaaaatgagggttataatgcccacctcacaggatagttcctatctgagaaggccggaggagtgcatgttattattcaaactgcatgctggaaagtgacatctgtttcaaaggcagcggcgctgattcttgtcaccggctcagagccctccctgatataatttaggagttctgggtttgggagtgatccccgggcagagctgccatctccctccacccctgacatccgcaggatgaataaaccctgcagcccgcctggaagggctcttgatgcagtggcctcagaagatgccctgggctgtcgtactctccaacaggccgtgctaaaaaatgactccaaggaaaagtaagcagacactccaggccataaatatttcagagcggaatgaacgtcctttccccagcaccgacttgccatatatattatttataaaaccataacttgagttgaaaggtagtcgtcgaaagaatagacagtctcactcagattcacccggcgattgggaaaaccatctagagggagagaaaataatgtctgggctaggtgaggtcataggaagtcacctggccccatcccctggccccagcctcgcctggagggacagcccagagaggccccagacccagcaaactgccaggtttgcacaacccagggcctccctggtttcttaggaaatctccatcaaatgccaaacaacagtcacaaccagacggttaaaaaaaaatctgttatcccccctttataacaggggtagggcttgtcatgagagagcgagttgagaagggcttcccagaggctattgggtgagcagactcactccccagctaatttttgtatttttagtagagacagggtttcactgtgttgaccatgctggtctcgaactcctggcctcaggtgagctgctcacctcggtctcccaaactgctgggattacaggcgtgagccaccgtgctcagccctatattgattttgtaatatgtatgtagatatgaggtaggaggtggggcttgacttaggaccagattgaggactggctgaaacagggaagcagcagcaaagcccctctccataagacacgcccaccagcaccatgtcagtttccctttgctacaacatccggaagttaccaccccctttctataaatttctgaatactccacctcttcatttgcatgtaattaaaacagagtataaacatgtacaagaagacagcttccactccctttgattttatccctgaccaatcagcactcctggctcactggcttcccctcacccaccaagttgtccttaaaaactctgctccccaaatgctcagggagactaatttgagtaataataaaactccggtctccggcacagccggctctgcgtgaattactctttctctattgcaattacctgtcttgatgaatcggctctatctagagagggcaaggtgaaccccttgggcggtttcagccccaccctgccatggtgtgcgtgcgcatgtgtgcagatgtatcttcctcccccatccaatcctcaggccgggcttgtgcccctgaaagcccgcagttctgcaggaggaggtccctgtgttccctcccagatcctcccaggttatgccaggaacccagggaggactcacagatgccctggaaactccagcttggctgcagtgggcccctgaggctctccctctcctgttgtaaggcactgcagccccctgagctactaggaccctcaagggttggccacactgcccacctcatggaggacgccaccacagcagacacaggtgaagtccgctccaaagtatgggtgtccaatgtcaacagcatcacaatcttctacgaggcaccatctcatcatcaccaaagctgccagccccgtgatgtggaagcacaagaggaaatggtctcagccggggacggtgactcatgcctgtcatcctagcactttgggagaccaagacaggaggattgcttgagcccaggagttcaagaccagcctgggcaccacagcaagacctccccgctccaaccatctctacaaaatatagaaacatgagtcaggcatggcggagtgcacctgtagtccaagctgctggggaggctgagatgggaggctcgcttgagcccgtgagtttgagaccagcctggacaacacagcaagacctccgtctccagccatctctacaaaatatagaaaaatgaaccaggcgtggcagagcacgcctgtagtcccagctactggggaggctgagatgggaggatcacttgagctcaggagtttgagaccagcctgggcaacatagcaagaccccttctccacaaaaacaattttaataattagccgggtgtggtggtaagtgcctgtggtcctagctacttgggaggctaaggtgggaggattgcttgaggccaggagtttgaggctgcagtgagctgagtttgtaccactgcacttcagcttgggcgacagagtgggaccctgtctccaagaaaagagagagagaaaaagaaacaaaatagaaatcactggtaactccatgacccagaggtactccaattaccattggcgtccatgtgtgtggatggacggttacatatgcataaatatctatacacatacatatgtatatatccctgtaggacagtatgaatgaactttaatttatttaaactattccctactattgttcccacctaaggcgtgggaatcccaggtcccagctggagagagactgtcccaaggcaataccgtggcagcactgaggtcagtgaatcaccagcagaaatgaaagccgctgggcgctgggccgaagggctcaggagactcagaagacacttctgggatcaaggcccagcccagctgtctgggggctgtgtggtatctggggacaaaccaaggaaggacagggcgctggtggggtcttcctggcactgtcgtgttcaaaatgaacacagcaccacctgccccggcccccatcccacagaccccctgctcagactcatgactccagtggggtggcccataccaccctacatgcaggctggggctcttctccttccccacagtggatcccaatctagcagccacggtcagcaggaccagggcacggcggagtggggcatggaaaggtgggagaagttgcggggagggcgcatgagctagggaaggtctcgaaggctctcagggaaggcagaagttaggctgctaagggcctggatggggcctgatacagagagaggaggggaccttccaggagagaaaaagtgcaccaagaccccaaccagaagcagacatcgcctggtcggcggggaggcccacaaggccacagtccctcaccaccacccaagggaagcccccaggatcggaaccactggccaccctcaggctcattggcttggtatggctctttattttattttattatgatcattttttaagagacagggtctcaacctgtcatccaggctggagtgcagtggaccatgatagctcactgcagcttccaactcctgggctcaagcaatcctcctgcctcagcctcccgagtaactgggactacaggtgtgtgccaccacacctagctctttttatttttcacagaggcaggggtcttgattgctgcccaggctggaatgcagtggcgctatcatagctcactgcagcctcaaattcctgggctcaaacgatcctcccgcctcagcctcccgagtagctgagactacgctacaggcatgtgccaccacatatggctaattttttaaattttctctagggatggggtctcacaatgttgcccaggctggtcttgaactcccagcctcaactgatcctcccacctcggcctcccatagcattggggttacagacatgagccctggcacctagccaactctttaagatcaaatgctggccgggagcaatggctcatgcctgtaatcccaacactttgggaggccaaggtgggcagatcacctgaggccaggagttcgagaccagcctggccaacatgacgaaaccccatctctactaaaaatacaaaaacatttagctgggcgtggtggtgcacacctataatcccagctactcaggaggctgaggcaggagaattgcttgaacccaggaggcagaggttgcagtgagccgagatcatgccattgaactccggcctgggcaaagagagccaaaccccttctcaaaaataaaataaaataaaatgccttttgtacccccaaccatttgaggcggggaagatacagggccctactgagaggcgaagggagcagggcccggggtcactgtgtgaacttgggtgagattcccatgctcagggggtaaaacagggtttgcggtacctaccttagggctagaagtaagaatgaaatgcaagagcaaacataaaaaccccaggctggaggactgagcccatcggtgccttgggaaccacaacagtgggatcattcacacacagacaggcctggtggaatcttccagaccggcgccttgtggcgaggaggcaatatgtcccacgaggcccactcaggccccaggagcccccctcacttgggcacagcccagctaccatgccctcctggttagtgggcacaagagtgtcgagggaatatgctttgtgggcagcaaagtccttcttcaaaggggtgtttccacctgcaggtctgagcaggacatgaggtcatgtcgggccccaggacagcagcccctgtcctaggctcggccctgacccaccttctctggaagccggtcctcgagtacccttagggcctttacacactcacacgtgcagacacacgcacatgcacacacacacgcacgcacacacacgtggacacacatgcacacacaaacacacacatgcatacacgcacacacggacacatgtgtgcacacacgtgcgcagacacacgtgaacacacgcacacactcacacacacatgcacacacagctgcctccactcactcaggcctccaccaaccccctccccagctgtagccaccactgccacccccaaaattaaaaagaaataaaaataaaaaactgaacgagttaatttgctaaaatgaaccagaatgcaataactgattacatttttccccaaaggaaacttgagttaatttggccggtgactcatatgactggctgccagtgacaacacacaccatcccagacacagctcctcgctgtgccccgttgccagccgtccccctcctggcttccctcctgcgggaggcggggacagaggggtggaggcagaagtgcccactggataaagagctgtttgaggcccttctgaaagtgcaggatacacacgcatgcacacagacatatatctgtacagaagtgcgtgtctgtgcactcacaccaggcacgcacatgcacacacacatacacacacgcctgcaccacagctgaactctggcatgggctactcagtaaatcattcaacgataacctctgcataccaatactgggggaaaatccaattaaagtaactcattactaagtcattcctattttcccagcatcttaaagctcctgggaagagaagggaaggacaggcagattactaatgtgacctctcctaggctgtggggaaaattcctggcccggctcccaggagtggaggccccagatggatttctctgtctgctcgggggtggggggaacactgggtacagcccccctccccatccctgtgccctgtatacggccatagcttctcctgaaaacagcacacatagctcaagctaagtcctagggtcagcagacccaagaggcccagccttgtcattcacatgctgtgtgacattgcacaaagcacttaacctctctgagccttaatctcttctctaaaatatcttcatctgatggttgttttttgggtttgttttgtttggtttggtttgagacaaggtgttcctctgtcactcaggctggagtgcagtggcacgatcatagctcactgcagccttgacctccctggctcaagcaatcctcccgcctcagcctgctgagcagctgggactacaggtaagtacctccatgcctggctaatttttttgtatttttagtagagatggagtctcaccatgctgcccaggctgcatcatctgaatttttttttttttttcctgagatggagtttagctcttgttgcccaggctggagtgcaatggtgtgatctccgctcactgtaacctccacctcctgggttcaagcgattctcctgcctcggcctcctgagtagctgggattacaggcgtgcaccaccatgcccagctaattttttgcatttttagtagagatggggcttcgccatgttggccaggctgatctcgaactcctgacctcaggtgatccacctgccttggcctcccaaagtgctggcattacaggcatcagccaccgcatccagtccatctgatttttttaaactagggggtgggggaaaaggaaaggcacacacttttcctctttcccccttgccactgggtgggttacagaagcgatggcgggagctgaagcagccatattggacccaaagatagaagtcacctactgagcaagacaagaaaacccagcaagatacaaggaaaacggaaaatggttccttgagattgtggagctgccgtggaagccctggattgtttgtcatcagactattatatgcaagagaaacaaacttctatgttgtctttaaaatatgttacgtatatttatatatcaccatcatccctatgctcacacatggtgaaatctaaaaccattcagaggccaggcgcggtggctcacacctgcaatcccaggactttgggaggccgaggcagacagatcacttgaggtcaggagctcaagatcagcctggccaacatggtgaaaccacgcctctactaaaaatacaaaaattagccaggtgtggtggtgcacacctgtaatcccagctactcgagaggctgaggcaggagaatcacttgaacctgggaggtggaggttgcagagagccaagatagtgtcactgcactccagcctgggcgacagagtgagactccgtctcaaaaaataaaataaaatacaaaaccattcagaggaggtcactcgataaagacaagagatgtccccagactcctcatccaggaatccagcgaaagggcactatccagggctccacgttttggcccaactcgcccagcaatttctgtgcatcctcaggtacatcattttcctccccatgcctcagtttccctacctgtcttttgacactaatatcagtctcaatttttaaatgctgaccaatgaaggactcaagccaaggatgggcaggaaatcactttgtaaagaactgggattgggcaaggtggctcattcttgcagcactttgggaggctgaggtgggaggatcacttgaggctaggagtctgagaccagcctggagaacacagaaagacccttgtctcaaaaaaaaaaaaattaaattagctaagcatggtggtgagcatctatagtatagcccagctactctggaggctgaggcaggaggatcgcttgagcccaggagtttgaggctgcagtgagctatgctcaagccacagcactccagcctgggtgacagagaaagactctgtctcaaaaaaaaaaaatattttaaagagcaagaaggctggacatccgggcatggtggctcatgcctgtaatcccagcactttggaaggccgaggtgggaggatcacttgagcccaaggagttcaagaccagcctgggaaacataggtagaaccccatcgctataaaaattaaaattaaaaaattagctaaacatggtggtgcgtgcctgtggtcccagctacttgagaggctgaggcgagaggatcccttgagcccaggaattggaggctgcagtgagctgtgatcacaccactgcagtccagcctggcaacagagagagaccctgtctcaaaaataaataaataggcggggggatcacaaggtcaggagatcgagaccatcttggctaacacagtgaaaccccgtctctactaaaaatacaaaaaattagccgggcgtgttggcgggagcctgtagtcccagctactccggaggctgaggcaggagaatggcatgaacctgggaggcggagttgcactgagccgagatcgcgccactgcactccagcctgggcgacagagcgagactcagtctcaaaaataaataaataaataaataaataaacaaacaaataaataaataaataaaggacagagaaaagcaggaactgggtctaggtgtctaggtgggaccgatgactggctggagcacagagggcagagacacccgcagccgccaggctggtttcaagctcacctgatcctgttgctaaaccacctggcaggagaggaagctgagaaagagagaaagctggaggagaagctggagtcccaccacccaagccctaccgcacagctgcaggtcgtcagagcctgccagagccctgtgcccgcctcccaggccagacaatggagacaaatgcaatcccaagtggggcacaggtcagggctccatgtggccccaggaatgatgacacctggggacggttctcagagtggccagacgtctgcctaaacgctgaacccaaaattcaagcacagaataactgtccaccatgaccttgaaggccagctctcacagccccggggagcaacagccaggcttctaggcagcgggcgccagcggtgcccaccctgaggcacacccaggccccaccgtccccttggtgtgggtctgagctcgtgcctttcggcaggtgcacagccttggggtcggtctagcctggattcgaatcccacctgtatcacttacaaatggagggaccttgatcgaggagggtttataacctcccagaggctcagccctgcccctacacccagggggctgctgagaagccgagaagctgaaggagttcagggcatagacagccgcgcatgctgtaggctcacacataggaaggggacttcccctccctcccaactgtggccatctgcaccctgactggcatgacaaaagggcaaaaaacattggtcaaatttctatgcagcatccttcagtgtccccaagccctgtcatttcaagatagaaatcgggccaatgtgcttcctcttctccagaaaacagactccaagcatggtcaaaaaaccaaaggtgactgggcacagtggctcacgcctgtaatcccagcactttgggaggcagagatggctggatcacctgagaccaggaatttgagaccagcctggccagcatggcaaaaccccatcactaactaaaaatacaaaaattagccgggcatggtggcacacacctgtaatcccagctactcgggaagctgagacaggagaatcgcttgaacccaggaagcagaggctgcagtgagccaagatcacaccactgcactctagcctgggcaacagagtgagactccatctcaaaaaacaaacaaacaaaaaacaaaacaaaacaaaacaacaacaaaaaaacgaacagcgagggaaccttaaggatccaagattcccactgagaaaccaggtctctatttctccttgttcggttctggagccgcacgttctggacttctccaggccccagacacagcagaaaaaaaacacgagaatcctcatgcacctggctttttcatttaattaattaattaattaattatttgtttgtttaagacagagtcccgctgtatggcccaggctggagtgtagtggcgcaatcttggctcactgcaacctccgcctcctaggttcaggtgattctcctgcctcagcctcctgaatagctgggattacaggtgcccgtcaccacacccacctaatgtttgtattttaatagagatggagtctcaccatgttgggcaggctgatctcaaacacctgaccttccatctcagcctcccaaagtgccaggattacaggggtgagccagcactcccggccacacctggctttttcttattatttatttatctatttagaaatggggttctcactctgtggcccaggctggaatgcagtggcgcaaccacagctcactatagccttgacttccagggctcaagtcattctcctgcctcagcctcctaagtagctgggaccacaggcacgtgccaccacatccggctgattttttaaattttttgtacagctagggttttggtatgttactcacgctggtctcaaactcctgacctcaggtgatccgcccccctcggcctcccaaagtgctgggatcacagacatgagccaccgcgccaggcccacattttgatttttaaagcaccaccacatagctttatgtcctcgttgttttccacctgaatctccttatacaagactcaggctgggccaggcacagtggctcatgcctgtaatcccagcactttgggaggccaaggcagatggatcacctgaggtcaggagttcaagatcagcctggccaacatggcaaaaccccgtctctactaaaaatacaaaaattagctgggcatggtggcgcatgcctgtaattccagctactcgggagactgaggcgggacaattgcttgaacccgggaggcagaggctgcagtgagccgagatcatgccactgcactacaacctgggcaacagagtgagactcagtctcagaaaaaaaaataataaaataaataaataaaaagattctcaggctgaagtcacgaagtagggtggccccgagcccccctcagcagacacagcccaagagggcatctgcaaagagcaggactgtgggttgtgggcaagacgtggaggggaggtagaggagagaaggggaccccctacaccaggaggaatttgcaggcttcaccagggaaataggacggcaccccctgccccagggaggtgggcagggcatagagggcattttccaaattgtacagccacagggaccactctgcccagagctccactcacaccctccaaccccctagtcacacagacacacattcacacacacacactacagtcgtacacacacagacatgcattcacatatacactcactgacatacagatacatactcacaggcacacacagacatgcattcacatacacactaacgcaaagacacacacagacattcacacagagtcacacttacacacagacatgtatttacacattcaaatgaatacacacactaacacacagagacacacacaaacacgcacacacagacatgtagtcacacaccagatgcacacacatacactaacacacagagctgtactcacatagaccacacacatacacacataggcatcatcactacgctaatgtctctcagtgtcctacgacaggtgtgcacggccttgccttgcactgaggtgcccctgcagtattgggggttccaccgtagtactggggtccactcacccatagaccgtgcatctccagcttcatgcgcaggaatttccccatgccacagggccttttgctggggaagatgctggtctgcccaaagtcagcgtggaatctgggcagagccagaatgacctgccccaggcttgggagccgagcatataacccccctacacacagcgccattcagaccctggcaccccaccgttgggctccctaaagatgtgtccattctgcttgacttaaacactgtattcctggttggtccttcagtgacaggggagggaggaagaggctgtggcttgcaggacagggggtcaggatatgggcaatggcttggggacctcctacttgtaaggaaccagggtggggtcctattgggtggacaggagaccctgcagccctggcctcactgccttctgggatcctctgccacaggtcccaccccgcagcccatcctgggcctgcgacccacagcctgtgcctcctgtcccctgtccctcccagtgcaccctgagcccaccctacccagcccccgccacccccctgctgcactcccagggctgaccttcttccctgcccaggctggacatccctcctctcttgcctcccccttgacctccctgatgggactaagtggaggcttctgttcatggaaactggggttaaggaagcaaactacagggcctggggtcaaggtcaggtccaaggtcagggctgagtcagccctgacttcgttcctcccgcctcttcccccgggcatccaggcccttaggtgtcaggagttacagccttgccctcgcctcctgctcctctggccgaggccagggcggccccttcctgtcccccctgcccgtcgttcgctccccgagggcgcagcagaagcacggggaactccggaccccacgaggcgctctggacgaggtcgagccgccgcttcccccgggcgccgctaggtggcgccgcgctaacgaggatcgggatagaccggaggggccgcgctgccaccaaccgggtctccactccccaggtcctcactccccgggccccagcctcgccccaaagctcaaacacgcacccgggaacccttccaccaccctccctccccacccctggcatccttagccctcggcagctcccgtggtcccaggggcctctccggtccctcagcaggagacctggagtgcacggcctgtccatgcctctgggacgcaggacaaagtggacgaataggaatcaaccgtggggagggaggggccacccaggagaatcccagtccacggagtaagggagggtggcatgctgagtgccaggcagaaggcctggcccaggcggtgctccctaagtgagggtgcaccctcacttacatccttccaggatagttcctcttcacatccttccaggatagttcctcgcctaggcgttttctccctcagtttgggggtagtggtttttagggccccgtgtgagagtgagaaggctcaagggtgccactaccccaaaaccaagggggcaaggggcagaccccaactgtaccaggagcaaggagctgctttaatttactcctcccaacatgtctattagacgttttaccaacgaggaagatgggctcaaagcccaaggtcacatgggggtggatggccaaggcagggttggaacccaggtctggcagatgcccttgccatgccctggggtgccccaactctgaccacaatgagcccttccagctccttcccagcccaaaactaaaccctgaggcagagtgactgggccacgctcagacgtccctgactctatgccctacctctgtccctctgagactcccactgtcctacagctgccttctggtctttgcaatggctgatcgtcctgccgggaaaggtgtttccccagcctcaccagggcccactctggcatcaactcctacaggaagccctccctgattgatccctaagtggaggcttcggttgggtcccagcctcaggaaactggcatcagagcctcccatggtcccgggctcagacacaatagtgaactcagccaggtgaaggaagagctgagagcccaccaccaccacgatgctggtccatcggcagcctgtagcacgccccaaacaccccatttacaaagaaacttttcaaagggcatttagggcagatgataaaactatggcccggagaggccaagtgactagtgcagagtcacacagcaaaccagaagagactagacaccaaccaagatctgtcactatattcataaaagaaaagaaggctgggtgcggtgttttgtgcctgtaatcccagcactttggaaagtcaagggggtgcagatcgcttgagcccaggagtttgagaccagcctgggcaacatagtgagacctcatcgctactaaaaatgtaaaaattagcccagcatggggacgtgtgcctgttgtcccagctactcaggaggccgaggcaggagaattgcttgagcccaagaggtggaggctgcaatgaaccgtgattgtgccactgcactccagcctgggtgacaaagcaaggccctgtctctaaaaagagaaagaaaataaaaggaagaccaaaagcaatgaatttaaaacacacacacacacacgcatgcatgagtgcatgcatgtacttaagtggtagggcaaagtgccgctgcggactgcagctgggagagagggtgactcagtgccaagatgccctgcacagggccccacgcttctgagaacagtccagaagccgctcccagcatgcagctaaagccattctctcctgtggacacatccctggggacagaccccgtctgtcatcacctggcttgggtaacccagaaagctctgccctccaggcaccaggacagtgtcacccataatgaatgccccagagcacctaggggtaagttgagctgggtcaggccgggccagtccaggcagcccccaggaaccccaaaacccactggcatctacctctcagggtctcaggctgagagccataggtgctgggggacattctggaaagtgcccccaagaaggcttcacagagcaagtcctacacttgggagttggatccaggttcagcccctgctctgccctacctcggtgtagccctgagtgcttctttgagtttcattaatacaaccatgggctggggtgggtgggcacagagtagcatgtgctggagagagagagagagagagagacagaaagaaagagaaggcacatgcatatcatacataccttcacactactacttcttttctttttttttttttttgagacagggtcttgctctgttgcccaggctggagtgcaatggcatcattgaactcctggcctcaagcaatcctcccaccttggtctcaccacacctggctcattttttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcccacctggctcatttttaaaatttttttgtagacatggaatcttgtgctatgttgcccaggctggtcttgaactcctagtctcaagtgattctcctgcctcggcctcccaaagtgctaggattacagggcatgaaccacccctggctcctctctctctctttttctttctttctttctcttctttgttccttccttcttcttttttttttttttttgagatggagtctcgctctgtcacccaggctggagtgcagtggtgtgatcttggctcactgcaacctccacctccagggttcaagcaattctcctgcctcagcctccccagtagctggaactacaggcacccgccaccacgccaagctaattttttttgtgttttttgagacggagtcttgctctgtcgcccaggctggagtgcagtggcatgttctccactcactgcaagctccacctcccgggttcacgccattctcctgcctcagcctcccaagtagctgggactacaggcgcccgccaccacgcccggctaattttttgtatttttagtagagacagggtttcaccatgttagccaggatggtctcgatctcctgacctcgtgatccgccctgccttggcctcccaaagggctgggattacaggcatgagccactgggcccggccccttccttccttccttctttctcttttcctttctctttcatgcttttttttttctttctttctttttttcttttgagagccagttgtaaatcatttacctgcaaaggctgagagagcagcctcttcctcctccagcccaggaagccagccagcagcacatccaagcagcaagcctccctccctgggtgtcgctgccgcccacccggctgcacagggaaagcttgcgacgtctgttcttcctcttgctttatttcttcgtttccttgcctaagcctgggaacctgcactgaccctgccctccctaccccctcttgacctctgccccatggatgtctggccccagagtgttcctggaggacatcctctctgatattttcagagtcacctctgggcctcccggggactatccacacgagggggtgatggtttctgaggttggaccccgggagaacacagcagcaagccctgagcctccaggacaatctccgcaccactcagcaagagcccgtgctgatcaatcagtgaaacgggaaggcagggccatcgctaattcaggcagcaccggcatggagttggcctaatttggaagtggcctctgctaaagctggccttcgaaccctccaaggggggaaaaaaaaagaaaagaaaagctttactaagctataagcagaagcaggactgcaatcgagctgcttcacctatttaaggaaagtgatcattttcaaatgcgtttaatgcatgaattcgggtacaccaaaaactgatatcctgattatgatctgccctttgtcccctaaaggtggtgacactctccttagcactaccagcagtcagaattgctgccagc

SEQ ID NO: 3 - DNA, 42544 bp, Homo sapiens
agatggccatgaatcctatgactgccgatggtttggtcccaggatgcattattcagatattttctccctgtgggcttatgtaaacagaaagctaggtgagtggaagtaacacgtcaaatactgatttgtggctgggtgcggtggctcacgcctgtaatcccagcactttgggaggccgaagtgggtggatcacctgaggccaggagttcgagactagcctggccaacatggcgaaaccccatctctactaaaaataaaaaattaaccaggtgtggtggcacacgcttgtagtcccagctactctggaggctgaggcaggagagtcgcttgatcctaggaggcggaggttgcagtgagccgagatggtgccactgcgttccagcctgggcgacagattaaaaaaaaaacaaaaaaaaaacctgatttgcaacaattttgtgtcctagcctagagtttaccgtgctattgaaaatatctttcggctgggcgcgctggctcatgcctgtaatcccagcagtttgggaggccaaggcaggcggatcacgagatcaggagatcgagaccatcctggccaacatggtgaaaccctgtctccactaaaatagaaaaaattagccaggcgtggtggcacgcacctgtagtcccagctactcaggaggctgaggcaggggaatcgtttgaacctgggaggtggaggttgcagtgagttgagatcgggccactgtgctctgacctggcaacatagcaaaactccgtctaaaaaaaaaaagaaaaatgaaaaagaaaatatctttcaatgatagttaatatatatatacacacacacactgtatttgaagtgacatgttacatggttttccactatatgatttctcatatttcatcaaattgatcctcttgaactcctgacctcaagtgatctgcctgcctcggcctcccaaagtgctgagattccaggcgtgagccaccgcacccggcctctgatctttttttcatctttttcctagtttctgacctcactaccactctgcaccacgttgggatgggtatccttgtacatccgtgcagcttttctatgatttttaatgtctgattattttcttaggttaaatttctaaaagtagaagtgaacagcgtgtgcacatttttatgactttgggtacatttcattacattactttccagaaaagttggaacaattcatagcagtaactgcactgaatttcttgtattacttacctatttttaagcaagtaatataatctcttggttccaaaaacaaagcataaagaaaagtctcctatctgcctacctccccacccactgttgttgattttttggtgtgtatccttccagagtttcattatatacacagaaacaaataccagtgtcaccctcctttgtatgcacatagtctagcatgccatgcacactcttgtgcaagtggctttttattttttaaaattaacaggccaggtacagtggctcatgcctgtaaacccagcactttgggaggccaaggaaggcggatcacttgaggtcaggagtttgagatcagcctggccaacatggcaaaaccccgtctctactaaaaatacaaaaattagctgggcatggtggcgggcacgtgtaatcccagctactggggaggctgaggcaggagaatcgcttgagcccaggaggtggaggtttcagtgagccgagaccgtcccactgcactccagcctgggtgacagaacgagactctgtttccaaaataaataaataaaaattaacagtgtttcttacacttttcaaatcagtacattaaaatccctccaattgtgtgtgtgtgtgtgtgtgtgtgtgtgtgtgtgtgtgtgtgtgtgtttattaaagcgacaccattgttcaatgatatgaataggtatattttatatacttggttccattttgatagatatttgggttgcttccttgaagaatgttgtgcgtacaccgtttgacacaggtgcaagcttatctgcagggggaagccgatttgcaggactcattcctatagggagaggggccaggcccagggacttaagtgtttgtgaatttgtaggaactgccagattacccacacttagcagtttacaccccttccctccagcactggctgcagagagccctgtcctccagggccctaatgctaattctttcatgattgcactaattatatgcacaaaaggaatgcaacagaacaaaacaaaccacctggttttgtaggtgtggaagtgcacattgcagtaaggagtcaatgaaatcggttactgtgggacaggaccctcccctccccttggttattttactaacttggaataacgccacttccttgtaggaaacaggcagtgactgtcagtgagtgtgcctaggagaggggtgggtccttttagagaccaacctcgcagttagccagcaacaggctgcagcttagctagatgaaaagttagtttgagaaactgaagtcattgccgctaaggctggcaaggtgtggctggggccaagttgggggaatgtcgtagtagccggccctgtgcgcctgagcccacaattactgcgtcgctgctggggtctggcaaagctcaccttgatgcagttttgttcaaagcagtgcaatttccaagcctcaaaacagatccttttcaatacaatgtgattgtaaaaattctattaataaagagaaaaaaaagacgcaccagcggcttacaagatctttttttgtttttttttttaagagacagagtttcactgtcgccgaggctgaagtgcagtggtatgatctcggctcactgcaacctccgcctcccaggctcacacaattctcctgcctgagcctcctgagtagctgggattacaggcgcgcaccaccatgccgggccaattttgtatttttagtagagatggggtttcgccatgttggccaggctggtctcaaactcctgacctcaggtgatcctcctgcctgggcctcccaaagtgctgggattacaggcatgagccactgcacccagtcacgagatcttaaagtagaagtaactatggtatttttttcgagaattctgttagggcgagaagcccattcgtactgtgctggaaccatcctgaccctgtctgttctttgcaggaactcaaaccaatatgcctaagtagctgaaactgttgcatttcaaagccttttcaagcatatcttgaggacactgttccctgcagctgaactgaagtcttgatctgagcccaggaggagtccctggggttgtcttgttgggggtggggaagtgggaatgcttgcattcagctttgggggaccttaaccctttgccccctcccctgtccccccttcacatagctttaacatgaaagtctttctgtctgtcagatctcccctgtacaaagagtggtctactctaaaaattgctttgaaattggaatggacccctccctctcttctattttagttctagtttctagctctttaaaataatgcaacttttcgctttattgttctaaatcaatgcattctagccgttggctgtcccagttgcttccctgtggctgggagcccctctctgtggttgaggctggtcttaccattgctccagttgtcttcggccttctggggcttacttcccctgccctggaggcctgaggccagccagccagtgtggtgtgacccccctgacagtgggctctcccggctggcccagttgagggtggtcagttccttttgtggccttgccttcttgggagataaaagtggccccctgggtgccgtgcggcccgcccgcagggtccttggtggagctccagcattgattcctgcaggttcagctcctgccccaaggcagcgtccagctggctgagtggggttctcccagggtctctaagcacaggacagcctgcttctgcatgggggcgcacaggggaccgcagagtcacactcagacccttctaccacctcctgacttgtggactttggtggtgggtggcgacaaactggagtccactgggtctgtggcagagaaaggtggctttgctgggggctgggggagaggaggcgtttttggatcgcagcatataccttgtctagaattgtataaaaacctacaggaaaaggctgatttttttttaaaaaggctgtttttttaaaaacaactaaaggcacaaaaactccaagtgtgagaatatccagaatgcgtttatataatgtgcaccgttgaaaggaacattgaaaaaacaaaaaactccgaggaacaggtcattgggtttggagaagtttccttcagaaagttctcttcctgctcgttcactgtggattaaagctcgttggcaatttcggaagttcacgttcttgagactcggtgcctttggaacaatggctcacaatgaggacggtgtgcgttcccggcaagaatgagtgttgggggcttttctccttatcacccgttccccagacccctctcatcggcctcataaagaaacagatggttcatcaggaatagctcagaaatcacgttggctcctggctgtataaagagtgggaactgggatcataaacatttgaaatctcctaggaaaggacaatgattccctcaatgaaatattgatgtaaagggagcattttaaaaggcagaaatgatgaacctgattcaggttgaagctggtagtgtactttaacctttttgagaattgttaatttaaaagcaaatagaatgaaatatagactataaaagcagattaaatcatttttatgcatcctgggttttttggtgtcacgtgacaggaagagttactacaaagaagaccaggttttggaaagacggtggggttcacgtgatgaaacacaggccatgacccctttaggaattggatggaagccagccaggccaggccccccacccccaggtcctgggttggggctctgtgggcccaaggagtcctccagaaccaaattctccactaagagcaatttttaaaagagcagctgctcatcccatgtccaattagagtgagcagatgtgtaagaaacatgtctttgattgattttgggggagccccccgcagtttgatgttttgcttggaggtaggggaaacgggggctgagttacaagtattacctgtaggaataaacaattcttttgaaacatcagtagggaagaaagaaaaaaaaaggcagcttaagatttatctaaaaaggaagtggaaagttaaagttgctggagatctcattactgtctgtctcgctcaccgtaattaaaaaatgcctgaatgatcttaataacacatgattcagctgacaagcactaattgccctgtatcaggccaagagaagatgaatgttgtcaagaaaaccacaagtcagacaagctgattctctgtgagtctgacaccgtgttcccatgtacagaccagtttttactggagttcttccagcatcaaaagcagcttcttaccttcccattaattttcatcgctggcaggcccgagttgtttgcgatacactgactacctcccttgattcctcctcgctggctcaggagcttgggtctgcagacaaaatgtaaatatgtttcttgctcttatctttgtcctttttcagggaagaaatctgttcccgtccaatatgaatgccctggtgggcttgtcagtaaattacatggtatttgggaattgattttctgacgccaccgatttgtcaagaacttatcctgccttttcagcgatttgacagcacctgttaggctgtagggccatgtgattgggcagttctcctgccgagaaaaaggggactgatgactttctgtcacttgggcatccagaaacctccttgaaggtggaggttattgagggaggaggcaggagtggaaaaatgctattggtttattcctgtgcttttatagaacagccgaaccattctatggcaataaaacagaatcacagggcaaggtgcagtggctcacacctgtcatcccagtattttgggaggctgaggcaggaggatcgcttgagcccaggagtttgagaccaaccttggcaacatagtgagaccttgtctctacccaaaaataaaaaaaaaaaatcagctgggcttggtggtgcacgcctgtagtcccatctcctcgggaggctgaggtgggaggatcgcttgaggctgggaggtgggagcttgcagtgagccatgatcacaccactgcactccagcctgggcaacagagtgagatcctatctcaaaaacaaacaaacaaaaaaggaacttgtttcctagcaagaagcagatgaggctggtgaataagcataccacataagtaagtaatccactaacttagggaatttcgtgaccagtcactcagcacgccctgttgcgaaggggacggctgaccaataggatcatctcagagtcaccctcacacaacttggcttagcctagttggcaaaagtgtctcttcagagacctaccctcctggaattgaaataaaatgaaaatttgtgatgacgatcagctactgtcagtgtgacttaggtgtagaaattaaaaaagaaaaaaagaccactctaaaggcatttgtgcatgaagacacctacagaaataaagccggggtgctgttcttggtcttcattcaggctagcacctctcgtcaccttttcactttggagctcagggcctggttcactcagcaagtgagcggctcacacagtagatgctctgcacatagtagtaggtactcagaatatagcagctgttgatgtacatgtttccagaggggccaacacaggaatctgcacgtgggtccttgctttaagcccagcccctctctgtgggcgtgggatggagtcttcctttcttggtgaccggcgccctctggggtcactggaggcctggacttccccactggcccagtgaccttgtttccttgttccctgtctcgcccagccttggctgtcctgggatttgaaaatctgggataaagcctttttcgggcctctaaggccctgctggttctgcgtgttccagagagctgcacctctgggtggtgagctggcctttttggcatttgatttatgtatagccctgagtcactgggagcggatctagagtccaggccagtgactgatttcaaacgctaacactcttggctcgggctcaggtttcaagtcagcctccaaaggggatgtttgctcatttcgtcatcttcctatcacagcaattttcgtcatatggaggggtggcactcatgtaccttgatgattattattcttttaaaaaatgaagttatttatgttatggggtgagttcgtcacacagtccgaatgtggaatgttgccatccttttcatcccaatcctgccagtgatttctgtttacaatagaactattgacattgtatggggatgaaactagcatgagatagtaattgttggcttgcctgagattttcatcatatgtacacatagacaaaacctggctttagttcctaaagctattgctgtttattgtacttttattagccaatgttacagatccccgtagcatgccagaggctattaagaaatacaggggtcgggggcaggaacgaagtccctggccatggatcatgcaattgatctatagttatttttttttctttttctttaatttttttttgaaattttaaattattattattactgttttttgcgatggagtttcactcttgttgcccaggctggagtgcaatggcatgatctccccctcccaggttcaagcgattttcctgcctcagcttccggagtagctgggattataggcgcctgctaccatacctggctaatttttgtatttttagtagagatggggtttcaccatgttggccaggctggtcttgaactcttgacctcaagtgatccaccgacctcgacctcccaaagtgctgggattacaggtgtgagccaccatgcccggcccaaattattattattttttataagataggatcttatcacacaggctggactgcagtggtgcaatcatagctcactacaacttccaacttttgggctcaggtgatcctcccacctcagcctcctgagtagctgggacagcaggtacatgccaccatgccaggctaagttttaaaattttattttgctagggggtctcgctgacttgcccaggctggtcgtgaactcctggccacaagtgatcttcctgccttggcctcccaaagtgctgggattataggtgtgagccatgcttttggccaatttttagttattgtggatacataatagttgtacatattcatggagtacttgtgatgttttggtgtaagcatacaatgtgtaatgatcaagtcaaggtaattgggatatatacttatagttagcctacagtcttattgataatgttctcagaaatttctgattccagagctccttgctcctctcccataattagataacgtgtttttctgtccggtaattgctgtagtactcatttttagatggggtagggaaggccatgtgccatgttggggcctcatcccaaagaccttgtgctgtttgggacaggtaattatttaaagccccgtgtgaggtgggtgggtgttaaactcatgctctccacaccttcccagagtgcctgggaaagcaggcttctctaagaaagtggaatagcgaataggcagtgatccctgagagagatacataatgattgtcagtttcatggattacagataattccgtttaatgtcctctgtttttcctttttgcaaatttctatgcattatatagaacattgactttagcatagaaagtgctaattcttgatttggcactttaatggatatttacacagtgacaccataaacattggataaacagaggatttgttttaatgagatcttcagtgatttgaagccgtttcccccttgccttctggaatccccggctccagttctgcctggttttggtgatgctgtcacagtggagggctgactgcaggcctcgactgtgagccctcactcggccaccttttcatttcctcgctgggttccaggctctttgaagcactcgcctcctcccctccctgcatgtcctctgtagtgcctatttctgtgatttgcatatagcggaaatatttgttgaactgaattatttttacacacaagcctttcatgtggccaacattttgaggtctgtctcctttggcgggagtctgggaggaatcttccagttcactaagaagtgtttgatagcttcagggcaggcgtgccctgattggggctggatgccctgcctccatccagaggctctctggccaggaggggaatgaaccaagtgcccagattcccccctacaatgtttatgcagaggtgactgtcacctgccaatggtgacagtccaccagggaagtccagaaggcctttggattaaagctacaggagaggcaggtccccttgcagggtcagaaggggaagagagaagagagagagaaccaaaggtgggaggttcagctactcgggaggctgaggtgggaggatccctcaagcccaggaggtcaagtctgcagtgagctatgattgcaccactgctctccagcctgggcgacagagcgagaccctgtcccaaaaaataaaaaataaagatggaaagatgggaggattctccccaggaaggcccaggccgcagggtccccttgtccttaaaggccagctcagtgaggctacaggtccccttgccccagttccccgagcacagagcaggcatggcctcggacgacttcctctttgtgtctcgtggactactcaataaatattaatgttttccaaagatatcttgcccagctctccctgacgaggctgttggcttgaaggaataacaacactcgttgcactcacggaatattaagtctgtgccagggcctgttctgagctcatgacttgtgctactcggtctctccccacaagctcacagggcggatgctattttcaccgccgtttataggagaggaaactgaggcacagggtccttcagtgactggcccccatgagcaggcagcacatctggaattccagcctgtgcagcctggccccaggatccccatgcctctgtgggggacactctggaagtgacaggtgaggcacttctctggcctctctggatctcctgctggccctgaatgtgggtggttacccttttggggtgctgtcggtgcctcttagatgtctcagtgaccctacagggtctgccttccataggactgcctttttctttttttttgagacaggttctcactctcctgccaaggctggagtgcagtggcacaatctcacctcattgcaacctctgtctcctgggctcaagtgatcttcctaccccagcctcctgaatagctgggactataggtgtgcgccgccatgcccaactaatttttttttgtagtttttttggtagagatggggcttcactgtgttgcccaggctggtctcaatcttctgggttcaagtgatctgtccaccatggcttcccaaagtattgggattacaggtgtgagccactatgctcggccggcacccacctttttgtggacttttctttgttgacttggcattcggcaaggtgtaggcttttgccaccaacacgatacccgtggggagaagccccctggcccccgtgcctgccagaagaaaggcttagggtcccctgcgaggacccacacgtgcactgcatgcctggatagcccatgccagcctcactgaggctggatttagttcctgagtatgttatggagaagtgcccagagcagggaccgcaggcacaaggtgggcctggcctttggaggaggctgggcacggccacctgctcctcctctctcccatgggcaggttggccttgttgccattggaaactcttgagagcccacgggtgttttctggccatgtgacctaaatgggaggcttgctgaagggtagatgctgcttcaccagcaacaagaggagcccccagacctcagccacaggatggcccccaccctgctgagcccccactcttcgctccctgctcagcagagcaacacataccattgcccaaaggatggaccaggcacagcgtggggcccaatagcaaatgttctgacgaaggaggtcagtgccggacactgctgggcgttgttgagtcctgggctggcagggacagttttgcagggacggtttggaaggggctttgctggggagggggtctctaggaggggcagcccctgaatagggcagccgagttgctgtgactgcatcttcctttctttccgatgggggcaaaactcactcttctttttaaccattctctgttatctgtttttcttttcttttgaaactttttttttctttctttttttttagaaagaggatcttgctctgttgccaggctacagtgcagtggtgtgattatagttcactgaagtcttgatctcctgggctcaagcagtcctcctacctcagcctcctgagtagctgggactacaagtgtgagccaccatgcctggataatttttattttatttttttagagatagggtcttgctatgttgcccaggctggtcttgaactcctggcctcaggtgatcctcctgcctcggcctcccaaagtgctgggattacagacatgagccactgcacccggccatgtcctctttttctgattcgggaggataagggctccccatgacactggcatcagcattgggagcatttccgtgaccccagggctgctgatacaccctgtggacagggcaggacccacagctaagtcagtaaagcaccatcctctgggagtggctgtcatacgacatttcttgcttttaggaccattggactgaaaaaccaaatcctgccactggatctggtgatgacggtgacatttttttttttttttttttttgtttagaagcttatgattcatcggatctcttttggtttacggaaagcccagatgtcgtggtccctctgcgtgcccactccaggagaacgcattcaatgtcaaggaaccctgtagtgtgcaaagttggatttagggatttcccgaataaagctcactgagggtcaatgagttccataaacattattgaggccgggtgtggtggctcacgcctgtaatctcagcactttgtgaggctgaggtggggggatcacctgaggtcagaagttccagaccagccctgccaacatggcgaaaccccgtctctactaaaaatacaaaaatctgggtgtggtggcacattcctgtaattccagctactcgggaggctgaggcaggagaatctcttgaacccaggagacggaggttgtagtgagccgagatcgtgccactgcactccagcctgggtgacagagtgaggctccgtctccaaaaaaaaagaaaaaaaaattattgaatgtcttccttgtacctgacactgtgctttatcccatttaattccactggataatatgttaaattcagacttgttcgtagatttatatacatgtacatatccctcttaatacattggtttattctgtttctccatgttcataccgccagcctgatgcctcatttgcacaaatgaatggatgagtgaatccagcttattcttttatttctccctgggatctggtatacaagagatggttattttttaatttgagacagagtctcactctgtcacctaggctggagtgcggtggtgcaatctcagctcactgcagcccctgcctcctgggttcatgtgattctcatgccttagcctcctgagtagctgagattacaggtgcacaccactatacccagctaatttttgtatttttggtagagtcagcatttcaccatgatggccaggctggtcttgaactcttgacctcaagtgatccacctgcctcggcctcccaaagtgccggaattataggggtgagccaccacaccttgccaagagatggtcattaaagttgaattttggcactccttgcagaaaccctacaaagttggtatcacctctattttgcatatcagaaaactgatgctggccaggtgcagtgactcatgcctgtaatcttagcactttgggaagctgaggcaggaggattgcttgaggtcaagggttcaagaccagcctgggcaacataaccagaccccgtctctacaagaaaattaaaaaatgagttggatgtgtacctgtgatcccagatacttaggaggctgaggtgggaggattggttgagtcctggagtttaaggctgcagtgagctatgattgcaccactaaattccaacctgggcaatggagcaagaccctgtctcaaaaaaaaaaaaaaaaaaagtgctgagagagatcgtttcccgagggtctttggatagtttgtagaaaaacagtactgttccttctaaagccctgtggctttttccctattccaggtgcacattggcctttgaggctggaaaacacttttacttacttccagctttatgggattcataaaatggccttgagaaaagtaaaccgatttttctccttctaaaaacatccagtgggccgggtgcagtggttcacacctgtcatcccaacactttgggaggccgaggccggcggatcacttgaggtcaggggtttgagaccagcctggccagcatggtggaaccccatttctactaaaaatacaaaaaattagctgggcctggtggtgagcacctgtaatcccagctactcaggaggctgaggcaggagaattgcttgaacctgggaggtggaggttgcagtcaggcgagatcacaccgctgcactccagcctgggcgacagagcaagactatatctcaaaaaataaataaacaaataaatacatctggtgcaggtaaagtgttcaggaagtaatttcctggaagagggaactctgcagttgcaaatgcggccttcttacaagtcgcttgtagggtgttgaaactctctcgcgatcccagtgacttgagatgctctgttttctggataactaagcttttccaccttcactacggggtaccagggtctttgtagcagagaggcagctgtcttgatggtggaaccactcccgccagtgtgtggtggggtgcccccggggatttagggagagagaaggccagtggcagggactggctggaggaagagggtgcgaggaaaaacactggctgacatttgaaggaacgtgcttcagtatcagggaaggccagcgtgggataatcggtgtcttggcagatggggttgacggggagccctgaagccggcagtgtttagagattcaaagaataggggggctgtggggagtcttttggttcacagcagtgtcccccaaaccggcacagtgggatgagagtgggctggccagggaaggggacagggtgttaggtccagaggtgctaaaatccccaaccatggtcaaaatggggacatgagagactcggggttcagagctcacatgaaggacagctgtccccccctccacccccgccttgggtcagagctggctaggagagcccaccctgcagcctgtccttcgtgggaatcagcatgaggggtgcaaagtgctcagcaaggcccggcaccaggtttgttcttgggatcagagtgatacacgcgccttacattttttttaagctgctggttttgcgggtcagtacggggaaaaacggtgagtcccttttataaaatatatagagaggttggtgcgaaaatagcagcttgtttctgcttgcgtatttctactcactgctgggacacaaatctacccttccattttctgaattgtattcattcaaactctgatttaacctctatttagttaatgaattgggcaagttaacctcgaactcttccaggctgtggctgttaccgtcaccatctcctgcttctcactttacacactcatatgagaccttttttatttctgaagtgtggcctgacattgtttaggctgttgtttggtgcttatttttaactcttcttttgtcacttaagtaggatgtcttaattcttgtttagtgttagatcacttagcaggattttttttttgagcgggggcggggggaatatgattttgtgtcacattttgaatacttctctatggcagtatcttttagagttattatggaccctgtaattctgttctcaaagacgtttaaccactagaggtagacaccaaaaatgttttatttgtctcatttgggactctaattcagtgaagttggctgtttggagtctgagactggatctgtttttttattttgttttgttgttgttttgtttgttttcagacagggtcttgctcttctgcccaggctggagtgcagtggtttgatcacaagtcactgtggcctctaacttacatgcttaagcaatcctcacgcctcagactcccgagtagctgggaccacaggtgctcaccaccaggcccagctgacttttgtattttttctagagacaaggcctcataagtagttgtttcccaggctggtctcaaactcctgggctcaagtgatcctcctgcctcagcctccccaaagtgctaggattataggcataagccctcacacccagccaggactggatcccttgactcacttaatgatcggggtttacttctgacctgcatggggtgctgtggggaacccagagatcaataagacagcccgtaccttcttggagctgtatttagcagggtcatagaagacgcactcataatcagaaaccatccagaatgtgacttctggccagggagaatagggaagggttcatggagatggtggcatttgattgaattctgaatttggggttgtaggtttcaaatattagtttggggacagtcatccttttattttcttctctaaatttaaagaaattgttatttttaaaaaccaattggaatgggtattgaatttctgtgtggagaatgtctaccagtttttcatcattttctccactgtatagtgtcaataagtgttaaatgcaatggtctgtaagactttgttgataattttcttttctttgacatagacttgtaaactggaaaccctgtgtgggtaatgggcagtgcctatttcagcatcttgtccccagaaatccactccctaaaaggcttgttgaatgaatcagatctaggagcatctcttttgtgttggagcggagagtcagtccggactttgagcctctccttgcactgaccagcggcaggctgaagacagttggacttaagaaaacttaatctagaagcaatatgctggtcagattctttcaatgctaggtttagtatcgttcctttttctcccatttggtgaagtggtgtaatgctttagcacattttaaataagattacacaagcggctgttagttggcatcaagaatcttttaatctggaaagaaaatgttacctccttcatttgcaaaacagagtgtttctggcaccgtatcccaaagtgtcttctggttgttccgtttttcagaagttactgtgggcttctaattaccctcatttagacatccaggacagaaattgccacttcaacaagaaaattttgtaatgagccggaggaaatggtctcatcattactacgttatacgcagtctgagagtctgcgtggatttttatttcacaagaatgtgactgtttaattgaaaaatgttgttaaaatgtgctttctgttcctatcagtatgttgttagtgcttatatagccatgctcgagtcagtgaaaattggtgccctttgccaaaatactttttttcatttttgtgtataattttgtgagctaggtctatttttgtttaattaggccctgcaagagaatgagggagagctgtatttaaatttttagaccattaaagggcctaaaactatcctagatgaaaggcccccaaaattagctttaatatttattatatgaataaaaccaatagggaaaacgtttccagaaaccctttctctttttccttcttcagacagctggctaagattaattgcaggcctgaacgtgggcatgtttgctagtttctcttgttctattactagaaaataactcttaagagaggagagaaataccgtcttcttgtatttggggggcaggctgtacattttataactcactaatataatgaaatgtttgttgataacatcatattttgctttctgatagaaaagtcctaatttctattttctttttactttttatttttattactttttttttttttttttttttttttttaaaatagagatgagggcttgctgtgttgcccaggctggtctcgaactcctgggcttaagcaaccctcctacctcagcctcccaaagtgctgggattacaggcatgagcaactgcgcctggcaagtttgttttactttttatttttattactttttgtttctaatttctactttcgacaggattccttttaaaattttaatctaggaaacaaaactgcacgtgaaagatttgccttgtagacggaaagttctggaaacatcagctccaggctgtggtgtgaagctgtcgggctgatttgcctggtaagaagcgcgtttggggcacggagggtcaccttgccccctgcgagcgttctttcatcatttgagcagccaagcatcccttttgggccaagtttgccaaagactctaacgtagagaagtggacagacccagttgcatgctgatgttaggggcagagagaaagaaggaaagatccttgcctagcttaagcccttgtgcttcttccccacatttatttgacttcccaaattggattggctgttgtgattgaaaagattgtcagcactatgtaaatgagaatgagaagtaaaagtgcatctcatgcagataatttctatgtagataatttgccataaaaatttatattgcagtccttaaaaccttcggagatttagtctgtctagggaaaggtgatttatacggcattaaaacactttttatgtcccagtcctttagatttaatgtttattgcctataaaaatgaaatcttcttgtggatatttaaaagttttggtactgtgtgaaatatatattcaagttagaaatgctttggagatgttgtaaataagtggcaaatctcttgctccaaagtccactgtgttgtctgggcccgcatcttggctttgcacgcctgaaaagctgcctcgggtaccggtgtctgatcatcaccatagtgggtcgccaagtaggagaatgtcttatgtagggatttggtggtttttcaaaaaacttaaattagctaacaaggatgggactattataattcctcagaaagttatgcaaggtctgaccagaatttcattccaatctgcagaaagactgaattatggtgcagtttttttggttttttgtttgtttgttttttgagacggagtctcgctctgtcgcccaggctggagtgcagcggcacgatctcggctcactgcaagctccgcctcctgggttcacgctattctgcctcagcctcccaagacgctgggattacaggcgcccaccaccacacttggataattttttgtatttttagtagagatgggatttcgccgtgttggccaggctggtctcaaactcctgacctcaggtgatccacccgcctcggcctcccaaagtgctgggattacaggcgtgagcccctgcgcccggcctatggcgcaggttttaaagagtggaagtgctttgacaatggaatgcccagtacaaagcagtttttaggcttttgcagccactcagattcataacagcacgcttcaattctggtcttaaataaaactctcatggagtaaataaatcatatggtggggacagcactgctgtggaggagggtactgtttaagcattttgggggttcctgaaagatgtttctcaaagtgagcgctggagaggagtacctggtttgattgttgaactaaagtgtggtttctgtaaccctagggagttcttccctaggtatgattgagctccctgagtgctgggatggtgacctccccagcctgtctccaagtctgagtactggtcggcttcctagactccttaaaacgctgggtattttatttaattaatttatttatttttgagatggagtcttgctgtgtcgcccaggctggagtgcagtggtgcgatcttggctcactgcaacctctgcctcctgggttcaagcagttctcccgtctcagcctcctgagtagctgggattacaggcatgcaccaccacgcctggctaatttttgtatttttagtagagacggggtttcgccattttggccaggctggtcttgaactcctgacctcaaacactgggtattttggaaatcatgtgggaagtgcttttcctagagttttgtagctcctcaaaaaggcagaactaatcaaccaggcttccccattgtagccacagttggatcccaatggtaagggccattgggaaaccaatggtctgggtgagtctggcctgggaccctggcatggggaagagactggacttgaaggctttggatgggcagcttggtctggacagtattggaaataaaggaacttgagactttgaaattgctgtgatgggggtgttctcacctgtccataggggtgggatgtcagtaagctttgccggtgtctgtcccatagatagactcagctctgcattgcagccccaaaaggagcctgcattcttaaaagaattttaggggaagaaagatttaagagagcacctgaagtcctgaatcccttcagaaggaattcatggctgcctgtagtcagctgccagtccaaccttgccctccaaaagaagtcacagagattgagttttcagcgaggcagggagaagttgtcacatttgcctaattcttgtagaggtgagagtgggagtaaactggaagggggcggattattggtgatttgggggccacgtttccttgtatcacctgtaaagttttttttttgtttttgttttttttttttttttttggagacagggtcttactctgtcatgcaggctggagtgcagcggcacaatcacggctaattgcggcctcaacctccccagactcccaagtagctgggactataggcatgcaccactgcacttggcgtatttatttatttatttattattttttcgaagagacagggtctccttatgttgcccaggctgttctcaaactcctgaagcaatccgcccacctcagcctcccaaagtgctgggattacaggtgtgagccaccatgcctggctcacccataaaattaaaatgaaaagagcagaccacagccagcagtcttccgacctccagctcttgcaggtcgtgggcagccttgccccaggtgcagcaggtaggccaggaagcaggtgtgggaacagagcaacagcaggcaccgtggggacatgcacagtaagcgtcgaatcggtgtgcgtttgcagaaacgtgaatgggtgaatgaaggggtgtatgaaggtatacacaggaacgccacagtcagctttgtgtaaagtagaccagtgaacagattgctggtattgcttctgacagctctgctcctcaggggaaaatggtgaactcactttagaactcgcctttgtcagctccaactagactctggaaggaacacaaatgctcagactttcccatgacagactgtgaagcaaagccagttacacagctaccgtcactaatatttttggtataaagcagttgctaaaattaattccagtctgaaacttgatgaattctcagtcatggaattaacaggtcttaaatgggaaaaataaaaaattagcttgattaaaaataaatgtttgtgctgaaatgtggagcctgtctgtccctaacaaactaaatatgaagtttttcatcttagaggatttgtttgcctttgagtattcatagggaaagtgcacgtttgtaccagaaatttagagacgcactcccagtaagttcttacgagcagtcagtattatcctttttgtataaggcaagcagtcagtccctatgaagagtaagatacaagcctggctcagcagtggcccccctgagcctatctgatgtgcagaaaagtcacgcacacacaactgtcttgaagggcagcctcctttcagccagcacccatttactccctcttattaggccttcctctgaatttatatcaagcaaactatttaggacgcatctgcgaagcaggaagtgtgtttcattttgtttttaatgaggtttcaaactctctcttcccatctctgtccctggatgacagcgcagagttttacgtcatttgctattagacatcttgtggcttgacatcctcgaaggctcagccacccctgggtgtgtttggttaatacagcaagtgaagcctgggcaacatatcgaggcctcatttctacagaaatttttaaaaagtagctgggcatggtggcatgcacctgtcgtgtcagctacttgaaaggctgaggtggaaggattgcttgagcccaggagttcaagcccgcagtgagctatgatcgtgccactgtactccagcctgagtgaagggcgagatgttgtctcttaaaaaaaaaaaataataataataaaataaagaaaagaaaaaagtagaggttctgtgcttagttagaagatatctaacaacacaattggttagactttcataattatttctgctgatgtaagtatcacagcgcaggtcatgggcaacagtttactgttttcacacccagcttcaatgttaaatgaaccccagactatagcgtgaccagtctgatattatcatccccaaatctttatagtttcagctcttgggatggatgactgaataaccattttttttccagtgtggtagtcttagggttttgcttattggtgtgtgtgtttcaagaggaacctctgttccttggacttgatgtgagtggcaagtcatgtaaaaagctggattttatcaatagtttttgtctaaaaaccaaaaccaaaaaattcttcccctgcagtaccagtgcttgaaataaaccaccctcctacccccacccagcttgcctgcgtggatccagtagcacccccggtaccccggtgggcctttttggcagccccgaggggtcagggatctagggggaagagtcaccgcagggagcgcagatgtacttggagtggggaatggttgggggggacccattcccctgctttaaaccgcaaatgtcttttgatatctagtaaaattggcaagcagaatgattgaaaacatgtgagaggtaactggatctaagtctcaagtagtacatatttgagaagttttccccaatgtaaaagtcactctttaagagccttaaactaataaaaacaaggaaattccatgttacggctgccactcggccttttcatttttattccagaagtcagatctgacaggtcagcagctcactggctctgggctccccgtgtttaaagcagtggggacgaggagaagaactggcagccgccgggacggggaagtctccaatgccattcagtgccaggctcgtaattccctcttagtttatcctattttctcttcttggggcggcttcctgtatgtactttattcactgaagctgcttcaggattccttcagagactattcttgtttctttccatttctgtgtctttttttttttttttttttttttgagacagagtcttgccctgtggcctgggctggagtgcagcggcgcgaccttggctcactgcaacctccgcctcccgggttcaagtgattctcctgcgtcagcctcctgagtagctgggattacaggcacgcaccaccacacctggctaatatttatatttttagtagagacagggttttgccatgttggccaggccagtctcgaactcctgaccacaggtgatctgcctgcttcggccccccacagtgctgggattacaaagcataagccaccgcacccggctaatttcctttatagttacactagcttggaagaataacatactgagtggcgtggaaggcgttggtgcacataggtgcagcatcccagagccccgtccactggcggctatttgtgcatggctgcctgtggcttcttatctgtgatggaacctcgtgggcagagaagtggctggagagcatcaccaaggctgcctggtccaagttgcttcctgttgctgcatgggcagtgttctggggtccagaccctgggtggtagtgataagatggtgatggggtggagtgggtccttatagatcccacattactgagcggccttttgcccttcaaaccagatcacaatcccctccctgccagttggtcctcaagaaatcacaggaggctgggcgcagtggctcacgcctgtaatcccagcactttgggaggccaaagcaggtggatcacttgaggtcaggagttcgagaccagcctggtcaacatggcaaaacccggtctctactaaaaatacaaaaaaaattagccaggtgtggtggtgcactcttgtgatcccagctactcgggaggctgagacaggagaatctcttgaacccaggaggcagaggttgcagtgagtcaagattggattgtgccactgcactccagtctgagtgattctgtctcaaaaaaataaaaaaataaagaagtcacaggaggcccagcatacagagaacatcaggtatagagagatgtccttcctgggccaaaacaggtggtgtcattttgctgagtttgatagagaaaaagtcctgatgtctgtgtacataagaactcacgtctagagttcgagaaaaaccggtgatgagaacttgaacttggaggagtcacagggggatcaggaaagcttggtaattggcatgaatttcctacctccaggggcctggcacagagacctaggatgaagagggggccatcgagcatggtgtgaacttggagagtgtggggagctgccccagccgagggcactggatgggaattcatgccagaaccccatcacatagcggcaggagctgtaattgtgcttcttaattgtgtatcttcatttagtgcgagtttaggtggttagcaatagctctttagctagctttacagagtctaagttaaagagcccctcagctatcttgtagaaagcgccctggacaggatggggtgatgctgacagctggagtcactgttccagcgcccagaatggggagactagcagcacttctgggcagcccatgaagcagcctcttccctctgccggtgtccctcatgtccagcagagctagaagtgaaactcactgaggattgacgcggcccctttaaaatcgcatctccgctcttggcaccaggagctgtcatgtagtgtaacctgttggtgcccagaacccaggtttcctggtggcagccagcggtaccggtgccaggaggcagcgaaggaatcacttgcgacgcccgctctctctgccatacgagtgtcgttctgcggggccatctggctggcagcagctctcagccaatggcaggatgttgtaaacctcactcgctgaatgcggcaccgaaaatcttgaaaattaactgcaaaaataaaatcaaagctggctccgagatggtatttcttggcctggcataagctctccggatgaacttgctctgccttgttgatagatgcctcttactagtttggttttttcccaggagccagttggcatcttttaacttttccttgtaaaatccaggcagaaacattttgagagctttctggagtttttatttgctaattaaaatgtgagaagggaacacagccacaggtagaggtctagtgcaagccacagctgtgattcagaagaagaaaaaaaaaagccaaacctgacagtttcaaaatgaagttattagcacttcacctataactgtgatagcttttaacttgcaggatccttctcttaggcaccacctcccatccgaaccccagagcatatgggaaaaggagctaatgtgtgtaagttgcaggatacttaatcactgaagaatctaacaatgataactactatctctctggaacttttttttaacttagaagagtatgtcttgttctgtttatatatcacatagttttccaacgaccctgggaggtgaggtaggtggtatattttaccaatgggaaaatcgatgctcaggaagtcttggtgacttgctcaagttcccataacaagaagctgctgaatccaggatggaacctggtctaccccaaatgcccctactgcttcctagggcccagtaatgcccctgggtagggcgggcccagctctggaagaaacagggctatacaaaaaatcacccaatattgggtcaccagccttcttctacttgcaaaataattaagcaaaatctgtttttatttgtttttgagacggagtcttgctttgtcgcccaggttagagtgcagtggtgtgaacttggttcactgcaacctccgcccctagggttctagcgattctcctgcctcagcctcccgagtaactgggattacagacttgcaccaccacgcccggctatttttttgtatttttaatagagacagggttttgccatgttggccaggctggtctcgaactcctgacctcaggtgatccacctgcctcggcctcccaaagtgcgggggttacaggtgtgagccactgtgcctggccacaaaatctattttaattctgcaagttataagcagccagtggcccaccaggcccagctgtacgttcccagggaccccccaaagcgtatatgtcccgggcacacacacactaggtggtttgttttgttccccagcagcccgtgtctgcccgggcagatgttagtggctgagaaaccccaacagggagtgagccacaggcccgcgggtggcttccctgccagtgttctcctcgcgaaggggaagggagcccttgccagaatggctggtgccctaacaaattgattcctgccaaacaatagaaataaatattttcagcagacagaaatgttggctctgaaagcattcaaattaaaggcttatttctgaaacaaatactttaggaggaaaacatttttaccagcgcagatgagatcagagctattccttttggcagtggcaggactattttgagactcagccacaacggtttccctgccccgtctcgtttttcactgatgacctcgccccctgtttcatggagacagggaattgcattcagctagaggcatggggaagagatggtggcatggtgttctgcagagagcctcagttggacagtcggccgaatctggattcaaatccaggccctacctctcctgctatgatttcgacaaagccggtcagtcatctgatcatcagcttcctcagtctgtgaaatggggcggtacctcccttgcaatggttaatagcgttatcctcaggacctgggggtcattggaggccagcactgtccctagaactctctgtgttgattaaatggtctctacccgccccagcaaatataattgccactagctacatgtaactgttgggcccttgaagtatggcagctggaatggagggaacaaattttaaatttaacttagatgaaatgtaatccacatgtggccactggctaccattttggatagtgcagttcccattcatccctctcatttcattagttggtccctggagccaattaattggttggagggtcaacccactacaaatctcccaagtctttctctgtgccggatgcagctgtgcggagttgggacctgtctgttgtctttgacatccgtcttctgtcctgcagacggagcagtggggcagtgccatcgagctgggcagaggaattaatatcttagccatcaactgcccaaatcaaccttcatgggcatcttttaattttctaagagatggggtcttgctgtgttgtccaggcaggactcgtgggctctttccgcctcagcctcccgagtagctgggactacagttgtgcaccaccacacccctctgatatttcttaattttaatggctgatgtttcttaattttaacaagttcccatatgtctcatgatgtatgctaagtataaaaagaataatagaaggcatgaagtcaaggggcctgtttcccgggcatttgtttcagtggtgagtcacattcatgcgaagcctcggtttccttatctgtaagaccagatcagcaaaccacctgtggctgcctctctccctggttgaccccgaggccaagaggaggggcagggtcagcagccggccagacctcagtagcccctccccatcctggtatgggggaacacaggaggtagaaaacaagtgacctacacaaggcgggtggtggcgggggaggtcccactttaacaggaggcaagatggccgggagaaagctatggggaacccaggcaaatggagatgagcagaccccaagaaccgtaccctagggggctggctgggtcgggggaattagggtcctttgggctccttgcagagccgaagattgtagtggggagcactgcgtgtgcctacaaggctgagcctggcaggcagaattatgtgttgttttgagataacagccacaacagagcagtaaagaatactgcttataaaacggacatttttttgtagctcaaaaatggtcaaatatcagtaatgtcatgtggtttgagtaatccataaagcagagcccttctcgttagggctgagaattatcctcccctctccctgccacagagccggccacgtggcctgccccggcacctgctccgccttacatgtcattatgcgtctccgagacagcgtctctcacatctcaccgtttaaatgagcgtctagcaagtattctatggagtgcaatgcttgttggatacatctggcattttcatcttcgcagagtgggtttgtgacggggtgaaaaacaaaaaaacaaattcttgattcttgatggaaatctctcagtcctgagagatttataaaaaaggcttgtaggaaaactttgtacgcgtagcttttatactcatagaccaaattataattttttattcaatcttattttctgaataggtaacgtgttcacttggtctaaaatcaaaattttagccaggtccggtcactcgcacctgtaatggcagtactttgggaggctgaggtggggggcggtcacttgagctcaggagtaacatggcaaaaccgtgtctttacaaaacacacacacacacacacacacacacacacacattagctggacatggtggcacgcgcccgtaatcccagctactcagatggctgaggcgggaggattgcttgagcccaggagttcgaggctgcagtaaactgtgattgcatcactgcactccagcctgggtaacacagtgagaccctgtctccaaaaaaataaaatttgtatgtttatatactgagaaattttgtctccgcctctgtccctttttacccctactgtctaccctaagtcagtactgtacttctgttcattttctgggggtgtgctgaacagattcaagagaatagcatttccatggaaaggccgcagcttgtaagtttcacttgaatttccgtgattgggagttaatatcaaatgatctctcagaatcaagaaatactgatacatatcaaatgtataataacattttgagaattttttttttttttttttcagacagagtctcactctgttgcccaggctggagtgtagtggtgtgatcttggctcactgcaacctctgcctcccgggttcaagcgattctcctgcctcagcctcccaagtagctgagattgcaggcgcccaccaccacacccagctaatttttgtatttttagtagagatggggtttcaccatgttggccaggctggtctcaaagtcctgacctcaggtgatccccctgccttgccctcccaaagtgctgggattacaggcgtgagccactgcgcctggccccattttgagaattaaaggaacatgggcacgtaatctttatacgaggtggagtcagattgaatagcaagaggaagagaaactgacatccacgctcattgcactgggcgttttatgtgacttaatcctcatagcagctctatgaagtatgtatcagagggtgtccccattttacattaaatcacttgcctggatcactcagctggggagggtgagagccaggtgacctacccaaggctgtgtctctgggtggggggctcagaacctctctccctcgttttgtagtagccagacattgtctcgtataaactatggcaactggaagtgaaccaggtcttttcctgccctctgattttggtggaaaatgtgacctacccaggaaaagcactatgattttaggagtttaggtgcacaaagctgttttccaagcatgctcattcttctcccaatttgggactcattgctgcctctctgccctcatctgtggggccaagggagctggagcgacagccacagcagaccgctctgcatccctgtatgcgttgtgggaaacagcagaggaggtggcgttgcgcccatcttacagactccagattctgttcacccgacaggcagtgggaatccgtgttcacgctggtcccttggatcagaaattcagtacccttccctccacaccatgctgtagacacctctttcctctcctagacccggaacatcctctcttcttcagggtactggggaccaagatccaccctcgtggtgagaaaaccacaccataaataaacacagccaccatacgccgtgagatctggccaagaggtttccttctgttcattcggccctaatctccaagatgggaaggcattgccagctgccctgtgtagagattcctgaacacgcagtgtttcttattaacccgccagataaccagaaaatcagtctttgggcaagcgcttgcttctccacgtgcccagtattttctttctttctttttatttttttaatgagacagggtcttgctctgtcgcccaggctgggctcgaacttctggcctgaagtgatcctcccacctcagcctcccaaatagctgggactacaggtgtgcgccaccatgcccagctaacttttttttttttttctggagagacagggtgttactatgtggcccaagctggtctcaaagtactgggctcaagtgatccccccacctcggccttccaaagtgttgggattacaggtgtgagccactgtggccagctattctttaatttactagcgctgactacatgcctgtgatgtccccaggaccctgcaaagttctgggaaattcatagtgagcaaaacacagtccttgctggagttcacagcccatggggagacggcaccaaccctgtgcccagactaggggagctgccacgcagccactcactgagctgggcacacaggttcccatctcttatgaggtgaggcaggaggagcagggagggagctggggaagtggcatcagaacattttttggaagagagaaaggaaaggtttccaagcagaaggaacagcctgtggcgaggtccgaggggaaaggaggggaccctgttctctggggacactaggggaacgtcagcatgccaggtcagggttctggggagccctgggtggtcttatcgatggcccccaggaagccacagaaggcatttgagcagggaacgccaagctcacggcgggctttggcagggcgcgccccgtgtcctggaggactgtgtcctggtcatctttctaggcccccactgcatgcccttgaggactaggaagggtgcctccttctctgcatcaggctgactcctagcagctgccgtatctccagctttcctgctttcaactatgctttttgtctccttttcctcattgcgagcctaggcctgtatggggtggtggagtgggtgggagctcactgcttaggtgggtgggcagtgtgggtggtggtggttccatgggtctctccctcccactgtcccatctttgccactcatctgctgccccccactcccggaccccttttggaggtcagtctcactctgtggaaaccctggcttcctggttcgagatgccccctctggcccacgtgtctgacatcacgtaccagaacactgtggccatcattcaggattttttatgacacagtagtgaatcaaagcctgtcagtgggaaagtgatggggaaggattaagtgtctgagaacatttgggaaatatgaggttagacctggttctcaaacttatgaaataagtgaaaacatgaacgttcactttgggaattctgcaagattctcctttaaatggagtccatgaagagaacaaaatgaattttctgcagcgttacgcaggagcacttttctggaaatgaggtttcctaggactttgtcagcccttcaaagcctaggtctccttactagaggggaagaaatgagattttatttatttttatttttattttttattttttgagaccagagtctcactctgtttcccaggctggagtgcaatggcacaatctcggctcattgcaacctccgcccccagggttcaagcgattctcatgtctcagcctcccgaatagctgggattacaggtgtacgtcaccacacccggctaatttttgtatttttagtagagacagggtttcaccatgttggcctggctggtttcgaacttctgacctcaggtgatccacctgctccagcctcccaaagttctgggattacaggtgtgagtcacccactggccacaaacttatgaaataagcaaaaacatgaactttaactttaggaatctacaaggttctcctttaaatggagcccacgaagagaacaaatgaactttctgcaaatgagatgtcctaagaccttgtcagcccttcaaagcctaggtctcattactggaggggaaaaaataagattttaaaatatcaatgtttagccagactcagtggcttacccctgtaaccccagcactttgaggaggctgaggtgggaggatcgcctgagcccaggaattcgagaccagccaggacaacatagcaagacttccatctattaaagaaaaaaaaaaccatcgtttacttttaggagtaggcattacatttacgtgcttaaaataccaaaggtacccaagtgtagatatgaaagactttcccacccccaagtgagtgcccagccccttaatttccctccctggtatggatactaccagtatcttatgtttgtagaaccttctacagaaccttgtgtaggtagaagcagattctatgcatctatgtgtatctgtgtatttgcagtgactagattgattgctgttgatggacatattctggggaggtgtttttacacttccattactgagcacaggctcacaggatggcactttcccgcatgcaccaccatgcctggctgatttttgtatttttagtagagacagggtttcgccatattggccaggctggtctggaactcctgacctcaggtgatttgctcgcctcggcctcccaaagtgctgggattacgggcgtgaaccactgcgcccagcctgttttttgtttgtttgtttgtttgtttgtttgtttgtttttgaatagagacagagtttcactgtgttggccaggctggttttgaactcctgacctcaggtgatccacccacctcagccttccaaagtgctgggattacaggtgtgagccaccatgcctggcctcaatttctcccttttctaagccccccacctctgtttttgtcccatggtctggtcctgtcggccttgttgcacctggcagtcagggttgacttatgatttacttgaaccatccctggagcccctggcctggtgggggatgctggtggcagaatgaattcgggtctgaggtcaagcatgtgtctgtgagagtacacaaaatgaactgtgatgtagcgtttactgtgccacaaacttgaaatgcatcatcttgattatgtccagagaaaggaagtcatttgaccacagccacacaggttacaatggggatgcggcctggagtccaggtgtgagctaccacacccagcccacatacactttaaagatgagcaaatccaggctgtaatcccagcacttagggaggctgaggtgggaggatcacttgaggccaggagtccagtcaggtttgggaggccgagacaagaggatcgcttgagcccaggagtccagtttaggtttggtaggctgaggcgggaggatcgcttgaggccaggagtccagttcaggtttgggaggccgaggcgggaggatctcttgaggccaggagtccagttcaggtttgggaggccgaggcgggaggatctcttgaggccaggagtccagttcaggtttgggaggccgaggcgggaggatctcttgaggccaggagtccagttcaggtttgggaggccgaggtgggaaaattgcttgaggccaggactccagtttagacaccactgtttgaagcgtcttgggatgttttcagtaggggacttggagaagtgatggaaagaggctggtgggtggggcttaggcccaccacccctttctagccagagaaccctccattcatcgaaatgattctgttgttggaaatgaaacaaaaaataaaatggtcttaggaccttcctgtgcaggaaccatggccgacctttccctttttgtacaagtgatctatttttttcactagcaaaaccatttaagatttctatataggatgtgctgtttgattagtttgacagataatcccccaagaaacatcagatgaactcactctttggattaaagtttgagataataaaacaagaatacttgtgttgctgatgtggccagcccaagtatgtttttcccaccagcccagaggttctgggggcaggaggcatcctttgcaggcctagaaggacccaccctgtgggagcctggccctgtgcatctgcagagagcacttgctggaaatctcagtgggaaatggtggggatgcctcccatacccccactttaaaacaaacaactgacaattacacttttttttttgagacagagtctcgctctgctgcccaggctgaagtgcggtggcaccatctcagctcactgcaacctccgcctcccgagttcaagcgattctcctgcctcagcctctcaagtagctgggattacagatgcacaccaccatgcccagctaattttttgtatttttagtagagatggggtttcaccatgatggccaagctggtttcgaactcctgacaattatactttttgagacagggtctccctctgtcacccaggctggagcacagtggcttgattatgactcactgcagccttgacctcctgggctcaaacgatcctcccgccttagccccccaagtagctggaaccacaggcacccaccaccgtgtccagctaatttttgtattttttgtagagatggggttttgccatgttggcaggctggtctcaaactcctgggctcaagcgatctgcctgcctagacctcccaaagtgctagaattacaggcatcatccactgttcccggcccacatacactttaaaggtgaacaaatccaagccaggcatggtggcttatgcctgtaattccgcactttggcaggccaaggcgggcagatcgcttgaggccaggagttcaagactagtctggccaacaaggcgaaaccccctctctactaaaaacataaaaattagctgggcatagtggctcatgcctgtaatcccagcttctcaggaggctgggtcttgaaccctggaggtgaaggttaaccagtgagccgagatcacaccagcgcaccccagcctgggcgaccgagcaagactgtctcaaaaaaaaaaaaaaaagatgggcaaatccaaatggatctctggtgttatttaaatacttatttgggaaaaaaattttcccactatttctgttaagcagacagctggttacatggtagttttttaattttgtttttaataacatctgaaatgcctggcagaaagtctggagttgggggcagagagaaacttaaaatgttaggagctttgccctttgaggagctgtcagaattggagaaaaaacatcccagaaattgtaatcagtgaggaaaacaggattccagctatccacagcaccccagctgtacaccagcgtcacgggatgagggatgcctgtgtgcacgtaggccttggtttctgagctgagacagcagcagccaggccgacttggtgataggcttggcctgcccctgcagtggactgcctgggcctgggcctgggccttggccttggtctcagccctgtggtggatgatctgagccttggctttggccctccagtggatggcctgagccttggccttggccttggtcctgccgtggatgacctgagccttggtcttggccttggtcctgcggtggacgacctgagccctggccttggccttggccctccagtggatgacctgaaccttggccttggccttggccttggccctgccgtggatgacctgagccttggtcttggccttggtcctgcagtagacaacctgagccttggcaattggccttggccctccagtggatgacctgagccgtggccttggccttggttctgctgtggacgacctgagccttggccttgaccttggccctccagtggatgacctgagccttggcgttgaccttggcccgccagtggatgacctgagccttggcgttgaccttggcccgccagtggatgacctgagccttggccttgacctctgaagctcttctgtgcaagggtggggctcctgttggtgacacccacttgctgagtctacacctgggaagctcttggtcagctcggcccattgacttgagagaggaatagttgccgtccagcgagtgctgggggtgtaaggtggaccgggccacacggctgccaaggggccggctgggtgggactgaaacctgacccatgtgggtcctcattgcctcctgacatcagtcatcctgtgatctcttagaaagataaaaacacattttctgcgttctggtgcctctccttgccagacattttctcagtaaagtttctgtctcagcctcgttggaagtgccaggttctgagcccatcagtttgctgctagcctgccagcttcccatgacctgggacctgggaccaggggcccgcaggtgtttctagctggagaggagttctagatagttctgagcggataccctcctctgtcttcctcgggagagttctggcttggtttggtggtgttttctgtgaccagcagtggccaccgccaccaccaacccctcttccccctcccctcttcctccccctcccccctccctcctcctcctctatctccatacgccctgagactggccagtgggcagcatattgtgcaagaaacagctgttgttttgacaaaggacataactcgcctcacttcttgcatcaccagacccacgaggctctggattccaggcctctgcccctgcaggtggacacacagaccgtaacgcagaggctcacgtgtcccacactatgcattaccatttgtgtacgcactcgttcacatttagcaggattgtaaaaattaatagttagtgcagttcttccagctagataatgcatccaaatggcaagacagtccaaagatacacactatggagtaagtccctctccccaccggcccctctggagagagccaggcacggtcacaagtttcttctgtatccctctggagataatcacagcctatgcgagcatatggatggatattcttttaaacattgtgttgcggtttgctttttggcttggatgtatctgatgattatcatatcccttacaatacaccgtgtttttttacattacaggaaaatctgtgaggggctaaaccgacagggtattgctgggcatggaggaggttgggtccaggttttttgctctttgtgactttttaatgcctttcagatgtgacgactctccatacgccttctcacacgtgacccgtgactcatgggccgaggcctctgcatttgggacccccacctccacctccccaagatgcatttcattcccttgttaatgtgtcattgtagttgacgctttgaggataggcaggcttggtagataataaaacagtatttggcttatgtttaatcgctgtctcctcccacagcccgagaacgccttagcgtccttcagaaaggatttggtggtactggtttttatgtgtgtgtgtgttgttttttaaaaaaattttctccaagggacctggtcaaagatttcaagccttaaatctgatcaacccaatctggggcggcggcagctcagtctctcctcctcgctgtgccaatctgtttctgtggtggcgggtggcggctcggtgttaaatttggagacccccgtgggtttcccatgccgacctgcatatgtctgcgggcacggggaagggagggccgcagacccccgttgagtccccgactgccagcagcaagttgccgcaggcgcactggctctgcagccccgggatcaaggtgggttcaccttgcaccgggccacctgtttcagtttcctcctccaccaggcaggctctctgacagatcagcccagggaggggcaggtggggaccgagggggacggagggggcccacagacccccggctcatctggcatcttccccagcgtttcccacacttgacccactagatgagaagttgggggtcggggctgggaccccaccttcatacctccagcgcctgccaactcaaagacatcggtagatgcccaccgattgggtaaccgcccccagcacccccaccctgctaggcttccatgcagtgggggaagtagggacccccggccaaaagcagtcatgttctttttgtttttctcctaagttttctgcgtctgtgtgcgattggaacgaaacatcaactctttttttttcttttttgtcatatttcaatattggctttcttccccgccttctttcgaaggctccttttataacttcggctgctgctgcttaaatatagatgtcaaagccctcctccctccagattgtttatatttttgaagagccttctgcttcgctgtgactgctcctctcttatctgtatcccccgttccaccacattggtttcggttgctcttttttttttttccctgccttcccttaactgggatgtgtttcaaatgtggtctatcaaaatgttcagtgtttcccttttttctgcaatagctttgacatctgttcttgaattctagcccacgttattccttaggacttgtttgcccaagatgtgcctgaatgccagaaatactaaggggagaagagtgtggtgtggtggtatcttcccctcattgcctggtttgttgttttattgtcgttgttgtttttttttctttcctcccctgctttttaaagtgtctacgattccttccttcttccttccttccttccttccctccctccctccctccctccctctttccctccctccctccctctttccctccctccctccgtccttccctccctccgtccttccctccttccttccttccttcccttcttccttccttccttcctttcttctttctttcttttttcccttgggacacctcactcatgcccaggctggagtgcagtggcgtgatctcggctcactgcaacctctgcctccccggttcaagcgattctcttgcctcagcctcctgagtagctgggattacaggagcccaccaccatgcctggctaatttttgtatttttagtacagacagggtttctccatgttggccaggctggtctcaaactcctgacctcgggtgatccacccacctcggcctcccaaagtgctgggattacaggcgtgagccacctcgcccggcctgtccatgatttgtaagttgacagcatgatttcatcccatccggcatggcccaccccgtagtagtaccaaggaagcatttcccagcaagaacttgtaacgtgtaggtttgctggttaacttccgccaaggaggaaagcctgtgggacttggtgagctgggtggtggggggtggagaaggagaggctctagtgtgcttcttggaggcgtggggataaatcttcagctttgatttttctcgccgttcagctctgcttggactcagggtgatggtcccaaccgtgagaagccagctagagcagccaccttggatcccagcagaacgggccgctgtgtgtctttagttgccaaagcgactcggcaggaaaacaggccttttggtggcgacagcagtgtgtccttgggcgcctgctgtcagcctggaacaccattctggggccatctgctgcaggcttgttggaaacagatcctcttggcagctcttcccagaatttctgaggccagggcacaggttctctggcgcgttgattatcggtttgttgcggggagcagagcccagagtcctcagccccctagcgggtccctcatggggaacccactcctggtggtcctcagcgttcccgcgcaagtaatgtgatttgagaataactgccaatggtgcctcatttctgaaaagtggttggaaataattattcttggaatttaccttttaaaggtccctcttgaaaactaatcataggttaatggtctcgaacatgatgcttggcctagagtgagctcaccataaacatttgttgaatgaatacaaaccaagtattactctcagagatcccaggcccacagcattgaaggacggccacacggaatgccccgaggaggggatagatcagcagaaggactagagctcaggagagtggctggctggaaggaacccttgggaagaagaggcgtggagcaacttggggaggcgggtgctttgggcattagcctgatagttgtggctggagcaagaaagatgtttctgtgtattctgttatgggaacagagtcatttatggcgtgcacattgaggcacgacccatgatgttgaacgagcttggttttctcaaccacttcacatgggacggacgtgggatggggggataatgttcatttctctgccttcccgccaccctccagcccctcctctgccaatgagttgatgttccttagagccactggggtctctcccccagtgttctggtcaaatgcaaataggaccctcctctagtacacagtgcaattacaatctcacttttcctttcctgtttccccaacagagagaactcgatgccaccgcaacggtattggcgaaccggcaggatgaaagtgagcagtccagaaagcggcttatcgaacagagccgggagttcaagaagaacactccagaggtgaggcgcgtgaccatcgtgttcgctttgaagggatcttagaatgctggtgcatgttcaggcgacgctccgtgagcgtttcattttcatcagatgaacgcacggccggcaaacaacccgtttctttccccagatgtcttcagccccatttccagcagaacgcatgccatcctgcaggctgtggggatgtggaaattgataggttgtctggaaatatgaaagtcagagccaattccaggtgcagatactggacaagcttggtctgtaagaacacgtgggcaggtgtgtgggtgtctcagaccctcgagctcatcccagaccctgtcccatgtcagttagcaagccaccaaagtccataagggatcctgtggggtggaaggtccgcggggcctgcttccctgttgctggtgcaggcggagtgtctgaaggctgcacgcatctgggcatagcagtgcgcctaacgcttcttgtaaaacagacatttcgcctgctaggccttttaaatgcctctctgtttcttgaaatatgccgtaaagggcaatggaaatgtgctttttatatactcctgttttttctctcgtgagtgtgcaatcgggggacagtgttgagttgctggggtggcgtttttctgctcgtttcctggccccttcttccttcccttcaaccctatcaggggcttactaagaaaaaaaaaaaaacatccaagcgtgttgcaggcagatgagcagtcgcgggaatggctttccgggtgacatctgccagtttggtccccatggcgctcatcccgcgggctcggaccccagcctctcttacatcttccccttgtatgggaaggggtcgcagcagcccacagcttcggcccggcttccgggcttggggaatcttctccgtatcgtagctcttggctcctccatataagacataggaacatgcctggaggcaaagctcctttctaggagagatgcccctctcttacttacataatattgctgggaaattatatgtgaattgcatttttaaaagcggactcatttaaaatgtttcaaaagaggcttgctagtcaagggactgctggcatgaatcattatggaaaacaaattaataaccttctgtcttcagaataaatattttgggagaaagcttggtagcagagtagaaagaaggcagcctttggccacagagccagctaagggttcaaatctcacacccgctgcttgcctcggctgcctctaaatgtgggtactccatgtttcacgagaccaaaaatgcaggtgggagtcactggtgcttgggggtttctgccttctctgccagtgttggggagtggggggccctattctccatgtcagccttgccatgagtaaaaacaggaggaaaaaaagagctggggacagaacgtccttcttctgttgcctccagcggcttcagagcagactttcctggaactccggtttcctgagcgcttgtccttgactcagtttccccagcccaagccccgccacatccatcgtgctagctcctcttagtgccgttcctgtagctagtggtcacccgccgttctgtattgtcactgccctttcctcggtgaccatatgtctgagggtttccatagaaaatcttagaggtttggctgggcgcagtggctcacgcctataatttcaacactttggaaggctgaggcaagcaatcacttgaggtcaggagttcaagaccagcctgggcaacataacaagactcatctctgttataaaaggtaaaaagaaaaaagaaaacgttttgtggttaaaaacaaacaaagaaacaaaaaaacccagaaaatcttagaggggcaaggccatccttcagaggccacggacaataaatacacgagtgtattcagccagagaatgaggttcctaagaggcctggcttcctgggtgttcctcagaagtccccaactcctagtgacagtggaggaggagcgtgtcccccttcggtcaccctgtccgttccttatgctctgctgataaatgccgcttgtactgcactgagccctatccctccatcgtgccttgtgtcgaattccacattagttactcaaagacacctggccttaaacttggcgtttttatctccttctgatcccaataaatggaagaggcttggggaggcggcagcctggtaggtacctgccacttggggcatttcttctcaggacacatttttagactctgtcttcatggagaaaggggctagaacccaccaggaagggga

SEQ ID NO: 4 - DNA, 158298 bp, Homo sapiens
gaattcttcatttcagccctctgaagtcaaaaggaatttacttttgtatctttagtacgtttaaagaagatatctaaagttctttaacattttgataggaaatagggagctgagatttctaaatagttttgcttggttttagttttttgatctaacgttatgaacactgaagcttcatattttcatggtttcttttgataatttggaatactttctgccaggttgagtagttgaattcttgagatcaatagattatttctgtaagaaggttttgaacatgaatcgcttaaagttggcctattataaaattaggatgagacagttataatcaaatgacaaagtgcagaaaaataaaattgtttttgtaatgcccttcaacttgagtctaaatgggagtactagcgataacttgcaactcatttgttaaagaaagggtgttgccagtggatgaactcctagctttaggggagtcttattttaaaaatgattttgtgtgctttaaaggaatgaaaaaatatcttatcagtttaagcagtttggatttagaaatcttcccttttttcacctccctaatcttggctttaaaaggaatccatattcttcagtttaataaatttatctttttgttgttttgtatgagaaaaatattaaattttcttttctttctttttcttttcttttctttttttttttgagacagtctcgctctgtcactcaggctggagtgcagtggtgcgatcttggctcactgcaacctctgcctcccaggttcaagcgattctcctgcctcagtctcttgagtagctgggattacaggcgtgtgccaccatgcccggctaagttttgtatttttagtggagacggggtttcaccatgttggtcaggctggtcttgaactcctgacctcgtgatccgcccaccttggcctcccaaagggttgggattacagacgtgagccaccatgccaggcctaaaattaaattttctaatgtgactgcaaatagtactcaattaaatatgtttaaacttagtgttattatttttatagatcatttaagtaaaatggttttaatttgtgaaaacttcagcctctggtgaaaattcagttagttagctctcttacagtaactataatactgtcttgactctttgtgaaggaagttacattcagctcatttggttctgaagaaaacagtagggaaataggttatagagatttttgctgttctagaaacagtataactagaatgtactacataattttctcaaaggatttcttaatctaaatggaacttgtaaatggtttttatcccactggaatagcatgtattggataaaacttgtttaacacattctgagattttataatattttatgttacctcaaagatattaggaaaaatctgcaaatatccattcctgaataaaagatgacaatatcctcaatattttaaagattgtagtatatacatattttcctcaaaagttgagagtactcaaatttaaaagctagcataatttacccagttatttttgtgatgcaaggaaagaagatatgtgggactttttaatcatgcatttattggttttctaaaagaacttcacaattgtttttaatcttgttatgtggacttctttattagaaaagattatgtggtaaatgtcaactttttctactataatagaatctaactggttctgtgacacatttcttttttatgatacgattctttattatttaattacaaagtttataaaaaatttcaccactattttgtctccttagattttagtacttagattttagcagcatatttttctttttatcagaaaagagaaattataagctactaaaaagaagatgattacttttacttggtaggaaaacctaactttaaggatggtataacacagtttgagtatgagtttatttttcatcttaacaggtttatttgtgagtatggctttttttctccccgctactccccaagacggagtctcgctctgtcacccaggctggagtgtagctgcacgatctcggctcactgaagcctctgcttcccaggttcaagcaatcctcctgcctcagcctcccgagtagctgggattacagatgtccgccaccacactcagctaatttttgtatttttagtagagatggggtttcaccatgttgaccagctgttctcgaactgctgacctcgtgatctgcccgccttagcctcccaaagtgctgggattacaggtgtgagccactgtgcccggcctcaagtacggcttctttattattaaactctgtttatacaggagagttgtcctgttttaagctttaattgctttgggtgatgggcagatgatgtattgctattaggataagtcttgctatttgtataggtgtgtatgtcagataactgttcaaaaacaacttttttaaagtatggctactaaaatctcacctcttgagttttaaagcaattatggagtagaatttgaaatcaaaataatcaaaattggcttgtggatatgcctaaaaccagtttggaaaaaaggtcaaggcaaaaaagcagtgtaattagaagaataaaatgctttcccttaaaaatagtagatgtgaccgggcacggtggctcacgcctgtaatcccagcactttgggaggccgaggtgggcagatcacgaggtcaggagttcgagaccagcctggccaacatggtgaaaccccatctctactaaaaatacaaaaattagccggatgggccgggcgcggtggctcacgcctgtaatcccagcactttgggaggccgaggcgggcggatcacgagatcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaataaaaataaaaaaaaaaaaaaaaaaaaaaaaaaattagccggatgtggtggcacgtgcctgtaattccagctactcgggaggctgaggcaggagaatcatttgaacttgggagtcagaggttgcagtgatctgagattgcgccattgcactctagcctgggtgacagagtgagactttgtctccaaaaaaaaaaaaaaaaattagtagatcttatattactgcttttggaagcaattagtgaatgatgtaggcaaaatagtgattgaattttgaaaagcaattagactgaaatttcaaaggggtggagcatttcttaatagcaacaaacagatacatggtcattaatccaaatatctgaaattttctgagcatgtttttcagagtttggaatagagtggaagtgctaattttctgattatttaagaaccttttaatccattcccttagagcatcaggtgttttcagattgtgaattttctaaaataacattggctgatttgtgttattgaacattggttgtagcaacttttttccttttttcccctttattttctgcctcccatattttgcttaagatttttttctgaagttttaaagtttcatcagttatttgggtggaccgtcttgtgaatgctctaattgaagttgtataatataaacagtgttgcttttagtagttaggattaatttttcttttcatggaaatcctttctatttgattatattttttcctctaacttttattaattcttagatgagaattaggattctgcattaaatctttctgcacacatttttcttactgtcagtagggacctatgtcttccttgggtaaagaaaatgttatattttggttttgccatttaaaatgatataaagacgttattagtacttttttaaaaaggaacttttggtatttttaaacaagtacaaaagtaggtatgaaccaccatgtacctgttactcagcttcaaatagtatcaaatcttagtcattacattgtttcattcgtactagaactttttttctttttttttttgagatggtctctgttgcccaggctggagtgcagtggcatgatcctagctcattgtagccttgaactcctgggttcaagcaattctcccacctcagcctctcaggttgctctgactacaggcacaggaccatgcccagctaattttcttattttttgtggaaatgggggctcaccctgttgcccagggtagtctcaaacttctgggcttaagtgatcctcttatctcagcctcccaaagtgctgggattgcaggtgtgagccactatgctgtgatataataaaatgtctttatttatttattttgagatggtgtccgactctgtcacccaggctaaagtggcagtggtgccatcccggctcactgcaacttctgctttctgggttcaagcggttctcctgcctcagtctcccaagtagctagaactacaggcgtgtgccaccatgcccagctaatttttatatttgtggtagagacagggtttcaccatgttggccaggctggtctcaaactcctgacctcaggtgatccacctgcctcagcctcccaaagtgctgggatcacagacgtgagccactgtgccctgccaaaatcaggattatttaatcaacttttctttttttcttttttttttttttttttgagatggagttttgctcttgttgctcaggctggagtgcagtggtatgatcttggctcactgcaacctccacctcccgggttcaagcaactctcctgcctcagcctcctcagtagctgggattacaggcgcccgccaccacacctggctaagtttttgtatttttagtagagacagggtttcaccatgttggccaggctggtcttgaactcctgacctcaggtgatctgcccgcttcggcctcccaaagtgttgggattacaggcctgagccactgcacccagccttaatcaatttttcaaacagactttgcttatggaaccacttcttggaaaatttggcagaacttctttgtagagtttatcttgtcaagaatactttcataatgatataattagcactgtccaaagcagtgtacaaataatgtgtgctacatgtagtatgagtccagtctgtttgtacagggatatcttcagactaaagtcctaggagtgaggcttcctgaagaagatgagtcttgaataaagattgtgtactgtttgtataggctagcatatccagaaggagaacattatgtacgttctatatagggaatggaagcatgtcttagttcattttatgctgctataacagaataccacagactggataattataatcgacagaaatgtatttggctcatggttttggagactgggaagaaccagaacatggcactggcatctggcagggccgtcatgctatgtcatcccatggtgtaaggtagaagagcaagagagggtgagagaacaagaacagagagggcagaacttgctttttatcaggaacctacttccattactatggcattatcattgatgagggtagagccctcatagcctaatcacctcttttaatggcctcaactcttaatactaccacactggcaattaaatttcaatatgcgttgtggaggagacatttagaccacagtaaagaatgagcaaacacttgataggggctagcatgatgtgagagggatgtaaggagtctgatctcaatggaataaagtgttaattactgggaatggtggcagataaatttgtattagtagggtagagctagattatgaaaggtgtattggtgtcaggttgaatttaatctgctaagcaataagaagccagtagaggttgatagggagcaggagaaaacatgagagtgtttttcagaaagattactcgttcaatagtgtgcagcatattttggggatgacacttgaggtttgtttgaaaaattagagaccaagccactgaggtaaggagctattctgagagacgttaggatggaaattctacaggatttgtcagataattgattccatcttctctttagtccctcatgccctaaagtgacaccaaggttttgaagagtagagaactgaggaaatgtggtctatttgtctttgatagtaaactgcaatttgttatccaagcccagcttttgcgaagaaagtgatagatgattactccaataacagcctcaaaaagttttagtaggcctggtgtggtggctcacacctgtaatcccagcactttgggaggctgaggtgggaggattgcttgaggtcaggagtttgagaccagcctggccaacatagtgaaaccccgtctctactaaaaatacaaaaattagctaggtatggtggtgtgcccctgtaatcccagctacttgggaggctgaggctaggataattgcttgaacccgggaggcggaggttgcagtgagccaagatggcaccactgcactccagcctgggctacagaatgaaactctgtctccaaaaaaaaaaaaaaaaaggtttagtagaaaatagctaccagtgtttctgaatgattttaaagatattcactggagaataggagcttggaaaataaagaaatattatctttattacagtaagagaaatgaacatacttcatttgtaaaagagaaatttaggccttacctgcttcctatgtgtatattagttcagaacggttattaggagactactcacatttgaatagctataaccagtaaatattactggatgactttaataatggcctgatcagagctaaagaaaattctgatgaagtagggcattaaatatttctagctaccaactccctttaaggatgaataaaggtaggtccgcaggctgaggaagtatacagggagattgtgaaatgaaaacttatcctttcatttacttcgatatctttgttttgcacaggtaatattaaaaaacaggtctggttaaacatcagccgtatatgttttggttggaattttttttttaagttctgtgaactttgaagtttttttaaaattctttgttttggtttgtagaccctgcattaagccaactttctggatctgttgtgtatttctggatagcatactttgttttgtgaaggaaaattttacattttattttaaagctgggggaaaaaagtaggtttcttgtttctcgctttgtaaatgaggtagatgaataatactgtattgccacttattatcttcatgcttccttcaatgtctcaggtattcctctactgtatgtagcatgtggatctcaaagtttcccacagttgagtctgaaaacatctcaactgtgcttataattttaaaattaaacttaattttaccattttctttattgttattataaaatataggacctatcataatgccatgaaccttttagtgacttgggaaatagtgattaagatgatgcaacatagtacattattgggaaataatctgtttttcgttggcatcgcctctatccttagacctctttcattctagtaatatgacagaaatatttttgacattttaaaacttcctcatttagctgatcgtgtgtgctgttgaaaatgaacactagaatagaaagccattgccttctccttggggggtttgttttaacaggggttttcatgttttaattttggatttgagatttgagttgagatatccattgtgtagagaatcttgagaaggattatccatctttcttgctgtcaccagtgtatgtgggagggaagaaaatatcaaatatagggtatacaggagaaccttagtatttgggaatatcttcagtgatagctgccaaatccagtgttttccccaggaaatttgtatttaatcccttggagtatgggatgactttatgagtgtcatagtgggttgctgtgggtgaggagaggagaaaacgactccctgtttgaaaaatcttttccaaactgttcgaaaatttgagattaaattgaattaatctcaaattattaatagtttatgttttttttttaattcatacagtgcttgggaagaaattggttttctgtgagtggccttgggggtgctttgggtgctttccttacccaggaaaagggaggagaggcagaaaataaagtggggaatagtagctgctgtggatttttgcctatggcattgatggcattgtttttttggctgcctttatctacataccttttcttttctagttaggctgtcattactagttggagaggctacatacagtctagccttttccaaatcaaactagcatccctgtcctggtttgtttgttttaatctctccgtctcaaaacctgacaaatttatgtcttttctctgcctgaagagcaggggaaactggaaagagaagaaaatgataatgttgtagttaggcactccctatcttttaatatgtcaggattgttttatgaaaaaaacctctgcctttggattttaattttatgtaatcagttataaaataattgaattctctatgagtccttagaaaccctttaaaagatcctttagccgtaaagttggtgataggttgaagagtacctatatgagttaaatgctttggggattttttttgcttctcctaaatttttttctgtcatacaggacctagtcctacatgtgaattttgcttccaaaaagtaaagctggcagtcttttaatcccagcactttgggaggccgaggcaggtggatcacgaagtcaggagttcgagaccagcctggccaacatggtgaaaccctgtctctactaaaaatagaaaaattagccgggcatggtggcatgtgcctgtaatcccagctactcaggaggctgaggcaggagaatcacttgaacctaggaggcggaggttgcagtgagccaagaccatgccattgcactccagcctgggtgacagagcaagagtccgtctcaaaaacaaacaaacaaacataaaaaccttaaagattgtattcaaatctattttgattctgatgaaagcaaggatgacagcaaaatactttaatatatgcatgctaacaaaagaacacaagttagacctgtgtcaccatatattctttgtcatgcctagcaagtattttcagctttggggaactctgaatgatagatggtgtattcatgtttgtagattgtggtttttattatatactattaaattctataatttaaacttgacatattagaggaaatattgttcttcattatttggagtaaaggattaattatacataactttttatagtcgaatgtagttttcagtttcagttttgaactctatttgtttttattacttctgctgggagtatgctgggtagtatggaaccaggcttcctacatatgaatgttggctctgccacttacttactatctgtgagacctaggttgttactttaccgtctttgggactcagcttcctcatttgtaaaatggagataccaggattgttgtgtggaataaatgggttaatgtatataaagtgcttggaatagtgtttggcacatcgtcgctttgtctaagtgtttgctgtttttatgcatttgaaggaataaaaaacattcatctttgaatttctctattaattcagtacacttgtaaaacaatgtctaagtactaaacatatattttcagtaatcacttgaccacatagaaatataaggaatgtgtttgaggggtaatcagaagattggcctgcctggaattcccttgaatcagagagtaatggaaaatgaacctgtatggatacaatagaaaatgattatcaagtgttttgaactaaggcatagaaaattagatgctgataagaggggaatcattgcaaggttgttattgttgtttttaagtcatgatagtgatgtgtttaaaatagtatttaggggaagatagttttgagtgtgcttggtagcatagaattatttcactataaggacagaggatttttttttggtctgttctgttcactaatctattttaagcaacccaaacagaacctggttgatggtagatgctcaataaatacttaattggatgaatgaaccttaaaagagactatcagagtcttttatttttattttgagacagtgtctcactctctcacccaggctatagtgcagtggcgcaatcttggctcaccacaacctccgcctcctgggttcaagcaattctcctaccttagcctcttgagtagctgggactacaggcacgtgccaccatgcctggctaattattgtattttttggtaaagatggggtttcactgtgttggccgggctggtcttgagctcctggcctcaagcaatccgcctgcctcggcttcccaaagtgctgggattataggcgtgagccaccatgcctgaccctatgagaatataaaaaagagctgggtgcagtggctcacgcctgtaatccaacactttgggagtccgaggctggaggatagcttgagcccaggagttcaagaccatcttgggcaattatagtgagactccatttcttaaaaaaaaaaaataattatccaggcttggtggtgcactcatgtagtcccagctacttgggagtttaagtggggaggatcgcttgagcttgatagatcgaggctgcagtgagctgtgattgtgccacttcactccagtctgggtgacagagactctgtctcaaaaaataaaaagaaaatgagagaacagtggactcaacttaaacttttggggttcctacgtttcaggggtagaagtgaaaaagatactttaataagaacagtaggagaggtaaaaggaagatggaaaagtttggctcagaatccaaaggaagagattttctaagggaaaggtcagagagagagagagagagagagaggaataggaagattcaggatagtgttgcttctaggtagccaaggaggtttcaggaagaagggcacagaggtggacatagtccaatacgaagttcgaggagaatgagaatagagaaagaatcctttggcttggtggtgtgagggtcactgaccactgagtacactaagaatggtaatataaatcagattgcagggaggttaaggaagatgttaagcagagactgataaagactattcagttggcctggcacagtggctcacgcctgtaatcccagcactgtgggaggccaaggcgagcagatgacgtgaggtcaggagtttgagaccagcctggccaacatggcgaaaccccgtctctaataaaaatactgaaattagttgggcatggtggtgcatgcctgtaattccagctacttgggaggctgaggcataagaatcacttgaacccgggaggtggaggttgcagtgagtggagattgcgccactgcactccagcctgggtgacggagtgagacctcttgtctcaaaaaacaaaaaaacaaaaactatttagtcaagaaaataactgtaaaaatggccagaaaacaatataataggagcattcacagtttttgtatgtatactgtaagagagtctgtttattagaggtgagaaaacttgagacagtttgtgctgaatattattctttttctttattttagagacaggatcttcctttgttttccaggctggagtgcagtggcacaatcatagcttattgcagccttgaactcctgggctcaagcaatcctcccaccttggcctcccaaaagcactgggatttacagatatgagctattgctcccacccagccctgaatgttcttaaatttctttctttctttctttctttctttctttctttctttctttctttctttctttctttctttcttttttttttttttttttttgacacagggtctcactctgttacctaggctggagtgcagtggcgagatcttggctccttatagcctcccagcctcaggtaatcctcccacctcagcctcccaagtagctggcactgtaggtgcatgccatcacattcagctatttttttttttttttttttttttgaggcagagtctcgctctgtctcccaggctggagtgcaatggcgcagtctcggctcactgcagcctccgcctcccgggttcaagcgattctcctgcctcagcctcctgagtagctgggactacaggtatgtgccaccatacccggctaatttttgtatttttagtagagacagggtttcaccatgttggctaggctggtctcaaactcctgacctcaggtaatctacccgccttggcctcccaaagtgctgggattacaggcgtgagccaacttttcgtattttggtggagatggagttttgccatgtttgttgcccaggctgctcttaaatttcataatagggaatatgtattgtatatgtttctccagggctctgctacttgctaaccatgtgatcttgggaaactttgcttaatatttttgtgcctcagttttctgccttgtaaaattgaggcaaaaggaacacctatttcatagatctattgtgaggatgtaattatttaatcttttaaaaacacttagaacaatgctatgtggtaaatgtttcataaatgttagctattatctactgctgaagtaaaatagagtaaaattggtggcttgaagaaaaaatagaagaagaaaagaatttaataatgcaaactagtggcaaaaactaaatggaaaggtcttagggccttttccgtggatgttattggagaaaactttggatgtctacttactctatttatttacacttggatttcaggcccctttcctctaatatttgcactgttttacttttttctataacacttatgttctgacatatcatataattaatatatatactgtttgttgttgatctctttccagggtagggatatttgttttgatccctgatgtattccaggttctgggaatagtacttggcacataataggtgctcaatatttgcttgttggattaataagttaatctaccatagaagtaggatggatggagaaaagtgttcaaagtgctggtggtcctttttttttttttttttttttttgagacggaatttcattcttgttgcccaggttgtagtgcaatggcgcgatctcggctccctgaaacctccacctcctgggttcaagctattcacctgtctcagcctcccgagtagctgggattacaggcgcgcactaccatgcccggctaatttttgtatttttagtagagacggggtttcaccatgttggccaggctggtcttgaactcctgacctcaggtgatctgcccgccttggcctcccaaagtgctgggattacaggtgtgagccactgcgccccaccgctggtggtacttttttagagaagccagttatcagattgatggacactatataggactttaccttctcaaagacttcctcctttttctcaaatgagttttctgagttttttttttttttttggtacaggttttcttgaaatcacttgggaaacccatttgtttaatagctgaggaaaggaatctataaatgaatctcttttttttgatacaatacttatattttacatttgattagctaatgaaagcctatccgtgagcgcttattagatgctaagcactattctaacctccttattagtttcaaaaaattctataacaccttacaagtgcattacagtttctttgcaagccattattaaggtttaaggggaggccctttgtactgagaccaaaagcatgattccccaaactgttgtcttccattgatagggagcttcaaataccacattccttcctccaattttcatagctttgacactgctacttgtattagtccaccaggactaatctcattatgttctttatcccttttccctttctatgtcagttactatcagtctcctaggtgatactcatgtccccacaaggtttggtgtatttactatttttttcaatcctcttcaactcttgaaatcttgccattgtgcaacccagtggaattcacagtctatcatcagcaaaatttcctgtatccttgtttgtctttctctgaacattttgtctacttcgtactttgctgaggatgctgcttcccctggctgtcttatacattggcaattaaatatttcatttttttctgataatgtcatcaaggatcagtggggagtcctatgcactgagacttgaaactacatttcctggccaggcatggtagtatacacctgcagtcccagctacttgagaggtagaggcaggaggatccctgagctcagttcaaggctagcctagcctacgtagtgagaccctgtctctaaaaacaaacaagcaaacaaaccaaccaaccaaccaaccattttcctgctctgacctactccatttgaacaggagtcaagagggagcctggttccactgactggaaccaagagcctggagatggggcagttgtccaccattctcttgttctccttaaaacttgtctattaacaggttatattgtctgtaaatcctcatgtttgctggcatctactgccacctgggttacaccttctcttttcttgactggtttagctcctggctcattgtcattcttctaacactaagtctcttttaattcttgataatttcaatatatatcactatggtccctctaatactgtgacctcttaatttctaaatgtcttctttaatgatcttactctctagcccatttaaggcacttcatggtcctatcttttttatgcattaaaaaagatattttgtaaagatggggtcttgctgtgttgccaagtctggtctgaaactcctggccccaagtagtcctcctttcttggcctcccaaagagctgggattacaagtgtgagccaccacacctggcctcatgatcatgtcatataccttgttattaccaataacttgaaacctcttcattttttctaagctctttctgactgctgtcttttcagcccattctacctggtacctcaactcaacagttcctcaccgtcattaaaactgtcagtccactgattctattgccttttcactgtaccttgccccgttgatttcctttcttccttccttaccttgtataattcaatcgccagtcatattgcatatgccttcaactcacttgctcctttctgcccttttcataattagttggcaaaaacctcaatcttaggtaaatcaactctttgagtacttcatatctacatctgtgcggctgaaagtgactgaagaaaaatacaaatatgataactgccctcactttaaattcatggtaagaaacctcaggtttcttgccctgaatattgtctggcaattgtactctattttcttagtccattcagtctcccctctcatagatgacttagtttgtacccttgcttttctcttcaaacttccaacactcttcctcatactcactctcagttgatggacctgatttctacttcattgtgagatgcaagcagtcagaagaacacttacagactcctgccaccacatgtatgtattgactacatctgtactcaatagaccttgctgctgtctttgtgttactatagatttactctctatgtttctatatagtcccaggtcctctgattaatgcattaatgaactgtgaagttctttttatgccaaccactctccaaggttctttatatacagtgtagtatgtaactcctatagatttactctggacattgccctagccattctcccttctctctgtatatcatcaatttgtctcttctggataattcctatcaatatacaagcatactgttacttttttcatcataaaaatatcttccctcaattgctctttcttgctctctttctgtagaaccccttaaagcattgtctaaatgcacttgtcctaatttctttctcatgttctctcttaagcctgttctaatcaggctttgcccctaccactgtgctaagattgcttttgttaatgtcttccaacaaatgacattattaaatctagtagtcagttctgtcattatcttgggtgatgatctacgtttgacaaagttattttctccttgtctttgatatacttgtttaacttgccttttaggctactacatttacttggttttcctcaaaattcattggctgctcctccttagtcttccttagtagattttcctttttgcctacctctaaacattggaatatcccaaggctcactccttggttatcttctctatctatgctcattcccttagcaatctctgttagtttggtggctttgaatatttttctctcaaatgaattttttcagcccagatctctctccgtaactccttatttttatatccagttgcctatgttgaatggctcttaaacatctcacactcaacatgtccaaaatggaattcttgcttttccctcccaaacctgctctctcatacctactctgtccacatagccttcctcatctcagttcttggcagctttccagttgctcaggccagaaaccttggagtcattcttgactcttgctgtcttatcttcttatccaacattcattccttcaggaagttgttggctgtaactttaaaacatatccagaattcaaccagttttcactgtctccacagccaccatcatctcttgctgggtaatggcagcagccttttaacaggttttcctgcttctacccttgccctctgtagtctgttattaacacagcagtcaaaaagatcctttgaaaatttaagtcatatggcaagagattttgtactacatttaagaagtttcatgagttgataagattagagattgttctagccaatgatattttactaaatcatgtcagccttgaaggcacatttaaggcaggctttgagtatgcttgttagtgtgggtggggaaggagtggtcagagatggttcatagcctccctcttttctcctcactaacacttgtcttttccaccatatgcacgaaaagacatatttgttcctgaggaaattagaaagggagggcctgagttggttgctattcctgaaagtttctttgaatggaatactggatcatcgtttttgaagaggtctgttgttacttcattattacctcctttagcagcacccttgttttcctccaacctctgccaagattagtgtcctgtgtctacatttgccccttctgtttctcatactcccagatggaaatattatatttcttaggacttttcgctctatttgaattagtattttctactcgaaacccttcagaaatcctgaaatccattcttatgctttctgtgtgagttggttttatgtttgttaataatgtttcaacagaatgatagtggacatgatagcaaaagggaaattgatactaatatgtaacgaagaaatcgtatgtgaaattgtgaagaaaatgaacgatatttttattgagtactttgaaggtacttgtcataaactaacaatgctaatgatttctagaggagttatatactacactgtatataaagaaccttggagattggttggcataaaaagaacttcacagttcatttcttttatctgtttacattcttttctctagaggcaaatgttctttaatcagttttcctttgattctctttcagagaggctcatgtagtgggatcaacaatcatatcactgttgtggcattgtatatatgttaactattgattttgcattcttttagattataatatttaatctcttggtctgaaggtagatatattcctgttttctcaagctgtttcttagacctttattgtccagtatggtacccactagccatatgggactactgagcacaggaaatgtggctagtcggccaggcgcagtggctcatgcctgtaatcccagcacttttggaggttaaggcgggcagatcacacgaggtcaggagttcaagactagcctggccaacatggagaaaccctatctctactgaaaatacaaaaattagctgggtgtggttgtggacgcctgtagtcccagctactcgggaggctgaggcaggagaattgcttgagctggggaagcggaggttgcagtgagctgagatcacgcccttgcactccagcctgggggacagagcgagactgtctcaaaaaaaaaaaaaaaaaaaaaaaagaaatgtgactagtctgaatcaagattgtgctaagtgcaaaatacacactggagttagaatccttagtaacaaaattttataaaatgtgtaaaatttcttattagtaattttaaaatatagattatatgttgagctgatttgttgggttaaataaaattaattttacttgtttcttcctattttttctaaatatggctagtggaaaatgtaaaattatatatgtgatttacgttatatttctcttggatagtgctgtcttaaacttatatttctctttggccctaaaacttctgaagttgtttttaactctgttaatgatctgttatatattcactaaaatatgattttggtattatgtattataaaataatttgtttttttctgaaggaacaacttttatgtttttatccttcttagtcctaactttgtcaagtcttgagttttctttttcctttttttaaaaactctttgacttttgccataccttacccatggtagtactacacaagcataacttattctttctctacattgcttgggagaattttgaaaagggaatttgaaattatgagtatcagagttgatcttttatttgctttccttcttaatattaggactattaataataatacattctataattcctccatgataacataattttaaaatttaaaataaaggcaaattttatcaggtaaaaaagtgcattgaaaaagttacatacgaggaaagagatttttgtctgctttaacataaaacccagcaaataaacaaaacacctgcactgactcattctactggcacacattttgtacatttttataaacttagactctaacagccactattagctttttttcttcaaagatttcagtttaggctgggcacagtggctcacgcctgtaatcctagcactttgggaggccgaggcgggcggatcacttgaggtcaggagttcaagaccagcctggccaacacggtgaaaccccttctctactaaaaatatagaatttagctgggtgtgatggcatgtgcctgtagtggtacgattgtcttgcctcaggaggctgagaatcgtaccattcagtaccagtcgtaccactcaggaggctgaggcaagagaatcgcttgaacctgggaggtgaaggttgcagtgagttgacatcatgccactcactgcactccagcctgggtaacagagctcaaaaaaagacttaagtttatggttttatttatattaattttttttttgaaaaaaatccttgaaaacaagatttatgtagagtttaaagaatgctttttttttcttttttttcttttttgagacggagtctcggagtctcactctgtcgcccaggctggagtgcagtggcgctatctcagctcagtgcaagctccgcctcccagtggttctcacgccattctcctgcctcagcctcccgagtagctgggactacaggtgcccgccaccatgcccggctaattttgtttttgtatttttagtagagacgaggtttcaccgtgttagccagggtggtctcgatctcctgacctcgtgatccgcctgccttggcctcccaaagtgctgggaccacaggcgtaagccaccgtgcctggcctctgtgctcgaaaagtatattttgaaagtattccacaatctaatatcaattctgatttttttttaaaaagcaatgcatgagataatagcaattcatattgctactatcaatgtactaacaaaagtggctttgttaggcatttggaaataatttatttactgaagaactattacttttattgttcttaccgactcgaaagagtccataatacctgcttaggtacggaaaattagacctggtgtggtgactcacatctgtagtcccagcacattttggggccaaggtgggaggatcacttgagcccaggagttccagacaagcctgggaaacatacggatactgcatctctacaaaaaatttacaaaaaaaaattatctgggtgtggtggtgtataccaccaccactacttgggaggctgaggtgggaggattgcttgagtctgggagatcaaggctgcagtaagctgtgatggcatcactgcactctagcctgggtgagagggagaccctgtctccaaaaataagaaaagaaaagaaaatcatataaaagcacatttacaggaagagtattaaaaaaaaaaaaagattgggccaacaagctaaaacttttttttaaatggttgtaattgagaaaacataggttttataaacaaggacaatgagagcaaaagatatctagggtagaaaaagtagagataatacagataataggaatttggggatgtaagaaaatagcaaagtcataaaagttcacatttttggatgcttaatggaaggataatcttgacagactgaaacaaaaattttactgcttgttaattttctggatcttagtactctttaccttttttattttttttatgctttgaggataggactgatttctgttggctctctagacctagatttggattgattcataaacatttctttaaaaaaaagtgttttgttttgtttttgtcctgtgacacagcctcaggagactattcttatgtgccccaaaagaacaatttctgagttcatctgtgttgtgtatattaactctttgttcctttttattgctgagtagtattcagtggtgtgggtataccacagtttagctatttacccactgaaggacattttggttcttttcagtttctggctcaactaaaagctgttatgaacatttgtgtacatgttttcatataacataagtttttatttctctcagataaacgcccaagagcaaaattgctgggtcatctggtaagtatgcatttagttttcaaagaaactggtaaagtattttccagtatgtgagtacaattttgcattctcataagcattaaatgagtggtccagtttctttgtgttctagccagatagggtgttaccagttttttcattgtagtcattccaagagatccgtagtgatattttattgtggtgttttcttgcattccctaatggctaaagatgttcaacattttttcatatgcttattggcctttcgtaattccttttcagtgaattgtctgtttgtgtcttttgtccattttcaaattggttatctttactgttagattttttttaatctcttgagacttttctagatatctcattttcttctctttcattttatttttatttttctattaagttattggggtacaggtggtatttagttaagttctttagcggtgatttgtgagattttggtgagtgctttatacagtctggatactagttctttgtcagatatgtggtttgctagtattttctccctggttttaacttgtcttttcatcctctttgtagggttattcacagagtgaaagtttttaattttgatgaggttcagtatagcagttcttccttttgtggatcgtgcttttggtgtcaagtgtaacaactctttacctagtgccaatgcctgaagattttctctaagttttttcctaaaagttttatacttttacgttttacatttaagtgtgtgataacatttggatctagtttttgtataaagtaagtggtttaggttgataactcttcccccttcttgtcccgcttcccttcccccttcccttccccatccatggaagtcgcaattgctctcgcaccatttattggaaaggctgtccttcttttattgaattgcttgtgcatcattgcgaaaaatcatttgggcatatttgtgtggacacatttctgggttttctgttctgttctattagtctatatgtctctttcttcatcagtaccacagttttaattgctgtagcggtatagttatccttaatatcgggaggcataattctacttgttttttgttttcacaaaactgctttaggtctcctaggacttttcctttccatataaattttagaatatgtttatctatatctacagaaacaaaaaaccgccagctgtggtggctcaagcctgtaatcccagcactttgggaggccgaggcaggtggatcatgaggtcaagagatcaagactatcctggccaacatggtgaaagcccgtctctataaaaatacaaaaattagctgggtgtggcggcacgcacctgtaatcccagctactcggaagactgaggcaggagaatcgcttgaatccgggaggcagaggttgcagtgagccaagatcgtgccactgcactccagcctggtgacagagcgagactccatctcaaaaaacaaacaaacaaacaaaatccttcctaagattttgctaggaattgcattatactatacatcaatttggggagtattgatatctttactatgttatattttcccgtcaatgaacatagtaagtctcttcagttatttagatctttgattttacgttagcattttgtaattttcagcatatagatcctgcatgtattttgttagatttatacctaactatttcattttctttggagcaattgtgaatagcattgtattttaaatttcagctttcatgtttattaatatatggaaatatgattgatttttgatttttgattttttttttttttttgagacagagtctcactctcttgcctaggctagagtgcagtggcacaatctcgactcactgcaacctccgcttcctgggttcaagtgatacttgtgtctcagcctcccaagtagttggaattacaggtaacaccaccacacccagctaatttttgtatttttagtataaatggggtttcaccatgttggccaggctggtcgcaaactcctggtctcaagtgatctgcccgactcagctctcaaagtgctgggattacaggcacaaaataagactgatttttgtatgttgaccttgcatcttataaccttgctaaacttaattattttggggggatttttgggtagatttcttgggattttctagatagtcatgtcatctacaaatggggacagattttttcttctgttctagtctgtttgccttttattttcttgtgctggctggaatttccataattatgttggataagattggtgagaacagccaatcttgttttatacttgaagttaggggaaaatatactatttttcttttttaaattaaattaatttatttattttgagatgaggtcccactctgtcacccaggctagagtgcagtggcaagatctcggcccactgctaccttcgcctcccagggtcaagtgattcttctgcctcagcctcttgagtaactggtactataggcgcctgccaccacgtctggctaattttttgtatttttagtagagacggggttctaccatgttggccaggctggtttcggactcctgatctcaagtgatccgcctaccttggcctcccaaattggtgctaggcttacaggcgtgggccaccacgcctggcctagctataggtttttttgtagatgttctttgccaagttgaggaagttcttctctattcccagtttgcttagagtttggttttgttttgtttttgagacggggtctcactctgtcatccaggctggagtgcaatggtgcagtttcggcatactgcagcctcaaccttaagtgatcctcctacctcagccccccgagtggttgggactacaggcatgtgacaccttgtccggctaatttttgtattttttgtagagacggggtttcctcatgttttgcaggctggtctcaacttctcggctgaagcaatctgccctccctgctctcccaaagtgctgggattacaggcatgaaccaccgcacctggcctgcttggagttttttttaatatcatgaattttgtcaaatgcttctgtcaatgatatgatcatgtgatttttcttctttagccttttgatatggcagattacattatttttcaaatgttgaaccagccttgcatacctggaataaatcccatttggttgtggtttgtaattttttaatatattgctttatttttatttttttttttgagacaaagtcttaaactgtcactcaggctggaaggcagtggcatgattatggctcactgtagccttgacctcctgggatcaagcaatcttcccacctcagcctccagagtagctgggagtacaggcatgtggcagctaattttttttttttttttaaatttttttgtagagatggggttccactatgttacccaggttggtctcaaatgccagggctcaagcagtcctcctgccttggcctcccaaagtactgagaaaacaggcatgagccactgttcacagactagattttatttgctaatattttgttcaggatttttcatctactttttttttttttttttttttgatatggagtctcgctctagttgcccaggctgaagtgcattggcatggtctcagctcactgcaacctccacttcccaggctcaggagattctcctgcttcagcctcctgagtagctgggattacaggcacccactaccatgcctgtctaatttttgtatttttacaaaattagagacagggtttcaccacgttggccaggctggtcttgaactcctgacctcaggtcatctgcctgccttggcctcccaaagtgctgggattacaggcgttagccactgtgctcggcctttcatctacttttatgagaggtattggtctgaagtttttctttctttctttcttctctctctcttttttttcctttttttgtgatatctttgtctggttttgatacctggctctctggaccaccggatttctttctttctttttttgatacagggtctcgctctgtcagccaggttagagtgcagtggtatgatcacagctcattgctccacctcccaggtccaagcgatcctttcactttagcctcctgagtggctgggattacagttgtgtaccaccacacctgaccgatttttaatttttttttgtttacagggtctctgcatgttgcccaggctgtttttggactactgggctcaagcaaccctcctgccttggcctcccaaagtgctgggattacaggcatgagccaccacactcctaggatttctttattgggagcttttgaactatgaattcagtttctttaattttttagattgatgcataatatcatatatttatggagtacatgtgagtattatatgcatagaatgtgtaatgatcaagtcagggcatttgaggtatctatcacctttaatatttattatttgtgttgggaacatttaaaattctttcttctagctactttgaaatacacagtactttgttgctgactatagtcaccagtactctgctattgaacattagaagttatttcttctatttgactgtatattttgtactcattaaccaacttctctttatcccctctcacccacccaccctctgcagcctctagtatctataattctatttgctacttctataagattaactcttttagtttctacatatgagtgagaacatgcagtatttgtcttgctgtgcctggcttatttcacttaatataataacctccagctcatctatgttgctgcaaataacatgatttcattctttgaatgccaaatccattgtgtatatatactacattttctttatttcatctgctgatggacacttaagttggttctttgtcttggctgttgtgaatagtgctgcaataaacacatgagtgcaggtatccctttgatacactgattcttttcttttgtataaatacagtggtgggattgccagatcatatgataatgatacttttagttttttgagaattctccatactgtttttcatagtagttgtatggatttacattcccaccaacagtgtttaagagttctctttttctctgcatgcttgccagcatctgttattgtgtgtgtgtccgtgtgttttaagaaatatccgttctaactggggtaagataatatctcattgtggtttagaattgtatttccctgatgattagtgatgttgaacatttttttcatatacttgttggccatttttatgtcttttaagaaatgtctattcatgtcctttgcccactttttaatgggattatctgtgttttttttaatggtttgagtttcttgtatattctggatattagtctcttgtcacattaatagtttgcaaatattttctctcattcaacaggttatctcttcattatgtagatattccctttgctgtacaaaagcttttcagtttaatatagtcccatttgtctatttttatttttgttgtctgagcttttgaggtcttagccataaaaatctttgcttagaccaatgtcttgatgtgttttttttttttccagttttatatttttggtcttacgtttaagtctttaatccatcttgactatttttttaatatggtgagagagagagagatccagtttcatttttttttttttttttgcatatagatatccagttttcccagcacaaattagcctgtcctttcttcagtgtatgctcttggcacttatattgaaaattagttgtctctaaatatgtggaattacatgaattaagtggatatatatgatttaggaattgtatattaagcttatagccaggaacaaaatggaaggaacatttgtattcctaaaagacatttcatgggacacagatgaatgcctctgcaatggttctaataggaatttgaaactgcaatgagctatgtcatgccactgcactccaggctgggcaacagagcaagacctcatctcttaaagaaaaaaatacaacatttccagaatctgcatttttgacaagactgtgtgatcaagtttggtaggcactggtttgaagggaagttaatcaaataggactgtatgtaagttgaattgtagatgagagaaattatggtaaggagtcagttcagattcctttatttcctatattagtaatccacttgaagtggagaccaaagggtatataaaaagagaattcttaaataaataagtgagatttaatgactaggatttagagaatggaggagagtgctatatcaatgattatgatcaagttttggcactgggcaactgaaaacttagtggaccagtgacaagaaatcaaaaccctaggtggcataaaaagccgtggtggttattctttactcccagactactagaaattattgttaacagaaaatcagcataataataacactggagaaaatcttgaaaaaggaatttagaaaaactcataatttcaaaatcctaaaataagcagcattttttgcaagttatcttattatgtgaactttttttagattgttagtagtttaaagccagtttgtacttttaatgtttcttatacttgtttcttcagaatttaattagttttaaaggaatttttctcattataaaatacataaactctcaaattctttttgttttgttttgtttttttgaatgtaagttctcactctgtcacccagcctggagtgcagtggtgcgatcttggcttactgcagcctccacttcccaggctcaagcaatcctcctgcctcagcctctcgagtagctgggaccacaggtaccacgcccgactaatttttgtgttttttatagagaccaggtttctccatgttgccaggctgatctcaaactcctgaactcaagccatcctcctgcctccgcctcccaaagcactgggattacaggtgtgagccaccttgcctgggcatttttctttcttttttttgagtcaaattctttaaaaaaatacaaattataaataataactccagtatttatgttttagattggtgaggattaaaatacttggttctggatgggcgcggtggctcatgcctgtaatcccagcactttgggaggccgaggcgggcggatcacaaggtcaagagatcgagaccatcctagccaacataacgaaaccctgtctctactaaagaaatacaaaaattagctgggcatggtggtgtgctcctgtagtcccagctactcgggaggctgatgctgaagaactgcttgaacctgggaagtggaggttgcagtgagccgagatcacactactgcactccagcctggtgacagagcgagactccatctccaaaaaaaaaaaaaaaaaaaaaaaaaaatttggttctaataggctttggtgagactgtaagattggtgcaacttttaaaatgacaatttggcaatgtttgtcaaagctaaaaaaatgcacataccatgcttcatctagcaatttcatttctaagaatttgttctacaaatatactggctctatgtcaattaatctgtagactgactcatcaaatttaccaattgtgttggtgtattcaggaaaatttttttaaatagatatggtcaaaattacatcacttcatggaaaaatttttcagtttattctgatttcttttcaatccttttaaaagttgttttatgtatttcccccagcatcttagtatttttaggattattttgtcaacttcaaagtgcctctaccaaccttattttttggtgtttttgactaaagtttacagctatttatattggatgggatgatcataatagattttgaagatttttgatgagatttggttgactttattgattaattgattgattcagacagagtttcattctgtcacccaggctgaagtgcagtggtgcgatctctgctcactgcaacctccacctcctggggctcaagagatcctcccaccttagcctccagagtagcagggaccacaggcacgtgccaccatgcctggctaatatttgtattttttgtaaagacagggtctccccagttgcccaggccggtcttgaactcctgaacacaagtgatctgagccaccgtgcccagccctattcttataatatcatttaaaggactgttcaacttgtaagctaagggtcatggcctggttgatttttatgaatatattcaatatccagaaaaacatttcacaaccagtaattattatttcactataactgtaacatacatctatcataaatgtgtgttgaaaaataggcaactgcatcagtaagaaatgccaatattttggttcttaattgctataaaaattaagagtgataccttagttgagacattggggaataataataaaattacctaacaactagaacaataaaatatttcattcaaataagaatgtgaaatatcatcatgaaagacaaattaagaattataatgactagaataattagcaaatatgttaattagtatagtaatacatataaatatgttttcacaaattaatatttataaacagataacaccacatttgattttaataaatacatatttgtgcatgttaataagcacatatctattatattaaatataaacatatttacacacagaagtacatatagtgtattcatatatcaacataaaatatatattggcataaaatataaggattatgtgtctgagtggttcagctctttgaaataaatatgtaaatttggtaatttccatgaatttattgttcagtgaatcaagttttatagaatgagtttgtttttgttatactaatatacaggattatatgttcaagtgcgtgtgttaccagattggaaacaggagggtcagttgtgtgtttattaaattatttgtccatctgtatgatgaaatgtattgcagccatgaagaagaatgaagtagagatgcatctattaagatggaagaatatgcagtgtcacgtaaagtgaaaaagggttcagaacaatgtactcattcttttccttttcaattaaaagagggtactacaagatatattcgtgagtgtatgtttatgtatatattgggtgtgtctataagaatacacaggaattgtttacagtggtttttattaggactttaaaagatggaaacaaaagttaatgggtgtccttttattgattttaattctgatcatccagtgagtaaaagtaaaattaattttttttttttttgagacaagagtctcactcggtcacccaggctggagtgcagtggcgcaatcccagctcactgcaacctctgcctcccaggttgaggtgattctcctgcctcagcctcctgagtagctggcactatgggcatgtgccaccatgcctggctaatttttgtatttttaacggagacggggttttgccattgttggccaggctggcctcgaactcctgacctcgagtgatctgcccacctcagcgtcccaaagtgccaggaaagcaggcgtgagccacaacatctggcctaaaattaaatatttatagcatatatattcattatagaatatgtgaaaaatgacaaaatagaagaaaagtcaaccaagataactattggtaaaagtttgctttaatgtgtttcctttcagtctttttgtttatttgtttgtttttgcaacttctgtctccctggctcaagctgtcctcccacctcagcctcctgagtagctgggactacagacatgtaccattatgctcagctaattattttgtattttttgtagagatgggatttcgctgtattgcccaggctggtctggaactccagagctcaagtgatctgcccgccttggcctcccaaagtgctggtattacaggcatgagccactgtacccagccttttctttttttaagagacagaatcttgctctgttgctcaggctggagtgcagtggcacaatcatagcttactgcagcctcaatctcctgggccaagtgatcctctggctttagcctaccccgtagctaggactacaagtgcataccactatgcccagctccttttaatctttttattttgtattatgcttttactttttaaatattagtttatttaaataacttaccataaaattagtgttttatagttttcgtatactccataacatacttcctaaaataagacagggacgtaaaatgaacttccaagcctcatttttagcagtcagttaataatttcattataatgataggaaggcagtgtgattgtgtgtattaccatatcagttgtctgggctgacggctgatcttgtgggttgggtaggtgtcctcttcctttctcatggttatatatctgtctctctcccagtttcgttgaagataacagcccctactggttaggaaaatcttctcccattggacacttagatttttctcaggtttttttgatgtcatagatattgtagtgatgtatctttttactacattttttgacccagtagactgatcctccaaggatgttgtcagtccagtttagtcagttttaccaaaattaatgaattttcatcagagtattttatatgaaaaattacactattttactggtcaattttctacatagcaactttaaacttttttagacttttttccctcatttttatttttccattgttgttattgagacagggtcttactctgtcacccaggctggagtgcagtggcgtgaacatagctcactgcagccctgacttcctgggctcaagctgtcctcccacttcagactcctgagaagctgggactacgggtgtgcaccaccatgcgtggctgatttttttgtgtgtgtgtaatggcagggtcttgccatgttggcctggctggtctcaaattactgggctcaagcagtcctcctgccttggcctcccaaagtgctgggattacaggtgagagccaccttgcccgacccatttttattttactcaaattatttttactgggattattttccttttacattttagaatgaacatttactttattgaactgatttttgttcttaattttgtaaattgtagctttgtgtttgttagttttcttttgtacttctaccaaattcagtttttttaattaaaaatttttttaaccatttcactgcacactgagagctttcctacgtcaaatattgcacactaaaattctgtaatcatcaatttttagacagcactgtagtttaccaggtataattttagtcatgatatgcttaattttgttgattgactctgtccttgagtctcagaatatcttcgtttcttttgtgttttatatgtttcttcaccctgaaagtctggtcataatgttggcctcttgttctggagcctttgtgttccagtgatgagattctaaaatgtagaatttggcttgtttgtgacttatccttagctttgataaagttttattttgtagatttcatttattgttctgtcatacagataacacctttggagtacaggatggcaatttggatgtctcaaatatagatctttacactgccttaaaccaggctctagctcatttaaattgactgtaatactgactctgaagtgctaggcaaggttgaaacagcgcacatttctttaaattttttgcttaagtaaaatttctagacatgaacttactagatcagaggatattaccatcattatggtttttgcattaattttcaaaaagattgaggcaatttgtaatgtcatcagcagtgtataagcatttcaattttaatatacttcctttaaaattaggtatcagtattttaaaatgttttacctaatgaactaagagtaaaatggtcttgtgaattttctttcatttacattttcagttattttgtatagaaatttaataggaacaagaagtcagaagcagattagagacatgcaaaacttaaactcctaaccagagcatttggctgtgtagactactatgcactgcactagtgttttgaatctaaacctgtttagtccaggcgccgtggctcatgtctgtaatcctagcactttgggaggccactgtgggcggatcacttgaggtcagaggttcaagacctgcttggccaacgtgatgaaaccccatctctactaaaaataagctggatgtagtggcacccacctgtagttctagctacttgggaggctgaggcaggagaattgctcgaacccaggagatgaaggttgcagtgagctgagatcacgcctctgcactccagcctgggcgacagagcgagattctgtctcaaaaaaaaaaaaaaaaaaaaaaaaaatttgggcacagtggctcatgcctgtaatcccagcactttgggaggctgaggcaggtgcatcatgaggtcaagagttcaagaccagcctggccaagatggtgaaaccctgtctctactaaaactacaaaaattagccgggcccagtggcaggtacctgtaatctcagctactcgggaggctgaggcaggagaatcacttgaaccagggtggcagaggttgcagtgagctgagattgcaccattgcactccagcctgggcaacacagtgagactctgtctcaaaaaaaaaaaaaaaccacaaaaagacaaaaaccagttaaaaaattaagataacttattagcatgatgaaatagagcatccctggaaagaaagccataaacagtactattctactcttggatgggggagtttgttcctttgtcttatgtccttctagaatctacttttatcctgcaatttgacagtcaccatgtgtaaatgtctctttgtcacatgtgcactctcactctccctcgctttctttctccctcttaagactctcttgctcaattacccaggctggagtgcagtggcatgatctcggctcactgcggcctccttctcctggcctcaagccatcttctcaccttagcctcccaagtagctgggactacaggcgtgcaccgtcatgcctggctgatttttatattttttgtagacacaggattatgccatgttgcccaggcaggtctcaaactcgtgggctcaagggatccacctgcctcgccttccaaaatgttggggttacaggtgtgagccaccacgccaggcctatttttaaatcttttgccaaattagtaatgctttaataaatttttcagagaaataaatggaaaactgtcatgtttattagtttttcttattttttttgttttatttagccaaaaattattagggtagctccactttttccttattgcttttgaatatggtttttaaattgtaaataacttaagagatgcttggaaatgatttaaaataaggcatgttattagttcatttattagaaaataaggaatacttatataggagtttgaattaatatttttatatactttcccatttatgacactttcaggactcttattcaaacctccaagccaacagttttattaaaacattaaaactacattctaaggtacgtgaatcaatcttttcttaatgtcagtaagactataaattgtattttttttttttgagacagagtcttgctctgtcacccaggctggagtgtagtggcatgatcataacgcactgcaccctcggcctcccggattcaggcaatcctcccacctcaccctcccatgtagctgggactgtaggcatatgccaccatgcctgactaatcttttggtttttttatagagacagggtctccctgtgtttcccaggctggcctttaactacagggctcaagtgatccacccacttcaacctcccaaagtgctaggattgtaggtatgagctactgcaaccagcctttatttgcatcttttaaaagatttattttttacttttaacctttgtttagaattttgtgtaattcgtgtgttgcatatttaaacttctttgttctgtctctttaatttcctttggagccaatcattatctcttagggacaattcgaaagggttaaattctgtgagatgctatgacgtacttatggtgaggatcactttatatatatatatatatatatttttttttttttttttttttttttttgagtcggtgtctcgctctgtcgcccgggctggagtgcagtggcatgatctctgctcactgcaagctccgcgtcccaggttcacgccattctcctgcctcagccttctgagtagctgggactacaggtgcccgccaccacgcctggctaattttttgtatttttagtagagacggggtgtcactgtgttatccaggatggtctcgatctcctgacctcatgatctgcccacctcggcctcccaaagtgctgggattacaggtgtgagccaccacgcccagcctgtatatttatatatgtatttttgagatagggtcttactctgtggcccacgctggagtacagtggtgtgatctcagctcactgcaacctccacctcccaggctcaagcgctactcctgcctcagccccccaagtagctggggctacaggcacttgccaccacacctggctaatttttgtatttttagtagagacagggtttcaccatgttgcccaggcaggtctggaactcctgagctcaagtgatccgcctgccttggcctcccaaaatgctgggattacaggtgtgagccaccgcgcctggccaatatattttatatagtaaggtctccttcagctagtcagcatgccttccaactctccgctaggcataggaacactgctcaaggtgccaatatattggctagcggagtgccaagatttacgtgtataacgtaagacacttatggaataatacaagtcattgtatatacaggagctgtagagttcagagaacggaagagtgtatgggtcaatagtttactttttattatctccgaccagctgataaaagacaataattaaatacatacatatatacataaaatagataatttactttcaaggctgctattctgcctataattttatgtcttgataatcttgcttcccaaaacataattttatcagatttctttaaacttagtctttaacttgattcttttatgcatacttattaccattttagttaggaagatgctacttttaggcatgtgtcacaactcataataattgccaaatattctacagtttaatggtttttttgaggaccctcatattttataagtttataataaagtgtcttaagtttcttaaaagtatcaactgttttcgtttgtttgagtggtctctgaatgaattggcatgcttagagactttttttttttaatcagaatctcttatctctgtttccatactgtctcccagtcttctcactgaaggtttactatgtaatttagcataactgttctgagtataccagagtttagagttgcaaggtatgttctaaaagactaccttttctccctcaacccactgttgggtgcttccctccaagtcttcaccaccagccgaatagtggaatgaaattacatatagtaggcttaactgtaagggttctgcagacatggaattaagtcgagattttgttcatggcttaaaaatctgttttaagtttgaggtcaattatatacaatgaaatacataatcttaaataagtgttagatgagttttgacaaatgcatgcacctgtgtaacccacatctttatcaagatatagaacatttccatcactccagaagcttctctcatactccttcctactcaatacctgtctcccagattcaacctttgttctgattctgtcaccagagcttagttgtggctgttatggagttttatataaatggcataattttgtgactggcttcttttactgagcataatgctttccaggtttgttcatgtttcacctattagcacttcttttttattgacgagtagtatttagtattctgttgaatgagtataccacagttcatttattcatttacatgttgatggacattgaggtttttttttttttaagacagagtctagctctgttgcccactctggagtgcagtggcacaatctcagctcactgcaaccagctcactgtgcctggccaggtggacattgaggttctttctgttttggctattatgaataaaactgcttatgaacaggccaagcatggtagcttacatctgtaaatccagcactttgggaggctgaggaggagcatagcttgggcccaggagtttgagaccagcctgtgcaatacagtgacgcttcatctctacaaaaaaattaagaaactagtggggcatggggttatgtgcccgtgggtcctagctactcaggaggctgaggtgggaggattgcttgagcctgggaggtcaaggttgcagtgagccataattgcaccactgcagttgctgtgcctgggtgacaaagtgagaccctggcctccccccccaaaaaaacacaccaggctcggcacggtgtctcactcctgtaatcccagcactttgggaggctgaggagggtggatcacttgaggccaggagttcgagaccagcctaaccaatgtgacgataccccagctctactaaaaatacaaaaaattagccaggcatagtggtgcatgcctctagtcccagctactcgggaggctcaggcaggagaattgcttaaacctaggaggtggaggttgcaatgggctgagattgctccactgcactccagcctgggcaacagatcaagactccatctcaaaaacaaacaaacaaacaaaaaaacccacacatcaccaccaacaacaaaaaactgctcatgaacatttatgtataagcccttttgtggatgtatgttttcatttttcttgggttaatatatagaattggaattgctaggccatataggaaatatatgtttaaaactattttcccctggaccatagtgatagaggggaatggaggtgagaggtaagtcaaggctataccataaatgaccttatagttcatattaaggagggttttatcccaagtataaaaaaagccatggaagtgatgtaggagaataaaaccatcaaattacatttttacacctgactgctttttgtagattgagttaaaggtgaagctgaaggaggaagatccagtgaagaggatatgtcagttagaactctgttgcaaagaacataaaacttgaccaaactagcttaaacaaaaagagaatttttgtttcacataaatgagaaggtccaggaagcattgttcattctcaaatatttaaatactaaatatgtctgattccaaaatttgtatttcatcagctcttacctctcttctgaactctagatatgttcactaaagagatccttatgcttagaaaagtacctgatgtatatactagatactcaataaataatttgttgaataaacaaatgttcttgggcatccctcagagttatactgggaatcatacttgaaacctcctgctcttccgtttatcatcatcaataattatctatcctcctgacagattatttgtcaaacttgttcagttttctcaatcttcactgccattacacccctgatttctttcctacccttcttgagaaccttctgtggtcttcacatgctgcttctttctccattacagtctctaaaggttggagttactcaggccttggtcccatatcctcttctatttctttgtagccttccttggatggaaggtaaatattaaaaaaggaagataataagtataatattaagagtaaggcataagggtttgaattgattctgacatatagtgaactacatgctaatttgctgtaaagggaaagtggaaaacataccaataggtactctaagcaaaagtccaggaaggaatagatgtgtaaatttttttttttttttttttttgagacagagtcttgctcgtcacccaggctagagtggagtgcagtggcatgatcttggttcactgcaacctctgcctcctgagttcaagcgattctcatgtctcaggctcctgagtagctgggactacaggtgcccaccaccacacctggctaacttttgtatttttagtagagacagggttttgccatgttggccaggctggtcttgaactcctgacctcaggtcacccacctgccttggcctcccaaagtgttcggactacaggtgtgagccaccacgcccagctgagatgtgtgaaatttttggtagagttcttcatggcttatctgagagtggggaaatgtattgttcctttgcttctggaatctacaaattaactattcaaaatatgaccttaggcacacatcaagtgatctgtaataatattttatttatttatttatttatttttatttcattttatttttttgagacagagtctcgctctgtcgcccaggctggagtgcagtggtgccatctttgctcactgcaagctctgcctcccgggttcacgccattctcctgcctcagccccccaagtagctgggactacaggcgcctgccaccaagcctggctaatttttttgtatttttagtagagatggggtttcaccgtgttagccaggatggtctcaatcttctgacctcgtgagccgcccgtctcgagctcccaaagtgctgggattacaggcgtgagccaccacgcctggccgatctgtaatgatgttttatttttgtaaaccaataaatgactttaaaatttgtgaaagaaatgaagaagtgtgcttaaattaaaaagtgtgcttaattaaagcaccaacttccttgttttctctctgttctttcctttatttttaccattcgcacactgtgtttcctcagatattcatattttccagaagttttttatctttttttaattaaaaaaatttttggagacagggtcttgctctgtcatccaggctggagtgcagtagcatgatcatagctcattgcatcctcaaacccctgggctcgagagccttttgctccaatctcttaactagctggaactacacatgcacgtcaccatgctcagctaatttattttttatttttgtggatatggggtcttgctatattgtccaggctggttttgaactcctgacctcaagctattttttcatctcagcctcccaaagtgccgggattacaggcatgatccactgtgccaggccccagaaattttatgtctttacatcataaagtgttgactaaagaaggctgactcacagcgatggtggggaaagtagtggcagcatggaatagacagatttgcacagtagtttgcatagctatgtttagaccgtagttgataaattttgaaatcttgaagaacctgaaatttgcctgtgtaaacagagtgaaggtgacagagtgcttagtaaatttattaatcattaatatttcttccttgagcaacatcatcatcatcatgatcatcataaatgcctagtattatgtctgatacatagtatatgcctaataggatctttcgttttaaaaagagttaaacttgagtttcctggaaaatacacagatggtgaaagataaaagtaaacattactaaaaactgaaatgctctgtcctagaaatggaaatgtacagttatgcatcgcttaacaatatggctagttttgaaaaatgcatcattagattattttgttgttgtgtgaatgtcatggagtgtacttacacaaacctagatggtgcagcctactgtacacctaggctatttggtatagtctatgctcctaggctacaaacctgtatggtatgttgctgtagtgaatactgaaagcatttgtaacacaatggtatttgtgtattatacatatctaaacatgaaaaaggtatagtaaaaatatggtataaaagattaagaaatggtacacctgtttagggcattcaccatgaatggagcttgtaggactggaagttgctctggatgagttggtgagtaagtgttgagggaatacgaaggcctaggacattactgtacactactgtagacattgtaaacactgtatagttaggctacactaaatttatattaaaaattttcttcaataattaaccttagcttgctgctttaacatttttatattataaactttttttttttttttttgagacagtcttgctgtattgcccaggctggatggagtgcagtggtgtgatcttggctcactgcaacctctgccttttgggttcaggcgattctcatacctcagcctcctgagtagctgggattacaggtgcatgccaccacgcctggctaatttttgtatttttagtagagacatgttgttggacaggttgttctcaaactcctgatctcacatgatccgcccacctcagactgccaaagtgctgggattacaggcatgagccaccatacatggtctatattataaacttcttaatttaaaaaaaattttaacttttataataacacagcttaaaacacacattgtacatatttacacaaatttttcttcatagtgttactctgtaagctatgtctattttaatttttaatttttatttattattatttgagatggagtctcgctttgttgcccaggctggagtgcagtggtacagtctcaactcactgcaacctctgcctcccaggttcaagcgattctcctgcctcagcctccagagtagctgggattacaggcacttggccaccacgcccggctaatttttgtagttttagtagagctggggtttcaccgtgttggtcaggctggtctcgaactcctgacctcaagtgatccacctgcctcggcctcccaaagtgctgggattacaggcatgagccaccacacctggccttattttaattaaaaaaaaaaattttttttttttgttaaacacacacacatgcttacattcgcccacacctaaacagggtcaggatcatcaccattgtcttccacctccatatcttgtcccactgggacatggaactggtcatctctgataacagtggccttcttctggaatattttcttaaggacccgcctgaggctgcttttccggttaactttttttcataagtagaaaggagtaaactctaaaataatgataaaaagtgtagtgtagtaaatacataaaccagtagcatagtttatcatcaagtattacatacttcaggcatccattggagatcttggaatgtattccccttggataagaggggacttgtattgaataattataaattactgatctctgccataaatacaaaaatgtgggcgtgtgttgtgggagtagcagtcagcgtgtatatacacatgtctggtgtgggtgaagattccaaggggagatccaatctaacagtaatagaaaaaaatattccattaaaatgggtgtgtgcttggatttttgtacatttatataaatataaacatgtaaatgaattaaacagtgaactctgtccttttatttaaaattaatttattaaaaccagagagcagcagcttaggaaatacaccagatttattaaaaatctactttaatagcccagctgtcttccaaattatctgtgataatttagtgtatcaacagcaatctgatgtataaaataatagtatgtatacaaaaaagtactctaattcagattctcttccaattcaaattagtagctttaatctgtgtttcttgtaccttaggaaaaactcatttgtgcaatatctttagatttttctggtcattactaaacactaaaagccatttattaacagtaaatatcaaaacattgggctttattaaataacagttttcttgagaatggtgacagacttaaattttagaattataaaattattttattttttatttttaaattttattttaaaatttaatagtattatagaatgttgattagcattgtattttattcctattgtaggttatcacagtttttttaaaaacttttaggtttgggggcacatgataaggtttgttacataggtaaacacatgtcacatcacccagatattaagcccagtacccaacagttatcttttctgctcctctccctccttccaccctccccacttccccctcaagtagagccatgtctgttgtttccttttttgtattcataagttttttatcatttagctcacatttataagtgagaacatgcagtatttggctgtctgttcctgcattagtttgctaagaataatagcctccagctccatccatattcctgcaaaagacatgatcttgttctttttttatggctacatagtattccatggtgtatatgtaccacattttctttaatctgtcattgatgggcatttaggttgattccacgtgtttgctgttgtgaatagcactgcagtgaacattcgtgtgcatgtgactttatggtagaatgatttatattcctctaggtatatactcagtaatgggattgctggtttgaatggtagttctgcttttggctctgaggaattgccatactgctttccacagtggttgaactaatttacattaccaccagcaatataagtgttcccttttcttcgtaaccttgccaccatctgttttttttttttttttttttttgactttttaataatagccatcctgattggtgtgagatgatacctcattgtggttttgatttgcatttctctaatgatcaatgatattgagggttttttttcatatgcttcttggtggcatatatgtcttctttgagaagtgtctgttcttgtcctttgccctctttttaatgagattgtttgtttttctcttgtaaatttgtttgttccttataaatgctggatattagacctttgtcagatgcatagtttccaaatattttctcccattctgtaggttgtctgtttattctgttgatagtttcttttgctgtgcagaagctcttaagtttaattagatcccacttgtcaatttttgcttttgttgcagttgcttttggtgtctttgtcatgaaatctttgccggttcctacatccaggatggtatttgtctaggttgtcttccacggttggttggttggttggttttttttttttttttgggtggggggatgaagtctcactgtcgtcccccaggctggagtgcaatggcgcgatctcggctcactgcaacctccacagcctgggttcaagcgattctcctgcctcagccccccgaatagccgagattacaggcgcctgccacaacacccagctagtttttgtatttttaatagagacagggtttcagcatgttggccagactggtctcgaactcctgacctcaggtgatctactcgcctcggcctcccaaagtgctgggattacaggcatgagccaccgtgcccggtcttccagggtttttatagttttgggttttacatttaagtctttaatccatcttcagttagtttttgtgcatggtataaggtagcggtccagcattgttttacctgacttcaaactatactacaaggctgcagtaaccaaaacagcatgacactggtacaaaaacagggacatagaccaatggaacagaatagagggcccagaaataaggctgcacatctacggctgtctgttctttgacaaagctgacaaaaacaagcagtggggaaaagattgcctaaccaataaatggtgctgggataactggctagccatatgcagaagattgaaggttatcacattatttaatccctttaaaatccgttgcttaactgcaagaggtttactgtaatcattttacatttttagagttttagaatggggagggatcttagaggtttatctaatacaacctcttcattttactgaccgaagctcaggtctatagaaacctatgacttgaccaacttctcaaaactcgttattttaagaatccaagactgtaacctagatctgctatacacaccttattttctaatgctttctaatttgaatctaattgaaatacagaatgtgcttaatatttgcatgttaagaggtcttcattagtaagaaaatcttcagaagctgtaatggacaaaagtataaggaagtattaggaaggaatcgagtttcctgtgagagtaagggtctttgtgacatgcggttgataactctcctccagtcttttattatttcattcctttcatccctttttttctcctggatccctcctatccccttttctcttagagtgcagatgggatgactgtaaagtggaccttttagagtagaccagaataaggagattggatcgagggagatggggaccagaactggtaggttaattatattttgtaacttcccaagctggttctaatgttccagcacagttactggaaaccaaaagaactaaaaacatagatttttttttttttagaagcactccacactctgctgaagttggtaatattataaaagttgtagaattctctgttagtcttccaacctacacctgtcaaatctaccttctttgttttctcttttaaaaattttccttttttttcaccttttttccttctgttcaaacattgtttgcttcaagtgatttttttttataagggaataggaaatataaagtgaatatactcttaagacggatattctttcttttaataaatctctggactgtcgcagttcttatccttttttaaagagaagagtttgctgaagtggttaaagatattttactgaaatttcatcttggtaaaaaaaagacagttaatttttaatattattttattatatttgctctgcttatttcctttttctattttattttatttttttgggacgggatctcgctctgtcgcccaggctggggtgtagtggtgcgatctcggcttactgcagcctccacctcccaggttcaagcaattctcccaccttagcctcctgaatagccataattataggggtgcaccaccacgcctggctaatttttgcatttttagtagagacggggttttgccatgttggccaggctggtcttgaactcctgacctcagataatccacctgcctcggcctcccaaagtgtttggattacaggcgtgagccactgcgcgcggcctctgcttgttatttccaaagatttaataggtaaagcagggacaggctgtgaactgagataaggacaattcgatgatgatatttattcctgtagctgtgaggcattatgtagccacagctgggtgtacagggtttgttaggagcagcctttcagggagtatcttctgaatatttgataaaagtagttgataagttatgaacattagcaaaaccatttattctaataagaatctttaaaattgcttatttcttttttgcacactagacttcataaaaatttaaattataaattttgattagtctgtgtgggggtgagaattaaaagaggatatatgaaatttatttcttaaatgtagctaagaatcattacactagactttcaaaaaaaaatgtgactggcacacaccacctaaaattgtggggtaattttctgctttcacagatctttgtagaatgtgatctaaaccaggtgttggcaaactatgccctgcaggccccatcctattcatggactgttttttgtaaagtccatgagctaaaaatgctttttacatttttacaggtttgtaaaaacaaaacaaagaatatagaacagagattgtatgtgcctatgtagcctgaaatacctacaatctggcccttgcagaaaaaaatttgttgaatcctgaagaaagctctacatggtcacatagaaccgtttcaggcaattcagaattacaggcgtaagttgccttattgtgtactgctttaataagtgcttcatagatactgcttttttttcaaattgaaggtttgtggcaaccctgagtcaagcaagtctatcagtggcatttttcaacagcatatacttgcttcatgtttcggtgtcacatttgggtaattctcacaatatttcacactttttaatgattattatgtctgttacggtgatctctggtcagtgatctttgatgttactattatagctgttttgagatgccgggaactgcacctacgtaaaatgatgaacttaattaatacatgttgtatgtgttctgactgctctggtgactggctattgctctgtctgtctccctctgggtcctcgttactccctgaaacacaatattgaaattaggtcattttctaagtgttcaaatgaaagaaagaatcacatgtctctcactttaaaccaaaagctagaaatgattaagttcagtgaagaaggcatgtcaaaagcctcatgagataggccaaaaagtagggcttttgcaccaaaaaattagccaagttctgaatgcaaaggaaaacttgagggaaattgaaagtgctactccagtgaacacatgaatgataagaaagcgagacagccttattgtggacatggagaaagtttgagtcgtctgcctagaagatgaaaccagtcacataatttccttaaaccacaacctaatccagagcaaagctctaactcacatccagagcaaggctctctcttccattgtatgaaagctgagagaagtgagaaagctacagaagaaaactttgcaactagctgagattggctcatgatgtttaaggaaggaagccatttccataacataaaagtgctaggtgaaacaccaagtgctgatgtagaaactgtagtcagttgtccggaagatctagctaagatcatggatgaagatggctacactaaacagattttcaatgtaggtgaaacagccttctaggactttgattactagaaaggagaagtcagtgcctggcttcaaggcttcaaagcttcaaaggataggctgacttttattaggggttaatgccgctggagacttcaagttgcagtcagtgctcatggactattctgaaaaccacagggcccttaggaattatgctaaatctaccctgcctatgctctgtaaatggaacaaccattttatatgagggactttatgatcttctgatttttgtatccataggcgaggaggtcctggagcaagtcctccaaggatactgacagatgattatatttcatgttatataccacagcttgtaaaagtacatccttagaatagagaaaccatttgcctaatagtcaaaagatttcttcagggagaagtataagagaaaaagatttgaaagaggtctaggatttagacttaacgatcaattgtgaggtttccttgttggtttagatcagagcagaggttggtaaactgcgtcccactgcagaaatccttcctgctgcttgtttttgtgaggcatgagagaatggtaggaagcctggctgacagcctatctgtttacagcatggtttagggaacgttaagcccgctgttgagacctactgctcagaaaaaaagattcctttcaaactgtccctgtgcattgccctggtgacctgagagctttgatggagatgtacaaggagattaatgttgttttcatgcctgctaacacaacatctattctgcagtccgtggatccaggagtaattttgagtttcacgtaatcttatttaagaaatacaatgttcataaggctatagttgctgtagatagtgattcctccaatggatctgggcaaaataaattgaaaaccttctggaaaggattcatcattctagaagccattaataacatccctgattgatgggaggaggtcaaaataccaacattagcaggggtttggaagaagttgatttcatctctcatggatgactttgagaggtttaagacttcatcgtgggaagtaactgcagatatggtagaaatagcaagatgactagaattagaagtacagcctgaagatgtgactgaattgctgcaatctcatgatacaatttgaacagataaggagatgcctcataagaatgagcaaagaaagtggtttttagagatgggaccttctcctgaagatgctgtgaatattgttgaaatgacaacaaaggatttagaatatttaataaacttatttgacaaagcagtgacagggttcgagaggattgcctccaattttgaaagaggttctacgtgggtaaaatgctatcaaacagtatcacatgctacagagaaatctttcgtgaaagagagagtcaatggctgtggcaaactttattgttgtcttattttaagaaattgctacagctactccatcttttggcaacgaccaccctgatcaatcagcagccatcaacatcaaggcaagaccctccaccagcaaaaagactgtgactgtctgaaggctcagatgatcttatcattttttagcagtaaagtatttttaaattaaggtgtatacattgttttctagatataatgctattgcatatgtgatatactacagtatagtgtaaacatacctttaatatgcaccacaaaaccaaaaaatttgtgtgacttgctttatagtggtaattgcaggtgcctggaactgaatctacaatatcttctgggtatgcctgtgtaggtgaaataacataaatgaggaaaatcaacacacatggagtgtttgttacaatgttgtttacagtagcaaaaactttgaaacaaatgttcagtaacaggagactggttaaataaattataggacatctgtgtagtagaatataatattcattgagagtcgtaacctatgatttacacaaaggagaaatgttgattgcattaagagtgggggagaccaaattccagaactgtatgcagtaagatcctttacaaaacacacacatgcacatacatatgctcttgatatctgtataggtacagaaagttttatatgctatagagtccttggtgacttagatattttttctaattgttgcttttatgtcattgctaattgtattacaatgaagttaaataatttccctttgggtatggaaaaattaagtctgaatagagttttaagtcacaaagagaacagatattttatgtaaaatagtattagaagcgtggaaacgccattgaaacttacaaatttttggaaaaattaaaatagaacttatctcaaaatcctattaatttattattcttaaagtttaaataggctgagcatgtaataatgatagttagttgtccaaagaaaatagtactattttaaggtatatcccttacaatgcgagatactgtgtatcatagagaatgcttctttccacaaaagcatgcattggtgttactttcagatacagattttgacttggccaaatcataaaattaaacctcaaataacatttcttccttccatttctctttccctttgtctccttcccttcccttcctttcctttcactgtgtattattgaagtacagttagataatactgaccttttacaactgtatgctccctgttgttgccaacatccagtcttctttcttttctctctctagtattttgtggtttaatgtaaatgtgttgcacatctcattaaatttattccttgatatttgatgttttatttgatgctgtttaagtgttattacaattttactttgtttccattgcatgttgctagtattttgaaatacagtggatttttatatattggttttatagccagtgatcttgctgaattagtagttatagtagttttctgttattttggggtttctatgtacaaaatcttatcatctgcaaataaagactagttttacttcttttcccaccttacacagtagtcctcccttatccatgggtgatgcgttccaagacccccagtgattgcctgaaaccgcagatagtactgaaccctttatatacagttggccttctgtatctgtggattcaactacccatggatggaaaattttcggggatggggtggagacaacaaaaaatataaataaaaaaattatgtcacagtaactgtttatatagcatttaccgagttataagtaatctagacatgatttaaaataaatgggaggattgcatatgtaatatgcaaatagtaaaccattttatataagggacttgagcttcctctgatttttgtatccatagaaggtggggaggtcctggaacaagtcctccaatgataccgacggatgattatatttcatattatataccacagcttgtaaaagtacatccttagaatagagagaaaccatttgcctaatatgtagtcaaaagatttcttcaggaagaagtataagataaaaagatttgaaagaggtctaggatttagacttatgatcagttgtgaggtttcctcgttgatttagatcagagcagaggttggtaaactgcatcccactgcagaaatccttcctgctgcttgtttttgtgaggcatgagagaatggtagaaaaaataattcaaaaaaatcagtttgttgtaatatgagaaaattaactgaaattcagttttcaatgtccgtagatagttttattggaatacagccacacttactcatttgtgtatagcctgtgactgcctttgcatttaagcagtagagtaattgtaatgtttgaatggccagtaaagcataagatatttactgtttagcccttcaacaaaaatgtttcctaatgtctagattctcttctgttttcgtcaatgttacatttttaaatgggggttttactttctaagatggcagtggtgtaagtgagtttccatcccacaattaagtaaacaaaatattctgttctcagatattaacacgcttatatgttttagtcttttttgtttgtttgtttttttgagacagtcttgctctgttgccccaggctggagtgcagtggtgcaatcttggctcactgcaacttccacctcacaggttcacctcgtgcctcagcctctcaaataattgggactacaggcatgcaccaccatgccccagctaattttttatttttagtagagatagggtttcagcatgttggccaggctggtcttgagctcctggcctcaagtaatctgcccgcctcagcctccaaaagtgctgggattacaggcatgagccacagtgcctggccttagtcttttttttttttaaacctgtgggcttaaacttcctgaagacacctgaagtatgtatttatttaaatagaccacttttgttgcaaaataaattcctttgtccatggttggtagacttttattttgaaattgttttttaaatgtgtatagaatatatttatgtgcaatttcaagattagtaaaactagcacccatatatgtattgaccattattttctatattcaaagcacagcctttgagttggttgaattggttggcattgctatatgaatttaaagctttttttttcgccacatagtttttttttttttaggagaacaggattttgctctcttcccgaggctggagtgcactggtatgatcctaggtcactgcagcctggaaatccttggctcaagtgatccttccacctcagcctcccaagtagctgggactacagacacacaccactgtgcctggctattctttctttctcttcctgtcctgtcctgtccttttttcacacagggcctcgctttgtagtccaggctggtttcctagcttcaagtgatcctcttgcctcagcctcccaaagtgctgggattactagtgtgagccactgtggctgaccaatttgttttccacaattgtaattgacctagtgttgtgtatatttggtcctttacgtttacagaatgttttgttttgttctattttattttattttgagacgctgtcactctgtcacacaggctggagtgaagtggcatgatctcggctcactacaacctcctcttcccaggctcaagtgattcttgtgcctcagccccctgggactacaggcacacgccaccatacccagctcattattgtactttttgtagagatggagtttcaccacgttgcccaggctggtctcaagctcctttttgtttttacttatttactttttttattttatttttttgagacaggcttttactcttggctcactgcaacttccacttcccaggctcaagtgattcttctgcctcaccctcctgtgtagctgggaccacaggtgcgtaccaccgcacctggctattttttttttaattttttaattaattttttatttttatagagatagggtttcgccatattgcccaggctggtctttaactcttgagctcaagcgatccacctgccttggcctcccagagtgctgagattacaggaatgagttgctgtgcccggccttgttttattcttgaaaacattctgacatatacattctctgtgagactttctgtagtaatactaatttggttaacattttctattggaaacaggaatgaactagttgtatgtggaccagaggactcttgactttactattttagttttatggtgtaaaacggttaccattctaggctttactgagtcataatagaagtgaggcacacatggagattagagaggaacaaatgttaaaacactaaattttttcttccttttaaagaagaattaatagctatatttctgtattttagaccccttaaaagtatttattatttagttttattccagatggctgcatggtagcatatctcagaaatcaaatatatatatatatatatatatatatgtatatatattttttatagagatagggtttcgccatattgcccaggctggtcttgaactcctgagctcaagcgatccacctgccttggcctcccaaagtgctgggattataggcatgagctgctgtaccagctatatatattatatatatatatatatatatataaaatatatatatttattatatatatttatatataaaaatatttttgtatccatatatatgttatattttatatatatataatataacatatatatataaaacaattccaggaaaatactcatacataagcttgattgttttatttttgtttaaattttcaaaaatcattttctttcatttaaaaatcattatttacccagatacctgttatctgccctcctttatgaattctgtcttactgtatgcttcctccatgtccccttatgccctacctagcccaaaacactgaaaggttggacttccatatgaatcaatgctttccatgaagtaaaactgcagaagtggtggcaggatattcctgaatgaagtttgagacagatacactggcagagatctcttggtgggcaaggagcataagattagtagcatgccatctggggttagaatttaggaggtggtagaatgtaagtgactagctgacagtttggctaaagaagtggttgcatttaaccaggctaataaacatgagcttaaaaaaaaattcactggctaagactgggtcttgcacttcattttcagtttagtgtttctccttggattaattttttctgttttatccattactagtggtatactgtaaattgatgtacagaagtatctctaaaaagactaatttataggagtccagacggactagaagggttttaagcttataatacctggatccttttgttttaattaggtttctagataagtccttacaaacttcaaaggtgttataccctatcagatactttttcccccaaataaaactgtatttttcagttgaaaataaattgatataggcaattccttttcttgacaaccttcacaagtgtggtagagaattatttagcacactaattagtctcagcttcgattttgtgcagatttgattttacctattaataacactttcatcgggcttagcctttaattttataaactcctggcctcctcctgagcattaaattgataggaaacatactgatgctaatctctaattatacattcagaagttttttcatctcatcactctattgtccctttttatttgtccgtcatacttaaattttcaactaatttaatatagtttttctcattatacaagtaaacgttcatttctaaagattgaaaaataaaaggaagaaaacgaactacctctagccctctactcagtgacagacactgctattttcttttctttttcttttcttgagatggagtttcactcttgttgcccaggctggaatgcagtgatacgacctcagctcactgcaacctccgcctcccaggttcatgtggttctcctgcctcagcctctcaagtagctgggattacaagtgtgtgccaccacgcttggctaatttttgtatttttagtagaaacagggtttcgccatgttggccaggctggtctcgaactcctgacctcaggtgatccacctgccttggcctcccaaagttctgggacaggcgtgagccaccatgcctgtccctattttcttacatgtcctttgatacactcttttattactattcctcttttagagacaggttcttactctgtcacccaggctatggtgtagtgacgtgatcatagctcactgcagccttgaactcctgggctcaagcgatccttctgcctcagcctccgcagtagctggcactacaggtgtgctcctccatgcccagctattttttgtactgatagggtctcgctatgttgcccaggctggtcttgaactcctagcatcaggtgagcctccctcctcagcctcacaaagtgctgggattacagctgcgagccactacacacaacctgaatcactgttttatttgtctctctgccatcctccatatacagttttgggagcatataatacttgcacatgtctatggtacagtgttgaataacataggcacagtctttgcactcaaaggattcaaagtttaatactctttaaaaagttgccttttaaaaattaattgctattatagaagaaatacataaatagtctcttgcaaaaacacatagataaggttcagctttctgattgctcccattcccttctttattttcctaaaggtagtcactattagcagtttatatgtcatttttccccctaagggtttgcttatactatacatatgtagagaacatgtatgatttcttttttagtgtaaaaacagttggctgggtgtggtagctcatacctgtaattccagcactttgggaggtcgaggcgggcagatcacttgagtccaggagttcgagaccaccctgtccaatgtatgaaaccccatttctactaaaaatacaaaaattagccaggtgtggtggtgcatgcctgtaatcccagctactaggaaatctgagggatgagaattgcttgaactcgggaggtggatgctgcaatgagccaagatcatgccatgcattccagcctggggtacagaatgagaatctctcgaacaaaaacaagacaaaacaaaacacaactgttatacagtacctgcaattagttttcttttcattcagtatgttagtatgtacagatttagttctttaaaactgctttatttaattttactgatacacagtaacttagcctttcctattgatagacattgggagtcccaattttttgctattgcaaaacattcctgcaattgcttctactcatgcagatgtgcaactgtttctctaaggctgttatgcagaggtggggttagtagatcatagaggattatgtgattacccttgaaattttaccattcatgttactctttttttaccctctttttctggagggtcttatttttcttttttgagtcagtctcactttgtcacctaggctggagtgcagtggcgtgatctcggctcactgcaacctctgcctcctgggttcaagtgattcttaatgccttgacctcccgaccattcatgttactctttaagttggaccttagagcagtttatcttatatcactcccctcccaatagacattttatcctttttaaatctaacaaattagacagacattgttgttattttatagagaaatgtgtgattcagtttacgcacatttttaccacatctttccttaccatgccttcaatttctgactgtagctgggcacagcggctcacgcctgtaatcacagcactttaggaggctgaggcaggtggatcacttgaggccaggagtttgagaacagcctattcaacatggtgaaaccccgtctgtaccaaaaatacaaaaattagccaggcgtggtggtgtgcacctgcctggtaatctgggtctaaggattacttttaaccagaatatcagctactcaggaggctgaggaaggagaatcgcttgaacccaggaggcgaaggttgcagtgagccgagatcacgccactgcactccagcctgggtgacagaggagtccctgacacaaacaaacaaacagaaaaaaaaatttctgactgtcctcctggattattttcctccttcttaaaaaacgtcttcagaagtttcattagtggaggtcttttagtggtaaattgccagtttttgtttatttaatttttaatttagcctcatttctctttttgagtcagggtcttactatgttgcccaggctggccttgaactcctgggcttaagcgattctcctgcctcagtttcccaagtagctgggattacacgtgttggccactgtgcctgactatttttagctgtttgtcatgaaaattttttaagcatacatgaaagtagaacaaatgctatgatgaaatccagtatttccattcctcagtttcctgttattgatactttataatttcatgtttaaagaggggtctctttacctctctgtgccttccttcctgctggaatatccaatcatgaagtctgtcaatactttagaatgtatctcctagagaagaggttaaaaaaaaaaaaacccacaatattctcattcctaactaaattagcagtaattccttaacatcatctaacacctagtccatgtcaaagtcttttcagttgtctttgaaatgtctttttataattttactctcattttttcagggttgttatttcctaggtgctcagttgtaggttgacagtaattttctcttcatatcttgaagatacattactgccttctggcttccttgctgttacttgctgttactctaattattccattgggtgaaggattctttctttatggcttcctttatggttcttgttgtctttagtgttgtgcaattttactactttgtatctggatgtgggcttattgatcacatttgggtatgttgtactttctgggtctagggattacttttaaccaattcttggaaatttgcagctagtgtctctttcttttaaaactttgaaaaaggaaatatacattcagaaacatataaaacaggtacagtttcgttataaagtagatgtctttgtagactcttccgaggtcaaaagcagaatatttcacgtacctcagaatccccctgccccatacaccccttgccatttgaagtcgtctccctttccttagaggtagtcactatcatgacttttatcacctttatgtgcatccctaaaatatagtttcctttgttgaaatttatatgaccagcatcgtatactttgtcttgctgtttttgctcaaattatgtttgggagattcatctgtgttacctgtagttcttcatttttatacagtcatcattacttaagatagtttttctttccctttccctctctctcctgtctgtttactactcccattatgtgtatgttcatgtgcttattgatgtttcatatttctctgaggctttgtatttttttcactcttttttttcctctctattctttggattgcatcatctctatcaatctgtcttcaagtttgccagttcttttttctgccagttcaaatgtacttttgagctcctctagtgattttttctagtcagtgttcaactccagaatttccatttggttctttttaataatgtctctttatcgatattgtccatttgctgagatactgtcattataccttccttctgcaattatggtatcctttagttctttggacatatttataatggcattttgaagttggttaaatccatcatctggttgctcttgcaggtagtgtcctttgcctttatttttctctggtatatgggtccttccttccttccttccttccctccctccctccctccctttttttttctttttttgacagagttgctctgtcacccaggctgattctctgctcaccgcaacctctgtctcctgggttcaaggagttctcctgcctcagcctccctagtagctgggattacaggtgcccgccaccacgcctggctaattttttgtatttttagtggagatggagtttcaccatgttggccaggctggtctcgaactcctgaccttgtgatccactcatctcggcctcccaaagtgctgggattacaggcgtgagccaccacactcagcactttcctatttctttacatggctcgtaattttatgttggaaactgaacatcttgtataacattgcaacaactctgggtactgatctttcaaccccctgctcgtttccagggcttgttacaattatttgcttatttgtttagtgacttctgttgccctgtttcagtgatttctgtttccccccttgcagtgtttaagcctctggtgttgctccttaggggcacagacttgtgtatgaccacatggcacctggcagtgttgggctgacagtgtttggcagagctttcttctgtttctttttctgaccacacccagctttttatctccactgatttctggctgactactctattgtctttaacaatactctaggacataaattgcttcttagccagatccatacgactctcgaggtcactgcatgtacaatttgttcagacccagaggggcgcatcccagctgtcttattccctggttctcacctgcaaactagctggcctacagtttagcctgtattttgaatctcttccaaaatgcctttcactcaaccttgggtttgatattgcttttaggcttgaacgtcacactcttgcaaatgaagtcagttccattgggaagagattcccagctatctctggggacatgttaaattatatgagatgctttgtatgctgcttttgatttacatcttagaaaagtaaaatacaaatattttactacataaatagcaaagtgatagagaggtagaaagcagccctacatagttgatttttttgtataacagcagtttaacctttaatgagagaggcttttagaagttgtcaggtagatagatttctaaaacataagtaagcttttcagcttcctaaaggtaatacagtcctgagttctatagtatatgaatattcatggtagatatacattggtactttggcacctaatttaaaaggggaaattaagtattagtgctgatatttaaactttttcacttagcacacagttcatagctactctttagtctgttttttgtcttcgtgtagtttatgtaaataacatgctttgtgaaatgtgtctaatacagaaacaatggacaacttaaagtgctgtttcttatttttgaaggttgtcatttataacagacatataacaatttattctttcttttacgtattcatcaaatgtgtgaatgcttagtatttactggaagccatgttgtagctgcatggtataccgtcataaatgtgatgcaccgggtcatttctgccctcctgaagtttccattctagtgggaacagactaattgtgaagtcctgaagtttccattctagtgggaacagactaattataaaaatatatagtttataattttatgtatatattgcatgtatggtttttatggaaactttttttttctttttctttttttaatctttttcactttttgatttctagctagaaccacctccagaatttatggaaacttttaagatgctatacaataccaaagtgtgcatgtgtgtgtgtgtctgtgtgtgtgtgtgtctgtgtgtgtatttcatatctataaatagctttcaattatgtataattacagtgtatatataactttggttgtatagtatatacttaggagtattatttctgtgtaactgtattcagtggagttgcactagctacatatttaagttattaaaaattcatctattagattggcaaaaaattaaagggttaattaaatatagtaatcatttcaaaattttataaaagtactgttatgtactatagttaagtgtatacagcatatttttctttttttgacttttttattgtggcaaaatatatgtaacatatttaccattttaaccattttcaaatgtacagttcagtggcattaagtacattcacattgttatttaactatcaccactgtctgtttccagaaattttttcagcatctcaaacagaaactctgttaaattaagtaataactccccattcttctcccccagccccctagtaacctttattccactttctgtatctttgaatttgtctattctagatacctcataaaagtgagatcaggctggtcatggtggctcatgcctgtaatcccagcactttgggagcctgaggtgggaggatcacttgagcccggagttcaagaccagcctttgcaacacagggagaccccatctatatagatatttaaaaaattagctgggtgtggtggtgcacacctgtggttccagctactcaggtggctgaggtgggaggatttgctttataaagcccagcattttgaggctgcagtgagctatgatcccaccactcactgcactgtagcctgggtgagagagtgagactgtgtctcaaaaacaagaaaccaaaagtgaaaacataaaatatttgtcctcttgttactggcttatttcacttaaagcatagtgttttcagggttcattcatgttgtagcatgtatcagaatttgattccttttgtgactgaatatagtctttttttattattatacaacattttatttaaaaaaattattttcatagaatacattttcacattagagattcccattgtgcgaaaataacaatttattacttatagttttatatttgtggacagattgttttagaacaagtagaacacatttgagaaataaatctaagtttacaattggtaatattttgatacgtctacaaggggaaacttgcccttaaatggaacttctctatattcagaagcactccaagcatttctccctaggatttagaaatttataatgtgagatatcagcacttcctaattttaaaatttccctggtatatgtaaccatcagtaggtggtatctactgactagagagggaagttttcgaaaattaatcactgtctaattttctacaaagtttttattcatgaattaagagtatttccctttgtccattattcccagggcaaatatggaagtttgatcatatactaatagtaataaagctggattctctttaagagattgataaattaaaaggcaaaagctcatatatcatgtttagctatactgtgagtcttataagaagctgggaggcaaccccattaactcaccagaatacagaactcagtctcacaatttcttagatatagttcctctcaaaccttttcctcaaagattaaattctgaaaataaccttgtgattaagagaagaaggctgtccaccaacagacttatctgttatttcttccttattgtgagcttaatggcatgacaaagcagaggcaaagaggcttacatcagttcttcaaagtatgaagtcaaaaaggtcagagcttccacagcatggcaacagctttgcagatgcccacatcgtgatagttgaaatagcaaagcccagcaaaggttaaagctgaaagtgccaaaagccctgccttggcagctttctgcgaggcatccccatgaacatagtcagtaacaacttgtccaaggccccagtgaccatgaagagtgagggtcgcagccagggaatagcccatcgcagagcaaggattcaaacaagcagccggaagcagacccaggagcaaaacactgaaaaccttcttgctagtccagtggagagatgcagccttggaaccagaatggtggctcggtgacaagtgtatgtgctgtactccacatcattctgggataggtcggtcctgaagaaatgctgagatataagcaggtctgaccactggcgttcacagcaacagagctcgacctccttgggcactgcaaagggcactcagcctccagagaaccgccatctcgttcctgaggatgactgaatatattctaatattccatggtatggatataccacattttgtgtatgcctccattgatggacacttggattactttccattttttggctattgtcaaaaatactgttaggaacactggtgcacaagtatctgttcaagtcctttcagttctttgaatatacacctaggagtgaaattgctgggtcatacactaattctaagtttaataaccaccaaactattttctgtagtgtctgtactaccttacattctcgtgtattattattattattattattattattattttttgaaacagggtttcactgtctcccccaggctggagtgcagcagtgccacatcacggctcactgcagcctctccctcctggctcaggtgatcctcctacctcggcctcctgagtagctaagactacaggtgtgcactaccacacccggctcatttttgtagagatggggtttcatcatgttgcccaggctggtctcgaacccctgggctcaaacgatttgcctgcctcagcctcccaaagtgctaggactgtgagtgtgagccattgtgtccagcctctcatgtattcttacacatatgttttgaacaggaccctaaacacaaaaccatgtatacagcaacactttataagtgataagacactttcaaacataattttgactgtatgatttttattttgtgtacttactacatggtatgtgagatttttggtgatgcaggagcattattacacatgtcacagttactagttacagttgatgtagagaacaagaactcaagtaataaggtaaaaatttgccatagagtttaactttttaaaaagtaatgtcgttttattctttgggcatttgcactccacttccaagtatacatagctccaacatcagtaatcgtgacttctgtcttaccatgttggaaagggcgtgagttttgaagttgggtcctgtttttgaattgtatctttaacactctagcttcagtttcatattttataaaggatatagtgtgttcttaccaagtcactgtgaggattggatcatgtatatgaaaaggcttttttttttttttgtgacaggatcttgccctgttgcccaggctggagtgcagtggtgtgatctaggctcactgaaacctctgcttcctggactccagcaatcttcccaccccagcctcccaagtagctgggactacaggcatgtgccaccaacccagctaatttttttttttttttttggtagagatagggtttcgccatggtgcccaggcaggtcttgaacttttggcctcaagagatccatctgccttggcctccaaaagtgctggggttacaggtgtgagccagcgtgcctgcagaattgttgttgagacagggtttcactctatcacccaggctagagtacagtggcgtgaacatggctcactacagccgcgacctcctttggtcaagcaatcctcctgcctcagtctcccaagtagctggaaccacaggcacacaacaccatacttggctaattttaaaaatttttcatagagacaaggtctccttgtgttgcccaggctggtcttgaactcctggcctcaagtagtcctgctgccttggccacccaaggtgctgggattacaggtgtaagccactgtttatggctaaaaatgaatattttttttccctgccaaattaattctaaggactcctgcaccccactcaggaaatcattggcttttcttgcccttttcatttaaaaagatatttctatgtcttgtaacattttaaatcttattatttgaatgcattgttttattttattttacttatttatttttgaaacgaagtttttgctcttgttgcccaggctgcagtgcagtggcacaatctcggctcactgcaacctccgcctcctgtgttcaagcaattctcctttttcagcctcccaaatagctgggattacaggcatgcaccaacacgctcggctaatttttttgtatttttagtagagacggggtttcaccatgttggccaggctggtctcgaactcctgacctcaggtgatccgcccgccttggcctcccaaagtgctgggatttacaggcatgagccaccgcgccgccctgtttagttttaattttaatattagttgtctggcttattaagtaaaattattctctaaatgaaaatttgggcaagggtattttggagagtcgatttcaaagcttattttaaaagcttagttcagaggggtactataatacatttaggaaattgctctgtacttgaaattccctaaaagtttgtgtttcttcattgttcagatgagtacatcaaattcttttggatttcttatccaagatcatatagttactttgtgaaagagatgagtcagtcaactctttgaccttgtattgcagtgttctaccacatcagactaccctcctcctaccttttaccatttatgagattacttttggcagctggcttctgtggcttctctccatggtgctagaggtgcacaaatgtacttacttttgtattaactatctaaaggatttctttctcctttgtcccttttttgttcctctaaacctccccctaaactgaaagatacatactcttgtaaacttttttaaaattaaaaaacagaaacaatcaaaaagcaaaacatcagtatatcattttttatttatggtaatttaccttctagaaagcttttctattcataagccccaatgtattgcttccttcccaacattccataaattatttcactaaaactctaataacctgtagttaaagaaatgatatcttagaatcaaaggtattagaagtgtagatagcaccatttgattatttcagtttacttttctgttattggaaccgatatttgagtctgtgttttctacaggtacagttttgggatgtttaaagttgggggacctattagaagtcagctttaaactcctaactgtatatatgctgaaaactgatagtggcaaatctgggcatttcttttttttttttttaattttattttgaaataattatagattcacaagaagacactatttcgttgtatttttcttctaattattagcaaaagtgagaagtaatttaatttttctaaaggaaatgaaaacaattttcctctttttttaaagggccatttgaattgtgaaaaaatacatttttttttgcagcaatttagataatatggatttttctttaaaaaattagtagtctcctacctccgttttgtcttttgtgaaataacaacattttggtgtctaaacgtccatgtgtttattttcttagcgaaacaaatatatatgcatacagggtttatatgttttaatgaaagtaattgtatgttcatacttgctgttctttaaaaagtataactagaatagacattctgatcaacataccttatgttatatcatatttacttaatgattgtcctattaatggacagatggacagtatactgtttgcagttttctactcttttttttttttttgagacggagtctctctctgtcaccaggctggagtgcaatgatctcggctcgctgccacctccgcctcctgggttcaagcagttctcttgcttcagcctcccaagtagctgggaatacaggcatgtgccaccacgcccagctaatttttgtatttttagtagagacgggatttcaccatgttggccaggatgtcttgctctcttgacctcgtgattcacctgcctcggcctctccaagtgctgggattataggcgtgagccactgcacccggcctgcagttttctactcttatattgctgtagaacatagccaaacacttggctatgttggtgtcactatttatgtagaataggttcttagttaggaggtggtataagggagttgttaagagcacagcttatctagggctaaaacttcctagaaggaatccaggctatactaataagtgtgtaacttaaggcaaaataatgtgttcccatttcctcttttgaaagaggcgctagatcattgatcctctctacttctctactttgggtgaaaactaacttctttgacaggctgatggggtaaaaaaaaaaaaaaagttacctggctgttaactaaagattgatcattttttcatgtattttaatcacttgtaattcttctgtgaactgcttatcattttcttgtctcttttttttttttttttaacttgtctgtgtcttactgctttgtaggatttctttatatattctggacattagcttttcattatatgtagcaaatgtatcttttactctgatgcttacttttaattttatttatgttttcttctttccatgttgctaagttgattgtctccttttatggcttctgaatttttgttttgcttatgaagaccttctatatcccaagatttttaaaaaaatctgttttttttttctttaacacttcaataatttattgatcactagatcatttgggattttttaaaattgcagtattagattatatactattttttattcaaatggatagcccagtgtcccatcactgtttataaaatggtccagccttcccatctttagttatacattttagcaaatattaaaacctcataaattcatgagtctgtttctagatttttctgattttgagacagggtctctgtcacccatgctggagtgtggtggcacagttatagctcactgcagccttgaactcctgggctcaagcgatgctcctgcttcaagcctcccaagtagctgggactacagtgtgcgccaccatgcccagctaattttaaaatttgttatagacatgagggctttgctatgttgtgctggtcttgaactcctggctggagctgaactcctgccctcaagcagtcctcctgccttggcctctcaaagtgctgggattacagtcatgagctgctgtacccagctatatctagattctttagtttctttgctcttttgtctaattttatactaataggaaattatttaaattactgtagctttatagtataatttagtatgtgacagggcaaattcccacttttttccccagaagcttcaaaatgattctcatgaatttattcttgtatgtaacctttaggatcagtttgtccaattatgtaaaaaattaccaccgtgattttggtggggattactatagaattatagattagtttgcaggctatcagattttaacttaaagtagtgatttcaagctaaccttttaatctaacagtggctttagcctaagatattacctaaaaatttaaaactccctataatctaaaaagttatattatttaacttttcaaaaagtgtttgtgatatgaaggtctatttcttcccttctttcttcctgtttattataggctttttgtagtattaccataggaatacttttgtttaatacgtgtttctcagaagttttcattagcatgttattacataattagttgtattgttaaggtctatggttctatggcatccgagtttttctgagttttagcctatgtttctctgtgtcaagagtttgatgttaacatgtaactatagatgattgaagtatcttgttttcattcgatttgttttataaattgtgacaaactttagaatagtgtaatagtcttctagttatgtggcaatattactaggttttaggtcaaagatgagacagttaagagtgctgttttaccttatttattcccctaaaatgtgttatgatgagtgctataggctagtactaaatgttttatcgagaaaaaagggaagttcttttttatttatgttgaatccaaaatattaatgatgccaggccttttgttttggtattcaaaattttcagaaatccattctgaaagccatttcaggttgataacagtagttgctcagtcagtatttttcaaattttttgacagtgtgactcttaattgttgactgtgccatttttccagtagtaaacaatgtattttgtttcttctcatgcaggtcgtcaaagtcctacattttagctttcagtgctgaacttccttctcccttaaattattataaacttttgctgctgtttaataaacgtgaagatgtctcgcagtcctgatgcgaaggaagaccctgtggagtgccctctttgcatggagcccttggagatagatgatatcaactttttcccttgcacctgtggctaccagatttgccgattttgttggcatcgaattcgcactgatgaaaatgggctttgtcctgcatgtagaaaggtaaatacttcaaaataacttcacagtgatttgttttaattgtatagttttttgctgccatgtatatgagcaagtgaatttaaaagactgtgcaaacctcacctgattatattattttattatgtgtattattttaagatataaggtggaaagaacactgcactgggagtcagggccacgtcttggctttgtttactagctgtatgatctttgtaaagtcatgtaacctctctgattggcctttagtttgttgttgttttatccgaggagaaagacatggtcagggatgcttactgcttaaggaaattaagaatagagaggtaatggatttggatagggttgcatgaaattagaaaaacattttatattacatgtgggaggaaattcaccttaattttctagtacaaaataggagagaagatgtttactaaaaagagttaaaatgacagagaaatcctcgttcagaagataataaatgctcatttcttgagcccttacagcaattttaggctggtattgatattcccattttacactcaaggaaacaggagcttagataacttgtccagagtcatcaacttgtgagtagtaggtaggaggtttggaattcaaatgtcttttgagtctatagtctgttctgaactattgtattattgaaaagcccctcctggccgggcgcggtggctcacgcctgtaatcccagcactttgggaggctgaggcaggcggatcacaaggtcaggagatcgagaccatcctggctaacacagtgaaaccctgtctctactaaaaatacaaaaaattaggcaggcgtggtggcgagcacctatagtcccagctacttgggaggctgaggcaggagaatggcgtgaacccggaaggcggagcttgcagtgagccgagatcgtgccacagcactccagcctgggcaacagagcgagactccgtctcaaaaaaaagaaaagaaaagaaaagaaaagcccctcctaactctaaaaatctattatttttaatattctatgggttttattgttttgataagcaccagcgacacaattacagtatagtgaattaactactaggataggtaggactgccacatttgtattttgaaggactttatttgtgaccttagggattaatgcaaatatctgccactttattatggaatttgccttttctttaatgtttgtataatttaaactttattttctaattacccattttaaagacctcttgtgttcattactatcaaattttgagaaagctgaaatgtgttttagatatctaaaatttaaaaaataatctgtctcttagttttcttacctgagaatttataataaatctcagaaggcaatgcattgtttatttatcattagatttttaaaattaatccagtggatatgtatatatattatatgaataaaaagttaggtctatttcaatgttaatatagtttatacatttatatagttacacatatattgtatatgtaacatatataatgtgtatatataatttgtatatatataatatttcatgtagttaatatagtttaagaaatgtgaatggaaaggtaaatgaaaaccctaacattagctttgggaaaaatctatataaatttttgtttatggattttgaacatttagttatattttagaaattggttcccagtcattccataagaagacaatcatattgaactatttcatcagaagtaatgactttcaggattgcccaagactctaaggattgtaaatttgattggcttcatgatgtttatctttttcttttcttttttttttccctttgggatgttatgaagtcaccttaaaaggaccagttattaaattgtcattacagaaaaataagttatttggggaaattacaaagaaagtcatttcaatcttaagagattacctaaaattacttcaagtccttagaaagtaaaaaaagaaaaaaaaatagaagattttaaaggtaaaaacagtataactttagcaaagcttgatatgaataaaagggtgtcctgggaataccagaaagtgtgaggaatctctgaaagacaagaaggacattggaatcaatttcaaggaggaagcattagatcagctcaagagaaaactgcttaaaaaaaaaaaaggaatgggcgtgcttcaggatcctaggaaaaatatcagtgtagttggtcaagtatggtgtcagagcagccttataggcctacccccttttttgtgccatgtagcagagacagatgccctctaattaaaacagtgattataagtttgaatcagagccttaaggttaatggaaacattctggaggaactaggagcttctatacctagcagattacctgtccagcaatatgtctttgctacagtcacaaaggagaatcttgcagtaaacagaaatagcatttataggcagattaccaggattctcacatgtaaaggtgtgcaaaaacaagaattaacacagatttcaggaaaaccaacatcatgaaagagaagcacaaaattcagtaaatagaagatcctaacacaggaaacaaaacagaaagacatttaaaactatcattggtacctttcggaaaggaaagagggaatgtcatattcatgtagaaagattatatttccttataaaaagaacaaattagagaggtcttgaaaaatcttatagatggcctgaaaagcaaaatggacacattacaagagcaaattgataaattggaagcttgtgctgagtgggtttttattgaagaatataatgcaacaagagaaaaaatagaaagcatgaaggaaaagcagcatagatccacaggttctaatctctgctgagagcagtctcagaggagtgaactaaaaatgaataggaagaaataatcaaagaactaatagaagaaaattcctcttagattagaagggccctgtgagtgtgaagcaaggtgactgaaaaagatcaatgtttgtttatataattgtgaaattttagaacatctagaatagagaaaactctaaaagcttgtgagagataaatatttagaatacaacagaacaagactaaaattaataccagtattttcattttacaattacagaaaataaactgagaaatttattttcccaaattttgagggaacgtaactttgaaaggagagctctcaattttattaatatgtaaaggcataataaaacattttcacacatgcaaagattcatgaactttattattcatgaaccaccatacaaacagagagttgaggatgactgcagggttttgactgaacacctgaaagtaaagaatggccattaactgaaatggggaagactgcaccaggaatagacatttgagaagatcagaaactcagttttagacatgttaagtatgagattcctattagaaatctaagtggagatattgagtagacaggtgaatgaatgttcatgtctggatttcaggtgaggttctggtatggagatacagatttcggtgtcagttagcatatagtgtgttatttataacatcacgagactggataagatcatgaattgaaaagatgaaagattgtctaaggataagcaccaaaactttccaacagttacagaaaaaagaaatccagattacagaggtctaagacaacctccctttagggtctccaagttacatctttagctgaggtggttattgttattaggccaggaataattttttggtttcttgttgacttaatcacatctcctagaacctcttcttggtctcctggtcaccagactttgtagtgcaggcagcagagagtcttggagtctgaatcctggcctagtcttgccagagaatctgctcagcgcagcgcaagataaatatttgtgaagttgcctgtgtcggtcggagtcaagcaagtctttggtctctggaggccatctacagtggttgtgttaacacagaggaaaaccacaatgttgctagaggcctagagagggtcatgaagctgccatttacatgtaacccaaccctgcaggcacagggtctacagaggccatgctgtgaggtggcatattgagatgatctgaagaaacttgggagatgcaagtagggagctagttgaatcaagctcctgttctcagtctacaatatgaaacgtgcctgttttgcagaaagtttctcttagctttaatatctttgaaaagcagagacagccagattttaaaaaccaagagagtttagtgttcccgtttggtcctagctgctaaacacaggcacaggctacttggttttttcttagactgctttcatgtgtccttttagtgcagcatttctgagaaagctactagtggaaacagcagatatgggaacataggtggtgcactgatgtggcattctgtattgattcccatggaaaccagagactatacaaggaaatgagaacttcaacagaagtattattatttttatttcagaaataatagaagataatgtatctatgaaacaaaaacaggatggtggtttggacatggtggctcatgcttgtaatcccaacattttgggaggctgaggtgggaggattgtttgaggttaggagtttgagaccagcctgggtaacatggcgaggcctcatctctaataacaataagaaaaattagctgagtgtggtggcacatgcttgtagtcccagctactcgggaggctaaggtgggaggatcgcttgaacccaggaggttgaagctacagtgagccatgttcatgccactgcactccagcctgggtgatagagcaagatactacctcaagaaaaaaaaaaaaaaaaaaaaaagaacaggatgatataaagaagaatattcagtgaacagtaaccattgtcattgaaaattaaaatataatagcaaaaataactgaaggaaagcattggaagatgaagttgagaagatcttttagtgaaacaaaaagcaaaagagatgaaatatttgggagaagagattaagttcatctgaggatcaatcaaggacgtcaaataccaaacaattaagtgctctaggaagagatcagtactctaggacgagatcacagtgatacagaggggaagtaatcatccaagaatatgatcgaagaatatttcccagaaccaaagaacacattttcagattgaaagggcctatcaagtgtccagcacaaacttttttatttggaaataccagaataccaaagacaaagaagttctacaagcttccacagaggaaaatagcagatcgcatgtaaaagacaaggaatccgaatcgtttcagatttctcaatagcaagacttcaagctagaagatacttctgaaggaaattaattttcatcttcggattccgtatctgctcaactgttaagtgtgacggtacactaaaagacattttcagacctgagaggcctcaaaatttttttaccttccatgcattctctcccaggaatctattggaggatgtgccccaccaacatgaaggaataaagaacagagaatcatgggataaaggaaacaggagatccaacacaggagagaagcccctggggaaaggatgacagctgtgcccaaggtaaagagggcaaccagtccaaaatgcaacacatcagagaactgcaagagggatttgtagaaaaagataaaattaaaagaatacttggtgttctaaaagagaaatgtcttgtgagaccattttgccagtttgaagagatttgatggttgaattagtaatgaacacctaaaaatataatcagacaaaacagattaactctaggggaaacaaaaagttgtagagaaatgaaaaagcaatcatattatgctgcagtggctcacatgtgaggtaggggtcatgatatatttttttttcctatgctgatattcttgttgcagcatcgtttgcagaaaagactgtcctttcactcttgattggttttgaaatctttcctgaaagtcaattgcccataaatgtataaatccaatactagcatctgtattctgtccattgatgattatatctatccatatgctcataccatactgtctttattattgtaagttttgaaacctggcgtgacaaggttttcaactttgttattctttttagaatttattttgggtattctaagtatttagcatttgcacataaatttttgaatcagtttgtgagtttcaacaaaaagcctactgggattttgatggagattaccttaactctacagttagatcattttgaggaaaaatgacatcttaaaaatgttgactcctttagttcatgaataaggtatatctttacatgttttaaaatttctttagcaatgttttatagttttcagtatacataatagtacagcatgttttgttatattatccatattttatgtttttgagctatggcccatgatattgtttctttttttttttgagatggagtttcactcatgtcacccaggctggagtgcagtggtgtgatctcggctcactgcatcctccacctgccgggttcaagtgattctcctgcctcagcctctggagtagctgggactacaggcgtgtgccaccacacctggctaattttttgtatttttagtagagacagggtttcaccatgttagccaggatggtctcaatctcctgacctcgtgatccacccgccttggcctcccaaagtgtcaggattacaagcgtgagccaccgcacccagcccgatattgtttctgaaatttcactttccagttgtttattgctaatgtaccacttttgaatgttgaccatctatatcaagaagccttgttaattaaataggttccttagaattttctacataggtgattatgtgttctatgcaaaaagacagttttactctttcctttccaatctctgctttttaaaatttttttagagacaaggtcttgctctttcgcccaggctatgaagtgcagtggcacaaatatggctcactgcagtctagaactcctgggctcaagcagtacttctgtctcagccttcccaataggtgggactataggcatgtgccaccatgcccaactaattgtttaaaaaatttttttgtaaaggtctcgctgtgttgcccaggctggtcttgaactcctggcctcaagcatccttccacctcagtcccctccaaaatgctgggattataggcatgagtcactgcacctggctctgtgtttttaaattctttttcttgccttaattctactgggtaactcctctagtgtttaatattaaaaagaagtgataagaaagatatctttgttcccaatcttagcaggaaggcattaagtattacatcattaagtatgttgctgtgtgtgcattttttggtctggtctttatcaggtcaaggaagttttcctttattcctaatttgctgagagattttatcatgaatggtgttgaattttgccaaataccttttttgatactattgagatggttatacttttttctccttttattctgttgaaacagtaatttatgttttttggttttttttaaagcgatagggtcttggtctctcgcccagggtggagtacagtggcctcattgtagctcactgcagcttcaacctcctgggctcaagcaattctcctgcttcagcctaccaagtagctgggactacaggtgtgcaccaccacacctgaataattaaaaaaaattttttgtctttgcagagactgggctcgctatgttgcccaggttggtcttgaactactgccctcaggtgttcctcctgcctcagcctcctaaagtgttgggattataggcacgaaccactacgtctggcctatgttaatttttaaatcaccttttcttctcaaagataaaccccacttgattataatgtattaccctttttatattcagtttgctaatattttcagaagatttttttgtctgtgtcataaagggtatttctttgtagttttatattgttttcagttttggtatcaaaggaatactgtccacataaaatgagttgggaaatgtttcctcatcttctgccttctgaaagagtttgcgtgtcattggtattctttttttatttcttttctaaattcatgattggcatggactctactaggtatcttcgttggaaagacacctatgaaaactagctatcacaatattcctgtatgtagacctctgggattctttttggactaactccatctttgtggtattttgcccccataaatttcagttgtctcagcctcttctaatttttatcactgtctgtcccctccattcattatgactcttgaacccagtaccaggatctggtaagtactggaagagcagaaagctggggtagttatggtattcatcggtcttatttcttttttctcagagatcacagtcctgctgttcattgcctgtttcttcagtgtctgaaaatagttatttcctgtattttgtctagttctgcagttgtttatgatgatagggcttgtctgttactttcatggctggaagtggaagagtgtaaataacatttgcattttataataatgtaatcctgaatattgccctaaccaaagttatggtagaaattagaaagtggactcgggatgtttgatgaggggaactgacagggctaaatcttcaagttgcataatgtgatgatatctaaaactgagaaatcaagaagtagcaatatggatgtattatttagagatatggtagtaaatgacaaagaatcatctaaaagagtcaacatgattatttctaggaagaggcaactggttgtagaggaggagggaaaatgggggactgctctttttcctaacaaaacttgtagaaccaattgactagaagcatattcaagaaaaatatcaatgaaaggagggaaaaatgatacagtgggcaaatggaaatcataggaaagaagatataatagtactgatattagtcataatgaaattcaagatcaaacacattttaaaagggcaaaagttccacaaagtcatgaatctttacttaacagtacaactttgaagtatattgagccattataaacaccaggagaaactgacatccgtatttataatggagagacttccccttctgtattttagaaatagatgaagtagacaatgaaaagaaaatggggaatttgaataacagaattagcaagatgtgtgtgtgtatgtgtgtgtgtataactttgtacagataaatttgtcacaatgttaaatacaagagcagtggtcaaaaaacaggccacaatggaaaaattagccaattccaaaaagtaaatctcccttgtgtattttaaaaatttatcctccttgggagactttttaaatctgtgggctggtgaatttccttagttcaagataaaaattaagctattatctcttcagatacaaccttttttctctttcttctcctctgtgactctgattaaatatttttcagattttttttcactgtcttctatgtctcttaatctcttttctgtatgttctttctccttcataccttagtaaatgatttattctgacctgtcctcatgtgcgaatagtttgttttgtatccaatctgccactaaatatgtctgttgagtttttaactattggttttgtgttttccatacctagaagtaatgtttgctcttttttttctcttgctgttattttaaaatagattattataccctgtaggtattttcaaggttatatattttttaaacagataagcattattttttaaaaacatctttgtccagtaattccagtggctgaagtctgtgggctatatttctgcttattgttgctcatgttgttgtatttctttgtgtttggttaatgtggctgcttccttgttattgtcattgagaaaattatttgtaaggttccccaaggactagtgtggatttgctcctctctagagaggcttcaatttggcttctgctgggtcctgtaagcattggcattagggctgccatgtgcttaaaggtttttggtctagccaagcaatatatctccatgctgcaactcttccctagggttgatttcaacaatttctccaggatggttttcccctacctactttttcttttttcttaccttagtctgacctttctgtagtttgttcttaccctgagactatcctctggtttgtaatttgttcttatcctgagcccaccgtttggaggtgggctgtatctcagcttatcatggaacctatcttgccaaagactaccatgagctggtgttgggctttgaatgttctttctccttagctctttctctccctaccccatttattgttaatcagattttctgtttaggctctgaatttttactttcaaccaaaaactggcttgggatttttccagtatagatttttaggatgttagtaagaagaaaaaaaaaatttttatgttgtatttttcatggtttttagtaaaagggttaattagctctccattactggaaactagaaccctaaaaaaatagtataatcaaattagatgttaacagcaaaaggattttttaaaagttcatttacctgtaatacataaattatggtcttccataactcttggtttaaagagatcaaaaagaaaattacaaattattctgaatcaatacttggaacatgatttctgaatatctggcatgaggcccaagtggtatgcagcagaaaagctaaagctgtgaatacatttattagaaaacaacaaaaattagaaaaagaatgagtactatgacagtggaagggatcagaaaaagaaatcagaaaaagaataagccctgggacagtggaagggagtgagtaagcagaattttgtgaaataggaaaaaaaaaagtttttcaataaagattagagctggttctctgaaaagaccaataaaataattaaagtcttagcaagtttaataaaaagtaaagagaagacacatgtaaatagcatttggaataaaagtggaaacataactacacatatggaagaaattaagtaattacactagaaactgcaaaccatgttaactagtaatttggcagtttataactattaaaaatagtgttatatctaaatacttctccataggcaacctattttgcattagtttgatatgtaatgcacaatagtatgtttaaagagaaattttctgcttgtaacttttcatttgatgctatattcctagaaatttaacatgtgttttagtcaaaaagttgatcagatatatttttccttttttttttttttttgagatggagtttcattcttgttccccaggctggagtacagtggcgtgatctccactcactgcaacctccatctcccgggttcaagcgattctcctgcctcagcctcctgagtagctgggattacaggcacctgccatcacgcctggctcatttttgtatttttagtagaaacagggtttcaccatgttggccaggctggtctcaaactcctgacctcaggtgatccacacgtctcggcctcccaaagtactgggattacaggcatgagccactgcacccagcctgtatttttctttctttctttctttttttttttttttgagacggagtcttgctctgtcacccaggctggagtgcagtggcacgatctcggctcactgcaagttctgcctcctgggttcacgccattctcctgctccagccttctgagtagctgggactacaggtgcccaccaccacacccggctaatttttttttatttttagtagagatggggtttcaccgtgttagccaggatggtctcgatttcctgaccttgtggtccgcccgccttggcctcccaaagtgctgggattacaggcgtgagccaccgtgcccagcctttgtatttttctttatatccatttttagtgctagtaatattagctttgtgagtctcactgtctgcagttattcgtgtttggtttcttcatttccccccgcttttttgaaatagggtcttgccttgtcacccaagctggagcacagtgatgccattgtagttcattgcagcctcgaactcctgtgctcaagcatccctcctgcctcagcctcccaagtagctgtgactgtaggtgcacaccaccatgcctggataatttttttttattttttgtagagatgggggtctcactgtgttgctcaggctgtcttgcaatcctcctacctcaccctccctaagtgctggaattacaggtgtgagccaccgcacccagccctcccatctttatagtctacctctcatccttattttattgtattagtttattgatattctgaccttgggtttgtgcagtaggtatatatggtggtgtgtgtagtgaaggaattgtaatggaggaggaacttttttggaatacatgcttttccaatgttttaataaaacttttggacaagttgaagttaataaaccttttgattatgcatttgctatttgtggttagcatattttatatttttcaggtgttaagattaaacaactagtatttataacttcttttaaggtactggaataagatatatttggagggtaaattgtagtagaatactctgtctcatgttcttctgtgtttgtatcttttgtattatgtagcagttatgttccaacaacgttgactttgaagcctgatggaaatatatttccataggaacaaaacttggaataacatagcaaaaagactaagagaacaatataactcagtgttatatgcttaacatgatggtgcatcagtaatatacttgataagagttgatattgcaacctgatttttcttacttgtcactctccctcaatttttttgtgttcatgtattttcaaatattacatgactttgcctcatgtgtaatcagtattttatacaacctcatagaatactaacaaaggaaacagcttgagggtgtttgtcacattctgccagttctcagaaaaatctaagtcccagaaaagttacatcattgcttcttacgccttctgtgatgaagaaccagattttttcttttctttcttttcccctgtctgttgaggactgatatagagtgctattatgcatggctaatattttgcttgtgtcatatgttcgactacattgaaaaatatcttattctgcagagtccactggtgatggacctgaatgttggggtagtgtcagttgctctgagagtttctgaacgcttatctcagtttttgtactttccttgttatggactggtaaaaggcagtgcgtcaactatactttgaaacaaaactaggagcaatccatgtgtttgatccattcaggcagtggagcatcggtgttttagatgtcagtgaagagcaacagctttcaaatgaacttgtttgtttttttttgtttgtttgtttgtttgtttgttttttgagaaggagtctggctctgtcgcccaggctggagtgcagtggcacaatctcagctcactgcaacctctgcctcccaggttcaagcagttctcctgcctcagcctcccaagtagctgggattacaggctcatgctaccacacccggctagtttttaaaaatatttttggtagagacatggttttaccatgttggccaggctggtcttgaactcctgacctcgagtgatctacccacctcagcctcccaaagtgctggaattacaggcgtgagccaccgcgcctggcctcaaatgaacttaattgtaaacacttcagaaggaactaccccaggaattctggggtagaaaacagcttttctaggtgcaaatgaaaaatttaagttatcttatagaaaagtgttttaattagttaatatatagttcacttttatatctttttacatttgtgcctttagagctccaaatagcttcaagggattaaaatcatccaggcagatagattaagatttgaaggagataattaatgtaggtacagaaaagtaagtggagaaggaagttttgagaggaaggcattaagaaaaaacttaaatgtggatgactacgtaattgattgaggaactgaacttaccttaatagatctttaactgtttggttccttaaattaaaatgcaagaaataatttatgttttaataaaaaatgtgtgtgtgtgtgtgtgtgtgtgtgtgtgtgtgtgtgtgtgtgtgtgtgttttgtttgtttgtttgttttggtggcttttaaaacacagaaatgctgagttttcaattctggagagtagcttttgatgtttgcttggcaaatatttattgagcttctgaaatcatactaaatttatatttgatcctggagaaagtcctaaaatcataccaaaatcaggtaaattactaagcaagctgctttgtatgccttccactcacaaataagttggagtatgccaaataggatccactaactcaaagatccagattcttaatgtgggaatcaagtggtatgggtgtttacatcctaccaaactgttaaggattaagcctgaatcttttttgggtcaccagctctaattcattgataacaaggtaaactatgaagagtattaagtttccttaaaaagcagtaagttaaaattgaaagtgggaataaaagggaaagaatagggttcttggagacctcagggtatatactagtaaaagttgtgaaatataaggtgaaatctcactttcttaattgccgtcagcgttactctgggtatgttcacaaatttgagaggttttttttttttttaacgcttatatatgaagagatggaggttaactttactttaaagtcatagcaaaatttcaggcctgtagctatttcttccacccttccttgtttccacctttttttttttttggtttggcattatttctgttgttccccactcttgtattctgataggtgccaatttccctaccagttgagtttccatccctggaatctcttcctatccagtttaaaatattatggcatttaatgtgcccctatgttggtgttgtgtgtgcacgggtcttgggggagggaagaaaagaaaaaataatgtgccgcatcttccatttatcattaactatttgtttttagtagtaataattgacttttgaatacttatatgtaggacacagtgttaatcactttataggtgaataaataaagtactgtagtttccattttacagatgagacgactgaggtagaaaggtcatatgactttaccaaagtcacttagatagtaatggctgagctaggatgcagtgccagcattctgtctccagagccctcatttggtagccctggcattagtagtaggaatgaaaatggtatcaaaatgctaagaaagaaaagatcacacaacagtgtgattgctagtattatactgagagattactatgttccaggcacagtaataattgctttaagaacaaatgtgtaacttcattaatctcctagcacatagtaggttttcattaaatatttgttaattataatgtgtaaaacccacatttagatgcaagaacttacactccttgcctcttacatagcaccttagaggcttaatcttcccataaactctgttggacactgcagggaatatcaagattggtttgttgttgttttgcctttaaaccgtagatgttttagcactaaaagtggtctgtgctaagctccaaaaaattttttattagaactattttatttaacatttgtcttttattaaataaaaaaattttttaagcagttaaaacattctaataaagggagaatgtaacagataaccacatacccaccaccaagaatcagataattacttacgcagtttttttaattaatttaatgctaacgtctatgtttgtttcatgcttaaagccatatccagaagacccagcagtttataaaccactctcccaggaagagctgcaaaggataaagaatgagaaaaaacagaaacaaaatgagagaaaacagaaaatatcagaaaatcgcaaacatttggctagtgtacgtgtcgtacaaaaaaacctcgtctttgttgtaggtttatctcagcgcctagcagacccagaggtaagtcctcttgttttctttgtcatcttgatattttttgtttccttgtcttacttggaaataatatttgaccctgctcaattgtgtgattcctctgaacacattatatcttgaaggagaaaataagaaattacttttgaataattgctaagaaaccaatgaccagtcgtgatcttaggcaagtgtcttaatctttcttggcttcaatttcttcagttctaaaactaaggagttgaactagatttctaacgtgctgacaatctgtgaaaattggataatgattctattttgctgttaaggctaacatgtgatatgaatactctgtttcttttctcttttctgtgtctgtataagaaccaacagtggaggtgcagttgtttgaactcactagagattttagtaacctagattttaaaaattctgcccaagtagtaattcttgatttttcagaactattactatactgtctcatcattcttattcattttaagccagtttggaggaagagggagaatttggtaatgtgaaataaaggacccaatttctttgtcattcattcgggggaggagagagcttaaccttgtttcttagtacctcttttttctcttcttccaccaaaagccttgttagagtaatgtcgaactcatactttccttgaggcccttggatatgcatctggtaatttttagtttctgatataccatgagatactgactagattgttgagaaggcaaatataagctgatcgacaggcagagtgtttacttggtaggagagagctggttttgggggatcttccaaaagatccttgagaagaatttaaagtaatacttggcatggtaccaatcagaggaaagattgtagtgaacatgcttaaggagataggaaccttgggaattggctgagcgtgttaaaggccccagaaggaggatctttaaggagtctaatgtctgtttttgtaaaaattaatttaaaaaatagttaatctttattgatgatgtgtttatatgtgttgcaactatacagggcttcaaaatcaatacgaaatgattttgaccccgcgaaagtcagggacattagtaagaaggatctgtcatggatgataaacatggaggcaactcaagagttactatgacctaggtccagggatgctggcagtcgttagttacatgatactcaggacatcaagttctttccaaatccaaaggagtggtctagagttgatatagaaaaagacttctttagtaagaaatgttcagcaaagagctcattttaggtgtatgactcaccttctgtagccagttctccctcttttttttttaagctagtggttattccccagacagagagaatcctgcctcacctttgatcgtttccttgaacaacatctcaactttgcactaatccttttatctccatcttttaactctattaaatagagttaaaaaggtgtgagctaccttttttctaaccacagggcacttctatatactcctctaaccaacgggcacttatgccagtatttacacctactaatcttactgattagaatctacttttttttttttttttttttgggagacatggtctcactttgtcacccagactggagtgtagtggtatgatcatggttcactgcagcctcagcctcctgggctcaagtgattcttgtgcctcagccccccaggtaactgggtctacaggtgcatatcaccacacctggctagtttttgtattttttgcagagatggggttttgccacattgcccaggctggtcttgaactcctgagctcaagcaatctgcctgccttggcctcccaaagtgctgggattgcaggtatgagccactgcgcccaacctagaatctactttcacgtttctgacgtgcagttccagtaaatgaatggaacttttatcttgatttgagagctcttggatatggatgtacctgtttttattaatacatcttggatctgatcaactgtagtactggaggcatatgaaatagcatccaaaatttggatgaaattaaggcatagactgaatatgatatttggattctaagtttcctacctcttttgggtatgtgatttataagagggttttttgtttgtttgtttgttttttgcccaggctggagtgcagtgcacaatctcagctcactgcagcctccaccccctgggttcaagtgattctcctgtctccaccacctgaggaactgggattacaggtgtgcaccaccatgcccagctaattttttttttttttttgatttttagtggagatggggtttcaccatgttgactaggctggtctcaaactcctgacctcaagtgatccacctgcctcggccccccaaagtgctgggagtacaggtgtgagccatcgtgttttttatttacttacagagatttcttatttccgtttcaagtaactctagtaagattaatcttaactattatattgaaggaaatgaatctaagtagtgactactgaaagagagtactttatcatgagtctaagtatacttaaaaatatactctactagttttatatgtttgaaataattctttcttggtgagaggacattggattttcttaaataaaataaatgggtgctttcctttatttctcttgaaagtccagaaagaccagagacaagtgttttgttttttattaaatagtaccacttctaaataatatcttttaaaattctcttaatttgtctttataacctttttagtatttggaaaacttctagtgaagttggcttcactcgaaaactcaaaagaaggttcaaattgttagcgtcactttcaagtcaaagaaggatccttgtaatttacttatctttggatgatgaacaatttgatatttgacctaattctgattaacagaataggagagtttaggaacaattaaagcttattttgaaaataatttttgcataccaaaaagcttcataccaagattgattgtcaaatgtattcatttgttggttgatagaaaaaaaaagatcttagaagacaaggtaattagagccaacgatagaggtaatgatgatcaccttctaagtcctaaaattttagatttgtgaggacctaattcatttattctgcaaattaattcttgaatacctactatttgctagatattttgatgccagggggcgggtaggggggaagtagttagcaagccagacacaagctcacagactggtagggaagacagaattttaagtgcttacatgtgtgctgagtatttataaaaggaaattacacagctttttgggaacatatattagaacacttagctagtgtggagattgacaaaaagcttctctgaggaaaggataattagggtcagcttgaagaattaataggtgttatctaggtagcacataaagaagagtgccctgggtatatgtgataacccaggaaagtagcttgtttgatgaactatgactttaaagacacaacatttcattttacaaataagaaactgaggcaaaaggaagttgagtgacttgtccgtggtcttacagttcctattccagtgtattctgtgttgattattggcatgttgttcatccatccattcatttattcactcattcatccatctgattaccaagtttttgttttttctctgtagtatctatgaatacttgactgttttcccaaggaatatatagcaagatattttggtactagacatgcccttgaacatttataattctgtcacatggcctcttattttctatgttagtaggaaaattggcccactgtgaaggacatcgtgatattgagtatatagataatgtgctgatgtacagggtgctctgtctactctccataacctttgttagttcactagaatactattttgctagttgtttaggcagaatctagagtgttcaagttaatatggaaagtttaaattcattgtttttcccttgcctgggcagcctatttcttatcaaccgcttgccatttgctaatactttgaggcccactattgttacaaatccgtgtaattctttttgtttaatgctaagatagttcagtttttcagtaagcatttattgagtacctgcaatatgccaggcattatttaagacactgagcatatactgataaacaagtagtagtagataaataagtagtaccttcctttttacgaaagtaacaatatgagaagtgttgtgtgctgtaaaggcacaagggatagcattccaccttgggagggaggtacgtacccctctgctgtctttggggtaaagtgcttctggcacagcaactatacaaatgcttctggatgagaaagacctcaatctgcaagtgatttgtttggctggaagattagattgcagtggtgagaaagaaggtgccaaggtaagtggtaatggatcatgaagggctttctaaccacgctaaggagtttgcactttatcctaaaggcagtggaaaacttttgaatgttcttaagaacagagtgacacggttagatttgtgctttgacagtaccattctggctgcagtgtgaacatgtcatcataattacataccaatctcatgttgttataaaggtgttacacccaaacgttggccagaaaacatttccataaaccagtggccttcaaccttatccgactcagtgtccactttttaaaaacgaatgtaacaccccttgactttcatgaaatgacttcttgataatataatttgtctacacacataatttgaaaaagtatatataaacaaggaataaaaggaaagtaattataaaaagactatttgtacatataaatgcttgtacattcctactctagaaccttgctattcaaggtgtggcctctgggcccataacattaatatcactggagagcttattaggcaaactcttcagccctgcctcagatctgctggatctgaatctgcattttaactagatcattgaagaatttctttacatattgaggctctaaagtactattttatcctatgtaataaagaagtaagatacttgcacctatttgtaaaatcatggactgtgacaactacaaatgcagattgttactggtgtatgaattggcaactcaggtaccatgaaacatcaataatgtgatttttctcagataactcttagtaaaattttaaacaaagctgggaaaatactttgtaattacgtataatatgaaatttgtttctggctcaataaacagcttcaggcatgtcttgcagggcattcagaaattgtgcgggacaattctttgttgtacggggctagcctgagccttgaaagacatctctggtccccatccactgaacaccagtagactctcccagtcactttaacagtctctcccaataaaagtttccacaaatttccagactgccccttgagtgcttccctcattgagaactactgccttaaacctatgatgctgatgcaggacaacctgtgaccggcctgactgaaccctttggagcaggtatcttagctatgacgctggttgctgagttggtatgcatccagctggaatattgaatgcagttccaaactccactagccagaaagctttttcaaactctgctcttctctctttatcaaccctgagactttgctacggatgacttaagggtcattgtgttagccactgttactatcatatccctcaggggttttgggtggaaaagagattgaaactcagtgtaattacctcaccttctaatccattagaattggctacagcaaacataacagttaccttatacttacaagcacctaaaataaaatataagattaattactttctattaattaggtagaagactaagaattaatgatacacccgccgtagatttattcatgaagctttgattctatcaaatgcagaacataatagctaaattataagtctgtaagaatattatcaaatatgaatgggaagaaatactttacacacacttgaatagttacatgcagaaagctttcatttgcagagagcttgctatttggagagattgtaggaacagtaggtctaaagatacattctaggaaaacagaattttgaggtagttgcaaattatgtgagatgtctttttatcaagttacataccggtcaaattagccaaggttaagtttgtaagagaagctgaaagaatgcatatagtgttcacaaagtaaaggagatgaggtacgtaggtaccaccaagaagtaaccaggtaataaagaaagaaacgtgtaaatttgtttccgaaatgttacgtaccaggaaaagtaggggaaaaagcctcatgagacagatgagaaatggtaaaactgattgacataggaaactgtatgcacaatgataaatcataaagcatagagggttgagtgggcactctgttacttgaaatttgtgatttataaacagtgttatatacaatagatttatgagtaaagactgtctttatgtctgttctttcaattctgtctttacctttaatacataattttggtcattgtttttgaagaattgctttcgtgtaaggatcatctcttaatggcaatgtagttaagatctttgatctgttttgtgtttaagaatactgtacaggtaagatcccatagactattggagcagttattagaacatagtagataataccaaatttagtgagtctcagtttctattcagtgggtcaagaaaaaaaatagcttttaaactcttttgatttcctataggttagctacacaaactgcaattaggaggtagtgacatttattagaaaactttaaaaattatctttttccttttgctgaagagggatgatgaatgtctccattataaggataaagttgtttaaaatggggacagtttataccttatttcttcttcttttataggttttaaaacgaccagaatattttgggaagtttggtaaaatacataaagttgtcatcaataatagcacatcatatgcaggctcacaggtaactaattttagggatttttgcttttcctccctatggtctgggcttgacctgggctgttccaaaggaaactttgctctctctgattagaaagcttgagggacaaggacttaagcaatgtagtgacctctgttggtggggtgggggattttttgctgcttatttgacccttctaggacttaacaattaagagggaaatagtaaaacaaacatttagtgatggagtgaaagggagggggaggtgagtctgttatcaggcaatgtgataaatagataaggattcgatagtgatttctaatctgtaatattttcagatattgaaaacttaatagtgtctttgttatgctgctgttatcagatagaatagttttccagattgcatttatttttgagaatgtaatttgacatagtctttaagtcttattttttaatctaaattttaatttttttataaccttgacgagcctgtcaaatgactgtctaaaaaaataaatcccaagccaaagtaatattttattcacatatattacagtattcttaaaatcagataaaattcattgagtattattactttcaaatgagctagtttagtgtttaatcaaaactgccactactactactaattattgtcattagtaatagtagtagtcagggtctttagaattccctaggttgcagtagaatttttttagtattgtttaatttttaactaataacagtggaatgggatgttttcactttagggtccaagtgccagtgcttatgtaacctatatccggtcagaagacgctctcagagccatacagtgtgtcaacaatgtggtagtagatggcagaacacttaaggtaatatagtcctcttgattcttttttttttttttaaggaagacgagataaaatatagtaagtagaggagaactgagcatttatatattaaatttgacagttaatttgaagagtctgtcttagggtccataccatctattatagctaatttgtttactgctggtttctgtagggtcctcttttaccagataggaataaacacattcattatcggggataatcttttaaaatatcgcatttttacatatatcttattttgtgaaaatagtaccattattttcttcagaaataggtagtccttaaatatatggacattgattccagtttcagtgaagagtcccactaagcaaagctttactgttagtggaaggaggatggcataattagaaaaaggaatttgccttcttatgtgtctaaccactcataacatggaacagatttttttgtctatctcctctccttatatctagggaagatttgggtgtccaagttttggaaaccatagacagatgtttcctttagttcttccaaatgagcctcatctataataaatcctgctaataagagtttagttctctagtatttgcagccatactttttgttttgtcatgtttgtgcctcattttaggtgacttgaaaacactaaacatggagattctcattgtgttgtcagttcacattgagtcttcttttcctttacccctcttcctcctgcaggcatctctaggtacaacaaaatactgcagttacttcttaaagaatatgcagtgtccaaaacctgactgcatgtatcttcatgaattgggggatgaggcggccagcttcacaaaagaggaaatgcaggtagtaaagtcatgtgaattatcaactttgcagaaaagggagtacttttttttttattagttaacattgcaaaagcaaaaactaaggatttgattttgtaaaaactgatattactttaaagtagcatttgccaaatgaagtgtttaatggaaggtcagttctgcaggatattgttacaataaaaatgtgtctgtggtcaactaagtttgaaatgctagattaatgaagatgaacaggtttctttgctttaagacttttcagagtccttaatatgctaatacagatccacgaagggaatttacaaccagaacttagttgaccaggaaaacactttgggaaatgctattttaaggggaatgtcaacaaaatgaaagctaagtgggatattgtaattatttgttaaaccaatctatactctttacagagcttgctagaaaataatactctgtcaaatgatgctgataaactaatattatttttacaaaattattctccccatgcaagtgtccactgagtcagtatcctgtaaagggtgaattgaaggcttaatatcctatgttcttttttacatttcctcttctcctgtctcctttcaatttgtagtataaacatgccttaacaagtaaaaatgttgaccatacgacatattaaatgaggattataccaaagcacaatgctgcacttccctattgtagatgaggacttcagattgttgctgtggacttttcaggaatacttctcatttagaaggtggggggcattcattgctacctttcattctccaggtcaaactagagaaagtctggtaggggctcctttaagaaaagattgttaaatggagtattaccttttttgacttggagctagtacagattaaatcccattataataaatacatcagagttttcttgatagtatggtctttatgattcaaaaacaatgttaaaataaggtctatctgatatttaataaggacagatgacttaactgatttcttaatgctttttttgtttgttttttggatgaaaccttcttgaatgaaatctaaggtagaaccatttcaaaatttcaaactaaattttaattagtagagggtatactttattcatatttcctatcaatttctgtctatcagaatttacaagtcccacccagttgatctcatttaaagtaatattaaacaataaaataaaatgtatgggctgtcctttggaacttgtttgtaatgggatttaacctgtctttatgaactgcccctgtagagtcattttaatatcaagttgagctctttgtggctttccacagatattagtgaaagccgtatctcctgaagtattgattccagaaataattttcaagacttttcatgttttaaaaatgtaaatttcacatctaatgtatttttaaaaataatttaaaactaatttcataatgttgtcctgaatttatcttgctcttctttagctgagtttaaaattactttaaaaggggattttttgttgttgttgttaaaacactttcttgatgtaggaaagaaatttgaataaattttatggcaaaaatcagaaaatctttccttgttactgcgtaactcaaaatttagtgccataggaaatatttataggtccaatagaaacaagcaagtggcctctcagaacagggaacgatgcattgtgcagtatatatgacacaattatgctcagttaagtgcaattgccctcacttgttattgttttgatttgttccgttttggttttgttgtataacatctatttagaccataatttaaaaaaattaacatccctgaaggtaagtaaaactattcttccagtctagatatacatgcaaatagtaaaatgttactcaaagtaagttcattatcatttatttttgtgtctaattagcttccacaggcaatctttgcctaatttttcccataatatttgtgacattgccttttgatgtacacctacccagctagagtaagcatagcattcttttgtgtatagagcagaggttggcaaactttttctgtaaaggccagttagtaaatattttagggtttctgagctatgtatgtagtcttctgtcacaactactttactctgcctctgttgcactactacatcctgcctttgtagcagaatggcagccaaatacaatatgtaaatgaatgggcatgaccatgttccagtaaaactttatttataaaaacaggcaactggctgtaaattgctgtccctgggtctagagtttaaaatacattaatttttattttacttttgataaccatagaagaaaagagaaagcaaaaactaattcatggaagttttacttagtgtcaagataatgaagaatatagtaaaactgttcatctatgtattacttcaagtgctaatctccaaattaaaaaggagactgatgaattttggctgattttcagaaaactcagtgttacaagacttaattcttacatgaaagacttggaagtaggtttaaagtggaccttacactttttgagctagcaaaaatgttcttcccttaatatgcttttctgtacaataaatgggttgatgaaatgccttaatttaatttatcagatatatatcatataattttgttggaatgactttcattgatctagaagttttgtgatatcctcacaggtttctcttacttgttttatttaaaaatttatttaaaatttatttaatattttattttttaaaaatattatttattttatttaaaatatatttaataattcagaagattattcagtattttataccagccattttgtgaatcccactgcagttttaatttcttctttcgttaggcgggtaaacaccaagaatatgaacagaagctacttcaagaattatataaattaaatcccaattttcttcagctatctacgggttcagttgataaaaataagaacaaagtgacaccactgcagaggtacgatacgtaagtattgatattgatagtcagacatcttaccttcttatcagttgatcttctcttttcattttagatctttttcaggcaaaatccttgttgacaaggtctgtcatttatgcagaattgtttagctgtatgggaaatgttgagtcacaatttctgggtttgaatgcttgctgcctacttttccctggtaacacttcagactagctttttgatgggttcaccttttgtggtgatttgcagactactctgagcccagggaatactttccttattaaagagaaaattaagggattttaggttttaaaaggctctgattacctaattgaggtcccccttgatataattagggggaaaatactataaaagggaaagtgggtgttatctgaaggaagccttgtgtgttggggacacagatattcaaggatatctagttccaggaatcactaaagtcatttaaatcctgtaaatgaacagggagtatcccatattcagaatcttacttatcttaggagaccagtcatctcctatcttccatctcattatggttttgggtaaactatattgtgtcaatgcttaaataaatttatggtcttcagtagagagtctcattctgatatgttgatcttcttatcttagattatattttgttatggcctgtcagaaaatcatattaataaatataatttgggaattttattctatcctgaactttctttaaatttgatgtgttaggttttttgttcttttttatttgataagtttaacttttatcaaatgaattttaaaagtaaatgttgtaattaatttatgttgatgatctttttttaaaattccaaagttaaatttgtttgaatgttgtcctaatgtctaggtgttaaatattatagcccagtgtctgtttggtgctaaaatgatgtaagaactattttcaaacatagctaataataactacatggattattgtttgagtccatttcttcaaaaacacattctttaaattttgaagtctgtgtttattcattttaagagtaaagattatttaaattattaaaaactaaattagacacatatatatgtgtatgtaaatataaataaatataaaataggccacaaagcgaaacataaagttactgtatagtaaactatgtaaaatttgtttatatcaaagctaagaataaaagctaagtcaaaacagatctttctggtttgacaagctttgatcttttcctttaatcttttgtacccacccttttctagccctgtatggttattttatggcaaaccatatgctattacttatcttattcgttgccacctctgtagtgcttggtataagaatgaagttattgtagtaaacagtggtttattttagatgcttttaacatttatgcacatcattgtttgcattaaaaatgttgtcattctaatttaagtcaaattctgggtttatagcccttggtgatttcagagcaaaaatttaaaatttagatgatctctttgagataacatttgttgtcttctgaatataaaactatatgtttttattgaaggaaatgtgaaaaatttatgaaagcatacagaataaaataaacattgcatatgatcttaatacccagagcttttcattgaaagtgatttgtgtacagcatgccctccaataacatcatttcattcagtattgttttgttacaatgttgatgagaaaaaaatcgattcccagttggggccactgcctctgtggactttgcctgttctccccatgtctgcgtggatttcctctgggcactctggtttcctcccacatcccaaagctgtgcacattaagtgaattgacatgtccaaattgtcccattgtgagtgagtgtgagtgtttatgtgtgtgaggggaccctgccaaggttggttcctgccttgccctctgatttgctaggatgagctctggccacctgcaaccctgaaccccagtaagtaggttggaagatgaatgcatgaataaatacaaattattgtaaaatgtaaatttgtaaagtgttagtgtattttcttatacactaattccttgcattgtgtattttcttagtcttttttcttcttgcgaatgcctataaaccattatacacagagtaccttggtttgttacatttgaaaacctaatacatattattatgaataatttccccatgtcactaaaacataattttttatggctatgtagtatttcactatatgttcatattttaaatttattaagccaaccttctgctattgtatgttatgttgactttgttgatattattgataacactgtgatatgaacatctttacatataaatgttatatggttgtaacatataaatggttatagctttgattgttcctacaagtagaactgctgagtcagaatatgagagtacctatttccttgaattgggtatttttttttgccttctgcatacttgccaatttgataaacaaaaaaagctgtctcattttaacttgtatttgattaacagagtgaactttttatgcactgtgagtttatgtttcccatgttttatttgtgtgtttaaaagttttatatgagttacagagatctgaacttccccaatcagttgatcaattatttgtgtttcaaaatacattgaatttgtaaatcagtattggaagaatttatattctgtgacactgaggccgggcctggtggcacatgcctgtaatcccagaacttcatgaggctgagacaggcggatcacttcaggccaggagttcgagagcagcctgggcaacaaagcaagacccctgcctctacaaaaaaacaaaaacaaaacattgactcctcccaaccattagcatagcatatctctcttgtgcctttcgggaaagattttctttttcctgttttattgtattggatctcctaccacctctttctatttgaaaaatgcttaggctaaacagtgcaaaaacagtgttagactggtagcactagcaggtgtttttacctcatacttactttcatggggatgtctctagtgttttatttactctttaaggatgattttgatcatttacttgagataagtatttttaatcacaatatgaaataccttgttattcctcatttattgagacttttttcctttagattggaaatgaatattggaatttaccaaaagtatctttggcatttataacaagtagttagcttttactgtgtttagtctgttcttgcattgctataaagaaatacttgagactggataatttataaagaagagagattcatttggctcacagttctgcaggctgtacaagcatggtgctgacatcacttggcttctgggcaaagcgggaggcaggcacatcatatacagcgatagcaagagggagggggttggagggggaaggtaccacacacttttaaatgaccagatctcctgtgaactcagagcaagagcttactcatcaccaaggggatggcccaagccattcatgagacgtccacccccatgattcaaatgcctcccaccaggccccaccttcaacactggggattataattcaacaggagatttggtggggacatacattcaaactatattatttacttatttttgtaatcagttattaatagatttgctaacattttattgactttgcctttattataaagtatagtcaggtagtccatagagcacattcataactgttttggtagcaggagaatgtctaggagccacaagttgtaaagggggaaacaggaggaaaagaggaaatataaaacaagtaaactcatttactaattttgcccaaagccagtgctacagaacctcagctgggaccagaataaaaaaaaatttttgagagtcttgctgtcacctgggctggagcacagtggtgtaatcgtagctcactgcagccttgacctcccaggctcaaacgatcatcccacctcagccttctgaatagctgaaactacaggtgcacatcaacacacccagttaatttttcttttttttggtagagatggcgtttcaccatgttgcccaggctggtctcaaactcctgggctcaagcgattcgcctgcctgtggcctcccaaagagctgggattataggtatgagccactgtgcctggccagagcaaattttgacataggtgtacatgctttgcataagtacatgatatagaaactttttttgggggggagggtggggggtggggatgttttctgttttaatgcggttacctaaatttattaagtgacaattaatggcccaagttacagagagtaagcctttggctgactgtcagtcatagcgaaaggtctgctttggtttccacttccacattgccatcacccagggtaggcatacagaggcagtaggtagtagggatgaatttgtagataagatttctcacaggaaagacttgccaaattagagtgatgagcagtgtaactctttccaaggtcatacccaagagataggtttcttcatcttcaaggtaagttaagacttaaactttctaggaaaaaaagaaactcaaagaagggtttcattttcacatttcttggcattcctagatagtgggtagcagttttacagaaagaattgcaagaaagcaaagcctggaaccttatagattgaggctctaagcttaaaatgtgattttctcttattttcaattaagccagagtacaaatcttaagattaccatagctattaatcctcactgaaccctgacatggtccccgatgcacatatatgttagtaagtgaatttttctataatatattttactcttatttcacctagtgagcagaaaattctaaaaaaggaatttgtcatctgctccccttcattattatgaattcgagagtatatcagtcttaatgagttttgaactatatatgtgctatgaataagttgctttgaaatgatgaatacaagtaatgagagtgggaagtgatgaagccttgcagtaatgatgcttaaggactggctattttattatttttaagtttggggctttgaaatatcttcatttagattgagatgctatttaagtaccaactgtgaaaggaatagtaactaggtaggctgctgtgcccaagacagtgagcaggcctagttacaagaattgtggggtatgcagatcactcggtgtaaatggttatgacttgatattgtggctgaaacaagccatcattgaggaaatggagatgttcggctttagatttaggtggagcgagggtggaataaaagttttgagtcttctgtgttcatttaatatcctcagtaggcctgtgtggtatatattgttttcttactacgggtaaaaaaacttaagtctcagagagtttaaacagtttaccagtatcccacacaaataagcagtagataagattctgaccaaagtctctgtgactttaaagcccaagctcatccctcattctcccactcttcagtctagaagaggtaaaattcttaagagcagctcttaggaaaaactttttaaaacataaatcaaatttgcttaaatgaaactgtgccataactatatgtagtttgatcttgaaaatatctggtgcttgaggcttgttatctgaagacagatgtagtggatggattgtaaattccaaggaattgatgttatttttagcaaactttccctgagccacagattggtggcagtaatggattagctagagaaatggtgaaagggaaacttttataggaatttatcgatttttattacatttgagaaggagtatacatcttttaaaataaaaatacagctatatccttactgaaaatcaagtataatctctgatagactgaaggtatagtcagagtaccttattagttcagcagaatgggaatggtcactgcatgaattccattctcataattatttggtaaaactcttgagatttagaaaagacatagttcatagatttgtagaaaaaggtctcaagcagagattgttagattatgaataggcctcaggatatttgtgaacactgaaattttatgtaacattttgcatctgtgcacattcttttgagagagagaactgacctaaaaatagttatcaattaaatattggtgtagagggcactggtctgctgatacttctagatagattagaggtgaattcaggagtggaataaagcagtgattctcaaactatggtcaggtgccatgagaaatggactagtaagttaagtgagtgagttaaagtttgctaagattttatatttacattaaaatcagcagattactaggtttttctgagtttatatctcagaatcttccttatttctcttgaaaagttttcttcctatgtcgtatttggctacatacagagttagtgcacatgtgcatatgcagtgaaaaactagtaagtgaactgggatatcttggcccacctaagtagaatatggtcaaaaaagaagttagaaccacttgtctaaagtatttgttccagagggtggggtgggatgagtgtggggttaaggggcagttaaagatagggttgtgtgtgcccttgtttactagaatgaaagcaaataagaagtgtgtttaggtttaacttaaatataaatcttttaaaaacattaagtgtattactgaaatacagaatttatgaataagtggagacttgctgtttttgaaggttaccacattttgtgatatgtttaggctagtagatgtccatattttgactttattattaaaaactaattctttgtacttattaatagttatcaaaacatttattttcattcatgttaagtaggtcatggggttatgagaaaattctaattaaaatcatatttacacaactacaatcgttctaggtttaataaggcacatttttcttacctttttattttatttttttattttttaatttattctaaagagagaggatcttgctgtgttgcccaggctgtcctctaattcccgagctcaagtgatcttcctgcctcagcctcctgagcaactaggactataggcacgtagtgctacacccagcttcttgttctgttaatagctaattatgccacttagatgtggccatgttttagtagcaaagtaatactttacatgttaatgtatatttgaagcctgtgtcaaagtatattctgtaaaggcaagttttacctgttgataacttaaatatatcctttgtgttagcaagtgtttaaaaatatctcagaattacttagggactctggtaaatggcaaaattaagatagattctttgaaatgaatatatgtgggagtatttaatttagaagcaaaatgaatatgtatttgtatatatggtaacatgacagtgaaatttggaaatgtctgttatgttgacttttatccttatgtaaattttatgtttaaaatgtaagtaaattttatattttgtttaaaatgtaagtaaaaatcagtaagtcactttgactatttttcttttgaatttggtaagcagaaaaactaaaccttctaacttttttttagtccacccccatccttgaaaatgttttttctttgattcttctactatgtaattggtttatgttgcaaatttttttaaagtaatcatgagactatatttagtgatttggttggcatcgccattaaggaagtagtaaaagaaagtaaaattcaagcattgcctttggagcaagttagaagaaatcaaaacttggctgaacatctacaaatccaaactgagacccagctgtgtcagtgactacctgtttgcagacagttttgaatttgatttctttatctgtaaaacaaagtaattaaatttgattttagtgatttcttctacttgtgaaatctaataatttgagagactttgaggagtcttgatgccttttcattgtgttcctcttctctttgtttcagtgatcatcaaataccagatgcctcaatttagactaattgaatagaattttcatgatggagtctggatatatactttttactttaaataaataaatatgtatatattttgtgcatatatgtattttaaaaagctctgttgcccttttgggttaatttacaaatatttcctcctaagaaatatgtaatctcagccaggtgccgtggctcacgcctgaatcccagcattttgggaggccaagcctggcagatcacttgaggccaggaggttaagaccatcctggccaacatggcgaaaccccacctctactaaaaatacaaaaattagccaggcatggtggcatacatccgtaatctcagctattcgggaggctgaggcacaagaatcacttgcatctgggatgtggaggttgcagtgagccaagatagtgctactgcactccagcctgggcaacagagcaagactctgtctcaaaaaaaaaaaaaaagatatagtcttaccatggatttttaaaacaatttaggactgaaactttaaaaaaaaggaaaaaattagatcgtttgtatgaaaatacatgtttttgaatttttatgtacagggatttatacatacagcattgccttctgggaatatcttgattgtgttaggagaccagattacctattagtatttcttagtcccacttgaatccagtacttttcaatgaactgcatggtatttatggacttcagcatcttgatttctctttctctcagatcatgacagtttcattgtatgagtgaagtacaggtgtagtcagtcttctcaactcctagccttttcagagatggttgtcaacctctgtttttgcctctctcattctttgttttagttcagtttatattatctgcttttctttgtttctatccttgaggatagaattggtatctccaatttttcaaacacctttctagctttttccctcactaggcaacttctgccccactgctttttgctgtacaaatggtttttttaaaaaaaatctctcccattgaaaatggaatgaacaacaaaactttagcttcaaaatgcaagtaattttgtttagtgcagtagaaagaggttgatttaagaaaacctgtaggttttttgtttgtttttaaataaaaaaagccctgtgcaatttatcttagtgcatccaaattcttatatctgataacattgggacctggcaatttttgaccagggaatagtccatgtcatggaatactctcttcatgagtaaatctggtatttattaaagtttttattttcttagcagttaccctcttggcatataatgggtgggtggatgctgaaagtaaaagaaatttattaatattaccagaccttaagcagactcagtgcttatccttcatgaggaaggaagttctacatctgatgccagtggagttgtgaggactctgacctgttggtcgtagtctcaagataactcttttctctccagccattagagaaacaaggagttcacatttattggaatttgtaaatatagtttttctttctattggggttacccatgtattttcagggattctcctatcttttattcttttccactgatagttccaattattaatataatatcaggtgtaactataattgacttgttatagtccacataggaagtaaaataatttctagttatatctttattctttaagcttttcatttattattcctttaatttgttattgttaaaataaattgagaagaatatagtaaaactgagtagaacatacatttactggtctcttgccataatcctgatttttcagcagctattatcggatagataacacaatctacaaattttaaaacatctttgataaaaccttcccaggtaaagcataaagtattatgaatcagtgcttaggaaattgcagttggatggtggacaaagtgatataagttattctaattagttgtaatccagaaacatccttaagatttgttttttatgtaatattttcatatgttgccctggccattagcaagacctcatttatattgaaaattattatttagtgctcttatggaagcactaaattgggtggtgactgggaagatgaaccaagatccagacactagaaatcgtcctttgtaatatctttcttttagccttttgtttctgttaccacctctttctctcagcttgaagcccttattaccatagtattaagtatagtgcaagtttatctcggattatcgctattatgttccgccaccaaattaatctttctaaaacactgcttatttaggtcatttctgtggcctatggagcctaacctgtgtttacctttctgtccttaccttccattattattccagctatcccatttgttagctaaactaaatacttagctctgccaacactgtctccgacattttgacccttgacacaggaggtattcctacctttaatgctgttctttcccctttccccctttcttctcctgctgagtaaatctttttaaggacctacttaaatttgaactcccctaggaaactttccctaattgccccacttgtagtagctttgctttcttcttaagatttatggcattgtttttgtttgtttgtttaaacaattcatttgatagcaagcactggctgatgtttgtttgtacttaccttatttgtgcacttatcttctttcccccaaatacttctaaacttagtgggctcaggtctatatctttaaactgtttgcattacccatattacttaatattcattgatttattgctattttatgaataataagtaaaaatgctcttcaagtaaatttcaaatctgtttgcttataatcaagaacatctggcaatgtctgcagaagatgcacctgggattcagattgggtactaacccttgttagatattttttttagagcaattaaatatggtatcagtaagttatctgtttgaaaggggactttgtgtatattttcagggaataaaataattttaagaaatttggaatcttaatttttttcctataacacagtatcagaacaatacatttgagtaacgatagtcatgtaccacatatcaatgttttgttttggtcaacaatggctggaccacatatacaatggtggttctatataattagaaataacttttttactgtaccttttctatttttagatatatttagatacacaaatgcttacccttatgttacattgcctacagtattcaaaacagtaacatgctgtacaggtttgcagcgtaggagcactaggcactatatagcctcagtgtgtagtaaaccatgccatctaggtttatgtgagtgcacactttaatgtttccacaatgacaaaatcacctatcaatgcatgtctcaaaatgtatcccatcattaagcaacccatgactatttttgttcatgtgctgagttaaataaagttagttataggaatgatatcagtacactgttgcagtgcttatagagattttattttgtctttaagtcagtcaagataaaatggatttgaagtcagtgatttactttaccatggtactacattttctctatgtgaaaataaaatgttaatgtttttgtttgaatcagtagtttgcccaaaaatgtttcttttgtctcatttctcatgccttctctagggttatgattagtatatagaaatattaaaagaaatatattgctcttaaaaacctatggagaatatttcaataacttttatgccctctgtcaaacacccagaaaaatgtcagtattatttcaaaaactgaaatggggaatatgtttatcacatgcacaaaaataaatggtaggttagacttttgatctattaattgtaagcagtgccttttttccccctataagctattattttacccattatttttggagaatctaattacaatgtaagttccttaagggtagagattttatacaggttgagtatccctagttgaaaactcttgggaacagaagtgttttggatttcagatttttttttcttttggattttgggatacttgcattatacttattgggcgaacaccccaaatctgagaatctgaactctgaaatgctctagtgaccaattttctttgagtgtcatgtcagcgtgcaataagtttcaaattttgaagcagttcagatattggattttctgatttgggatgctcaacctgtacttctttgtgtcctcaacatttaatcaatgttttacttcccagtacctcaataaacatttgttaagatggtaaagtgtctgttttttttttcttttgaggagtttgtttttattgtaggagaatgactatattctgattttattcaatttgttttctttttatagccccattgacaaaccttcagattctctcagtatagggaacggtgataattcccagcaggtaagatttgaatacagtatcacttgatttatttgccatactccgaaagcaaggtattccatgcaggtgaattttctaaaaatactttactgacacaatttaaaaacacttttgatgctcatctttattaatcatattataattagccttttccccccagaatatatagagcatagtttaacccttttttaatgactcagaattatcattatgaccagatactattatctttagagtctgctaatgttattggtctttttgaccctgtgccaaatggtcccaggctagcatgccttctgagttccttgcttttaagaattgtattcctaccacatactccccttgccccttgctctctactttaattttctatttcagtcaagtgtatttgtccctaacagtttcccttcttattttccccaaatgactatcagctttgaaatgataagccagataaactgttcaaagttgaatttactatgaaactgagtggagtgctgaccgttagagcatgctttatgtcatcttgtcttattgctcagggaacttttttttttttttggctagtttttcactccttgagtttctatacatcttcttacattggtaaaataaatgtaatttagtatctcccgtaaagaatagccagtcctccacagtatttatgaaaaacttagagatctaaaatataatgcttttctaaacgttttaatgacacattttgtaaaagaatgtggaaatctgtatatccccttgtgtattttagttgcccattaacattttttacaggttaaaagtagttgcaaaggatgtaatttccagcatattagaaatattggtatctgaaaaatttaatgttttgatatcccatcataatgttttgatcataattctgttttattatcctgtcatccatttaaaaaaatacacatgctctcctttaacaatcagaagttttacattatttttttctctgtgaactaatttttctctttctgttattatagagttttatcttaatgcaatatagtttttgcttgaaaagcatatacttctcttaaaaataggcatataaatttttggattgaaataatgtttttgcggtcacagacttcaaatgccaatttttgtcttctgaattttattagtagtattgaggtgacactatacataaatgtagattttaattttaaaaaccatgaaacaaatagtaataaatggtaaaattatagtaaaataaatagtaaaattatattgaaaattcatctcttaatgaagaatagggccattttaaaattggtttgtatgtatctctagattaatgttacctattggtaaaagagccaagtttcagcagcaatctagtcgtgttttttatttttctaagatgtaagtgttgagataagatactttgttcatataaataggtagatggaaggaggagtattatatcagtaccactcaagtctttcaatttcttctgaattaaatacctaagaatcatacaaagatttattatacaaaattatttttaatgatgtgtttggaaacaacttaattagatctttctttaataatctagccattacagtcagtttctctcttatcaccaatccatgtatttctaataatttgtgtttggcctctggaaagttttcactcttgagcaaaatactgtagtaaaatcccagaatgagaaatattattttcttggtaaagtgagagaagtaaagttgtaaaaagagaggagatggccgggcacggtggctcatgcctgtaattccagcactttgggaggccgaggcgggtggatcacgaggtcaggagatcgagaccatcctggataatgcggtgaaaccccgtctctactaaaaatacaaaaaattagccgggcgtggtggcacgcgcctgtagtcccagctacttgggaggctgaggcaggagaattgcttgaaccccggaggccgaggttgcagtgagccgagatcaagccactgcactccagcctgggcaacagagtgagactccatctcaaaaaaaaaaaaaaaaaaaaaaaaaaaagggagactagtttaatcttttcaagcagattaatatttttttaaagagtatatatgtaagttaatgatttaattgattttctgaaacttcatgctttcattaaaacatcttcaagtaccagtttgtgaaacactattctaatacatttagttgcaaactttttaattgtggcttattaaaatgtataaacatgtagttattatttgttttccttttttattcagatatctaacagtgatacgccttcaccaccacctggtttgtcaaaatccaatccagtcatccccatcagttcatccaatcacagtgcacggtccccttttgaaggggcagtaacagagtcacagtcgttattctcagacaattttcgccatcccaaccctatcccaagtgggcttcctcctttccccagctccccacagacatccagtgactggcctacagcaccagaaccacagagcctcttcacatcaggtatataacaatagtaccaagtattagtaattatgctcttaacgtattgttgacagactaacatgaaactcattagtggatatatttttatcttttgcctctctgttctcttttaataaaaatgcaaatatttaaactacatggaattaggtggttaaatacattgttgtgtgacctagaaattactgtgttaaaagatttctccagaagtaaagggttatattgttttcccaatagtaaaaagcttattccattgggaattttttttttcatttttactttttggaggtgggggtcttggtctctcgcccaggctggagtgcaagtagtgtgatcttagctcactgcagcctcacaccctgggttcaagtgatcctcccacctcagcctcctggttaatttttaaattttttgtagagacagggtcttgctatactgcccaggtcttgaactcctggcctcaagtgatcctcccacccaagccttccaaagtgctgggattacaggcatgaagcactgcacctggcaaggacttagatttttaattagatttgtatttttatttaattagatttgtgtttaagattttccacccccagctgacaaagatgcaagttatttagtagtaacagttatgttactactaaatggtatttttgagccatttccatttttatccattctaaattctctcattactatgaaaggtggcataaaagtaacattgacaacagcacctgtggagcagaaaattccaaaaaactatgaggattaatataaatactttttaattaaattatatttttcttagtatagaaagcatgtattcaaaattatttgtactttgataaatacatttaaaaatcaactttactaattaaaatctgttatatttatttgatggaagtgacttaggttggttttgattatgaatggttgagagtataattggagtgtcaagtacatttttttcctttgattagcaaaatcagtctatatcattcagtttttgaagctagtagcttaagagttaagtaactgctagaagtacataaggccttttgtaattatatgatcttaatgtgctaagaagtcacatagattttgcacttacttaatttgtaaaatgacatgtttaactttcagtagcatggatacttattaagtatgaaaatgagctatgtatctgatattcatcactatttttcttcccaacagaaacaatcccagtatcatcctctacagactggcaagcagcttttggctttggttcttctaaacaaccagaggatgacttgggttttgatcccttcgatgtcactcgaaaagccttagcagacctgattgagaaggaactgtccgttcaagaccaaccttccctttcgcccacatctcttcagaactcctcttcacacactacaaccgccaaaggtccaggctctggattcctgcatcctgctgcagctacaaatgccaattctctcaatagtaccttttcagtcttgccccagaggttccctcaatttcagcagcaccgagcggtttataattcattcagttttccaggccaggcagcccgctatccttggatggcctttccacgcaatagcatcatgcacttgaaccacacagcaaaccccacctcaaatagtaatttcttggacttgaatctcccgccacagcacaacacaggtctgggagggatccctgtagcaggtaggttcatttaaaacctttgagaatttttgaataactcatattttcaggttgttgggtggatagggaagtaactgttgaataacagagtactaatatcttggaaatgtctatgaaagattagtctttatcataatctaaaatatatttttcattagatggatgccttgtaatattgagctttataaggatggtggagtattatacttagttattgtttggaagcattttacttaataggataattaccaaactttcatatggtatacaaatgcaaaatgaaaaagaagaaaatggtctctgaagttatggtactataaagtccaacaccgtggtgaaaattatgattttatattctaaagtaaaatggttaaaaaaaatttttttaatgataattaatgagagcatgagggataaagccactctctacctacttttggcagttacttgttttgactgatacacatatacacacacatcaaaactgagctataaatatacttgagaactctgattatgaatgttttacaagcatatcatatgtctcagacatacaccttttgtgaaagagaaattgttcaaggaaagatactttcccatttgatctgccatgtttttatttctggggaaaaatatgaagtatatataaggtaatgtaagtttgatgccaaaatacttctggttttgtactgctacccttgaaggtttcagaacttttgggttttaagattctatataacattttttagatgaagtatcttctatatccaagggaattattcttgttaatagtgttctcgtttaactttggctttagagatgatgttaaaaaaagtttgttcctctcctgattctctcaagtgaaatagaatcatgattctctcaagtagaatacaatagtttctcactctgttagaatagttcttcacataattcatacaatttattgttatcccttgttagatatggcatggcacagtattattgtaataggatacttttatcagcagcaaggcagaagttcacgctcttgtttttagcaaggctgatagatgaatttacagaggtctatcaaagttctttatccctgttccctttgggaccgacaatatttcatgttttagacgagtagctaggttaagagaatatagtttgtggctgttgacatagtgaaatttaccgttgatacagtgaagttaatttacataagcctggtagcacgtaatgtagttatttgcacagtgttgggactcttatttgttcatttaattatggtagttagtaggtaaccagagtgacatttttcagtcctcttcttctacatgatacttacttgtgaaacaatgtgttcagttccaaatactccttttatttccttccattcaagatagcatattagaatgtaattttctttgattttactttgctttaatcattgagaaatgtggcattttaattactagtagtgtatctcataattgattataacattcattaaaagttaaagttaagttctcccaaatcatcaaaacatttaaagattagaccaattacagtgaatatgagggtttctatttgcccatcaatgaattaggacatgtgggacatattaaagaagtttaagatatggataaaatttccacatacttaaagtatagttgaagagactaaaataatgtgtgaaagtggtaactatatgtctgatactggaatctgagagacatcaatgtaccctgtaattcttggggaggcttcattgaataagtaggaattgatccctactatcatatcttagatttgggttagcagcaggggaagagaaaaggccttttatgttgatggaaacctcctgagtaaaggcatagaggcaggaattagtatgtcattacactgagaagtaatacagctacctcctataacaggaggtgcttcttaaattgtatagatataatggatgagaagatgagaaactctgacaatgaggcaaaatggcatggacttggtatgataggaagtaggatgcctttatgggattctagttattagctgtcttctttttgtgcttttatttgacttattacatgacgatctttgattttcttccaatttcatagtcatcatattatcccttaacactgaagagtagttttaacccatatatttcaaaccaatagttattctcagaatgctgcactggtaatgagaaatcgctgattagtgagtttagttgtccttgtgttagtcatttaaaatcttcgtctaaagtagatgctttatggaaaaaatgtgacattaaattatggtcctatctttggggattttgttttaatttttaaaacttttaccatatactcaaccatagagcattaaaacctttttttttcagagatgctaaaaatgtagtcaccaactttaggttctgaatcgttgtactatttggtagggggtgggagagtttattttggattttcaatttcaggctatgcagacttgcttggtatttaaatggtgacttatggttaagcttattattaggttatttttaaggctctgatgactagagaaattttagttttaatgtaccaaaatttaaaaatttttatcaagagtctcacacaccagaatcctctcatggattataaatatttggtagatgccccagaaaactgaatgtgctaagcctcagagcagaatagctgtgtaattgtagccagaagatttaggattttcagtaactattagaaatgacagaaagtgaaaatctggaaaactatatatacatcttaactatgtctaatttgaagcaatttaatacctaggtaactcacttgcacctgttgtaactcacttgcacctgttctaactcacttgcacctgagttgaactattggcaaggattcaactttgtatcttttagcagttatcatgataccttacacagggcagctgtcactaaatgcttcagtaatggagagagtacatacctgtttcagtgggacctgctactttaagatatacttagacaatattttaaactaattctacactctggaaaggcatatatatttattctctaaatatgtagagcaaaatatggtctctgacctgtaagagcaaatcagagatggcagtcacctacatgtcatacagatagaatgtctacttactatacatttgtacttaaatttttatttttggtttgttcagtgaattattaaagattgatatatctgaaacctatcatgggaaaaacagttgccagaggtttgacagaaatgcagcttcctttttttttgatctaattgaaaactgtttttgtgtttaaaattagtagttatttccttaattaggtgaaacctttttagcagcatttggtttactaagtttagaaatatttttgacataaatattcttgggggaaattttgattcgtgggcagttacagtaaattcattcagatctttttttttattattattggaaccatatgccttctttctacaaagtgatttaccttgtcctaaaacgtgactttgttttgtccaattccttcattttaggaatagcaaagctgtgttccagaaagcttaaataatttgttaaaggtcacatattcaattagtagtggaattagggtaagaacttagatctcaaattcaacaatggaaatgtttttgcactacaacgatgccctccttcataaatgaagatcactggcatgtgctttaacttggctttcttcccgtggcaattgtttttgctctcacacacttggaagtccctttttatcaacagtatcaactcatgagacaaattggcccagctcaggggtatatggcctattgttacaaaatagaataaagtgtatcgacttgatagaaatcagtctgtttcttagtccttattaatatcattttcttgctaagaactctagtctaaaatttgctttacagagcctcattgctatgttcttggggtttataagaatctttgccaatttactgaaaaactacaaaatcacctgttatatggattgacagactctttttctgctttgtcagttatccttccacatgtaattttgcagatttgttttaaaaaaatattctgccaagttgtttttattccatgatgttaaaaattatttggaaaacataagtcagtaagcattgttgtttcaagatatacctcaaaaaatatttgaaactgtttaggattggatttttttttcatgtttttgttaatttaaactttacttttgagagggcatttttctttaaattttctttgtttctatatgaactgttttaacagtgacttgattgaattttgagtcatgtgtgaaacaagtcacttgtagtcaaataattttcagttataatcaggagaaccgatctcattcatctataattctctgacctagaagtagtcatctcattttcagtcaattggatcattgtactgttaacattttactatttttcaattacttcaaaggtaaacttgaattttcagatgctgtatgttaatttactagttaatatttatagctttcatttttttctagtacataatttatgctttcaaaacatggatttgtaatttttctcttttaaaaatcagttttcacttctgtaaatcaaggtttaattttttcacaaaatactattaatatttaccatttactaggagtatctataagggtccaattggcttggaaaagaatggctttcctggtctttttttttttttttttttttggtagtatgtttataatctaaatctattgtatttgttcttatctctgaaacaatattactcaggatatatttagaatataactactttccaagtacttgataagtttatattttaaaattcatgtcttgtttcacacctggattccaaacattaagcatgtattttatatatgtatcagtattccaaatattgaaatcatttttaaagggactgttaacaaatctagtcacccataactagaccatgactgacttttccacaggggaagaagaggtgaaggtttcgaccatgccactgtcaacctcttcccattcattacaacaaggacagcagcctacaagtctccacactactgtggcctgacaacagaactgagaggagaggattagactctggggtgcttgcatgggcaactggatttttgcatgattcctttatgattttgcttttaatgtatacacccagaagagccaatataaacgttcctcatgcctacagctagcgtgttacctcatagttgttgaagtatttcttcattaggcctttaacataatttattagtgtaataattttggcattgtttctcatgagtgatactatttctaactcataaaaataaacttgtagtactaagatcccaggtgagattagctagaattgttgttattttggctctattattgaaaaattatacaaaggtaatggtagtatcttgttttctggtttgtgaaaaaataatatcctaatctcctgaaaaatttgcaaattaacaagataatgtctattgcattctttgaatacttgagaagaaagaatacatataaaacaaaattggtggtgtcatttaatgctcagaaggatattgttggcgattgtttttagttgtctagatttatatcctataatatgcattacatgtgtagaaactgttttgttctctctcttttgcacttttgcactttctttttgaatggttgttaattatgcatagatggttaaaagataattattgtagcctaaatgcaatataatctcttttctaatgcactgcctagtataccaggaaatgcctcaaaaacagaacttcagtttatagttgacttatctgaaacttgaatacggagtaaacattgtatgtgaaaagcaaccatagtagtgtaacattgttactgaacaaacagtaattctgtgggatattgttttggaaatttcattgtttaataattaaaatgtgataataagataatattataagtacctgaccagaatgagactaaggcagaagagaagagattggatgtcctctaaaattaggccacctaaattgtaacagataattttaatcaagagaatagtggagtactttattaggacataatgtgaagtgatatttctgaaaacacatttcttgaaatagttttaaaactgataataaaggaaaagaaactgtacttagtttggagcagtggacaaaagctgattcagaaaacatgaccatattctgcctacatgaagcatctgggaagaaagggaaagtcacacctcctcttgagattttaaccattatgataaaaggtatgctttaatttgttaaaaaaaaaagtatgaaaggggataatgtgaatacaaaaaagaagcctagagattgaaaataataataatagaacatgttattaggagatggtacttaatgtcatgttggagttggagttttcaaggttacctctatataaaattggattattttctgttagatataatgtgacaaattaaatgactttatcccaaggtacagtgaggtaaacaaaaatctattcttagatatccttagatttggttaactgaggctgtagaagcagcgaattgtctagccatttaagttatttttgtaaaattcagcataaacatcaatctctaatttctatctagaagtgactttaagacattacaagaacattgccaatttggaaaatgccaataaaatactgtagacttattaatattgtagacttattatcagtcttttactatagcttaatttaattatggttttcatttttactgtaagaggccaagacttcacttatgtctcctgtggagctgaggctcctgggccttgttattataacaaactctggctccttgtaatgatgcttactagctcaaagccttctccagcattcagtgttagcctttcattgcctggatcttcttacaaagccatagcaagtggttagtgccatttgctccttggtgttgagagatatacctgttaaatgaggcttctttcctttctcattcatagccagcgtggaaaaatcatcaggtagtttgcccagatttgccagagccctaaataaaatggcaaaacgatctccctgcctgcttctcctccccacccccacttacatgcacaccgtaaatcaagaaatgctttttgggcaattagactttatcttctaaatttttgcatagaatggctaaacatgatcttttgcctcactttgtaattcctacaccttccttgtaagcttcaccttttgcttacctaatattttatttaaacatggttttttaaaaaacctgaagctttaaaatcatagtcataattaattttacactaaaggacagaagaaattatacaacacataaaagaagccactacttcacatagcaagcaaattgaaatcaaaatgacagaagaatgagaaattctttaaaagcttcaggttttttttgttgttgttgttaaattcctgcatgatgcatcatgtgcaagggaaacttaatatacttttacacccttaacctttataaagtgactagcttttcccttaacataggaatttttaacctggagcccagatttttgagagtacacacaaaattgtgggtttgtgtgcatgtacatatgtccatttttttctagggagaagatccatagttattagatttgtgaaggatttggtgatccccttctcccatcaaaaaaaaaaaaaaaaagaacagctgccttgaaatgtttttagtgccactttagggaatagagtcctgtactgaatgattttagttgtatgatgaattataagtgcctctttattttctctaagattgcattagaaaatatttttttctgactaataatgctttccttttcattctattattgaattttggttttccatgtgttacttttgtgtactctccccttatttcaacaaagcattaattaaatatttgagtacctattctgtatagtgacttgaactagatctttagagatacaaggttgaataaaatacagaccctatcatcaaggataattctagtgacaattatatttcacattatttctgtcaggtcttgataatattttaatcacaggaaccaccatagcagtccagactcattttattatttatcatctctcagtaactgctccgacagtggcaacaaagggtattgaatacttatatttcaaattttaaaatttatgataatttggagggaggtgaaaaaaccttactaggaaagacaaacattcattattctacgtgtgtgtgagctcatgtctcttactcttagcatcctgggaattaagtacagccttctgtgtaggtgttcctttaaagaaaacaactttaatgcattttacctctgcacatttccactgacatgactcctatttgtacagtgttccgagcactgtcaagactgccagagctctggcacaggtccataggtactcccacggaggcattgcctcagaaagacaagagaaaggaacaaattgcagttgctttgaaatcttacagtggcctataggggttgggatagggcagtaaggagatccagctgaggtattcagggactggtaagcttggataagtgtgtttaaagccttgaaaaccagaagaagagtttagtcttgatgtgaatgtggtcagtggtagagaactataggttcttagcaaaaatgtagtttgaacatagcattttaagaacagtaatggggtaatagatttgattgcaattaggaccggttagaaagtaattccagtagtatgccattgctggccagtgcaaataagttggcctagactagcacagtagtggttggaatggagaagaactatgaattagaaattggaaagcgtaagagctgagaaaaggccttcagatttggttagatctgtggtgatctttaaaaatgtaactttcatagaaaagtacagaatggaagcccatctttttaggattaatgaggaaatgtcaagaatatgtatgcagcaatcattgactacttagtgttaagacatttgacaacaaagttgaagagacaaacaagatgctaactagatgggtggtagatcttagaaggcattgctttaatataagggagaagaagaacctaaatatttaaaggcagaccttacagagccaattgagaggaagagactggcagtgctgaagagaagataggctcattcagtaggataatagatgtcgttgggtggtgggaagagataagctcctgagacgagcatgtggtatagcttaagagaggtgggtggccaggcgcggtggctcacgcctgtaatcccaacactttgggaggccaaggcaggcagatcacaaggtcaggagatcgagaccatcctggctaacacggtgaaaccccgtctctactaaaaatacaaaaaaattagctgggcgtggtggcgggcacctgtagccccagctactcgggaggctgaggcaggagaatggcgcgaacccaggaggtggagcttgcagtgagccgagatggtgccactgtgctccagcctgggcaacagagcgagactccgtctaaaaaaaaaaaaaagagagagagagaggtgggctgtatcttcctctgtgatttgtaagagcaagaagaacctggggaaagagaaagatgagaattgatgaaatggctcatatcagtaattattaaaatgcattattgtttgaaaatgggggtgaaagagtggaattgggagccttttaaaggagattaaaaaaaaaagtcttttgtgagaataagcccagaaatgcattgtaaatagttgcttagttgctgtgtaaagataagagtcagcttaaagatctctaaagttgaaatatatgtggttttgtatgttcctctggtttagcacaaagcataggagtggaaaaagcaaatattgagtgtttggttaaaaggggaaaaagacaaagacatagagacattcaagaagaccacattgaaatgtataatcttgtgccaagaagagtctattaaggcagtttcctgtgaagaaatggtgtgagtgatgtggaagggagttgaatgattttagattttgatgatgacaaagaacatgttagtgttgctaagggaatgataactaggagactgtggtcactgtgataaccactcaatgctaaatgatgattgagaaccagagaaaggagagggttggagtttggaaagaagcttgtcttttgtaaccgtctatatactctctcctccagtcttttcatgagtgtaggaagtcaaactatgtgagagagaattgtagaaagcatggaagcaattccatgcatccgcagatgggcacctggggaatcctgggtcaggtttccggtgagttgaaaatgatcaaaggaaaacttccttcctcaaagcatcgtaggaggacatggaagcagagtgctggggaggaccaggaaaagccagggatgagatgagacagctataagtaagcagccccagtgagtgtgaaggattggtagaaatgttactgatgatgtgaaagatttagacagagggattaggtatggtgtatgatgagtttggtagttgaggagcccttattacaggtaatatagcttctcccaagctcacttgcaaactggaaggcttttttaaaaatgggcttccacctatgaagggaaagcatctgctcttacatagttggattaggtcattctgctttatttatatatcaaataagaattaaggtactggtgaacagtagagataaagtaagaattttattgtaacaggttataaaaatattttgataatttcccccaaagagaatgtactatgaataaataaaatctacaaattattgggctaatttacccttgtttcttatattttgtatacttaggaggtgacctctaggaaaaaattgctgaagaggtagtagagcagggtgaagataccgtatactgtcatccctaggtatctgtaggggattggttccacagattgcaaaattcttggattctcaagtcctcgatataagacagtgtagtatttgcatatgacctgcacatatatgggaggccgtatattttaaataatctctagattacttctaatacctattacaatgtaagtgctatgtaaatagttgttatattgtttagagactgacgaaaactgcatgcttaataaattacacaattttttaaaaaaattttctattcgatgttggttgaatccacaaatgtgaaaaccccaggtacctggagtcaactgtgtggttttttttttttctcctagtcagttaataataaatattcctagttgggaagcaaccttagttttctaataaagtatttttaaactcactccgtatatgtcagctgaaatatcaactgtgacatttaaaaaggataatgcttctaagtacaaatcattgctcaagaatactatgtttgtgaaaccttgttgatggattgagaagccactgctccttggacctccttatcaccctgtgatttgcttctgtgagttgtaatcgagccacctaacattgctggtcatactagccttccacttcttagtcaggggctatttccaaagaatcactcttgataatttgagatgcattaagaagattctactctgttttttggtcttttagagagtcttctctgggtttgagttatagagaaatcgagtttaggtacctagaactgagagcaagcctaagtaatctaagctacttgagaaatggactgaagtacccagatgttccataaaacagcttgaagttgttgaccttgagaaccatttccttttctccaggttcctcttgaccacacttcctaaggcaactattgacttcactgtcttagcccactttacctttattctaagagttcctggaagctccaagctgaccctggaacatactgtgcttataacaattttgttattccagctcctgttgggtgctcagacccactattgtgctgttacaaagcaacatcccaaaatgctttttcccctcatataatttataaagcctttaatgcatccattacctttctttcttttttatagcgggtgaaattcacataacgttcaattaattattttaaagtgtatgatgcagtgacccagtaccattctttaattatctcaatggtaaaatagcagaataagtaacctactctgctgtaagaattttagtctgtttcttgttggggctgtgtggtaatagctgcttctagactctgtgttttacccagtaggctgaaaggcaaatgttaaacctttaaatattccgtatttctgcatagcagtactactgcaacttaagcttgtgaatcaagcaacttgtatacacccttttaaaaaggcagtggggggcagtgcaaggagctctaggcaagagacacttaggagatttgcttttctgagtggtaaaaatgtttgaagcaactctaaatagctttgaaatgttagtaatagtcttactgtcttcatgacatttaattagccttctgtaagagtttgtttagcaaaggtaaaccataagcctagccctctatatgcatgtgtggttgaatgtgtgtgtgtttttatataaagcaattttcagttaaatcttccacttaatgaatgtagtagaggaagatgtctcatgtcattttggtgaccttgacctatgtctattcatgtatttattcctgctgtgttccaattcatatcttcaggataaatgctgtatctttattgtctttctccaatttttttttgaaggaaccaaagaccctatttaaatgcaataaggttgtaatactggtgcccacttcaccctggtcatgttttttctgaacaaatatttcatagcttttcctcatcaaattccagtcccactgctaagatacccatctatgacacagtctttgggaaaacccaaggaaaggataacttaaaaatgatttgtattggaatcatgacacttcagagctatgaataaataaatataacatagtctttaacaatccttttaaagaccgaataataacaatgatgataataaacaaggctccgaaggattgtgatttgtctatagtcacattctaacaacaatttgaagtttctttagccctcaccatgtaaatgtgctttcatgcttgaaactcagtggaggaaaaaacttttaagattcagaatctgtctcctctaaacattattgtgtcttacagtttaattacagtaggtctctcaagtcaagggcttacaactgtgaattcattgcaaacaatgaatttgaaggctttcaaaatgaccaccagctggcagctgttgtcacagatcctgtaggttttcaaagctgccctgtgttaaatgtggcccagtgatcttggagtaactttagcaggggaaaaaattgtaaccttgcaggtactcttaaaataaacttatgctaactacaaaattaccagtgtcagaactaaaagagtaaaacagaggtggagtttaatttacccaggatttttctgcagtaccaaagagaatttaggggacaacacacataaccactagactggtgacatctctaatattacaaaatggaaaaaaacttatagtaatggcaccaagatgtattacatacacactaaagatgtgatctccgattcatctttgaagaagagctactctgtgttacaccatatagaatgatagcatatctgctgggtttttttgtttttttttttttccccaatcaaggaagttgttgttttcagcataaggaaacagggagagctccacaaggatttaaagatgataatgctatcactatctccaagaagaaatgcaagaaatctgcaatcaacttttgtagcctagcactgttgcttaatatagtgagatcttggcccaaggactgcacttagcatttacactctgttatcagacttgcatactgaaataatgtattgatggaacagaaagtagtgatctagaactttagtggggtgttctaaaacattgtcgtgttatatgttggcatttgcaagaagtctgacaactgtatatttttatttttttcctttattacatctatatgtttagtatccagctaaaggtaaaagattgcctgggttatgacttcatcagccaagtaggaagttttttcattggccttaaactgaatgtttggttacagccatagttccacttaggttttctctgttaaaaatgttagtttctattaaaattcatgattatggatggctgatatatatatacacacacacatatatatatacacatataggcacacacataccctactccccccaatccccatttagagtaagatttgctaccagatatgtttccttttccttctaaactgttaacatttactaattttttccttctgaactgttaacatttactaattaatctacccatagtcttcccctggttccagacaagtgtaacatatcttgatttctagtctgcccagtgttgatagatgtaaactcctaagacctcacctgcattttaaatggtagatggctatttaattgaaacgaagcatattattaaaataaggattttatataatctccaaatacacaagccctgcaacctttgttttataacacgctttcaggttttaactattgactattataagtctctttccggtagcttgtgttcacccctctaaccagtttctttttctttggacaaaggagaagtttctttctccatgtaatttaccacaattaacagggaagtcatgactaaatcttaatgtacactacctatatgattttcctactggattcctgacaggcttgaattttcccttttcttcttcttcttcctttttttttttttttttgagacagggtttcactgtgttgcccagattggagtgcagtggcgcaaacacggcttactgcaacctcgacctccctggctcaagtgatcctcccacctcaaccctccaagtagctggggctacatgcacacaccaccatgcgtggctaatttttgtattcttctagagacagggttttgctttgttgcccaggctggtctgaaactcctgatctcaagtgatccatgtgccttggccaccaaaagtgctgggattacaggcgtgagccaccgtgcccggctaattttccctcttcaaaggagagaagtctctctccatcagtctactgagtgttttctcagtctttttactcaattaccagagacagcagaccctctatctgcccaataactactcttactcacctgtcagcttattccaaactgcctttgatcctaaacgagtatattcttgtttttttttttttccttctctctatttttattttgtttgagacactctcgcccaggctggagggcagtggtgcgacgtcggctcactgcagcctctgcctcccaggttcaagtgagtctcctgcctcagcctcccaaatagctgggattacaggcatatgccaccatgcctggctaatttttgtatttttagtacagacagggtttcacattgttggccaggctggtctcgagctcctgacctcagatgatccacctgccttggcctcccaaagtgctgggattacaggcgtgacccactgcgcccagccttctctctcttttaaatcaaaacaacacatttaaacagaaatcattgtctggattttggagcagattggggaacgactaatgcactgtaaaattttgaatcaataaagatttttgacattgtaaaacttgggcattactcagttcccccaacttctaaggcaattctagctcctataaaaagtcacttaacacttctcaacaatatgactaaaattcagagttttacaaatgtgtttttaaataaatttttttcagtaaaacagttctagttcagtatacagcactagcatatctgaagtaagaatattgctgtttctgatttgtatttgaaaatattttcatctagtcttcttttttgccttccttcttttgaaataagattcaatattgactgagaattggttctgtcaagattgtctctggcaatctgtccctaattcaactataaattattgctaaggatgaacacttaaataactatatattttaaatgatttttaaactacggccccttagtaatgtgatgagcatataaatttttgaattcagggtcttttgaaactctggagtatcatatgtatgtataggtctggaaaagatgtgttcctattaagacttaattctttttagattgagacaccatttgcaaattgaaaacaaaaaaagtttgtatttcccctagtatgtgaatatactgtaaggaggaataaaaagcaaaatagtctttttaaaacatagactttaatttttagagctattttaggttcacagcaaaattgagtggaaggtacaaagacttctcattatattccctgccccatcacatgcacagtgttctccattatcagcacccccaaccagggtggaacatatgttacagtggatgaagccaattgatgatctgggtggtgaataggtggaacacagaggagtttcagggtcatttttagaaagtacagattagttttaaaactaatgtagctttttcagattgtcttcttccacttagtaatgtgtgtttaagtttccccagccttaatttttaatcacacattttacatagctaaaatcattgagtttcagagtatcaatgcttattttaaaaaacagacatttattttaaaaacaatctgaaaaccttaataaatgtctcttaatgagaaagaactcttaaagatgtatgcttggtctgttgtattggaaatgtttatcaccataactatatccatctccatttcatttagttttataaaataatgaagttatatcattttctgaaataatttttcttctgaaatgaaagtaaatactcttaatgcctaaaaaatactttctaaaaatgaagctcataaccagattatgaagaatctgtcaattaaaatgatgtttcagaaaagtgggaattttattcttatttgggatttttgttatgttgatttaaatgcttaagacaattcgatgctgataggagccatcttttagaaaaagataagactgactgatctttactattgaaataaagtgtgctgtgtaatcaaatgcagtaaagaactgggtttgatgttttaaataacttttagataacttggaagtcttttaatttggaaaattatgaacatcagcaatcttaggaatttcaataggtatataaccataaagggatttgctgtataggaagcacaatactttcatctgaatgtaaacctgaagagagacttctgccccaataaaaatattttattgctactaaacaatcaaaagctattactgacttcatttggaatggccagtcccgagtgatctgctaagaaataagcagaacttcacttgactagccttaaaaaaataaaaattaaaattaaaaaaaagcagaacagcatggtttttaggaggctgttagagaatgtcatagcatcaagctccagagaaaatggtacagagtagacatgcaataaatgtttgtggaaggaaaattgatggttgagagtggtgtcaattcggaggcctgcaaagcatggatgtgttttgtgttaatctggacttttggaagcatttagtcagattcatttgcatatgagttataattcattttttggttcttttctggcattgtggtgttcattgttaagattttcccccctacacgtcacccactgactatgtgattaatttttcttctttcttttttttttttcctctgaacttgggaaacctcagctcttggcttttccttatttttccaacaccccctgtacacacatacacacacacacacaccccttttgtatatttttttgagacggagtctcgcactgtcacctgggttggagtgcaatggcacgatctcggctcactgcaacctctgcctcccaggttcaagcaattctcctgcctcagcctcctgactagctggaattacaggcgcccaccaccatgcctggctaattttttgtattttttagcagagacggggtttctctatgttggccaggctggtcttgaacccctgaccccgtgatcctcccgcctcggcctcccaaagtgctgggattacaggcgtgagccaccgcgcccagccctaacacacgccttttaaaaaaactaacacatttttcttaccaggcgagtcctcatttttgatacccataaatctatatataaatattggtaaatcttccgtgaaacacatacacacagtccagaaagtctctctaatacctactttttcagaatgctttgtgtgtactcctgaggtgttttaggagcttcgtactttgctaggtagggagttctttagggtaaaattgttagaaattttttatcttggttaagccaaaatcttttaccaattgaacttcttaggtgcattttgttaaacacatgaagaccacttgtctgttaacttctggatgtagatcaaacatcctcacttctttgaaccattccacacttaacatgattttgagttctctctcagtttaattctctttacacatattttgacttgacgttgcctttcttaaaatatgaagctcacacttgaatataataatccacatatgatcagataatagatgtcttttattaaaacttgttactaatatttgtattttcccttatgtttatagagttcttttataaaaatatcttgtagggtagttatctccattttataaatgagaagcagagtggttatcattcgtgatttgcctaaggtcacacaattgctaatttagacattgcacatctatttatatggtcttaatgttacttttttaggcgttccattacccagttgccagaccaagtatcataagcctatttaagtaaaaccactagaccttttcccatacacgctaatggttagatcttttccattctaccctttccaaatgcatttttaaaaatctaatgtatatctgttaaattgtatgtgtttggatttggtattgttagaatgtatcctgtttgtatatccccatcttagaagtttgtcttatgaaagaactatgtgattataaagtgatagatgcatgttattaataacactattaataacatttgtcaaagaatgttaagagttttaatttaagaatagttgtagtttccactattctgtcatctgtagaattggctttccgtttgcatatttaaatgaactttgtggcttttgttaagtataataaaaagcatggagtcaaatataagccaagagtattacagagacttttaggctgactcagtatctcaagttctgtgtagattcatctaaacactgctgttatccatgctatactttaccatgttatcccaaaagggaatcatcagcaaattttaccagaaactgctgaattcaagatatattcaatatatattatacttctgacatcctaggaagcctatccaaagaatacattactttgatagaatttgttctttatgaaaattcattttgactctcattgataactttattccattttgggggaggactgaggagtcagtgggatgggaacagagctaactacaaagtctttgagtttagatgggcagcagaaggggaaaggaagtaggccgtgggatatataaggacttttccaatggaaaacaattgtcagtggaacctctatgactacttgttcaatttcagaattaaacttcctgtatattttaggtggaatcaagctgagttctagtcaaaatgctacgcattatttcccatgaaaaatacccccaaacacaagcagacagaacagtggttgataaacccatcatattcatttctgaagaaatcatcaagccccaaatctgttttagaaatttctcaagaactaattctagactgggcatggtggctcaggcctgtaatcccagtgctttgggaggcaaggcaggcagatcatccgaggtcaggagttcgagaccagcctggtcaacaaggtgaaaccccgtctctactaaaaatacaaaaaaaaaattagctgggcttggtggcacttgcctgtaatcccagctactcaggaggctgaggcgggagaattgcttgaacctgggaggtggaggttgcagtgagccgagatcgagcattgcactccagcctaggtgacagagcaagacttggtttcaaaaaataaaaataaaaaaataataattctaagcatgttgcttccagagtctgccatttcttccatttttggtatttttctaacagtggttttgtgctctcacctgtgaggattaagagcctgggataaaactcattttcagtctaaagatttaaactcatttgaattgagtagatgcttttgtgatgtctgcatctctcctgacttaatagttcccttcttcacattacttaatctgtccttttggttagaagatcatgctcttacctaaagatagaaggaagcaaaatgaacatggaactacattgccctgccttttctcctgtcaaatatggcatcaggctgagcagtggcttttcctgatattccccttattcaaaacatagctttgtcttgctttcagcatttttcactttcctcagatcattatggattctgactttaatccgtaagaattatacttttatatacaagtaggttatatacctctacccatcttttctaagctttctttgcaaatctagtcccatcagattctccctgtgtaacaaccaacttctttagatttcctctaattttcttttctttatagatttatttattattgcatagtcaaaatgtcatgttttagcatcttctgtggcctgtgaatcactgctctctataaccatgggaccactcccacccatccttttcccgatctttttttggatagttgttttcttaaaaaatctggaagcaggctgggtgcggtggctcacgcctgtaatcccagcactttgggaggctgaggctggtggatcacgaggtcaggcattcaagaccagcctgaccaacatggtaaaaccctgtctctactaaaaatacaaaaattagccgggtgtgatggtgtgtgcctgtaatcccagctatttgggaaactgagataggagaatcgcttgaacttgggaggcagaggttgcagtgagccgaggtcgcaccattgccctccaggctgagcgacgcagcgagactccgtctccaaaaaaaaaaaaaaaaaacatggtagcaaacgttcagggttatatgtttgggaaacatacagtacctactgaagttcaggttcaggcattgtctactacatagaaatgattaaatcgaggtccttttcctagaggagctcaaagtctagttgtaaaaagtgaacatgtaaacaaatgattgcaatacactgtggtatttgcaaaatagcgaggatgcctgccatctaagaactcaagtataattattcccattccctgcttttgtaaatcctaaaattatgtcacgtcctcctagggtttctgttatttttgcagtagccaaccagttcttcgttggtcagaattaagtccagaataaaagttttcttccttattttcaccttggaaaagagagatcgtcagcaaggaatgttttatcaggtttattttcttctagcagatttagacttcagagatgttagggtggttgacaccttccatgtctctgcaattttgtcatttgttttggggaagtcatattgggtgaatattctggtgatctgtgtattgtctccttattttacattcccttgaccatctttggatctcctcacagggctttgttcatggtcactgctcagttttgtgctcacttaaatgcttccctccaggctgctgctttgacattgtaaagatcttttatatgaatcttttttgtcccttgccctcatttagcgttgcttacatattgtggttcgggtctcatctgatccagtctgtgatgcttctaagggtgtatgtatcttttatattcaaatttgtttactttttcctctttttttatataactttgcacatgagtattttttattttttcttagattctgattccttagatccttcataaagcagccatctagattcttctggttctcattaatctgattctccttcaaaacagatttatagatccttataggaacattctagaagagaataatatacattatgccaaacacatctacttatattttgaaatgttcccactgtatttttttatttgctgcctgtggcctgtgtgtccctgtgttctctatgggatgccctttccctcttccacatcagctgcttttcctctgatgttttcttatttgccgtttatcctatatgtagaatgcaagaattattcacttccttctttatgccccacaggtctttgctcatactagaatagcatttattatcccatgttgtagttatttaaacacatttacccttcccttctgttagcccatgagtcctgcccaggacacagactggttggtagtatgcttgatggcaagaacaaatgctttggagtcagactgattttaattgctgactgcaacactttaactccataacctgggacaaattctttaaactacagtgcctactcagcctcctcatttgtaaaatgaacctactacttaacctcattggattgttgatactaaagtaattgagtacataaagtgcatagtgagtgaaggaatagtggctgaagtagaaaaatgggtagaatatatcttagagacttttgtttaatatcctgcacacataatagtggagtatttgtttttaataaaacattccttctttaaacagttttgatgcgttggactttttaaaagtatatttgcttgtgttttggcatctgaaatgctattaaaaacatatcatcttagtggctcatttccttcagtttacatgcagagaagcctcttggaccccaaaacttaacttactcctcctgcattcagaaagctttgcatctttttggagagaaccatctgttcacagcattatacagataggaattaaacagattttgttggggaaagtcactttccagctcttctcaagtcagaccctcaagtctgcagatatcttgatgcatgtaacatttctaagacactgagctacatgctgaagaatgtacaggatgtccccatcaggcttttcagcggagttgtgagaggtgaagtatgagtacatggaaaggtggacagtaactgcagtcgatggcacggcatggtgtgctgccagatgaatggttcagaccctaagtgctggccggtggaagagaagagacttcgacttttggaagattgaataaaaactacttgaagacaacaagaaagcattctagacaaggagaatggagggaatagagtttagaaagcacaagtcatatgtagcgtgtctggatttttctgtttatgtcatttctactagtataaacaaatattgccttttttgttgttgttgttttgactgttactttgttcaggttgtttctgctcagaactctttttttatttgggttcttaagattttccacttaatccaaattctactttcagatctattaaacccttaagattttgtctgtgacctagtggagtattggtgtgtcttgcattataataaacatagttgtagaaatttgagtgtaaactatttaactagaataacatttttcataaactgattttataatgtctgtaacaattcatttactaagaagtcattttctctgcatattttagttaataaattggaatgatttttgtgactgccctgatgtacagtacacttatattatgaaggctttcttttaaatttcaatatgacaaattttgtctgctctttgaagtttagttcagcataaatcatttgtatttgagtcacttagagattcattgttgaggtttgaggaagtttagactttgttcaaaccaagactttgttcaaatgaagatgcactccatcccaaaactggtaggagatcacttataagagggaggggtgtgggggcaggagctaagacaggttattccctatgtccttctaaaaatgcacaaaggtttgaaagtctaaatggcaaagagttaatcatagtaggaaccaggtttctgatgtgtagccacttgcaccaagaaagatttgtttaagtaacctaaccactggagcctgtggttttgaactataaaggacaagagtgaagatggaagacaaaagctttttagaaaaacaaaatcaggttgtattgagccttttggtaatgaccattaaatatggctcaaaaactgaagtgcagaatgaacacttttaggtgtattttgtgactgggtctctcagtgactgcttcccggtaagggtgtatttattatttaatgagaagaccttgaggagggggtaccaaaaataaagtagaggcaggaattccaagacggcacaatatcctgatttggtggcacaaaaatatattcgtggaaacaaatctactggaattgtcttgtttgggggcttgttttacatgtgttttatctcactatgttgtcattatgcagaggtcttctatgtctttacccttttagcagcagatcttatttttcaaattaaatctcacatggtaccctaatacataaactacatgaaatcaaggtgggaaggaaggatgccttgtcctggtaccctgcgccctcaacttcctccattctttcaggtgaagcttttatgccagctggaaaaacccattggcttgttgccagtgacagcttactgttaggagcaggttttcccacattttgctatttatagttttacatttcataataattaacatattgcagttaacctggagaacagaaagatacccactaggtgctattaatgctaactgtgtgtgcttgcccaagcagtgttctgtgaaaggtctgatgtcagaattcattctgaaatatagtctataatatagtcattgtttaaatgcctttctgtgtacttcctagtaaacagttggactagtacagtgttatgcagaaatactaaagatagtttcaacctgttgtgagattttcaggtttggtgtgggaggagggggagaactatgtagtaactaaaatggcaactccacgaatgctactgttcttcacccaccaacacgttcccgtacgtgtcccaactggcctgtctttttactgaacttatgtagcatattttaatcatataccaccttggcatttcttctgttgctttttcagatgtagttgttgaacttttcatttattttcattgtcccactgtctagacttttagccccttgaaaacagaggtcatggtttaagtgcttttgtgtttcatgtacactgtaggcattcagtaaagagttgctttgatttttagcttggtggtcttaatatatgcattttatactgattttttaaactgttgttgttgctgaaaactagatttgtcagcttgtttagtgcatcatgatccgcttactatggattctgctaccttgaccagcaaggtagcagaattttaactataatttcttctttaataagagattttctgaggcaggtaaatgaagtatactgatacttcttacacagtggaatatgaaagtgtgacacgtcgcatcttgaaagtactttaaattgtgtgcctttgagttctacactgaaaaataaaactactaccaacaacttgaccccctttcctgccctcaaattgtttagaactgaaaatagtgttttgttgtacatatttatattccataaagtaaaattttttaacatcagaaaacatctaaatatattatcgattccttaagaataaacactcattccctgatgtattatactattactaattactttaaaaataaaattagtattgtttttaatataaaatgtttggtaacttgatttttattatactttgcttgctcttttcacatcttatggtaagtttaaagtactttctgatgtcttgtctttgtaatgttttagtgataagaagactcattattagattgactacacagtacctctgagaatgatgagtgttattcctaagttcctaaaaggagactagtacacagggctagttcctggcagtaagaaataccctgccttagctttttggaaaatttactatccgttcggtattgaccaccagtggagcatttgttattaggcatttagcaagtaatgtgctccaaattgcatttccttctcttatttttatccttatgagagagtaaggcaggtgttatttttagttatgaattaggtgtcctgggatgtacatgcctaaattatattgacagccagttgtggagatgccttatacaacctatctacaatgttttctctatgagacctgctgacttatacacgttgaattgtactccagtgtgaagtcctgtataaagaaaagaaaatgaacttgcatcaggttgagtcagtgggcaaagtcctggtactatttgaaaggaaaatgtgcattcaagcgagatcatataagagccctggaagcaaaggaatcgacttcctttgtgtcagtggttctcaactggatacaattttgcttccctagaagacattttcaacgtctaaagacaattttggctgtcaaaactggggagttggtacccctagcatctagtgggtagaggccggcaatgctgctaaacctcccacagtgcttaggacagcactccacaacagagaattatgtagttcaaactgtcagtggtgatgaggttgataaaccctgctgtatgtgtttcaaagaatggaattgccagaagccctcgcagccaagacaaatttgggtcattgggagggaatagggaatcaaggaggtgacttcaggtatggccggactgagatgagctattttgatgggattaggattaagctgatttcctttctctcaccttttctttgtgccagcacttggcatagcaatagtggaaaaaacagtgaaataaacttcttttcttctaagctagatggaatatgatcctattccaagagctaaaagcatcattctacttccctgtaacacttgcagctttactgtaagccacatccatgctgtcaggctcccaaaatgtcaaggcacatgattttttggggggaggaggggtgtcaagattgttgcattataaactagctaatttatttcccaaggtatcctgtaatcatagtgcacctgggaggatctaaatttctcactgtttctgcatcatgcgccatatgtgaatttctttggctaacatacccttgcttgatttgattagtctacaattacaggaacaaatgtcccttaaaacaaacttttttttagagtagttttaggttcacagcaaaactgagtgggaagtacggagaattgtcatataacccctctgcctttattgtcttttgactttattactaattttcaaataagatttatgtaaatgcaagtgttattctgttaatgcaaaattaaatgttcctcaatttcattgctcactaaatggtaaagcaaaagttaggagcagattgttctgagttatgggatattctctctctccctattcagtagaagagggaggatcatttgcatggacttcttagggacattagccactcggtgtaagatcatttatcttcctgaaaatgttcccatgtagttatacatcatctttttatttctgggggattttatttttcttataggaagttttccctgtttggcttttgcctaattggaagctattttaagttattcagcatgacagttggactgctttaaaagttagagtgtaccatagccgtaaaccttactgtgattgagagtgttctaggacttatatttatgatcttgtttgtgtctcatgtagttagggagccagggggaaactagtgattgtattcttgagcagaatttttttctcctggccgcaaagtccagtttcttcctttagtcacaatatctctctgctaatcatggccagtggacacactataatgcttctctgtttctcttttgcctcctttcaatcatgggtcttcccctgggctttccctttaatatttgaccctttaaaaaattaatagtctgtacagagtagatcgagagagtaagcttgcttgcttttttttggacagggttcaggttcaagttctcaataacaagttttgtgaacttggggaaattattcctgagtctgtttctttatctagtaaatgcagattatccttgtctgcctgcctctcagggctgttgtgaagatcacatgaaataattgactatgagagtgccttgtaaaccatacaacacatacaaatgtaaaataatatgcaaatataagaaatgtgaggtttagtaaattacatcaattatgaaatgaaagtagggttggagaatttattttcccttctgttgtcttgttttgtgtagcctagagtgctttttcttcatcttaataagttcagagatgaccttcgttcagtttagttgtagtattttatctttttataaataaggtcaatggagttaaatgcttcacttgttgaaacatgatataatttggcattgatcagtggttggtgcaaagcaaaatttcaaaaagaaaattctgattttgctaagcagcatagatcgtgcctgttagaacatcctcctcctccttctcagcgttgattgccaatcagttctgtagaatcagaaatatttttctatgcttttttcaaaaactccagtttgtaatttgacttacattgctattttcaaaaataaaacaactatctaagtatttgttaaacaattaggtagttgttatacttcacccttttccccctgaccagttagttatactatgtggattctggatggtagtagatcaagttagtttagtcacaatttataaaaagccttttctatgttgctagtgtcttctcatgtttgatactattcataaatagacccagaaaactgtaggactccgggactcaggctgtgaatgattggtagaactgttggagtagagcaggcagactgtgttggaaattattccatctagaaggtatcagctcctgcctcctcggccttagtgactaaatggaaatacattcttttcaaattcaaaagcctgttcattactgcttttctcttctgaagcacattgccagttttataatgaagaaggacttaattatttttctttttctattgcttatgtttattcttcataaaatgacttttcttccatttgtgggctgagaaataagctcctatattggactttgcagacttggcttttttatagcgtaaatcagctcagctggctaaatgcctcactaagtgaaacttcataatgtagctggaactaaattgtgctctggagcattgagtctttaaactggattattgatttacaaacccataatggtgaagaggctatagagagacaagaacctccctctcctgatccttgagcaagtttttcctcacttcaggggctctgagctcacgagcaaaatcctaaccaagtttatcttgctaggacaacccattttaaatgctcaataaagaaagaataattcttctcccgcttaaccactaaagaacaacatatgtttctaacgtagctttttatttttttttatctttcatttttttgttggtaaatctcccagacaacagcagttctgtagagagtttaaatatgaaggaatggcaggacgggctaagggcacttctacccaacattaacatcaactttggtggactgcccaattcttcttccccctccaacgccaaccacagtgcaccaacgtccaacactgccaccaccgacagcctgagttgggacagccctggcagctggacagacccagccatcatcacaggtaactttctcactcccttcagctacactgactgccaacagtttggtttttagcacagattaaataaaaaccgagatctcttcaacataagctgcttttcacaaagccgcaagtgtggagtggcttgtttcgttctcaggggtcatgcaagtctcatttaacctgtcggcctagttgtgtattaataagaaggcatttggtgcctaggttgcttcttctggtttaagtaggtgttaacatctggcaacatgtgtattttgtaggcctcaaagtacaaaaggatctgacttactcttcttataaggaacatactccactttttgttataaagctctctattataaggctgtcttgttacgcatccctgtcagaggaggaaaagcacaattgggaaatgccatcaggcagttctggggaatggtgttttgcagctctgcctctgtgacagtgggggactaagtctagttcaagaggaggcatgggtttgtgtgccaataagttctttgttgttgggggaggattggtttaaaaggtcttatttggaaggagtttaaaattgtaatctcttctgtctttccttttggttttgctttctaaatttgtttgacgaccttattctttccacacaaactcaccattttccctcttttttcttttctctccttaggtattccagcgtcttcaggaaacagtttagactctcttcaagatgacaatcctccacactggctaaaatcccttcaggccctcacagagatggacggccccagcgctgctccatcacagacccaccacagcgcccccttcagcacacagatcccgctgcacagagccagttggaatccctaccctcctccttcaaacccttccagcttccactccccacccccaggctttcagacagccttcagaccccccagcaaaacccccacagatttactacagagttcaacactggaccgccattaggcaaagaggagcaaccattagaaaatgcaggatttgtcccactctgttttctccctctcagcccaccacccacagctcccttctcatctcttatgttctgaagaatccagtacctgatcaattttttttctccctaaccccagatgcaatgcgatcacagggtcattaccatctctttattaattgtaaaaatttttgttgatccagcatttgagtgacactgttgaatgtttctaaaaatgcctttttaaggagagaaaaaaaaatcaaagggcagtctcaatacttagaaaattattgtttgtctgttgcgcataataatgacataatctgcttagcagaaaatgacgattaatctataggaaagctcaagtaaatgcattatcaactgcagaagtttgaaaaccaggttcatttacgtgagattgctaaatgcatgggggaaagcagtggtcctagcatccatcttgtattcagcttatcattattgcagggaaaatgcttttaatttaaattaatttttaattcttttggcaagttgatggcaaggacttgattgtgtcattaagcaaaagaatgtattggaagttgatggaaagacaaattcatctgtagtagtaactggccgattgctaaagagttcataaggaggtgagaagtaatttttttaaaggagaaaaattttttggctttagatttaaagtaaattgaaatgttttaaagaaaaaagtattcacagatttaatacctattaataatataagagctgaaatgtaagtcatttcttcagtccttctcctctgtcggaatcttttttgttttaccataaattcacctgacgagggcactctgagatagcactgctctggggccatctgatcaccatcgggagcaaatctctgacctccttgcctgcagcttttacttaaccctgtagtttctggacgtttgtgcagtattgaaaagacaggagaaaagaaaacagaaacctggttataacctgacgctaaaactaaaaacaaggaaatgtacctctttcttcagaattaaaactaaaatcttaaataaaacagaaaacttgatggtgacacttgggttgtccttgtttttgtttttctgttttgttggatgtgagtttgaaaggttttgtgacaagtagccatcagatgtttccatttgattttacatcttcaacagtggggagggaggatggtttagaagaagaaagtggaggagaaacaaccattttattgacagtcaatggcatccttgacgttcagcccatcttgtcctcaagaatccctcttccagtgcctttcagtagaagattcctctttctgctattgtattatgcatgccaagccttcccaactgagaagccttatgtgccagtaatggagaggttattgacatgttgagatgttggttctctttagggagacctggcaggagcagcagtcactatgtcacacaagtggcatctctttgtgagtgccatgatgggaaagagatcgggaaacactgatgtagatgatccacagacacatcttttatgactgaccattttaggaagtacctgttgatggggcaacgatcgcaccactgaccaaaagagggtagaggatgaaagttacctgttccccaacagagcaccaggatctgtgtggtttgtatgtcttgccttgggctgcattcagaagcccaaagctggaactggcatattttcagccatgtccattaagggatgtgatgtaggatcaactaaatagatctagatcgtacgttctgtgctttcaggtgggtttttttcgtccttacctttatgctgtactttaatttgttaaaatttcaacacaatttttagaaacttaaacatgatattctcaaataaatgtcaccagaaatagatggtgatcaagtggatagtaaattgttttgtaaaactaacaaaatttccctggataagaggagaggactagaaatgacaggctctctttgcccttgaacttcacttcagtctcctgaaccttcacattgtactgcaaagtgatggaccaatgcacaaataatattcagatggcagtgaattgtaatcaaggctttttgcggggatgcggggaagtcctgagatgggcatatcaataaaaatgttgctttttttgtaaaaggagggaactcctaccttataaggctgtgctgtaattgtgtgtgtgtttaatcagtcatacagaagagtttataaaaagcatgactttataaaaagtatgaagaataaaaataataatgacctgtgtgtctaccaccaaggttaaaaatagaggccaggcatggtggctcatgcctgtaagctcagcactttgggaggccgaggcgggcagatcactgtaggtcaggagttcgagaccagcctggctaacatggtgaaaccccatttctactaaaactacaaaacaaaaattagctgggcatggtggctcgtgcctataatcccagctattcgggaggctgaggcaagagaatctcttgaactcaagaggcagaggttgcagtgagtccaggtcacaccattgcactccagcttgggcaacaagagtgagactccgtctcaaaaaaaaaaaaaaaaaaatagaacctctggtatcttaaaacctctatttgcccctctccagttagacttcattttctttccttatcgtttcaaatagccattattttaatttctgtgttaatcttcaagagttttgccagctatgtatctatccctgaatagattgtttagttttaaaaagctgagtaataaacccaagcctagctgtagaaggaaagggtgatgttgtcactattagattaagacttttaggagaagataaactaaatagcttctggggcatagtttggctttagcgtctccgatcatttatgaaagcctcttagaagtacagtggccaaggttatgagtgagtcctggaaaacagggcttgccagttggggtaggtcttgtcaggtaagcacagtcccgggtgtctggcagaaatatggtctacaggatcagtttgctgtctgtttcctgtgtcaaagctcaccatggcttccctccagtcctctggagtagatcacagacacttagtgaattaagtgtcttaaccctttggggtgagaggtggcgtttaaaacaagtgtcagctgattaattcaccagggcttggcggggagaacagaaacttcattaggcttggtcaaaatcagaaagtacagaaaagagcctgaaccaaactgacttaggtggcctgttggtataatagcagtgggaatcttggtactgacagctgtcatggatcacagtactcagatctggtacacagcagtcatttctagtatttcactttgttctctctcctttcttagatgatcttcctatattcctagatcccacaccctagtcttttatttatttatttttttaaatctcattttgctggagtgacatcctccagtatttcttaaggaaaggtacatgggagataaatattttagtaatttgcttgtctgaaataatatttaattctacttctcagttgatagtttggtaaatacaggattctaggttgtagatatttttttctgcagaaatttaaaggcttattaacttcttgcttccagttttgttgttgaacaatccaaagatgctgttatcctctatatgtaatctttctccctctctggaagcttgtagcaagtttgtcttctgtgttctggaattccatagtgatctcccttggtgtgggtctattttattttatttatttatttatttattattttttttattattatttatttatttattttttgagacggattctcactctgttgcccaggctggagggcagtggtgcgatcttggctcactgcaacctccgtctctcaggttcaagtaatccttctgcctcagcctctcaggtagctgggattacaggtgcccaccaccacgcctggctaagttttgtatttttagtagaaacagggtttcaccatgttgaccaggctggtctcaaactcctgacctcaggtgatctgcctgcctcggcctcccaaggtgctgggattacaggcgtgagccactgctcccagccaggtgggggtctattttaattcattttggtggacacagtggacatacagtatgcttttaaaatcttatgactcattttcttcagttctcggaaattttcttgaattattagtgaacttcttgcacttactttctctgttctccctttctaaaaattctattatttaaatactagactttctggacttctgctttttaaaatattttctccaattttctatctgtgtctttggctcttctttctaggaagtttttttctcaactttatagttttcttctccttgtgttgtctgtttctccaagttgcttttatctgtttgctttaatttctgtcttccttgttagagacttctacacttgatcttagccaaaaggccaagaactgattatattagagactccttagatgcttggtaatccttggttatctgctcatgaatgagactgaaaaattgatggggagttttgaaaacatgagtagggcttgccaactttgaattcccctattggaagatctgggtggtctattctactgggtaacctctgttgttattgtctttagcacttgtctgttggccttggctggtcagattccatacagaagagtcttataaggactggcctagctggtaagggtctgactgccagggcttcagagctgtgttggtggtctctgtatgtatttaatattatactacatataatctctctgacgtagtctttacctcgccagagattaactctccaaatatctgcccatttatgagaagaatagcatggagttggggaatgaatctagagagttaacttcttaaaacaactttcttagtaatctttattttagccccactgcacacactttatctctggttttagtggtatctggtcctgtcagtttctgcgcccttgaggattttgtgatgtaaattggattggatctcagttttccccactgttagcttaaggttggactttctcaaatgagctaggtgagtccatccaaccatctactttctaggtccttggattttattgctgtcatttcctcttgttattcctgtttttctttttttcttacttgaattttaatggacctttaggaagaagtaaaattaggtatgtaagttccatctaccatcttagcccagatctgtgctctgattttataaaccagtaggcatagtttgtctgccagtgaagtagagtaaagtagaggaagcactatcttttgctattggccaatgagtgctagccaccatgttgtatatcccttggtttgaatagtacctgtttgtttcatttaccttgaagtggtttatctttttaacatccccttttccaatgaagctcttttggtttacagtcagtagtttgtttttcaaggggtaatcacacgggattttaatggttaaactttaggctggatgttaaaggcaaattttactgcagttttagggtcatttgcaagtcagatggatgtgggtcattttctaggcttagctgaattagaacaagtctagaaattgcagagagggaaaaaataacctactcttacttatgtatgctgtgtatgaattgtgttttaaggatgcataatagtaactgtcaacaatgtatatctacagagattatgctataatataataatacagagctgaattcagttagcactctccaacaaggtttgcttaaacttgaagacgcaaggtatactgcacatagcttttgagttttatgttaaatattgaccaacatcatttaattttagatccttattattatagtatgtaaactcttaaaagttagaaaacttacatatcttacacataattttgagatatgccctttgtcagatgtatttacttctgcccaagtgatctatccaagtgaaagatgactgcaggtcattagcatgaactatttgaagaaatcaaatatttatcttgtcccaaaaatcagtactctattttttgtgcttgttcagtaattgttaatttagttcagacagtcacaggcagttttccagcaggaagagctgcctttgaaaatacttctttttttttttttttttttttgtttgagacagagtctcactctgttgcccaggctggactgcagtggcacagttttggctcactgcaacctccgcctcctgggttcaagcaattctcctgcctcagcctcccaagtagctgggattacaggcctgcaccaccactcccagctgatttttgtatttttcgtagatacagacagggtttcaccatgttgaccaggttggtctcgaactcctgacctcaggtgatccgccttcttagcctcccaaagtgctaggcatgagccatcatgcctggcctgaaaatacattttaaaagttgtttctgaacaaattgatttaaggaaaatgagtaaattatttaataaattcctttaaggataaaataattatttttattggtgataacccagatgatttgggaaagaacatgcatcttggcccaaatgttcttggtacaccttggccattctcaagaatatattctgcgtttagtccatctctgtgggaaactgagggtattaggcaggtgttagtaggtgtagtcattggacctccttccatggcttcctttcatgggtgaaggaagtaagtgtacaagatcaatagaaatgaataggccaaatagatatttttggtggtctgaatgatttaaaaactctaataaagaactcaagagcatttaaagggaaacaataatctgtggctttcttattttcagctttagtatttgcaaagctaaagtactcttaataaaatgcctggcatatgacatcaagtccagagggactttttaatgttgttggtttcgtacaatgaattccagtactcttatcagatcccatcatgtgggaaaagtaggcaaacaagctcagagatgttgagggtttgtcctgggtcacaggttttatagccgagctagaactacttgttacccatcctcacctacatttgcatgactttggttgtccttcctttctgtttcaaagttgcaagtgcttttctagctttcttgtttatggaattaccaagatgagggtggtattatttgtccctcccttatattaggtaagtagtggtgagtgaaacggtgtgaagagtttctgtatttgttcatcccagatcataaggtatgagtcaaagtttagttgggaagtcccttagggacgtcttgattattttgtgctcatattgagttgtatttacattcagttgaactaccaggttagccagtatcttctggattttatgtagctaaggaacagggatgagatacttgtcttccaatgtttgaaagacagttatgtagaagaagaaataggatgaagcatgcagcttagactaagacgcggaagagagatttgggttcaatttagttttaacaatttttaaaaacacctttaggctcagcattatgccaggtgcttcagggggtacaaaagtaaaatgatccctgctctcaaactgaaaaggtttggatagtcagctctaaaactgaaactgagtgtgagaagtgcaaagtgccaaggtagctcaaagaaagagctttggacaaacttgagacttggattggagggaaccctgaacaaacaaatacatcaacatgagcctgcctggggcatgtttagggcttgcaacttggggttcctgtaggggacttgtgagaaataaggcgggaaaggtgagttgggcactgcttttgtttcaaatgacaagttctaagtaggagaaaggtaggataggattttgctttaaattctaagtgggaatgatgaagatgagagagccactgaggtagggttctttgggtctatgaacccaaatgaatgaaaggacccaaagaaatctaaggtttggccacaaagggactatcagtgaccctagaaattgtatgcaaaaatggtatgtgcattagtgttttatcaaattatttttaaaatgtgcatgatgcaataaagtctagtaagctaaaagaatagttctggtgaaggttttgggctgataaagggattgaaaatggaaagaagaagaaagaattcaagagagatttcagaagggctagagatttttgcaagttactagccatagttaaaggatgaaagaaaaacaatttcacagccatcttagatttcttagatcctccactagactgtaagctttttgagagcagacagcatagctagcttgttaaacattacattctcagaacctagctagcacatagtaggctctcaataaatatttcagtaattcttgcctagaattactgaaatatttgcatatttcaatactgcatattgaaatattacatgttttgagccagatgattggaataattgtatcatgttgaaaataaatgtggactcatgaaaaggaacaattgcaaaaagatgaatttgttgacttgaggttccagcaggtcatccagccggtggtgtccaagaaatactgggaaatttgtgtgggatacagagaagaaatacagtgttggagtcatctttatagggagagaggtgagatgggagccttgggaaaaacagatgctagtggtctaaaatgaaggagatctgagtgtatggtgtcggtgtagaacagtttctaggcaagaattactggaaattttgtgctatgtcagtcagggtttcatcaggaagccagggatttcacaaccactggaagggctaggagaagaaaggtcaggaagacctctgctggctttcaggagctccagaagtacaggagtcatagaaaaagcctccaccaaaaagccctcagctgcctacagcaccaagtcggtaattcccaggaagactctgggaagtggctacccaattccccgtttgccaggggccacctgcctgcccagagatgacacccgtagaatgatggctttttctcttctatctttcaaattgccctaagtacctcccattagtagggtctaagctgggatcctgttggaaggaatcggaactgtgatgcaggagagagtagcaggggtggggctgatgtcaacagacaagctagcccctgtggtttagagcatctcacagggtcatgtcacctctttgtcaaaactttccaaaaacatctctcccccaataccttcataatgaaattgaagttttgctggtgtgggaccgggtgccctctcccctaacaggaggatattctttatttcatgcgactgtgtctctgttccatgcatatctgttttctcagccttgagtgtatatactattcctatcctcaccctggcaatctgcttattgcagctttttctcacacctctaggcagatttaggtggccctccctgcctttgtatcttatatacgccttcattatactgcaacctccttgaagaagggatacttcttttcatctgttatacacagggcttagcacagttcccgacattcggttcgaatgaatgacggaaggaaagcagctttgggtagtggggtgctattaagttttattggcaggagatcagtcatgaactttaaggcactggttttaatccagcggaggtactggaagccaattgcagtatgttgaagaatggagaatgactaggagttaaagaactggagatagtatactctgaagaagtttgataaagggacgacaataacacagtttcaagtaaaggaaggtttttttcctcaaaggaagtcataagccttttagaggagacaaaattatttgaaaaagtaaatttgaacaagaaagctgagttgaatggaatcaggaacatgcatagttgcttccctccaagataacagcaaagaaaataagagagagggttttaagataaagaaaggaatattaagggaaccccttggcttagcatagtgaggtagcaggagatggaataggaatttggagcttgagaatatagaaaatgtgcaacactttggggaatgtaacaggaaggtaaaagaatgggttaaaagagtagtaagcaagcagcaagggcctagttgaggttagcatgtatagatccaatcagaataattcgatgtggcctggactgttcatcaatcttagagagaagcaaaggaggcatggagattaccaggagcttctgaccgatagaaatggtggaaaatcatggagttgaggctttgggagggggcgaagttatcagagtgctcaccacggcccatagtatgaaggtaagtgctcaccatggcccatagaaggccagcagtagggagcgctcttcaatcattctctcaatctggaaatggattggtgcccacagctgggcacacagaaagtacacaacctattgtcagagcacacagccatacagttggacccctctatgatccctaaagtttctttcaacatttaagattttatgataattttgggattactgcataaattaccaatagccagaattcaaaagtaagtgtaaaatttaacagaatgtatacagaatgaattattttataaaactcacaatagcctaattcatatattgaatttcttccagggatacctgtattttcttgttaataatacccacatcattctttttttccccatctgctcagtaaccatttattgagcatggaggtactagactgtgatatgcgttaggtatgcatttgggaatagaacagtcataactctgtctttcttagagctcagacttaggtggtgaagatgatcattaaacaatccagtaaatatactagtcatatgaaaatgcagaagaatatatttgaagtatgtgtaagcaatatccaaaatacaagaccccaaagaaaaaatattgacaaacttatcctgaatttaagacttctgtaagacaaaacaccataagcaaaattacatcaacaatggactgggagaaaatatttgtaacccatgtaaaagactgaggattaatatcccaaatatataaagaatgcctacaaatcagtctttcttaaaaatacaaaaagccaaagacaactaatagacaatgattgctgagaatgttttgttcttatgattaagaaaaacaaaaaatattggtaagggtataagaaaatggatatcttatgtacttttgctgtgattataaattggtacatccttttatggggaatgaactgacaatatgaaattgcctgttcacttcaacccaataatttccctgcttgtactcatacacagagaggaatacataaaagcattcattatagcattgtttataagaagaaaagtacaaaatagcctgaatgttcaacagttcattggtaggcaatactacagaacaccatgagaagaataattacatgtatttctgtgtgcagtcacacgatgaaaaaactatatgtaaatctacgggttttttgtttgtttgtttgtttttgagacagagtctccctctgtcacccaagctggagtgcagttgcgcaatctcggctcactgcaacctccgcctcctgggttcaagtgattctgcctcagcctcctgagtagctgggattacaggcatgcgccaccacacccggctaatttttttgtatttttagtacagacgaggtttcgccatgttgcccagactggtggtctgagctcaggcaatccaacccccttggcctcccaaagtgctaggattataggcgtgagctactgcacccggcctaaatatacttctaaagtatatctgcgatattttctacattaaacacacaagaactaaaactacatatttgtatgtgcctatgtaaatggcaatgcatagacaagacttggaagagttcatgccaaaatgatgatagcaattacccctatgaagaggagtgatgtggggttggggctggccaggagacattaaggagaatttcttgcatgactgatactgtttgatctttttaaaccataataatgtattaatatattataaaatgaaaacattcatataattaaaaattgtgctttgaaaggaaagtataacatgcaataagtatgcaatggagaaactgcttttcatcagggcatcagaatgtaaacgcacttggagaacaaacagaatatttgactttgtttttgtttttgtttttgaggtagggtctctgtcacccaggctggagtgcagtggcatgactatggcttactatagcttcaacctccccaggctcaggtgattctcctacctcagcctcctggtgcaccaccacccccagctaatttttgtattttttgtatagatggggtctcaccatgttgctcaggctggtctcgaactcctggctcaagggatccacctgccttgccctcccaaaatgctgagattataggcatgagcaattgcgcctggctgcgtttgacttgtttactcctgtctacccagcaaagacatcagatggatgggtgggcagatggatggatgagaaagcctcactgagaaagtgacatgccaatcaaaagaagaagaacaaggaaccagccctgccagttccaccagcaggagagcagtgtgtgtgagcatgtggagagaccccaccacgggaacctgggcatatccagctggaggcagcccatgttgcctggtgcacaatgaataaggaggagaatggtatgagatgaagtatgtgagttgatcaggggcctcaggcctcatggctttagtagctagtcatgaggagttctatttagtctaaaaagtgataagtataccctgacaatgactgaggtgtctctatggaatgagtatactcactaagaaaaataagtagcataatgtgatttgacttgctgccagtcacagtgagtgggtgatgaggattaagacttggttcttagcagccagtgcagtggctcacgcctataatcccagcttgaggccaggattttgagagtagcctgggcaacatagtgagacctcacctctacaaaacattagaaaaaaaaagacttggttcttagaaataccagcccacattctgtccatttagcacatgaagcttttttttttttcttttttctttttttccttgaactggaggtagggggatagtgtagagaaagaatagaaaagggtttatagtataatggtttagagttacgaactcagactgcctgcatttaattgccagagttcccatttgccatctgggtgacaatggataagttacacaaatctctacacctcaacctcctttccctgtatgtcaaatcaggataataaagtgtgtactattatctcagaggtatggtgaggtttaaataaaataacccacattcaatacttagcattgttcctgtccagtggctcgagggatctggaacatttgggccagaaaagagaggagaccactcacaaatatttgaaagttttgctgagaagaggaattatacattatttgcatgtcctattgaggacaatgaagagttatggggagaaattcaagggcaagggcaacccaaggtaaggaagacccaacacagccaaggatcaataaagg

SEQ ID NO: 5 - DNA, 24 bp, Artificial Sequence
  Synthetic Construct
catctaatggcttcagagcgataa

SEQ ID NO: 6 - DNA, 24 bp, Artificial Sequence
  Synthetic Construct
gcaatctcttagtggcaactgaac

SEQ ID NO: 7 - DNA, 22 bp, Artificial Sequence
  Synthetic Construct
gccctagctttacgctcatcac

SEQ ID NO: 8 - DNA, 24 bp, Artificial Sequence
  Synthetic Construct
gctgtaagaaccgcataaaacgat

SEQ ID NO: 9 - DNA, 20 bp, Artificial Sequence
  Synthetic Construct
cattgcaaactcaggagata

SEQ ID NO: 10 - DNA, 20 bp, Artificial Sequence
  Synthetic Construct
taacagaggcatgaaaacca

SEQ ID NO: 11 - DNA, 20 bp, Artificial Sequence
  Synthetic Construct
accttgactgtggtagttgg

SEQ ID NO: 12 - DNA, 23 bp, Artificial Sequence
  Synthetic Construct
gttcacctcataggaaaatcttg

SEQ ID NO: 13 - DNA, 20 bp, Artificial Sequence
  Synthetic Construct
gttgacagacaagcacagac

SEQ ID NO: 14 - DNA, 18 bp, Artificial Sequence
  Synthetic Construct
agctgtttcccatttcca

SEQ ID NO: 15 - DNA, 16 bp, Artificial Sequence
  Synthetic Construct
acactccgccaccttg

SEQ ID NO: 16 - DNA, 21 bp, Artificial Sequence
  Synthetic Construct
caacaactgttcctaaagcct

SEQ ID NO: 17 - DNA, 24 bp, Artificial Sequence
  Synthetic Construct
gggagttactaccctcacttaatg

SEQ ID NO: 18 - DNA, 16 bp, Artificial Sequence
  Synthetic Construct
ggactgggcagcaaag

SEQ ID NO: 19 - DNA, 20 bp, Artificial Sequence
  Synthetic Construct
ccagaattgaaaactcagca

SEQ ID NO: 20 - DNA, 25 bp, Artificial Sequence
  Synthetic Construct
attgattgaggaactgaacttacct

SEQ ID NO: 21 - DNA, 22 bp, Artificial Sequence
  Synthetic Construct
tctcatctgtgaatgcactatc

SEQ ID NO: 22 - DNA, 21 bp, Artificial Sequence
  Synthetic Construct
gcagttgggttatttcaagtc

SEQ ID NO: 23 - DNA, 18 bp, Artificial Sequence
  Synthetic Construct
cgaaccggcaggatgaaa

SEQ ID NO: 24 - DNA, 23 bp, Artificial Sequence
  Synthetic Construct
tctgggacgtcaatcaatctttt

SEQ ID NO: 25 - DNA, 18 bp, Artificial Sequence
  Synthetic Construct
ctgcgcctgtccctgaac

SEQ ID NO: 26 - DNA, 18 bp, Artificial Sequence
  Synthetic Construct
ggaagtggcggagcatgt

SEQ ID NO: 27 - DNA, 24 bp, Artificial Sequence
  Synthetic Construct
cacattcgtcctcaaggtagagaa

SEQ ID NO: 28 - DNA, 18 bp, Artificial Sequence
  Synthetic Construct
cacgcagccctggatgtc

SEQ ID NO: 29 - DNA, 20 bp, Artificial Sequence
  Synthetic Construct
cctcattgcaaacttcactc

SEQ ID NO: 30 - DNA, 20 bp, Artificial Sequence
  Synthetic Construct
agcccacattttctgtactg

SEQ ID NO: 31 - DNA, 21 bp, Artificial Sequence
  Synthetic Construct
ctcacccaatgcttccagttc

SEQ ID NO: 32 - DNA, 20 bp, Artificial Sequence
  Synthetic Construct
atggtgagcgggatggtaga

SEQ ID NO: 33 - DNA, 21 bp, Artificial Sequence
  Synthetic Construct
ggaaaatgggtgagtgagtag

SEQ ID NO: 34 - DNA, 20 bp, Artificial Sequence
  Synthetic Construct
gtaggcgattttggtagtgg

SEQ ID NO: 35 - DNA, 20 bp, Artificial Sequence
  Synthetic Construct
ttgaaaccactccacccacc

SEQ ID NO: 36 - DNA, 17 bp, Artificial Sequence
  Synthetic Construct
gaagtgattgaagaagt

SEQ ID NO: 37 - DNA, 20 bp, Artificial Sequence
  Synthetic Construct
gacccatgatgttgaacgag

SEQ ID NO: 38 - DNA, 20 bp, Artificial Sequence
  Synthetic Construct
aagatcccttcaaagcgaac

SEQ ID NO: 39 - DNA, 20 bp, Artificial Sequence
  Synthetic Construct
ggccttaaccaatgtttccc

SEQ ID NO: 40 - DNA, 20 bp, Artificial Sequence
  Synthetic Construct
caccacagctctgtgtattc

SEQ ID NO: 41 - DNA, 20 bp, Artificial Sequence
  Synthetic Construct
tctgctcctcccttgtttac

SEQ ID NO: 42 - DNA, 20 bp, Artificial Sequence
  Synthetic Construct
agaatgagccaaaaacccac

(19) United States
(12) Patent Application Publication
(10) Pub. No.: US 2013/0267432 A1
(43) Pub. Date: Oct. 10, 2013

(54) IN VITRO DIAGNOSTIC METHOD FOR PATIENTS WITH SPLENIC MARGINAL ZONE LYMPHOMA

(71) Applicants: Jesús María Hernández Rivas, Salamanca (ES); Juan Luis García Hernández, Salamanca (ES); Cristina Robledo Montero, Salamanca (ES)

(72) Inventors: Jesús María Hernández Rivas, Salamanca (ES); Juan Luis García Hernández, Salamanca (ES); Cristina Robledo Montero, Salamanca (ES)

(73) Assignee: FUNDACION DE INVESTIGACION DEL CANCER DE LA UNIVERSIDAD DE SALAMANCA (FICUS), Salamanca (ES)

(21) Appl. No.: 13/702,915

(22) PCT Filed: Jun. 7, 2011
(86) PCT No.: PCT/ES2011/070406

§ 371 Date: Jun. 24, 2013

(30) Foreign Application Priority Data
Jun. 7, 2010 (ES) P201030872

(51) Int. Cl. C12Q 1/68

(52) U.S. Cl. CPC C12Q 1/6886
USPC 506/9; 506/16

(57) ABSTRACT

The present invention refers to an in vitro SMZL diagnostic method based on analysis of the expression of a series of genes located in a very specific deleted region of human chromosome 7. The deleted region in SMZL patients has been defined by means of high-resolution array CGH and is located at 7q22.1 between bases 99925039-101754718, preferably between 99925039-101348479, and more preferably between 100260234-100596921 and/or 101244026-101754718, which is the location for the following genes: CUX1, SH2B2, EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17. The present invention further describes in vitro SMZL diagnostic kits that comprise at least one probe that recognizes the deleted region at 7q22.1 or hybridizes with at least one of the nucleotide sequences of the genes mentioned above.

### FIELD OF THE INVENTION

The present invention refers to the fields of biomedicine and pharmacy, more specifically, relates to an in vitro method for diagnosis of patients with neoplastic disease: Splenic Marginal Zone Lymphoma (SMZL). Furthermore, the present invention also relates to a kit for in vitro diagnosis of patients with SMZL.

### BACKGROUND

The Splenic Marginal Zone Lymphoma (SMZL), is a tumor disease that can involve any organ, but the most common are the spleen and lymph nodes. It has been considered a distinct clinic-pathologic entity since years. (Schmid C et al. Am J Surg Pathol 1992, 16: 455-466). More recently, the classification of hematological diseases of the World Health Organization (WHO) has designated to SMZL as a subtype of non-Hodgkin lymphoma (NHL) (Harris N L, et al. Histopathology 2000, 36: 69-86), based on their morphology, immunophenotype and clinical features, together with mucosa-associated lymphomas (MALT) and nodal marginal zone lymphoma (NMZL). These three subtypes of NHL: SMZL, MALT and NMZL lymphomas are included within the marginal zone lymphomas because have their common origin in the marginal compartment B cells of lymph nodes and spleen.

In the diagnosis of lymphoma, it is important to classify each within subtype it belongs to give the patient the right treatment, as each type has a different treatment lymphoma. Thus, for example, in the case of SMZL, the chemotherapy should not be applied in the first instance, being the most appropriate treatment of these cases, the removal of the affected organ, the spleen generally, so it is very important to make the diagnosis correctly. Diagnosis of NHL and the differentiating between each of them are based on the morphological, immunohistochemical and molecular features, being the genetics changes in general and the specific translocations in particular, one of the features that are helping to us to distinguish some lymphomas from the others. Most NHLs are characterized by the presence of specific translocations, which help for diagnosis of these diseases. However, SMZL not known a specific molecular marker, unlike other lymphomas, has not been identified in them a characteristic translocation, which makes a diagnosis. Although cytogenetic analyses show that in these type of tumours can be detected losses in which are involved chromosomes 1, 3, 7 and 8 (Dierlamm J, et al., Blood 1996.87: 299-307; Solé F, et al., Br J Haematol 1997; 98: 446-449), there are few studies that demonstrate the existence of a specific alteration that is characteristic of SMZL and leading to the identification of a high proportion of patients suffering the disease.

Conventional cytogenetic studies showed that one of the most frequent alterations in SMZL were the losses on the long arm of chromosome 7 (del(7q)), which could suggest that the del(7q) is associated with SMZL (Brynes R K, et al. Mod Pathol 1996, 9: 995-1000; Hernandez J M, et al. Leukemia 1995, 9: 2140-2146). In these first studies was estimated that deletions in the long arm of chromosome 7 were not present in more than 25% of patients suffering SMZL (Franco V, et al. Blood 2003, 101:2464-2472), it was not considered that the development of diagnostic methods based on their detection could be interesting, because these methods identify only a very small proportion of all patients suffering SMZL. Thereafter, loss of heterozygosity studies demonstrated that the presence of an allelic loss in SMZL patients (40%) is higher than the allelic loss observed in other B-cell lymphoproliferative disorders.

The most commonly technique used for the study of chromosomes changes associated with SMZL, has been the Comparative Genomic Hybridization (CGH), which determines the gains and the losses in the copy number between two DNA samples, by competitive hybridization of a DNA normal (control) and a tumour DNA (each DNA samples labelled with different fluorophore) on metaphases of control (normal) subjects, then evaluating whether there are areas in which an increase of the signal corresponding to the fluorophore which has marked the tumor DNA (which is considered indicative of gains) or decreases the signal, increasing the signal corresponding to the other fluorophore, the normal or control DNA (which is considered indicative of losses). CGH studies confirmed that there are regions of the genome involved in these lymphomas, such as 5q, 7q, 9q, 12q and 20q and found also that there is a correlation between the loss of genomic material and shortening of the survival period (Hernandez J M, et al Am J Pathol. 2001 May; L58 :1843-50).

Using these techniques it has been found that in particular the 7q31-q36 region appears to be closely related to SMZL and deletions are often found in this region in patients suffering this disease. This region comprises a very wide genome area in which candidate genes have not been found which would help for the diagnosis of the disease (Algara P, et al., Blood 2002, 99: 1299-1304; Leisure E M, et al., Clinical Lymphoma 2005.4: 241-245). Despite having been very useful in understanding the genomic alterations in tumor processes and especially in lymphomas, (to whose knowledge, classification and prognosis has been very useful), the CGH method has a limited resolution capacity: firstly, changes involving deletions of less than 7 Mb are not detectable (Bentz M, et al. Genes Chromosomes Cancer 1998, 21:172-175) and on the other hand, to determine the exact point of chromosome in which the amplification is inserted or are the limits point of the deletion, is difficult, since the minimal deleted region is too wide to be analysed with a in situ hybridization probe (Hernandez J M, et al. am J Pathol. 2001 May; I 58:1843-50).

Losses of the large arm of chromosome 7, as already mentioned, is one of the more alterations which have been detected so far in the SMZL studies, either by CGH method or by Fluorescent In Situ Hybridization (FISH). However, the wide deleted regions found by these techniques, have shown problems when defining or delimiting them, because the probes used were large and not showing enough specificity and security for use in the diagnosis of the disease.

Spanish patent ES2284408 solves this problem by using genomic arrays, also called CGH-array or array of CGH. The CGH-arrays are a method to identify alterations in the genome, without being able to detect reciprocal translocations. This is a technique that combine the technology of micro-arrays with the CGH-technology, wherein the hybridization is performed by replacing the metaphase of the chromosomes with mapped DNA segments, whose set represent all chromosomes or chromosome arms, divided into fragments cloned into Yeast Artificial Chromosomes (YAC), Bacterial Artificial Chromosomes (BACs), Artificial Chromosomes based on P1 bacteriophage genome (PACs), cosmids or other cloning vectors. Thus, the level of resolution increases from chromosomic levels (distances measured in megabases) to be able to be measured in kilobases. The advantages of this method are the higher resolution and the ability to perform a direct mapping from the clone or the clones that are involved in genomic alterations. Spanish patent ES2284408 discloses the use of a specific BAC/PAC array which includes 3500 clones with a resolution of 1 Mb. The data disclose in this Spanish patent show that patients suffering SMZL have a deletion in the 7q22.1-q22.2 region which is defined by the probe carrying the sequence corresponding to the fragment of the human chromosome 7 included in the BAC/PAC array, called RP11-44M6. This patent also discloses the optional use, in the same assay, of the other different probes that hybridize with another different deleted region on chromosome 7, the 7q32-q33 region, increasing the percentage of patients with SMZL who may be diagnosed in the same test.

The present invention discloses an in vitro method for diagnosis of SMZL based on the identification of a specific deletion, located specifically between the bases 99925039-101754718, preferably between the bases 99925039-101348479, in the 7q22.1 region of the human chromosome 7. More preferably, the deleted regions in the 7q22.1 region are the regions located between the bases 100260234-100596921 and 101244026-101754718. The deletion detected by the method discloses in the present invention has an approximate size of 1.4 Mb. Preferably, the two regions deleted, 100260234-100596921 and 101244026-101754718, have, between them, an approximate size of 0.84 Mb. The genes: EPHB4, SLC12A9, TRIP6, SRRT, ACHE, UFSP1, TRIM56, SERPINE1, AP1S1, VGF, TSC22D4, HRBL, LRCH4, MUC3, MUC12, MUC17, CUX1 and SH2B2 (Table 1) are located in the deleted region detected by the method of the invention. To obtain the results shown in the present invention has been used different CGH-arrays and methods to detect loss of heterozygosity (LOH), which have a high resolution, allowing to define a specific region of chromosome 7 located between the bases 99925039-101754718, preferably between 99925039-101348479 bases and more preferably, in the regions between the bases, 100260234-100596921 and 101244026-101754718, in the 7q22.1 region, which is deleted in patients with SMZL.

Therefore, although there are information in the state of the art about different deleted regions of chromosome 7 in patients with SMZL, the method described in the invention, discloses a very specific region, which is specific of the said tumoral process and also, is frequently enough among the patients suffering SMZL, ensuring the diagnosis of a significant proportion of said patients. The obtention of the specific deleted region, in a way so accurate and definite, by the method discloses in the present invention, allows a differential diagnosis of patients with SMZL compared to other types of lymphomas, preferably, chronic lymphoproliferative lymphomas or non-Hodgkin's lymphomas, which are not characterized by the deletion of the region defined in the present invention. On the other hand, the more the deleted regions are defined for the diagnosis of patients suffering SMZL, best known genes involved in the development of these diseases, promoting the design of specific treatments to stop these new tumoral signaling pathways. In this sense, the present invention demonstrates the presence of genes which may function as repressors of proto-oncogenes, tumor suppressors, growth regulators, and which are located in the deleted region in patients suffering SMZL.

### DESCRIPTION OF THE INVENTION

### BRIEF DESCRIPTION OF THE INVENTION

The present invention discloses the association between SMZL and the existence of deletions in a very specific and defined region of human chromosome 7. The present invention relates to an in vitro method for diagnosis of patients suffering SMZL based on the analysis of the expression of a number of genes located in the deleted region of human chromosome 7. The deleted region in patients with SMZL has been defined using high-resolution CGH-arrays and LOH, and it is located at 7q22.1 region between the bases 99925039-101754718, preferably between the bases 99925039-101348479. More preferably, the deleted regions in the 7q22.1 region are located between the bases 100260234-100596921 and 101244026-101754718. The following genes are located in the deleted region detected by the method of the invention: EPHB4, SLC12A9, TRIP6, SRRT, ACHE, UFSP1, TRIM56, SERPINE1, AP1S1, VGF, TSC22D4, HRBL, LRCH4, MUC3, MUC12, MUC17, CUX1 and SH2B2 (Table 1) and can be used as candidate genes for disease diagnosis. In this sense, the present invention reveals that patients with SMZL show a differential expression of at least one of the gene selected from: EPHB4, SLC12A9, TRIP6, SRRT, ACHE, UFSP1, TRIM56, SERPINE1, AP1S1, VGF, TSC22D4, HRBL, LRCH4, MUC3, MUC12, MUC17, CUX1 and SH2B2 compared to healthy controls or to other patients with a different type of lymphoma, by analyzing samples from tumor tissues, bone marrow or blood. The approximate size of the deleted region is 1.4 Mb. Preferably, the two regions deleted, 100260234-100596921 and 101244026-101754718, have, between them, an approximate size of 0.84 Mb. The data disclose in the present invention show that a 49% of patients with SMZL included in the present study showed the deletion of the 7q22.1 region, detected by the high-resolution CGH-array as described in the present invention.

TABLE 1
Genes located in the 7q22.1 deleted region in SMZL patients.

| Gene | Start (bp) | End (bp) | N^{o} GeneBank |
|---|---|---|---|
| TSC22D4 | 99902078 | 99914838 | BC010406.2, BC001486.2, |
| | | | BC031622.1, BC001966.1, |
| | | | BC002972.1 |
| HRBL | 99974770 | 99979594 | BC009393 |
| LRCH4 | 100009570 | 100021712 | BC018529 |
| EPHB4 | 100238122 | 100263079 | BC052804 |
| SLC12A9 | 100288293 | 100302570 | BC000154 |
| TRIP6 | 100302885 | 100309012 | BC028985, BC021540, |
| | | | BC004249, BC004999, |
| | | | BC002680 |
| SRRT | 100310636 | 100324221 | BC109117 |
| UFSP1 | 100324279 | 100325275 | BC067904 |
| ACHE | 100325550 | 100331477 | BC143469, BC105060, |
| | | | BC105062 |
| MUC3A/B | 100388685 | 100397831 | AF007194, AB038783, |
| | | | AF143372, AJ606308 |
| MUC12 | 100434795 | 100448936 | AK024856 |
| MUC17 | 100450083 | 100488860 | BC126315.1 |
| TRIM56 | 100515505 | 100520609 | BC005847, BC048194 |
| SERPINE1 | 100557098 | 100569262 | BC002488.2, BC008045.2, |
| | | | BC026916.1, BC003049.1, |
| | | | BC020555.1, BC019273.1, |
| | | | BC017449.1, BC10860.1 |
| AP1S1 | 100584405 | 100591277 | BC003561 |
| VGF | 100592509 | 100595572 | BC063835, BC044212 |
| CUX1 | 101247602 | 101680064 | BC066592 |
| SH2B2 | 101715166 | 101748898 | NM_020979 |

In the present invention the term “high-resolution CGH-array” is defined as a molecular cytogenetic method for the detection of chromosomal gains and losses that combines the CGH technology with the micro-arrays technology. The high-resolution genomic array is preferably a solid support to which is adhered by a biorobot and orderly clones and/or DNA sequences known, as a matrix of thousands of equally spaced dots. The affinity or resolution of genomic arrays is determined by the genomic distance between two consecutive clones. This technique is based on the co-hybridization on the array of two DNAs, tumor and control (reference), labeled with different fluorophores.

The high-resolution CGH-arrays used in the present invention are: BAC/PAC array containing 3,523 clones from BACs and PACs that cover the entire genome with a resolution of 1 Mb. To corroborate the results obtained from this platform have used FISH assays. Subsequently, to further ensure these results are made use of two commercial genomic arrays platforms: Agilent 44K CGH-array and NimbleGen 72K CGH-array. The data obtained by these techniques confirmed that patients with SMZL have a deletion in the 7q22.1 region of human chromosome 7. Moreover, a human CGH 385K Chromosome 7 Tiling Array, was used to redefine the specific area deleted in the 7q22.1 region on human chromosome 7 in SMZL patients. This array includes approximately 385,110 oligonucleotides or targets covering the entire chromosome 7 and leaving an average distance between each oligonucleotide of approximately 355 bp. In addition to high-resolution arrays mentioned above, other high-resolution array was used, specific of the regions between the cytogenetic bands 7q22.1 to 7q33, both inclusive (Chromosome 7: 99158137-137302483). This array including 710,953 probes with an average size of probe of 54 oligonucleotides and an average distance between each nucleotide to −0.7 bp, allowing to map that region with a very high accuracy.

Another aspect of the present invention refers to the kits for the diagnosis of SMZL patients comprising at least a probe to recognize the deleted region at 7q22.1 or that hybridizes with at least one of the nucleotide sequences of the genes mentioned above (Table 1), wherein such probes are bound to a compound, preferably a fluorophore, which allows detection.

The in vitro method discloses in the present invention allows to delimitate with a great precision the deleted region in 7q22.1, and a to do a better differential diagnosis of SMZL subjects versus subjects with other chronic lymphoproliferative disorders or non-Hodgkin lymphomas, since the specific deletion in the region 7q is highly specific of the SMZL and not of the rest of chronic lymphoproliferative disorders or non-Hodgkin lymphomas.

### DESCRIPTION OF THE FIGURES

FIG. 1. Common genomic regions of gains or losses on chromosome 7 of 57 patients with SMZL. The figure shows the genomic regions that present gains (G) expressed as percentage, and losses (L) expressed as percentage, on chromosome 7 of SMZL patients. For each region, it is shows the cytogenetic location and the gain (% G) and losses (% L) expressed as percentage.

FIG. 2. Genomic regions involved in SMZL patients showed or not alterations in chromosome 7q. The figure shows the gains (G), expressed as a percentage, and losses (L), expressed as percentage of patients with losses on chromosome 7q (black bars) and patients who do no showed losses on chromosome 7q (white bars).

FIG. 3. Analysis of chromosome 7 by the specific high-resolution Tiling array of the chromosome 7 from NimbleGen (385K) in patients with SMZL. The figure shows the deletion of the 7q22.1 region on three of the four cases discussed, shown as thick black lines. C: Control; SMZL 1, SMZL 2 and SMZL 3: patients with SMZL.

FIG. 4: Analysis of the commonly deleted region of chromosome 7, 7q22.1 in three patients with SMZL, by different CGH-arrays used in the invention. (A) The figure shows the three clones covering the region 7q22.1 of chromosome 7 analyzed by BAC/PAC CGH-array. The three patients with SMZL (SMZL 1, SMZL 2, and SMZL 3) show losses (in black) in the region 7q22.1 and no changes were observed in the control subject (C). The region 7q22.1 in this array is mapped by three clones with a resolution of 1 Mb. (B) The figure shows the 53 oligonucleotides included in the present CGH-array NimbleGen 72 K for the 7q22.1 region. The three patients with SMZL (SMZL 1, SMZL 2 and SMZL 3) showed losses in the 7q22.1 region (black), while the control (C) showed no changes in this region. (C) The figure shows the 3.553 oligonucleotides covering the region 7q22.1 in the specific Tiling-array of the chromosome 7. It is observed that the commonly deleted region in three patients with SMZL (SMZL 1, SMZL 2 and SMZL 3) analyzed is delimited between the loci D7S2120E and D7S740. C: control.

FIG. 5. Representation of the patterns of loss of heterozygosity (LOH) obtained by analyzing the microsatellite markers in SMZL patients. The figure shows a photography of agarose gels showing losses or not of the heterozygosity in SMZL patients. A) No LOH and B) LOH. T: tumor sample (spleen), NT: no tumor sample (bone marrow or peripheral blood).

FIG. 6. LOH analysis in chromosome 7q, specifically in the 7q22.1 region of patients diagnosed of SMZL. The left of the figure represents the chromosome 7 including cytogenetic bands. To the right shows an enlargement of the 7q22.1 region and the microsatellite markers analyzed in this region. The white boxes represent heterozygosity. The black boxes represent LOH and gray boxes represent non-informative samples. The letters A, B, C, D, E and F represent the patients analyzed. It can be seen as 4 out of 6 patients analyzed (B, C, D and F) show LOH for any of the markers included in the 7q22.1 region (D7S662, D7S2536, D7S515).

FIG. 7. CUX1 gene expression in patients with SMZL. Analysis of CUX1 gene expression by RT-PCR in spleen samples, bone marrow (BM) and peripheral blood (PB) of SMZL patients and healthy control (Control). A shows CUX1 gene expression (exons 2-4) and B shows CUX1 gene expression (exon 24). On the Y axis is expressed CUX1 gene expression in relative terms based on the ABL control gene expression.

FIG. 8. SH2B2 gene expression in patients with SMZL. Analysis of SH2B2 gene expression by RT-PCR in spleen samples and bone marrow (BM) to SMZL patients and healthy controls (Control). On the Y axis is expressed SH2B2 gene expression in relative terms based on the ABL control gene expression.

FIG. 9. Analysis of 7q22.1 region of the chromosome 7 between the bases 100260234-100596921 by high-resolution specific array from NimbleGen for the 7q22.1-7q33 region in patients with SMZL. The figure shows the deletion of the bases 100260234-100596921 in the 7q22.1 region in patients with SMZL.

FIG. 10. Analysis of 7q22.1 region to the chromosome 7 between the bases 101244026-101754718 by high-resolution specific array from NimbleGen for the 7q22.1-7q33 region in patients with SMZL The figure shows the deletion of the bases 101244026-101754718 in the 7q22.1 region in patients with SMZL.

FIG. 11. Alignment and comparison of CUX1 sequence gene in patients with SMZL respect to a consensus sequence of this gene obtained from the database (NCBI/HG18). A) The alignment of both sequences reveals that the intron 1 of CUX1 sequence in SMZL patients shows an insertion of a thymine and a cytosine at the positions 101345904 and 101345905 (boxed) from the reference genome HG18 obtained from the NCBI database regarding the CUX1 sequence obtained from SMZL patients. B) The alignment of both sequences reveals that the sequence of exon 3 of the CUX1 gene in SMZL patients includes at the position 101458144 in the reference genome HG18 obtained from the NCBI database the substitution of a guanine for a thymine (G>T) (boxed), ie, the letter “n” represents a thymine, regarding to the CUX1 sequence gene obtained from patients with SMZL.

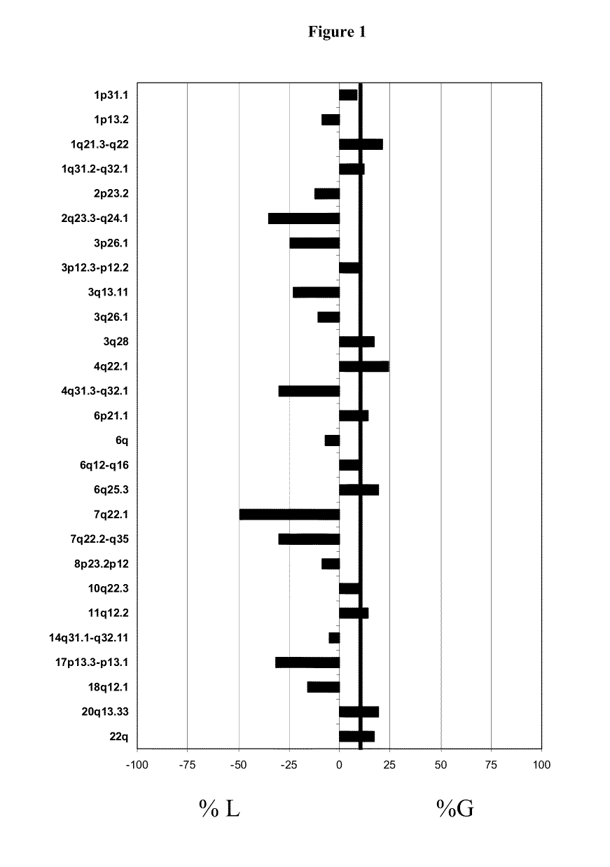

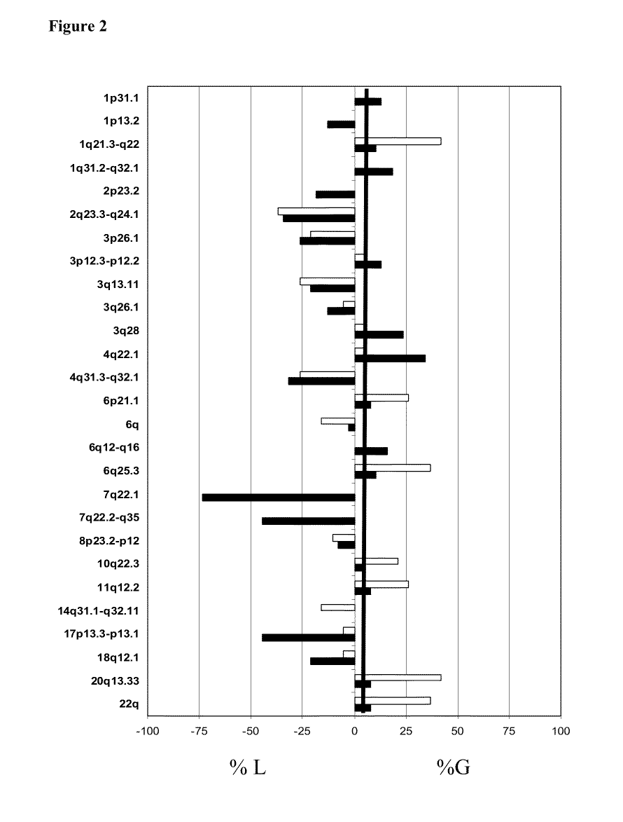

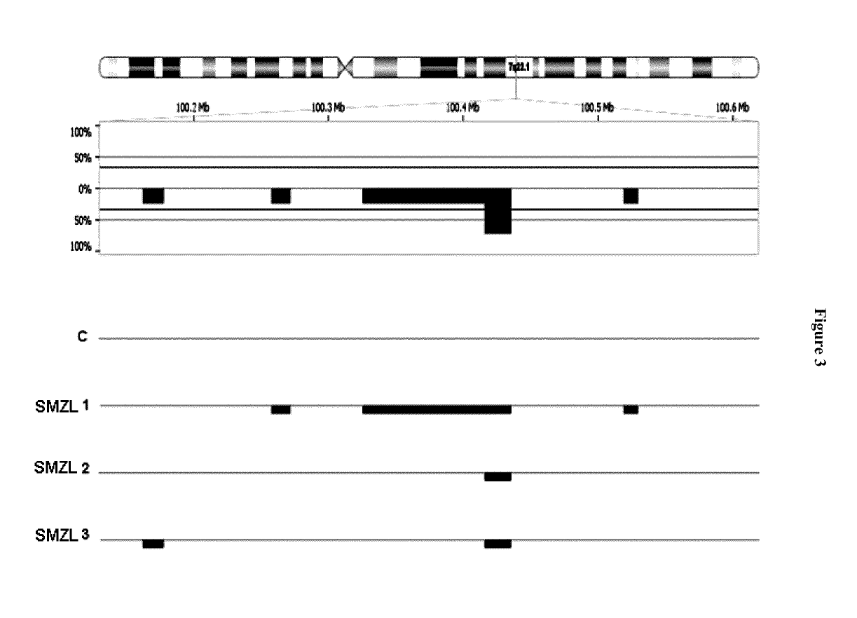

### DETAILED DESCRIPTION OF THE INVENTION

The first object of the present invention refers to an vitro method for diagnosis of splenic marginal zone lymphoma (SMZL) by high-resolution genomic microarrays, comprising a step for detecting the deletion between the bases 99225039 to 101754718 located in the 7q22.1 region in which is located, at least, the CUX1 gene.

In a preferred embodiment, the method of the invention is characterized in that the deletion is located between the bases 99925039 to 101348479.

In another preferred embodiment, the method of the invention is characterized in that the deletion is located between the bases 100260234-100596921 and/or 101244026-101754718 in the 7q22.1 region.

In another preferred embodiment, the method of the invention is characterized in that the CUX1 and SH2B2 genes are located in these deleted regions.

In another preferred embodiment, the method of the invention is characterized in that further in said region are located, among other, the genes EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17.

In another preferred embodiment, the method of the invention is characterized in that additionally comprises the detection, by high-resolution genomic microarrays, of the deletion between the bases 102949624 to 1459599694 in the 7q22.2-q35 region in which is located, among others, the genes RELN, LHFPL3, PBEF1, THAP5, IMMP2L, FOXP2, TES, MET, ST7, PTPRZ1, GRMB, UBEH2, MLKN1, BPGM, CALD1, PTN, SVOPL, AGK, KEL, TPK1 and CNTNAP2.

In another preferred embodiment, the method of the invention is characterized in that it is performed on a DNA sample selected from: a tissue sample from a lymphoma cell infiltrate, a bone marrow aspirate or a whole blood sample.

In another preferred embodiment, the method of the invention is characterized in that the high-resolution genomic microarrays are selected from BAC/PAC CGH-arrays, Agilent CGH-array, NimbleGen CGH-array or CGH Chromosome 7 Tiling Array from NimbleGen and CGH 7q22.1-q33 region Tiling Array from NimbleGen.

Another object of the present invention relates to an in vitro method for diagnosis of splenic marginal zone lymphoma (SMZL) which comprises measuring the expression level of at least the CUX1 gene, located in the 7q22.1 region between the bases 99925039 to 101754718.

In a preferred embodiment, the method of the invention is characterized in that it comprises measuring the expression level of the CUX1 gene located in the 7q22.1 region between the bases 99925039 to 101348479.

In another preferred embodiment, the method of the invention is characterized in that it comprises measuring the expression level of the CUX1 gene located in the 7q22.1 region between the bases 101244026 to 101754718.

In another preferred embodiment, the method of the invention is characterized in that it comprises measuring the expression level of the, at least, the CUX1 and SH2B2 genes located in the 7q22.1 region between the bases 101244026 to 101754718.

In another preferred embodiment, the method of the invention is characterized in that it further comprises measuring the expression level of at least one of the following genes selected from: EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17, or combinations thereof, in the 7q22.1 region between the bases 99925039 to 101348479.

In another preferred embodiment, the method of the invention is characterized in that it is performed on a DNA sample selected from: a tissue sample from a lymphoma cell infiltrate, a bone marrow aspirate or a whole blood sample.

Another object of the present invention relates to an in vitro method for diagnosis of splenic marginal zone lymphoma (SMZL) which comprises a combination, in any order, of a first step for detecting, by high-resolution genomic microarrays, the deletion between the bases 99225039 to 101754718 located in the 7q22.1 region and a second step for measuring the expression level, of at least, the CUX1 gene.

In a preferred embodiment, the method of the invention is characterized in that the deletion is located between the bases 99925039 to 101348479.

In another preferred embodiment, the method of the invention is characterized the deletion is located between the bases 100260234 to 100596921 and/or 101244026 to 101754718 in the 7q22.1 region.

In another preferred embodiment the method of the invention is characterized in that in the second step the expression levels of the CUX1 and SH2B2 genes are measured

In another preferred embodiment, the method of the invention is characterized in that further comprises measuring the expression levels of at least one of the following genes selected from: EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17 and/or combinations thereof.

In another preferred embodiment, the method of the invention is characterized in that additionally comprises the detection, by high-resolution genomic microarrays, of the deletion between the bases 102949624 to 1459599694 in the 7q22.2-q35 region in which is located, among others, the genes RELN, LHFPL3, PBEF1, THAP5, IMMP2L, FOXP2, TES, MET, ST7, PTPRZ1, GRMB, UBEH2, MLKN1, BPGM, CALD1, PTN, SVOPL, AGK, KEL, TPK1 and CNTNAP2.

Another object of the present invention relates to a kit for the in vitro diagnosis of splenic marginal zone lymphoma (SMZL) comprising a set of probes selected form the group SEQ ID No. 1, SEQ ID No. 2, SEQ ID No. 3, and/or any probe that hybridizes wholly or partially with the deleted region between the bases 99925039 to 101754718 by high-density genomic microarrays

In a preferred embodiment, the kit of the invention is characterized in that the selected sets of probes hybridize wholly or partially with the deleted region between the bases 99925039 to 101348479.

In another preferred embodiment, the kit of the invention is characterized in that the selected set of probes hybridize wholly or partially with the deleted regions between the bases 100260234 to 100596921 and/or 101244026 to 101754718 of the region7q22.1.

In another preferred embodiment, the kit of the invention is characterized in that it further comprises at least a probe that hybridizes wholly or partially with the deleted region between the bases 102949624 to 1459599694 in the 7q22.2-q35 region, by high-density genomic arrays, preferably the probe is SEQ ID No. 4.

In another preferred embodiment, the kit of the invention is characterized in that the probe hybridizes in the 7q33 region.

Another object of the present invention relates to a kit for the in vitro diagnosis of splenic marginal zone lymphoma (SMZL) characterized in the detecting the expression level of at least the CUX1 gene, located in the 7q22.1 region between the bases 99925039 to 101754718 by RT-PCR with the primers defined as SEQ ID No 5 and SEQ ID No. 6.

In a preferred embodiment, the kit of the invention is characterized by measuring the level of expression of CUX1 gen by RT-PCR, using define probes as SEQ ID NO:5 and SEQ ID NO:6 detected the deletion region between 100260234-100596921 and 10101244026-101754718 on 7q22.1.

In another preferred embodiment, the kit of the invention is characterized in that the expression level of at least the CUX1 gene, by RT-PCR with the primers defined as SEQ ID No 5 and SEQ ID No. 6 it is performed in the deleted region between the bases 99925039 to 101348479.

In another preferred embodiment, the kit of the invention is characterized in that the expression level of at least the CUX1 gene, by RT-PCR with the primers defined as SEQ ID No 5 and SEQ ID No. 6 it is performed in the deleted region between the bases 100260234 to 100596921 and/or 101244026 to 101754718 of the region7q22.1.

In another preferred embodiment, the kit of the invention is characterized in that the expression level of the CUX1 gene it is further performed with the primers defined as SEQ ID Nos. 23-24.

In another preferred embodiment, the kit of the invention is characterized in that additionally comprises the detection of the expression level of SH2B2 gene by the primers defined as SEQ ID Nos 25-28.

In another preferred embodiment, the kit of the invention is characterized in that further comprises detecting the expression levels of at least one of the following genes selected from: EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17 and/or combinations thereof by specific primers for the sequences of these genes.

### EXAMPLES

The following examples are presented to illustrate the invention and not intended to limit the scope thereof.

### Example 1

### DNA Extracted from Samples of SMZL Patients

To obtain the results shown in the present invention Seventy eight patients with the diagnosis of SMZL were included in this study. The diagnosis was established according to the WHO classification. Thirty six patients (56%) were women. The ages ranged from 44 to 86 years (median 69 years). The study was approved by the local ethical committees. Informed consent was obtained from each patient before entering the study.

DNA was extracted from spleen samples, lymphoid node, bone marrow aspirate or samples of whole blood provided that the tissues were infiltrated with cells of SMZL. All genomic DNA was extracted from fresh-frozen samples using the standard phenol-chloroform method. In 53 patients, Tumour DNA was isolated from the spleen, bone marrow and peripheral blood. Normal DNA was extracted from the human placenta of healthy donors. All DNA was quantified using the NanoDrop spectrophotometer (ND-1000, NanoDrop Technologies, Wilmington, Del., USA). DNA quality was assessed by the 260:280 ratios and its integrity by agarose gel ethidium bromide visualization.

### Example 2

### Characterization of Genomic Imbalances in SMZL by High-Resolution BAC/PAC Array

After genomic DNA of SMZL patients and Normal DNA from the healthy donors was extracted, a genomic-wide analysis of DNA copy number changes of patients was performed using BAC CGH-array. To confirm the gains and losses assessed by array-CGH was performed FISH analysis.

In the present stud of SMZL was analyzed using BAC/PAC CGH-array, Agilent CGH-array, NimbleGen CGH-array and by tiling path array specific from chromosome 7

The first genomic-wide analysis of DNA copy number changes of SMZL patients was performed using BAC CGH-array. This BAC/PAC array contained 3528 bacterial artificial chromosomes (BAC) and PAC (P1-Derived Artificial Chromosome) contains targets spaced at ≈1 Mb density over full genome. Slides were produced at the Centre of Cancer Research (Salamanca, Spain) as previously described. {Robledo, 2009}.Briefly, 2009). Briefly, first we proceeded to the preparation of genomic SMZL DNA patients and healthy controls, to hybridize to the array.

Briefly, 2 μg of genomic DNA of and and reference material (placental DNA) were digested separately with DpnII restriction enzyme (New England Biolabs, Beverly, Mass.) and subsequently proceed to purification by decreasing gradient centrifugations ethanol. The samples of digested and purification genomic DNA (patients and control) were labeled using random primers (Bioprimer labelling kit, Invitrogen) and Cy5-dCTP and Cy3-dCTP (Amersham Biosciences) fluorophores for paired hybridization samples, respectively. The labelled nucleotide incorporation was quantified using the NanoDrop spectrophotometer (ND-1000, NanoDrop Technologies, Wilmington, Del., USA). Labelled test and reference DNA were mixed equitably, co-precipitated in presence of Cot-1 human DNA (Roche, Indianapolis, Ind.) with ethanol, washed, and resuspended in hybridization solution (50% Formamide, 10% Dextran sulfate, 2× standard saline citrate, 10 mM Tris pH 7.6, 2.7% sodium dodecyl sulfate (SDS) and 10 μg/μl of yeast tRNA). DNA mixtures were cohybridized to the arrays in the GENETAC (Genomic Solutions) for 48 hours at 42° C. according to the manufacturer's recommended protocol. After to array hybridization, images and signal intensities were acquired using the GenePix4000B (Axon Instruments, Burlingame, Calif.) dual laser scanner in combination with the GenePixPro4.0 (Axon Instruments) imaging software. Intensity of Fluorescence of every spot were calculated after subtracted the background. Fluorescence ratios were normalized by using the median of fluorescence ratios of every spot computed as log₂ values.

To validate the array were a series of hybridizations man vs woman controls and also marking the DNA used as control hybridizations with samples to be tested with different fluorochromes, to eliminate these clones in hybridizations are altered because they considered that are not affected by to analyze disease. Gains and losses in copy number of regions genomic DNA obtained by array-CGH platform BAC/PAC were identified by creating specific samples thresholds, determining a value of cutting 0.4 (Robledo C et al, Cancer. 2009, 115 (16):3728-37). To determine which clone is altered to calculate the average values the logarithms of the ratios of the signal corresponding to Cy5 (tumor DNA) versus corresponding to Cy3 (control DNA) of each clone triplicate. A cut off value of 0.4 was used based on the ratios of clones in ten hybridization of normal male versus normal female DNA. The clones with log₂ ratios above or below a control sample's threshold value were considered as gains or losses, respectively. At least two contiguous BAC clones with a log₂ ratio of −0.4 or less were defined as a loss region and log₂ ratio of +0.4 or more was defined as a gain region. Furthermore, spots with weak Cy3 or Cy5 intensity (below R²<0.2) and clones with a standard deviation of more than 0.3 of triplicate spots were excluded from analysis. In total, approximately 10% of clones were excluded.

All data sets were carefully reviewed for frequently affected chromosomal sites of physiologic copy number polymorphisms (CNP). Therefore every clone on the array was compared with the ‘Database of Genomic Variants’ (available at: www.project.tcag.ca/cariation; November 2009) and that of chromosomal imbalances and phenotype in humans using Ensembl Resources (DECIPHER: available at: http://www.sanger.ac.uk/PostGenomics/decipher/; November 2009).

The results obtained show that 57 (84%) of the patients with SMZL showed genomic changes by BAC/PAc array. The median of changes per patients was four (with a range from 0 to 12). The most frequent changes were chromosomal gains involving 4q22.1 (14/57; 25%), 1q21.3-q22 (12/57; 21%), 6q25.3, 20q13.33 (11/57; 19%), 3q28, 22q (10/57; 18%), 6p21.1, and 11q12.2 (8/57; 14%) while the genomic losses were located on 7q22.1 (28/57; 49%), 2g23.3-q24.1 (20/57; 35%), 17p13.3-p13.1 (18/57; 32%), 4q31.3-q32.1, 7q31-q35 (17/57; 30%), 3p26.1 (14/57; 25%), 3q13.11 (13/57; 23%) and 18q12.1 (9/57; 16%)

Only three cases showed losses on chromosome 14q and only one case had trisomy chromosome 12. Moreover, minimal common region deleted was observed in 18 patients (32%) on chromosome 17p13.1, where TP53 gene is located, which is a suppressor gene tumoral processes involved in induction of cellular response to DNA damage, arresting the cell cycle even if mutations.

In the 19 patients showed no alterations in chromosome 7q median 10 changes was 4 (range 1-8) and more frequent gains were located at the level of 21.3 1q-q22, 20q13.33 (42%), 6q25.3, 22q (37%), 6p21.1, 11q12.2 (26%) and 10q22.3 (21%) while losses in 2q23.3-q24.1 (37%), 4q31.3-q32.1 (26%), 3p26.1 (21%), 6q, 14q31. L-q32.11 (16%) and 8p23.2-p12 (10%).

### Example 3

### FISH Validation of Losses Identified by BAC/PAC CGH-Array

As methodology confirmation of the results obtained by High-resolution BACIPAC microarray CGH-array, fish studies was performed in a total of 20 patients diagnosed SMZL. By FISH technique can observed by fluorescence microscopy, whether or not a region is deleted, and that the chromosomal region that hybridizes to each of the probes used is duplicate in each of the cells, whether the deleted area is to be noted in cell two signals corresponding to the color with which the fluorophore is labeled the probe. In contrast, if it is deleted, only a single observed color signal corresponding to the fluorophore is labeled the probe.

In all cases FISH analysis confirmed by the results obtained by CGH-array: 7q deletion To corroborate more specifically those results 8 SMZL patients were selected, three of them showed losses in the region BAC/PAC 7q by CGH-array and five showed no genetic changes in this region. The criteria for selection of patients were based on the availability of the sample (spleen sections embedded in paraffin blocks).

The BAC probes used for FISH were RP11-44M6 mapped to 7q22.1 (99873610-100038722 bp), RP11-333G13 mapped to 7q22.1 (101175494-101390682 bp), RP11-36B6 mapped to 7q31.31 (130078078-130270796 bp) and RP-371N6 mapped to 7q33 (134684519-134842787 bp) as previously described (NCBI16/hg18). (González MB et al,. Cancer Genet Cytogenet. 2004; 150(2):136-43).

These clones were selected from the same BAC CGH-array clones library used for the BAC-array studies (Wellcome Trust Sanger Institute, Cambridge, UK). DNA from the BAC clones was isolated and directly labelled with either Spectrum Green-dUTP or Spectrum Orange-dUTP (Vysis, Downers Grove, Ill.) by nick translation.

FISH on 7q22.1 region was analyzed in seven of the eight patients Four of these patients showed a loss in these region and the result was confirmed by FISH technique, while the remaining three patients showed no loss by BAC/PAC CGH-array neither FISH technique.

On the other hand, other four patients were analyzed by FISH 7q33.1 region. Two of SMZL patients showed deletions using both techniques: BAC/PAC Array-CGH and FISH. The remaining two patients showed no alterations in the region of 7q33.1 by either techniques used, BACIPAC FISH and array-CGH.

### Example 4

### Analysis of SMZL by Commercial CGH-Array

In order to confirm the results provided by the BAC CGH-array analysis, two types of oligonucleotide CGH-arrays platforms were used. Those platforms comprise more affinities and resolution than BAC/PAC array. Those plataforms were Agilent's Human Genome CGH

Microarray 44 k (Agilent Technologies).and NimbleGen human CGH 4x72K Whole Genome v2.0 array (NimbleGen Systems, Inc).

Eight SMZL were analyzed with Agilent's Human Genome CGH Microarray 44 k (Agilent Technologies). The microarray contains 44,255 in situ synthesized 60-mer probes spaced at 43,000 bp intervals throughout the human genome and including 3,877 controls. The probes are, according to the manufacturer's description, enriched for cancer relevant genes representing both coding and non-coding sequences on the chromosomes. Experiments using Agilent arrays were performed using placenta human genomic DNA as reference and following the Agilent recommended standard protocol. The arrays were scanned using the Agilent scanner, data extraction, filtering and normalization were conducted using the Feature Extraction software (Agilent Technologies). The Agilent CGH Analytics Software 3.4 trial (Agilent Technologies) was used to export the CGH-array data for usage in Nexus Copy Number Professional trial software (version 4, BioDiscovery Inc) (Baumbusch, LO et al. BMC Genomics 2008; 8; 99:379).

Eleven patients with SMZL were analyzed with NimbleGen human CGH 4x72K Whole Genome v2.0 array (NimbleGen Systems, Inc). Placenta DNA was used as reference. The CGH-array protocol from NimbleGen Systems was followed. Briefly, 500 ng of tumor and reference DNA were labelled with Cy3 and Cy5, respectively. Labelled material was co-hybridized to microarrays consisting of 71,341 oligonucleotides probes spaced approximately at 40,000 bp intervals throughout the human genome, washed and scanned at 10-um resolution using the GenePix 4000B dual scanner (Axon Instruments, Burlingame, Calif., USA). Raw data were extracted using NimbleScan software v2.5 (NimbleGen Systems, Inc), which allows for automated grid alignment, extraction and generation data files (Selzer R R et al,. Genes Chromosomes Cancer. 2005 November; 44(3):305-19)

The CGH-array methodologies employed in the present invention, BAC-CGH array, Agilent's Human Genome CGH Microarray 44 k (Agilent Technologies). and NimbleGen human CGH 4x72K Whole Genome v2.0 array (NimbleGen Systems, Inc).confirm the genomic changes observed in SMZL patients. The most frequently changes, were gains on chromosomes 3, 6p221.p21.1, 8q,17q, 18 and losses on 4q28.3-q31.23, 10q24.33-q25.3 and 17p13.3-p13.1. In additional, CGH-arrays showed trimosy of 3, 12 and 18 in one patient. All methodologies of genomic DNA, BAC/PAC array, or Oligonucleotides-Agilent 44K and Nimblengem 72K, showed changes on 7q, it confirmed deletion on 7q by three different techniques.

In order to better localize the lost region at 7q, were analyzed by tiling-path CGH-array. These arrays were designed at higher resolution with fine-tiling analysis of the chromosome 7

### Example 5

### High-Resolution Analysis of Chromosome 7

Fine analysis by 385K “Tiling array “NimblemGen, confirm and define the specific deletion region in SMZL. Four patients with SMZL were analyzed by tiling-path CGH-array (three showed losses on 7q22.1 confirm by previous techniques and one SMZL without deletion). These arrays were designed at higher resolution with fine-tiling analysis of the chromosome 7 (chromosome7: 117,711-158,805,222 bp). This array was constructed by mask less array synthesis technology (NimbleGen Systems, Inc.), with up to 385,110 oligonucleotides, with a median probe spacing to 365 bp. These arrays were designed at higher resolution which allows designing oligonucleotides that are often placed overlapping with each other to thereby obtain a high resolution of the area to be analyzed in the present invention, the entire chromosome 7.

After the hybridization, arrays were scanned at 5 μm resolution using the NimbleGen MS 200 Microarray scanner (Roche NimbleGen Instruments). Data were extracted from the scanned images using NimbleScan software v2.5 (NimbleGen Systems, Inc), which allows for automated grid alignment, extraction and generation data files Chromosome 7 tiling array NimbleGen 385K was performed in four SMZL patients. In the analysis of tiling array we detected that three patients showed losses on band 7q22.1 (FIG. 3).

Analyzing the results of all studies performed by CGH-array on samples SMZL patients and specifically into account the data obtained through the specific array Tiling chromosome 7, shows that the deletion majority of SMZL patients—49% (27 of 57 cases)—is located in the band 7q22.1 and is mapped by clones or probes of array-CGH BAC/PAC: BAC RP11-44M6 (SEQ ID NO:1), PACRP5-1059M13 (SEQ ID NO: 2) y BAC RP11-333613 (SEQ ID NO:3), more specifically between bases 99925039-101348479. This region involves genes such as EPHB4, SLCI2A9, TRIP6, SRRT, ACHE, UFSPI, TRIP56, SERPINEI, APISI, VGF, TSC22D4, HRBL, LRCH4, MUC3, MUC12, MUC17, CUX1 and SH2B2. It should be noted that the size of this region is around of 1, 4 Mb

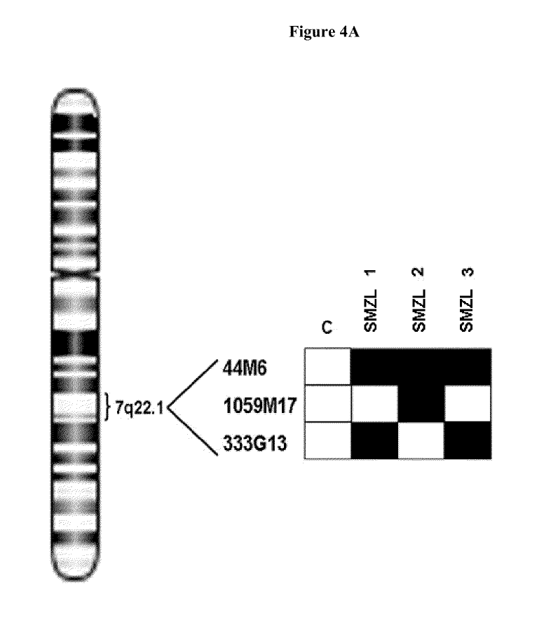

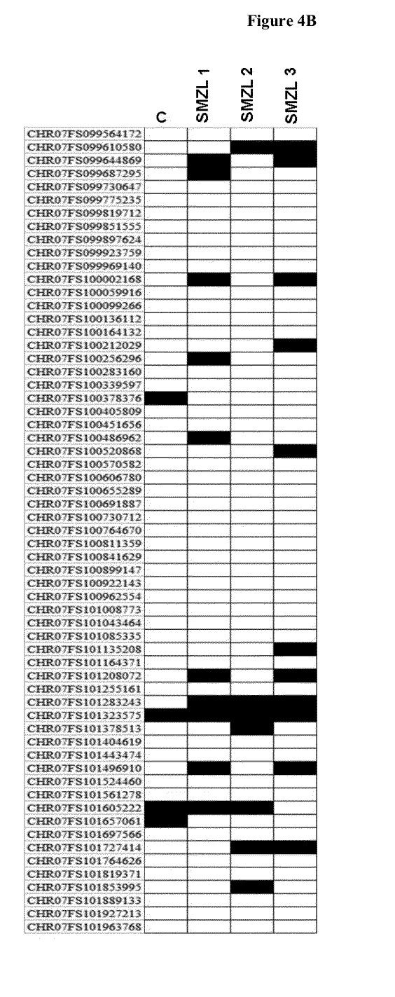

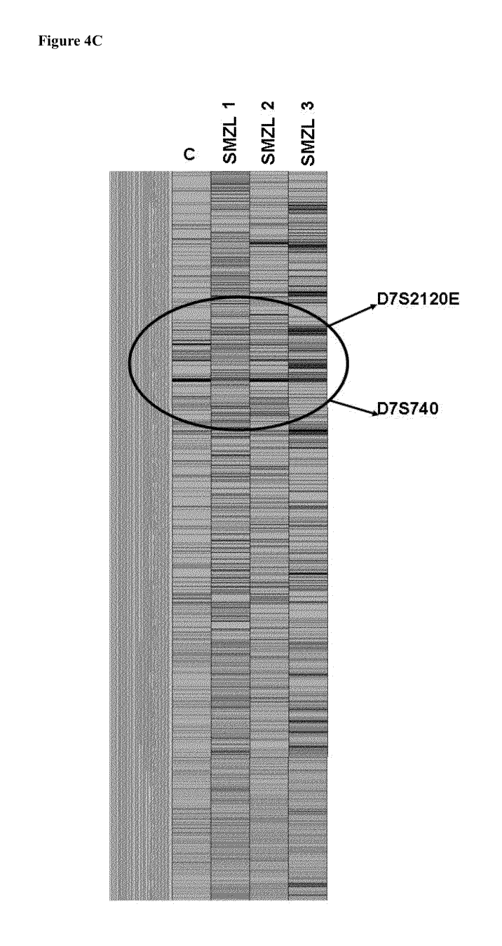

The accurate detection of the deleted region in band 7q22.1, which involves the 99925039-101348479 bp, has been made possible by the difference between resolutions limits of the different methodologies used: BACIPAC CGH-array, Agilent 44K, NimbleGen 72K and Chromosome 7 Tiling array of 385K. As shown in FIG. 3, Chromosome 7Tiling array, this region was possible to refine the mapping of the region better than the other different platforms, 3.555 oligonucleotides (FIG. 4C) versus 53 of NimblenGen 72K (FIG. 4B) or three BAC/PAC CGH-array (FIG. 4A). As it can be seen, Chromosome 7 Tiling array of 385K due to the fact that the focused array has more probes covering the same genomic area, it can offer a better and more precise estimation of the exact break point of to deletion on 7q22.1.

FIG. 4A show the BAC/PAC CGH array analysis of three patients. As such observed that these patients had a loss, shown in black in 7q22.1 region, whereas in the control change is not observed. The 7q22.1 was mapping by 3 clones with 1Mb resolution. FIG. 4B show the date of NimblenGen 72K analysis with 42 Kb resolution, and 7q22.1 was mapping by 53 oligonucleotides, in three SMZL patients. The results show that these patients had a loss in 7q22.1 region, whereas in the control change is not observed. FIG. 4C shows the probe-level view of CGH-array NimbleGen 385K all zoomed in on the area of the deletion (7q22.1).This region were mapping by more than 3500 oligonucleotides. As it can be seen it was possible to refine the mapping of the deletion region between D7S2120E and D7S740.locis. Therefore, Chromosome 7 Tiling array of 385K allows more accurately detect the exact location of the deletion occurs on chromosome band 7q22.1

By the method of the invention, has also revealed deletion at a distal region of chromosome 7, the 7q22-q35 region, between 102949624-145959694 bp. The size of this region of 43,14 MB is mapping between BACs RP4-672011 and RP4-558L10. This region involves genes such as RELN, LHFPL3, PBEFI, THAP5, MMP2L, FOXP2 TES, MET, ST7, PTPRZI, GRM8, UBE2H, MLKNI, BPGM, CALDI, PTN, SVOPL, AGK, KEL, TPKI and CNTNAP2. This region is detected among other by, clone o probe BAC/Pac CGH-array: BAC RPI 1-371N6 (SEQ ID NO: 4), mapping in 7q33 (135034979-135191247). So, using all the probes that recognize the specific deletion of the 7q22.1 region, defined as SEQ ID NO: 1, SEQ ID NO: 2 and SEQ ID NO: 3, together with the use of the probe recognizes deletion 7q33 region, defined as SEQ ID NO: 4, could achieve an increase in the number of patients diagnosed with SMZL.

The results obtained through the use of commercial platforms, NimbleGen, Agilent, and array-CGH BACIPAC confirm the partial loss of chromosome 7 as the most frequent change in patients with SMZL. Furthermore, we show the existence of a new region commonly deleted in patients with SMZL, which allows it to be useful for clinical use, located at the level of the band 7q22.1, in a 49% patients analyzed. In this specific region deleted in patients SMZL, located between 99925039-101348479 bp in 7q22.1, the genes are located EPHB4, SLCl2A9, TRIP6, SRRT, ACHE, UFSPI, TRIM56, SERPINEI, APISI, VGF, TSC22D4, HRBL, LRCH4, MUC3, MUC12 MUC17, CUXI and SH2B2. The analysis of expression of these genes in SMZL patients versus healthy controls or affected from other types of lymphomas, will detect whether any of them may be used as a diagnostic marker for the disease.

### Example 6

### LOH Analysis

To analyze the extent of the region identified on 7q22.1 for CGH-array, previously described, and trying to define the minimum region of molecular allelic loss studies were carried out in SMZL patients. LOH technique is also one of the most widely used methods in the prior art, from the molecular point of view, to determine the loss of genetic material (Matthew M, et al. Am J Pathol. 1999, 154:1583-1589).

The LOH using microsatellite markers allowed to detected relative loss of one allele in a tumor sample relative to a sample of the same patient tumor presents both alleles. In the present invention, defining the allelic loss is limited to informational markers. The markers were considered informative when heterozygosity detected in tissue samples of normal controls which results in the presence of two bands on the agarose gel corresponding to two different alleles.

In the case that a patient will show a LOH the PCR product only will show an amplified band, from one of the alleles, because the other allele will be deleted in the tumor simple. By contrast, the non-tumor sample will present both alleles as two amplified bands (FIG. 5).

In order to assess the loss of heterozygosity the nucleotide repeats from six patients with SMZL were analyzed by PCR. A total of seven microsatellite markers were selected on chromosome 7. These markers were selected based on the data from the databases Genethon, GDB and RHdB. The genome location (NCBI36/HG18) of the markers was performed by Electronic-PCR (e-PCR) (Schuler G D. Sequence Mapping by Electronic PCR. Genome Res 1997; 7: 541-550 (. The microsatellite markers used to better precise the location of the deleted region on 7q22.1 in the chromosome 7 in SMZL patients are described in Table 2.

TABLE 2
Molecular markers used to microsatellite amplification.

| Micro- | Chromo- | Amplicon | | |
|---|---|---|---|---|
| satellite | somal | size | Location | |
| marker | band | (pb) | (pb) | Sequences |
| D7S527 | 7q21.3 | 273-297 | 95.453.048 | SEQ ID NO: 9-10 |
| D7S2480 | 7q22.1 | 189-225 | 99.893.591 | SEQ ID NO: 11-12 |
| D7S662 | 7q22.1 | 204-234 | 100.950.585 | SEQ ID NO: 13-14 |
| D7S2536 | 7q22.1 | 118-141 | 101.235.892 | SEQ ID NO: 15-16 |
| D7S515 | 7q22.1 | 128-190 | 101.490.495 | SEQ ID NO: 17-18 |
| D7S500 | 7q33 | 188-210 | 134.759.259 | SEQ ID NO: 19-20 |
| D7S550 | 7q36.3 | 177-200 | 155.210.270 | SEQ ID NO: 21-22 |

PCR was carried out in a final volume of 25 uL, containing 30-50 ng/uL of DNA template, 5×PCR buffer (Promega, Madison Wis., USA), 25 mM MgCl₂ (Promega, Madison Wis., USA), 2.5 mM from nucleotide mixture (dNTPs: dATP, dGTP, dCTP and dTTP, Roche Diagnostics, Indianapolis, Ind., USA), 10 uM from the primers used for microsatellite identification and one unit of Taq DNA polymerase (Promega, Madison Wis., USA). The conditions used for PCR were as follow: 94° C. for 5 minutes, 35 cycles of 94° C. during 30 seconds, 55-65 ° C. (in relation to the primer used) 30 seconds, 72° C. during 30 seconds and, finally, the samples were placed at 4° C. The PCR products were separated in a 3% agarose gel (MSG Metagel, Conda, Madrid, Spain).

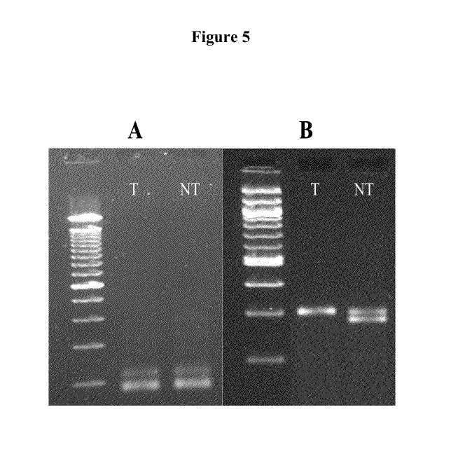

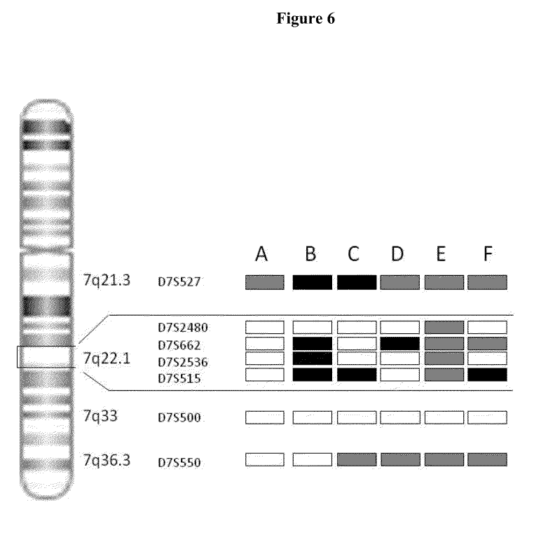

The LOH studies showed that from the six patients with SMZL analyzed, four of them showed LOH at 7q22.1 (67%). In addition LOH showed that, for the markers D7S662, D7S2536 and D7S515 (located at 100.950.585, 101.235.892 and 101.490.495, respectively, in the region 7q22.1) the percentage of losses was 50%, 20% and 60%, respectively (FIG. 6).

In the previous studies analyzing 13 microsatellite markers covering from region 7q21 to 7q36 of chromosome 7, it was observed that the marker D7S487 located at 7q31.3 was predominantly lost (45% of patients) while the microsatellite marker D7S518, located at 7q22, did not showed any lost in the analyzed patients (Mateo M et al. Am J Pathol 1999; 154: 1583-1589). In the present invention we were able to in deep study the region 7q22.1 allowing us to demonstrate the LOH in the microsatellite markers D7S527 (7q21.3), D7S662 (7q22.1), D7S2536 (7q22.1) and D7S515 (7q22.1) (FIG. 6). It should be noted that two of these markers are placed in the CUX1 gene, located in the deleted region 7q22.1 in SMZL patients. Thus the marker D7S515 is located in the intron 3 of CUX1 gene while D7S6626 is located in the intron 6 of CUX1 gene.

### Example 7

### Detailed Analysis of 7q22.1-7q33.1 Region of Chromosome 7 by a Tiling Microarray

In the previous examples we have demonstrated that SMZL patients had a deletion on 7q22.1 on the chromosome 7, located between the bases 99925039 and 101348479. Moreover these patients also showed a LOH of the microsatellite markers D7S662 (7q22.1), D7S2536 (7q22.1) and D7S515 (7q22.1). In order to better define the deleted region on 7q22.1 we had studied this region by using a tilling-array with high density that we order to NimbleGen System Inc. This microarray was enriched in probes from the cytogenetic band 7q22.1 to 7q33 (both bands were included in the microarray, mapping the positions Chr7: 99.158.389-137.302.483). This high-density microarray included a total of 710,953 genetic probes, with a medium size of 54 nucleotides (range: 49-74) and a medium distance of −0.7 bp (range: (−51) to 59217 bp). This CGH-array allow us the possibility to map this specific genome region with a high precision.

This tilling-array was used to characterized the 7q22.1-q33 was applied to study a total of 15 samples from SMZL patients, two cases with chronic lymphocytic leukemia (CLL) and an additional healthy control. Seven out the 15 patients (46%) with the diagnosis of SMZL showed losses at 7q22.1, and the microarray located the losses in two regions: the first region of lost in SMZL patients ranged from the genetic positions 100.260.234 to 100.596.921. The second region was located between the genetic positions 101.244.026 to 101.754.716 (Table 3). Therefore by using this tilling-array it was possible to better define the lost region at 7q22.1, and the size of the commonly deleted region size was reduced from 1.4 Mb to 0.84 Mb.

TABLE 3
Location of the deleted regions assessed
by a tilling-array in SMZL patients

| | Cases with losses |
|---|---|
| Chr 7 region | (n = 15) |
| 100.260.234-100.262.201 | 7 |
| 100.288.318-100.289.733 | 6 |
| 100.398.197-100.398.435 | 6 |
| 100.593.128-100.593.653 | 7 |
| 100.595.970-100.596.921 | 6 |
| 101.588.268-101.588.318 | 4 |
| 101.698.131-101.698.723 | 4 |
| 101.713.340-101.713.623 | 3 |

### Example 8

### Gene Expression Analysis of CUX1, SH2B2 and MUC3 in SMZL Patients

In the chromosomal region 7q22.1 there are located a high number of genes. The deleted region in SMZL, between the bases 100.260.234 to 100.596.921 located at 7q22.1, included the following genes: EPHB4, SLC12A9, RIP6, SRRT, UFSP1, ACHE, MUC3A/B, MUC12, HUC17, TRIM56, SERPINE1, AP1S1 and VGF. In addition, the region located also on 7q22.1, included the genes CUX1 and SH2B2, but between the bases 101.244.026 to 101.754.718.

The gene CUX1 encodes to a protein that is a member of the proteins binding DNA. It has been related to the gene expression, morphogenesis and differentiation, and also it can play a role in the progression of the cell cycle. The presence of a monosomy of chromosome 7, with a loss of the genes located in 7q22 such as CUX1, is frequent in patients with myeloid hemopathies, such as myelodysplastic syndromes or juvenile chronic myelomonocytic leukemia. However the mutational analysis failed to demonstrate the presence of mutations in none of the analyzed genes. Therefore it has been considered that CUX1 could be not involved in these neoplasms (Hindersi S et al. Leuk res 2007; 31: 1323-1324). To our knowledge no studies showed the CUX1 deregulation in SMZL patients. Moreover, the gene SH2B2 encodes for an adapter protein from some members of the tyrosine-kinase receptors family and it has been involved in several cell signal pathways, mainly from B-cells (Masanori I et al, Biochem Biophys Res Com 2000; 272: 45-54). The expression analysis of this gene in solid tumors, such as esophagus, lung and kidney, revealed that this gene is usually not express in the cell lines studied. By contrast, a high expression of SH2B2 was observed in normal tissues (Yokohuchi et al Oncogene 1999; 18: 759-767). Therefore the role of SH2B2 gene in SMZL patients remains unclear.

In the present invention a gene expression analysis of the genes CUX1, SH2B2 and MUC3 in samples from spleen, bone marrow and peripheral blood was carried out by PCR. A total of sixteen SMZL patients and six healthy controls were studied. By using PCR (Polymerase chain reaction) it is possible to achieve a large amount of RNA from the samples. The material from both the patients and the controls was previously frozen in trizol (Invitrogen, Lifetechnologies, Gaithersburg, Md.) at −80° C. before the RNA extraction. RNA purification was performed by using RNeasy Mini columns (Qiagen, Valencia, Calif.) in order to avoid the possible DNA or protein contamination. The RNA integrity was assessed by the Agilent 2100 Bioanalyzer (Agilent, Palo Alto, Calif.) by the RNA 6000 Nano Labchip. To synthesize the cDNA a Superscript III kit was used (Superscript III First-Strand Synthesis SuperMix for qRT-PCR, Invitrogen). In all cases lug of total RNA was used. The cDNA was purified by tha cDNA Cleanup kit (Affymetrix sample Cleanup Module kit).

The primers used for cDNA synthesis were designed by the software “Primer Express 3.0” (Applied Biosystems). In addition, the software “Oligoanalyzer” and NCBI BLAST were used to avoid the dimerization and to chek the specificity, respectively. The primers used for the gene expression analysis for the exons of the genes CUX1, SH2B2 and MUC3 are described in the table 4. In all cases the ABL gene expression was used as a control.

TABLE 4
Primers used for gene expression analysis (RT-PCR)

| Gene | Primer | Sequence |
|---|---|---|
| CUX1 (Exon 2-4) | Forward | SEQ ID NO: 23 |
| | Reverse | SEQ ID NO: 24 |
| CUX1 (Exon 24) | Forward | SEQ ID NO: 5 |
| | Reverse | SEQ ID NO: 6 |
| SH2B2 (Exon 6) | Forward | SEQ ID NO: 25 |
| | Reverse | SEQ ID NO: 26 |
| SH2B2 (Exon 3-4) | Forward | SEQ ID NO: 27 |
| | Reverse | SEQ ID NO: 28 |
| MUC3A/MUC3B (Exon 1) | Forward | SEQ ID NO: 29 |
| | Reverse | SEQ ID NO: 30 |
| MUC3A/MUC3B | Forward | SEQ ID NO: 31 |
| | Reverse | SEQ ID NO: 32 |
| ABL | Forward | SEQ ID NO: 7 |
| | Reverse | SEQ ID NO: 8 |

To optimize the annealing temperature, a gradient test was performed by using a control cDNA from a multiple myeloma cell line in the iCycler (Biorad). Moreover a control gene (ABL) was used to assess the relative quantification of the target probe in relation to the referred gene by the fluorescence measurement by SYBR Green (Applied Biosystem) and the Pfaffl analysis method (Pfaffl M W, Nuc Acid Res 2001; 1: e45). In brief, in following this method the samples were studied three times; a total of 10 ng were used. For each gene both a positive and a negative control were used. The reaction was adjusted to an optimal temperature for each primer. The PCR was performed in a total volume of 25 uL and contained: forward and reverse primers, cDNA, 2×Fast SYBR Green (Applied Biosystem) and water. The obtained results were expressed in relative units (CI, Threshold cycle) in relation to the expression of ABL gene.

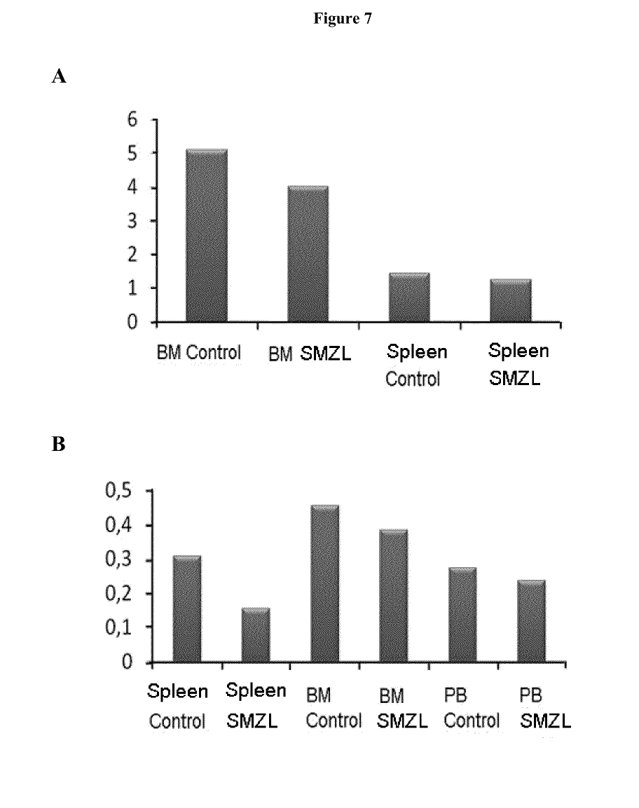

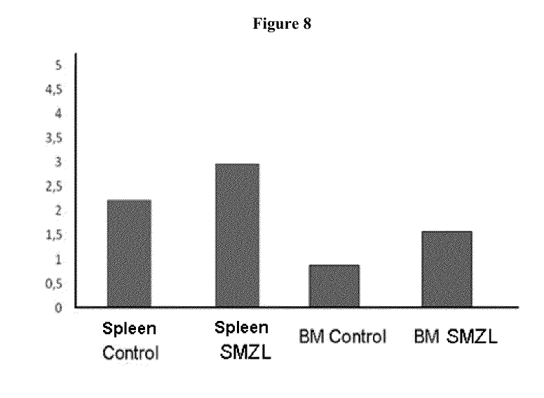

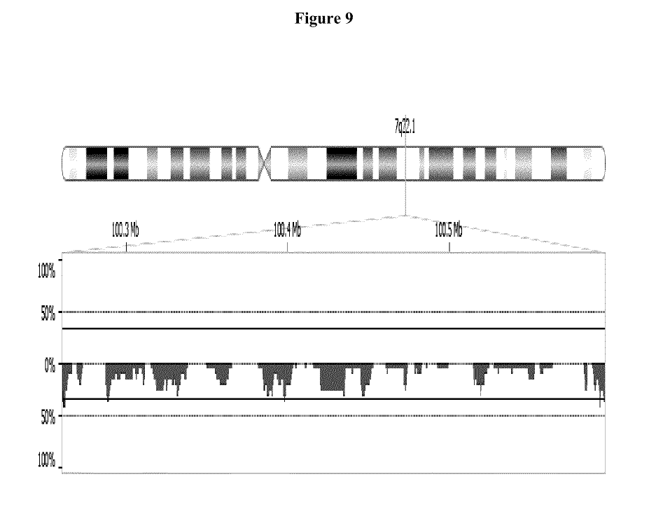

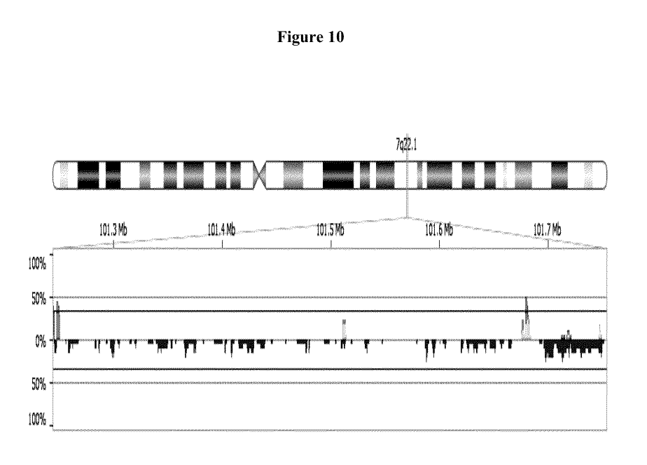

The results showed that the MUC3 expression is unmodified in SMZL patients. By contrast, the gene expression of both CUX1 and SH2B2 was different in SMZL and healthy controls. Thus CUX1 showed downregulation in spleen, bone marrow and peripheral blood of SMZL patients in relation to controls/FIG. 7). In addition, the SH2B2 gene was found overexpressed in spleen and bone marrow from SMZL patients in relation to controls.

In the next step a DNA sequencing of the candidate region was carried out. The aim was to look for the presence of possible DNA variations present in SMZL patients. A total of 23 DNA samples from SMZL patients and fourteen controls were analyzed. The target DNA (the DNA containing the genes CUX1 and MUC3) was amplified by PCR: several primers specific for the 7q22.1 region were designed according to the data from NCBI36HG18 (Table 5).

TABLE 5
Primers used for sequencing studies

| Gene | Location | Primer | Sequence |
|---|---|---|---|
| MUC3A/B | 100387324-100388418 | Forward | SEQ ID NO: 33 |
| | | Reverse | SEQ ID NO: 34 |
| | | Forward | SEQ ID NO: 35 |
| | | Reverse | SEQ ID NO: 36 |
| CUX-1 Exon 2 | 101346114-101346225 | Forward | SEQ ID NO: 37 |
| | | Reverse | SEQ ID NO: 38 |
| CUX-1 Exon 3 | 101458097-101458145 | Forward | SEQ ID NO: 39 |
| | | Reverse | SEQ ID NO: 40 |
| CUX-1 Exon 4 | 101500338-101500417 | Forward | SEQ ID NO: 41 |
| | | Reverse | SEQ ID NO: 42 |

After PCR the separation of the amplified products was performed in an agarose gel (1-2%) and a purification of each isolated fragment was carried out by the Kit High Pure Product Purification (Roche).

The sequencing of each fragment was performed by using the same primers used to amplify the DNA. All samples, both from controls and from SMZL patients, were sequenced from 40-60 ng of each fragment in a final volume of 8 uL. A sample containing both a sense and antisense primer at a concentration of 3 pmol in a volume of 2 uL was prepared. Therefore each isolated fragment was sequenced with each primer and several sequences of each fragment were obtained. The sequencing reaction was carried out in an ABI 3130XL sequencer (Applied Biosystems) in the Center for Cancer Research in Salamanca Spain. The generated sequences were analyzed by the software GENE15 (Discovery Studio Gene v1.5, Accelrys Inc). After the analysis the obtained sequences were aligned according to the consensus sequences (NCBI/HG18). The alignment and the analysis of the similar sequences were performed by the informatics tools Clustal W and BLAST (Basic Local Alignment Search Tool).

The sequence analysis of the 7q22.1 region containing the MUC3 gene (chromosome 7: 100387324 to 100388418) is a 1140 bp long. The results showed the presence of five polymorphisms in the region with the same frequence in controls than in SMZL patients. Therefore these polymorphisms could be present in the normal population and could be not related to the SMZL.

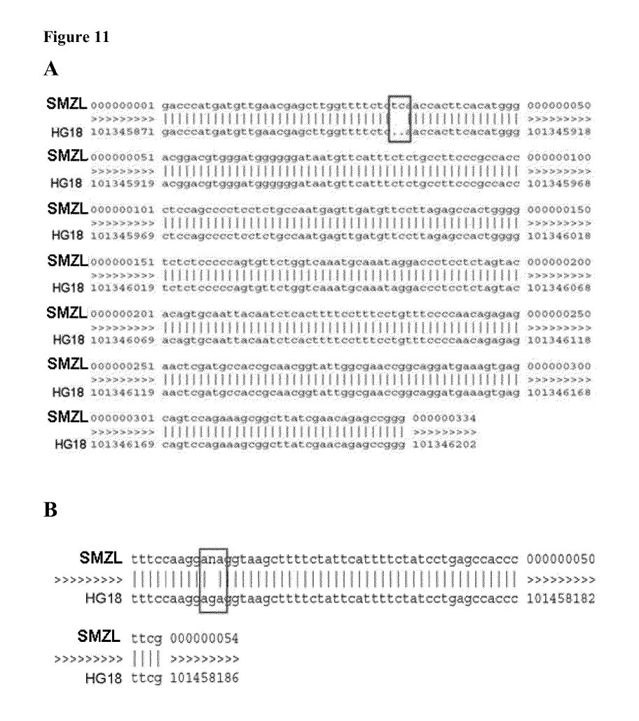

By contrast, in the sequence analysis of CUX1 gene, the presence of new variations not previously described were observed. The first variation was observed in the first intron and it was the insertion of one thymine and one adenine in the positions 101345904 and 101345905 of the reference genome (FIG. 11A). This variation is not present in the consensus sequence obtained from NCBI/HG18. Moreover in the third exon of this gene other variations not previously described were observed. Thus a mutation in the position 101458144 of the reference genome was observed in SMZL patients (FIG. 11B).

1. In vitro method for diagnosis of splenic marginal zone lymphoma (SMZL) by high-resolution genomic microarrays, comprising a step for detecting the deletion between the bases 99225039 to 101754718 located in the 7q22.1 region in which is located, at least, the CUX1 gene.

2. In vitro method, according to claim 1, characterized in that the deletion is located between the bases 99925039 to 101348479.

3. In vitro method, according to claim 1 characterized in that the deletion is located between the bases 100260234 to 100596921 and/or 101244026 to 101754718 in the 7q22.1 region.

4. In vitro method, according to any of the claim 1 or 3 characterized in that the CUX1 and SH2B2 genes are located in these deleted regions.

5. In vitro method, according to claims 1 to 4, characterized in that further in said region are located, among other, the genes EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17.

6. In vitro method, according to any of the claims 1 to 5, which additionally comprises the detection, by high-resolution genomic microarrays, of the deletion between the bases 102949624 to 1459599694 in the 7q22.2-q35 region in which is located, among others, the genes RELN, LHFPL3, PBEF1, THAP5, IMMP2L, FOXP2, TES, MET, ST7, PTPRZ1, GRMB, UBEH2, MLKN1, BPGM, CALD1, PTN, SVOPL, AGK, KEL, TPK1 and CNTNAP2.

7. In vitro method, according to any of the claims 1 to 6, characterized in that it is performed on a DNA sample selected from: a tissue sample from a lymphoma cell infiltrate, a bone marrow aspirate or a whole blood sample.

8. In vitro method, according to any of the claims 1 to 7, characterized in that high-resolution genomic microarrays are selected from BAC/PAC CGH-arrays, Agilent CGH-array, NimbleGen CGH-array or CGH Chromosome 7 Tiling Array from NimbleGen and CGH 7q22.1-q33 region Tiling Array from NimbleGen.

9. In vitro method for diagnosis of splenic marginal zone lymphoma (SMZL) which comprises measuring the expression level of at least the CUX1 gene, located in the 7q22.1 region between the bases 99925039 to 101754718.

10. In vitro method, according to claim 9, characterized in that it comprises measuring the expression level of the CUX1 gene located in the 7q22.1 region between the bases 99925039 to 101348479.

11. In vitro method, according to claim 10, characterized in that it comprises measuring the expression level of the CUX1 gene located in the 7q22.1 region between the bases 101244026 to 101754718.

12. In vitro method, according to claim 9, characterized in that it comprises measuring the expression level of the, at least, the CUX1 and SH2B2 genes located in the 7q22.1 region between the bases 101244026 to 101754718.

13. In vitro method, according to claims 1 to 12, characterized in that it further comprises measuring the expression level of at least one of the following genes selected from: EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17, or combinations thereof, in the 7q22.1 region between the bases 99925039 to 101348479.

14. In vitro method, according to any of the claims 9 to 13 characterized in that it is performed on a DNA sample selected from: a tissue sample from a lymphoma cell infiltrate, a bone marrow aspirate or a whole blood sample.

15. In vitro method for diagnosis of splenic marginal zone lymphoma (SMZL) which comprises a combination, in any order, of a first step for detecting, by high-resolution genomic microarrays, the deletion between the bases 99225039 to 101754718 located in the 7q22.1 region and a second step for measuring the expression level, of at least, the CUX1 gene.

16. In vitro method, according to claim 15, characterized in that the deletion is located between the bases 99925039 to 101348479.

17. In vitro method, according to claim 15 characterized in that the deletion is located between the bases 100260234 to 100596921 and/or 101244026 to 101754718 in the 7q22.1 region.

18. In vitro method, according to any of the claim 15 or 17 characterized in that in the second step the expression levels of the CUX1 and SH2B2 genes are measured.

19. In vitro method, according to claims 15 to 18, characterized in that further comprises measuring the expression levels of at least one of the following genes selected from: EPHB4, SLC12A9, TRIP6, SRRT, ACHE, UFSP1, TRIM56, SERPINE1, APIS1, VGF, TSC22D4, HRBL, LRCH4, MUC3, MUC12 and MUC17 and/or combinations thereof.

20. In vitro method, according to any of the claim 15 or 19, which additionally comprises the detection, by high-resolution genomic microarrays, of the deletion between the bases 102949624 to 1459599694 in the 7q22.2-q35 region in which is located, among others, the genes RELN, LHFPL3, PBEF1, THAP5, IMMP2L, FOXP2, TES, MET, ST7, PTPRZ1, GRMB, UBEH2, MLKN1, BPGM, CALD1, PTN, SVOPL, AGK, KEL, TPK1 and CNTNAP2.

21. Kit for the in vitro diagnosis of splenic marginal zone lymphoma (SMZL) comprising a set of probes selected form the group SEQ ID No. 1, SEQ ID No. 2, SEQ ID No. 3, and/or any probe that hybridizes wholly or partially with the deleted region between the bases 99925039 to 101754718 by high-density genomic microarrays.

22. Kit according to claim 21 characterized in that the selected set of probes hybridize wholly or partially with the deleted region between the bases 99925039 to 101348479.

23. Kit according to claim 21 characterized in that the selected set of probes hybridize wholly or partially with the deleted regions between the bases 100260234 to 100596921 and/or 101244026 to 101754718 of the region 7q22.1.

24. Kit according to claims 21 to 23 characterized in that it further comprises at least a probe that hybridizes wholly or partially with the deleted region between the bases 102949624 to 1459599694 in the 7q22.2-q35 region, by high-density genomic arrays, preferably the probe is SEQ ID No. 4.

25. Kit according to claim 24 characterized in that the probe hybridizes in the 7q33 region.

26. Kit for the in vitro diagnosis of splenic marginal zone lymphoma (SMZL) characterized in the detecting the expression level of at least the CUX1 gene, located in the 7q22.1 region between the bases 99925039 to 101754718 by RT-PCR with the primers defined as SEQ ID No 5 and SEQ ID No. 6.

27. Kit according to claim 26 characterized in that the expression level of at least the CUX1 gene, by RT-PCR with the primers defined as SEQ ID No 5 and SEQ ID No. 6 it is performed in the deleted region between the bases 99925039 to 101348479.

28. Kit according to claim 26 characterized in that the expression level of at least the CUX1 gene, by RT-PCR with the primers defined as SEQ ID No 5 and SEQ ID No. 6 it is performed in the deleted region between the bases 100260234 to 100596921 and/or 101244026 to 101754718 of the region7q22.1.

29. Kit according to claim 26 characterized in that the expression level of the CUX1 gene it is further performed with the primers defined as SEQ ID Nos. 23-24.

30. Kit according to any of the claims 26 to 29 which additionally comprises the detection of the expression level of SH2B2 gene by the primers defined as SEQ ID Nos 25-28.

31. Kit according to any of the claims 26 to 30 characterized in that further comprises detecting the expression levels of at least one of the following genes selected from: EPHB4, SLC12A9, TRIPE, SRRT, ACHE, UFSP1, TRIM56, SERPINE1, APIS1, VGF, TSC22D4, HRBL, LRCH4, MUC3, MUC12 and MUC17 and/or combinations thereof by specific primers for the sequences of these genes.
